(19) United States
(12) Patent Application Publication
(10) Pub. No.: US 2024/0294465 A1
(43) Pub. Date: Sep. 5, 2024

(54) NOVEL ANTI-FIBROTIC DRUGS

(71) Applicant: HELMHOLTZ ZENTRUM MUNCHEN - DEUTSCHES FORSCHUNGSZENTRUM FUR GESUNDHEIT UND UMWELT, Neuherberg (DE)

(72) Inventors: Gerald BURGSTALLER, Munich (DE); Michael GERCKENS, Munich (DE); Oliver PLETTENBURG, Hannover (DE); Ali YILDIRIM, Munich (DE); Ma HUILONG, Hannover (DE)

(73) Assignee: HELMHOLTZ ZENTRUM MÜNCHEN - DEUTSCHES FORSCHUNGSZENTRUM FÜR GESUNDHEIT UND UMWELT, Neuherberg (DE)

(21) Appl. No.: 18/567,733

(22) PCT Filed: Jun. 9, 2022
(86) PCT No.: PCT/EP2022/065773

§ 371 Date: Dec. 6, 2023

(30) Foreign Application Priority Data
Jun. 9, 2021 (EP) 21178481.4

(51) Int. Cl. C07C 235/38; A61K 31/167; A61K 31/245; A61K 31/343; A61K 31/353; A61K 31/357; A61K 31/36; A61K 31/37; A61K 31/396; A61K 31/404; A61K 31/415; A61K 31/4184; A61K 31/4192; A61K 31/42; A61K 31/4245; A61K 31/44; A61K 31/4402; C07C 317/32; C07C 325/02; C07D 209/08; C07D 213/643; C07D 213/75; C07D 229/02; C07D 231/20; C07D 235/08; C07D 249/04; C07D 271/12; C07D 307/81; C07D 307/87; C07D 311/20; C07D 311/58; C07D 317/60; C07D 319/18; G01N 33/50; G01N 33/58

(52) U.S. Cl. CPC C07C 235/38; A61K 31/167; A61K 31/245; A61K 31/343; A61K 31/353; A61K 31/357; A61K 31/36; A61K 31/37; A61K 31/396; A61K 31/404; A61K 31/415; A61K 31/4184; A61K 31/4192; A61K 31/42; A61K 31/4245; A61K 31/44; A61K 31/4402; C07C 317/32; C07C 325/02; C07D 209/08; C07D 213/643; C07D 213/75; C07D 229/02; C07D 231/20; C07D 235/08; C07D 249/04; C07D 271/12; C07D 307/81; C07D 307/87; C07D 311/20; C07D 311/58; C07D 317/60; C07D 319/18; G01N 33/5044; G01N 33/582; C07C 2601/02

(57) ABSTRACT

The present invention relates to new cinnamic add amides which may be used for treatment of fibrosis and neoplasia and to cinnamic acid amides for use in the treatment of fibrosis, neoplasia, arthrolithiasis, familiar mediterranean fever and pericarditis. Further, the invention relates to a pharmaceutical composition comprising said cinnamic acid amides and to a screening essay for identifying compounds suitable for the treatment of fibrosis.

### TECHNICAL FIELD OF THE INVENTION

The present invention relates to new cinnamic acid amides which may be used for treatment of fibrosis and neoplasia and to cinnamic acid amides for use in the treatment of fibrosis, neoplasia, arthrolithiasis, familiar mediterranean fever and pericarditis. Further, the invention relates to a pharmaceutical composition comprising said cinnamic acid amides and to a screening essay for identifying compounds suitable for the treatment of fibrosis.

### BACKGROUND ART

Fibrotic diseases affect nearly every tissue in the body, account for over 45% of all deaths in the industrialized world, and progressive forms of the disease rapidly lead to organ dysfunction, organ failure and ultimately death (1-3). Due to its ubiquitous existence and high mortality, fibrosis, or “scarring”, has become a high medical need for novel drug discovery strategies (3, 4). However, effective antifibrotic therapeutics are missing in the clinics. The lack of antifibrotic therapies and its concomitant high medical need is best exemplified by idiopathic pulmonary fibrosis (IPF), which is a rapidly progressive and fatal fibrotic disorder. Patients with this common form of interstitial fibrotic lung disease face a median survival time of 3-5 years (5-7). Currently, only two approved anti-fibrotic drugs for IPF are on the market, Pirfenidone and Nintedanib, however, both substances partially slow down the rate in lung function decline but do not stop disease progression (8-10). Therefore, new therapeutic strategies and approaches are urgently required. In fibrotic pathogenesis repetitive and constant injury leads to a sustained and self-perpetuating activation of fibroblasts, leading to their transdifferentiation into synthetic and highly contractile α-smoothmuscle-actin (αSMA)-expressing myofibroblasts, that massively deposit extracellular matrix (ECM), which stiffens the lung and destroys normal lung architecture (3, 6, 11, 12). The matrisome of fibrotic ECM was shown to harbor a disease- and progression specific signature of fibrillar collagens (types I, Ill, and V), proteoglycans, fibronectin, glycosaminoglycans, matrix-Gla protein, and microfibrillar-associated proteins (11, 13-16).

Of all pro-fibrotic signals reported, multifunctional TGFP1 is the most intensively studied and central player in various fibrotic diseases capable of triggering transdifferentiation of fibroblasts into myofibroblasts (17-21). TGFP1 binds to its TGFβ1-receptor and downstream signaling occurs by post translational modifications of cytoplasmic members of the SMAD family, which act as transcription factors in the cell nucleus, regulating the expression of common profibrotic genes, including ECM proteins (22-25). Plasminogen activator inhibitor-1 (PAI-1) is an essential downstream target of the TGFβ1 pathway, suppresses the fibrinolytic system and is considered as a therapeutic target option for fibrosis (26). Additionally, in IPF profibrotic IL8 was recently found to be secreted by a special fibrogenic mesenchymal progenitor cell population with autocrine effects on proliferation and motility, as well as paracrine effects on macrophage recruitment (27).

Tranilast is known as a mast cell degranulation inhibitor developed by Kissei Pharmaceuticals and was already approved 1982 in Japan and South Korea for the treatment of bronchial asthma, keloid and hypertrophic scars. The drug appears to work by inhibiting the release of histamine from mast cells but its molecular target(s) remain unknown. Even though the antifibrotic properties of Tranilast have also been reported in the prior art, its potency is very low (IC₅₀˜150 μM) and would require high-dose administration in humans, which reportedly causes liver toxicity. So far, medicinal chemistry optimization efforts failed to significantly improve the antifibrotic activity of Tranilast (28).

Thus, there is a general need for antifibrotic drugs and assays for identification of suitable antifibrotic drugs. Tranilast may be a suitable lead compound for further medicinal chemistry optimization.

### SUMMARY OF THE INVENTION

The Invention is directed to a compound for use in the treatment of fibrosis and neoplasia, preferably a fibrosis or neoplasia located in the heart, the lung, the renal tract, the liver, in the skin, in the pleura and retroperitoneum, more preferably the fibrosis is selected from pleural fibrosis, retroperitoneal fibrosis, atrial fibrillation, myocardial interstitial fibrosis, idiopathic pulmonary fibrosis (IPF), interstitial lung diseases, chronic kidney disease, non-alcoholic fat liver disease, skin scars, keloids, tumor-associated desmoplastic reaction wherein said compound is a compound according to formula (I)

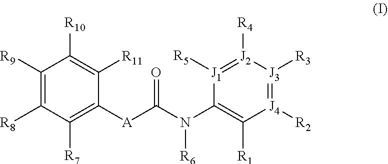

wherein,
R¹ is selected from the group consisting of —OR¹², —O(CH₂)ᵤ(C₃-C₁₀)aryl, —O(CH₂)ᵤ(C₃-C₁₀)cycloalkyl, —O(CH₂)ᵤ(C₂)alkynyl; —(CH₂)ᵤ(C₃-C₁₀)aryl, —O(CH₂)ᵤ(C₃-C₁₀)cycloalkyl, —(CH₂)ᵤ(C₃-C₁₀)cycloalkyl,

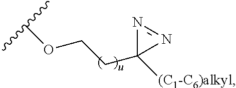

—(CH₂)ᵤ(C₂)alkynyl;
u is 0 to 6;
R² to R⁵ are independently selected from the group consisting of H, —OR¹², —(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —CHZ₂, —CZ₃—CH₂Z, —OCHZ₂, —OCZ₃, —OCH₂Z—N(R¹³)(R¹⁴), —N(R¹⁵)(OR¹⁶), —S(O)₀₋₂R¹⁷, —S(O)₀₋₂OR¹⁸, —OS(O)₁₋₂R¹⁹, —OS(O)₁₋₂OR²⁰, —S(O)₁₋₂N(R²¹)(R²²), —OS(O)₁₋₂N(R²³)(R²⁴), —N(R²⁵)S(O)₁₋₂R²⁶, —NR²⁷S(O)₁₋₂OR²⁸, —NR²⁹S(O)₂N(R³⁰)(R³¹), —C(═X)R³², —C(═X)XR³³, —XC(═X)R³⁴, and —XC(═X)XR³⁵, —OR³⁶, —O(CH₂)ᵥ(C₃-C₁₀)aryl, —O(CH₂)ᵥ(C₃-C₁₀)cycloalkyl, —O(CH₂)ᵥ(C₂)alkynyl;
R⁶ is selected from the group consisting of H, —(C₁-C₁₀)alkyl, benzyl and —(CH₂)₁₋₅(C₃-C₁₀)cycloalkyl; wherein —(C₁-C₁₀)alkyl, benzyl and —(CH₂)₁₋₅(C₃-C₁₀)cycloalkyl optionally are further substituted with at least one substituent selected from the group consisting of Halogen, preferably F;
R⁷ to R¹¹ are independently selected from the group consisting of H, —OR¹², —SR¹², —(C₁-C₁₀)alkyl, halogen, —(C₁-C₁₀)alkylO(C₁-C₁₀)alkyl, cyano, isocyano, cyanato, isocyanato, thiocyanato, isothiocyanato, azido, —(C₂-C₁₀)alkenyl, —(C₂-C₁₀)alkynyl, —O(C₂-C₁₀)alkynyl, —(C₃-C₁₀)cycloalkyl, —(C₃-C₁)heterocyclyl, —(C₃-C₁₀)aryl, —(C₃-C₁₀)heteroaryl, —(CH₂)ᵥCHZ₂, —CZ₃—CH₂Z, —OCHZ₂, —OCZ₃, —OCH₂Z, —N(R¹³)(R¹⁴), —N(R¹⁵)(OR¹⁶), —S(O)₀₋₂R¹⁷, —S(O)₁₋₂OR¹⁸, —OS(O)₁₋₂R¹⁹, —OS(O)₁₋₂OR²⁰, —S(O)₁₋₂N(R²¹)(R²²), —OS(O)₁₋₂N(R²³)(R²⁴), —N(R²⁵)S(O)₁₂R²⁶, —NR²⁷S(O)₁₂OR²⁸, —NR²⁹S(O)₁₋₂N(R³⁰)(R³¹), —C(═X)R³², —C(═X)XR³³, —XC(═X)R³⁴, and —XC(═X)XR³⁵, —O(CH₂)ᵥ(C₃-C₁₀)cycloakyl, —O(CH₂)ᵥ(C₁-C₁₀)alkyl and —O(CH₂)ᵥ(C₃-C₁₀)aryl,
wherein two adjacent rests of R¹ to R⁵ and R⁷ to R¹¹ optionally may form a ring attached to the underlying aromatic ring of formula (I) according to formula (III) to (XI)

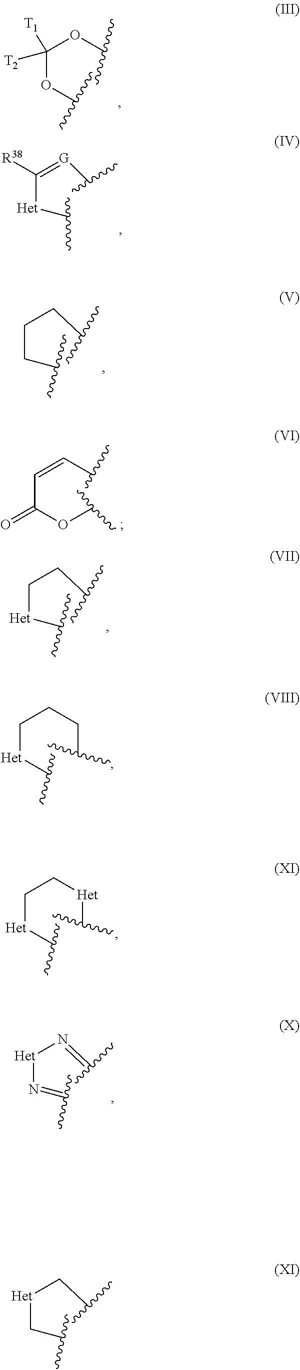

wherein T¹ and T² are independently selected from the group consisting of H, —(C₁-C₁₀)alkyl and halogen;
wherein each hydrogen in formula (III) to (IX) is optionally substituted with halogen, or —(C₃-C₁₀)aryl, —(C₁-C₃)alkyl, preferably F;
Het is selected from O, S, NH, N(C₁-C₁₀)alkyl;
G is selected from CH, N,
J₁ to J₄ are independently selected from C or N, preferably J₁ to J₄ are C;
wherein if any one of J, to J₄ is N, the corresponding R¹ to R⁴ attached to the respective J₁ to J₄ which is (are) N is absent;
R¹² to R³⁶ are independently selected from the group consisting of H, —(C₁-C₁₀)alkyl, —(C₂-C₁₀)alkenyl, —(C₂-C₁a)alkynyl, —(C₃-C₁₀)cycloalkyl, —(C₃-C₁₀)heterocyclyl, —(C₃-C₁₀)aryl, —(C₃-C₁₀)heteroaryl;
R³⁸ is independently selected from the group consisting of H, —(C₁-C₁₀)alkyl;
R¹ to R¹¹, independently selected from the group consisting of —(C₁-C₁₀)alkyl, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —O(CH₂)ᵥ(C₃-C₁₀)cycloalkyl, —O(CH₂)ᵥ(C₁-C₁₀)alkyl and —O(CH₂)ᵥ(C₃-C₁₀)aryl and R¹² to R³⁵ optionally are further substituted with at least one substituent selected from the group consisting of OR¹², —(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —CHZ₂, —CZ₃—CH₂Z, —OCHZ₂, —OCZ₃, —OCH₂Z, —N(R¹³)(R¹⁴), —N(R¹⁵)(OR¹⁶), —NH C(O)(C₁-C₁₀)alkyl, —S(O)₀₋₂R¹⁷, —S(O)₁₋₂OR¹⁸, —OS(O)₁₋₂R¹⁹, —OS(O)₁₋₂OR²⁰, —S(O)₁₋₂N(R²¹)(R²²), —OS(O)₁₂N(R²³)(R²⁴), —N(R²⁵)S(O)₁₋₂R²⁶, —NR²⁷S(O)₁₋₂OR²⁸, —NR²⁹S(O)₂N(R³⁰)(R³¹), —C(═X)R³², —C(═X)XR³³, —XC(═X)R³⁴, and —XC(═X)XR³⁵, —OR³⁶, and —O(CH₂)ᵥ(C₃-C₁₀)aryl;
v is 0 to 5;
Z is halogen;

X is selected from the group consisting of O, —NH— or S;
A is selected from the group consisting of

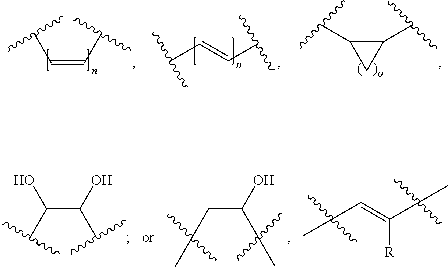

n is 1, 2, or 3, preferably 1;
o is 1, 2, or 3, preferably 1;
R is H, (C₁-C₆)alkyl, cyano, —(C₃-C₁₀)cycloalkyl, benzyl or part of a ring wherein R is connected with R⁷ or R¹¹ by

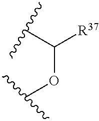

preferably H or benzyl, most preferably H;
R³⁷ is H or —CF₃;
with the provision that if n is 2 or 3, A may be

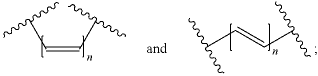

with the proviso that R⁵ is not —COOH.

The invention is further related to a compound according to formula (II)

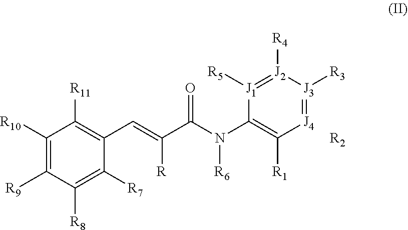

wherein,
R¹ is —OR¹², —O(C₅-C₁₀)heteroaryl, —O(C₃-C₁₀)aryl, —O(CH₂)ᵤ(C₃-C₁₀)cycloalkyl,

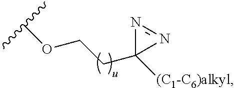

—O(CH₂)ᵤ(C₃-C₁₀)aryl,
R² to R⁵ and R⁷, and R¹¹ are independently selected from the group consisting of H, —(C₁-C₁₀)alkyl, halogen, azido, cyano, —O(C₁-C₁₀)alkyl, —(CH₂)ᵤ(C₃-C₁₀)aryl, —(CH₂)ᵤ(C₃-C₁₀)cycloalkyl, —(C₂-C₁₀)alkenyl, —(C₂-C₁₀)alkynyl, —(C₃-C₁₀)cycloalkyl, —(C₃-C₁₀)aryl, which optionally are further substituted with at least one substituent selected from the group consisting of Halogen, —OH, —NH₂, —NHC(O)CH₃, —CN, —N₃, and —COOH, —C(O)NH₂;
R⁶ is H, —(C₁-C₁₀)alkyl, benzyl and —(CH₂)₁₋₅(C₃-C₁₀)cycloalkyl; wherein —(C₁-C₁₀)alkyl, benzyl and —(CH₂)₁₋₅(C₃-C₁₀)cycloalkyl optionally are further substituted with at least one substituent selected from the group consisting of Halogen, preferably F;
R⁸, R⁹ and are H, —O(C₁-C₁₀)alkyl, —SR¹², —O(CH₂)ᵤ(C₃-C₁₀)aryl, —O(CH₂)ᵤ(C₃-C₁₀)cycloalkyl, —O(C₃-C₁₀)cycloalkyl, or, —O(C₂-C₁₀)alkenyl;
R¹⁰ is H, halogen, —O(C₁-C₁₀)alkyl, —O(CH₂)ᵤ(C₃-C₁₀)aryl, —O(CH₂)ᵤ(C₃-C₁₀)cycloalkyl, —O(C₃-C₁₀)cycloalkyl, or, —O(C₂-C₁₀)alkenyl;
with the proviso that if R₉=H either R₈ or R₁₀ is —OR¹²
u is 0 to 6;
R is H, (C₁-C₆)alkyl, cyano, —(C₃-C₁₀)cycloalkyl, benzyl or part of a ring wherein R is connected with R⁷ or R¹¹ by

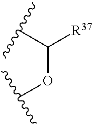

preferably H or benzyl, most preferably H;
R³⁷ is H or —CF₃;
R¹² are independently selected from the group consisting of H, —(C₁-C₁₀)alkyl, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, (CH₂)ᵤ(C₃-C₁₀)aryl, —(CH₂)ᵤ(C₃-C₁₀)heteroaryl —(CH₂)ᵤ(C₃-C₁₀)cycloalkyl; preferably —(C₁-C₁₀)alkyl, more preferably —(C₁-C₄)alkyl;
wherein two adjacent rests of R⁸ to R¹⁰ optionally may form a ring, attached to the underlying aromatic ring of formula (II) according to

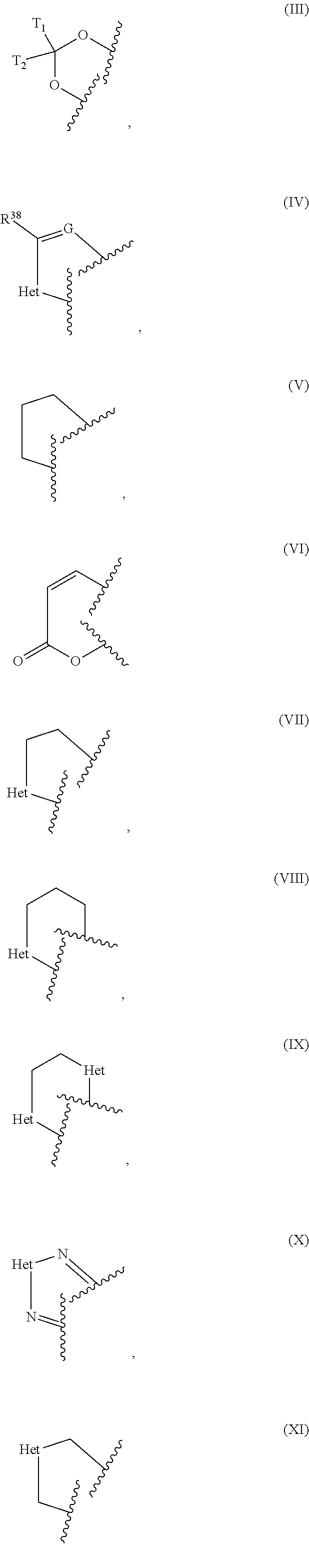

wherein T¹ and T² are independently selected from the group consisting of H, —(C₁-C₁₀)alkyl and halogen;
wherein each hydrogen in formula (III) to (XI) is optionally substituted with halogen, —(C₃-C₁₀)aryl, or —(C₁-C₃)alkyl, preferably F;
Het is O, S, N(C₁-C₁₀)alkyl or NH; preferably O;
R³⁸ is independently selected from the group consisting of H, —(C₁-C₁₀)alkyl;
G is selected from CH, N;
J₁ to J₄ are independently selected from C or N, preferably J₁ to J₄ are C;
wherein if any one of J₁ to J₄ is N, the corresponding R¹ to R⁴ attached to the respective J₁ to J₄ which is (are) N is absent;
with the proviso that
if J₁ to J₄ are C and
I) if R⁵ is —(CH₂)₃CH₃; R⁹ is —OCH₃, —OCH₂CH₃, —O(CH₂)₂CH₃, —OCH₂phenyl or —O(CH₂)₃CH₃; R¹, R², R³, R⁴, R, R¹¹ and R are H; and
a) R⁸ and R¹⁰ are H; or
b) R⁸ is —OCH₃, or —OCH₂CH₃ and R¹⁴ is H; or
c) R¹⁰ is —OCH₃, or —OCH₂CH₃ and R⁸ is of H;
then R⁶ is not H;
II) if R⁹ is —OCH₃, —O(CH₂)₂CH₃, —O(2-propyl), —O(CH₂)₄CH₃, —O(CH₂)₅CH₃, —OCH₂(4-chiorophenyl), —O(CH₂)₂CH(CH₃)₂, —OCH₂(2,6-dichlorophenyl), or —OCH₂phenyl; R¹, R², R³, R⁴, R⁷, R¹¹, and R are H; R⁸ is —OCH₃ and R¹⁰ is H, or R¹⁰ is —OCH₃ and R⁸ is H; R⁵ is —(CH₂)₃CH₃; then R⁶ is not H;
III) if R⁹ is —OCH₃; R⁸ is Brand R¹⁰ is H, or R¹⁰ is Br and R⁸ is H; R⁵ is —(CH₂)₃CH₃; R¹, R², R³, R⁴, R¹¹ and R are H, then R¹ is not H;
IV) if R⁵ is —(CH₂)₃CH₃, R⁹ is —OCH₃, R⁷ is —OCH₃ and R¹¹ is H or R¹¹ is —OCH₃ and R⁷ is H; R¹, R², R³, R⁴, R⁸, R¹⁰, R¹¹ and R are H; then R⁶ is not H;
V) if R⁹ is —OCH₃, —OCH₂phenyl, or —OCH₂(2-fluorophenyl); R⁸ is Brand R¹⁰ is —OCH₃, or R¹⁰ is Br and R³ is —OCH₃; R⁵ is —O(CH₂)₃CH₃; R¹, R², R³, R⁴, R⁷, R¹¹, and R are H, then R⁶ is not H;
VI) if R⁹ is —O(CH₂)₃CH₃; R⁸ is —OCH₂CH₃ and R¹⁰ is H, or R¹⁰ is —OCH₂CH₃ and R⁸ is H; R⁵ is —O(CH₂)₃CH₃; R¹, R², R³, R⁴, R⁷, R¹¹, and R are H, then R⁶ is not H;
VII) if R⁹ is —OCH₂(2-chlorophenyl); R⁸ is Br and R¹⁰ is —CH₂CH₃, or R¹⁰ is Br and R⁸ is —OCH₂CH₃; R⁵ is —(CH₂)₃CH₃; R¹, R², R³, R⁴, R⁷, R¹¹, and R, are H, then R⁶ is not H;
VIII) if R⁹ is —O(2-octenyl); R⁸ is Cl and R¹⁰ is H, or R¹⁰ is Cl and R⁸ is H; R⁵ is —(CH₂)₃COOH; R¹, R², R³, R⁴, R⁷, R¹¹, and R are H, then is not H;
IX) if R⁹ is —OCH₃; R¹, R², R³, R⁴, R⁷, R⁸, R¹⁰, R¹¹, R are H; and
R⁵ is -(2-fluorophenyl), -phenyl; then R⁶ is not H;
X) if R⁹ is —OCH₂CH₃; R⁵ is —(CH₂)₃CH₃; R¹, R², R³, R⁴, R⁷, R⁸, R¹⁰, R¹¹, and R are H; then R⁶ is not H;
XI) if R⁹ is —OCH₃; R³ is —OCH₃ and R¹⁰ is H, or R¹⁰ is —OCH₃ and R⁸ is H; R⁵ is —(CH₂)₃CH₃; R¹, R², R³, R⁴, R⁸, R⁷, and R are H; then R⁶ is not H;
XII) if R⁹ is —OCH₃; R⁵ is —O(CH₂)₃CH₃; R¹, R², R³, R₄, R⁷, R¹¹, and R are H, and R⁸ is —OCH₂CH₃ and R¹⁰ is H, or R¹⁰ is —OCH₂CH₃ and R⁸ is H; then R⁶ is not H;
XIII) if R⁵ is —(CH₂)₃CH₃; R⁹ is —O(CH₂)₃CH₃, or —OCH₃; R¹, R², R³, R⁴, R⁷, R⁸, R¹⁰, R¹¹, and R are H; then R⁶ is not H;
XIV) if R⁵ is —CH₃, —(CH₂)₂CH₃ or —CH₂CH₃; R⁹ is —OCH₃; R⁸ is —OCH₃ and R¹⁰ is H or R⁸ is H and R¹⁰ is —OCH₃; R¹, R², R³, R⁴, R⁷, R⁸, R⁹, R¹⁰ and R¹¹ are H; then R⁶ is not H.
XV) if R⁵ is —(CH₂)₃CH₃; R¹, R², R³, R⁴, R⁷, R⁸, R⁹, R¹⁰ and R¹¹ are H; then R⁶ is not H.

In a further embodiment, the invention is related to a pharmaceutical composition comprising the compounds as defined above.

In a further embodiment, the invention is directed to the compounds as defined above and the pharmaceutical composition for use in medicine, in particular for use in the treatment of fibrosis and neoplasia, preferably a fibrosis or neoplasia located in the heart, the lung, the renal tract, the liver, in the skin, in the pleura and retroperitoneum, more preferably the fibrosis is selected from pleural fibrosis, retroperitoneal fibrosis, atrial fibrillation, myocardial interstitial fibrosis, idiopathic pulmonary fibrosis (IPF), interstitial lung diseases, chronic kidney disease, non-alcoholic fat liver disease, skin scars, keloids, tumour-associated desmoplastic reaction.

In a further embodiment, the invention is directed to the compounds as defined above and the pharmaceutical composition for use in medicine, in particular for use in the treatment of inflammatory diseases, such as arthrolithiasis, familiar mediterranean fever and pericarditis.

Moreover, the invention is directed to a screening assay, comprising the steps
a) culturing adherent cells which deposit at least one protein in the presence of at least one test compound;
b) staining of at least one protein deposited by the adherent cells;
c) fixation of adherent cells and the at least one protein;
d) microscopic detection of a signal of the at least one stained deposited protein;
e) data analysis of signals detected in step d) comprising quantification of the amount of the at least one protein deposited in the presence of the at least one test compound;
wherein step b) is carried out before step c).

In dose-response relationship studies, applying the assay of the present invention, the inventive compounds proved to be >100 fold more potent compared to Tranilast in inhibiting ECM deposition (see FIG. 14A-B). It has been found that the compounds of the present invention displayed a dynamic inhibition of ECM deposition without showing cell death (see FIG. 14E). In difference to Tranilast, it has been found that upon exposure to the compounds of the present invention, to patient-derived primary human lung fibroblasts (phLFs), cell-morphology switched from elongated to round cells, including an extensive rearrangement of the actin cytoskeleton (see FIG. 14F). These substantial morphology changes, observed with the compounds of the present invention but not with Tranilast, strongly indicate an exclusive mode of action (see FIG. 12H).

For the inventive assay, it has been found that immunostaining according to step b) prior to fixation according to step c) identifies exclusively the extracellular deposited proteins, and does not lead to “false-positive” hits due to staining of intracellular ECM precursor proteins. The assay can be used for the quantification of deposited ECM of any adherent cells that produce ECM (primary patient derived, primary animal derived, human cell lines, animal cell lines), derived from any organ (healthy or diseased) or from various animal species and/or animal disease model. In one embodiment patient derived human primary cells (for example lung fibroblasts from IPF patients) are used. This generates efficacy and potency data with the highest clinical relevance possible in vitro, especially when compared to assays that use immortalized cell lines or cells from different animal species.

### BRIEF DESCRIPTION OF THE FIGURES

FIG. 1: Live labeling of phLFs ensures exclusively extracellular fluorescent staining of ECM proteins. (A) Untreated phLFs were either PFA-fixed, PFA-fixed or Triton-permeabilized, or stained alive. Only live staining without fixation resulted in an exclusive extracellular staining (white arrows) without cytoplasmic signals. Scale bar=100 μm (B) Western blot analysis of medium supernatants of phLFs that were treated with or without BrefeldinA demonstrating that treatment with BrefeldinA commonly inhibited the secretion of the ECM proteins collagen I and fibronectin. (C) Confocal fluorescent microscopy of BrefeldinA treated phLFs that were either PFA-fixed, PFA-fixed or Triton-permeabilized, or stained alive. Immunostaining of collagen I (red) demonstrated that PFA-fixation with and without Tritonpermeabilization leads to a positive labeling of intracellular collagen I (white arrowheads), whereas staining of living phLFs exclusively labels extracellular collagen I only (white arrows). Phalloidin (green) stains intracellular actin-stress fibers and nuclei were stained with Hoechst (blue). Scale bar=100 μm.

FIG. 2: Protein analysis of intracellular and secreted ECM proteins in the human 3D fibrosis model. (A) Treatment of phLFs with TGFβ1 led to a significant increase in soluble-intracellular as well as secreted collagen I proteins. (B) Treatment of phLFs with TGFβ1 led to a significant increase in soluble-intracellular as well secreted collagen V proteins. (C) Concomitant treatment of TGFβ1 together with the prolyl-4-hydroxylase inhibitor Ethyl-3,4-dihydroxy-benzoate (EDHB) resulted in a statistically significant suppression of the expression of intracellular collagen I, collagen V, and surprisingly also the non-collagen fibulin 1. (D) Concomitant treatment of TGFβ1 together with EDHB resulted in significant suppression of secreted collagen I. ns=not significant. * p<0.05, ** p<0.01 and *** p<0.001. Statistics: Oneway ANOVA with Bonferroni-correction. All quantitative data represent means±SEM. 3 (three different patient phLFs).

FIG. 3: Image analysis by FANTAIL applying a deep convolutional neuronal network (CNN), CNN training and hyperparameter optimization. (A) For data augmentation in the training set each original image was fragmented in np×np sized tiles with % overlap and saved in 0°, 90°, 180°, and 270° rotated orientation. (B) For original image classification each image was fragmented in non-overlapping np×np sized tiles. Each tile was classified separately as “hits” or “others” (see also FIG. 3A). (C) Learning curves of the deep CNN (FIG. 10A) of training accuracy in dependency of np. (D) Learning curves of the deep CNN (FIG. 10A) of validation accuracy in dependency of np. (E) Receiver operator characteristic analysis as performance measurement for classification problem based on the share of tiles classified as hits using deep CNNs as specified in FIG. 10A in dependency of np and number of training iterations (=epochs). (F) Image clustering is achieved by flattening the image pixel matrices into linear vectors that is projected into a bi-dimensional space by using UMAP.

FIG. 4: Cytotoxicity of Tranilast in phLFs and its dose-response relationship in inhibiting ECM deposition. (A) MTT assay exhibiting cellular viability in phLFs+TGFβ1 as well as phLFs which both were treated with various concentrations of Tranilast (75 μM, 150 μM, 300 μM). Cell-death was mimicked by treating the phLFs with 10% EtOH. (B) Protein analysis by Western blotting demonstrating a significant dose-dependent reduction of cytosolic αSMA in phLFs+TGFβ1+Tranilast. (C) Protein analysis by Western blotting demonstrating a significant dose-dependent reduction of soluble fibulin 1 in phLFs+TGFβ1+Tranilast. (D) Protein analysis by Western blotting exhibiting a significant dose-dependent reduction of soluble collagen I and collagen V in phLFs+TGFβ1+Tranilast. (E) Analysis of gene expression by qPCR showing a significant dose-dependent down-regulation of αSMA and collagen I, but surprisingly not of fibulin 1 and collagen V transcripts. ns=not significant. * p<0.05 and ** p<0.01. Statistics: unpaired t test with Bonferroni's multiple comparison test. All quantitative data represent means±SEM. n=3 (three different patient phLFs).

FIG. 5: UMAP regulation pattern clustering (UMAP-RPC) of transcriptomic data and network analysis. (A-C) The twenty highest and lowest deregulated genes of phLFs+TGFβ1 (A), phLFs+TGFβ1+example 84 (B) and phLFs+example 84 (C), all compared to untreated phLFs controls, are displayed as a heatmaps of three different patient samples (n=3). (D) Overlapping 279 genes which were counter-regulated in phLFs+TGFβ1+example 84 compared to phLFs+TGFβ1 (>2 fold, FDR<10%) (E) Illustration of UMAP-RCP based on different transcript abundances (“a,b,c,d”) for various conditions (phLFs, phLFs+TGFβ1, phLFs+TGFβ1+example 84 and phLFs+example 84) exemplified for one arbitrary gene. The normalized four dimensional vector subsequently gets reduced to a two dimensional vector by UMAP. (F) Two specific example as described in (A) for MMP1, which is highly abundant in condition “d” but not in others, and for MYH, which is highly abundant in condition “c” but not in others. (G) UMAP clustering of all differentially expressed genes (>2 fold, FDR<10%) according to their similarities in gene-expression patterns between the four various conditions in phLFs, phLFs+TGFβ1, phLFs+TGFβ1+example 84 and phLFs+example 84. Thus, the green and red boxes highlight the clustering of genes with similar expression, whereas a blue and red color codes indicate low and high transcript abundances, respectively.

FIG. 6: Cluster analysis of transcriptomics and network analysis. (A) List of deregulated genes within cluster A that were found to form an interacting protein network based on its analysis in the STRING Database. (B) List of deregulated genes within cluster B that were found to form an interacting protein network based on its analysis in the STRING Database.

FIG. 7: Proteomic analysis of N23Ps treated human precision cut lung slices (hPCLS) identifies upregulated profibrotic target networks. (A) Heatmap displaying abundancies of the twenty highest (red) and lowest (blue) deregulated proteins of fibrotic cocktail (FC) treated PCLS (n=3, three different patients: P1, P2, P3) as fold-change of the ratio of FC versus CC. Proteins related to tissue fibrosis are indicated with red asterisks. (B) Illustration of UMAP clustering based on different protein abundances (“a,b,c,”) for various conditions (hPCLS, hPCLS+FC, hPCLS+FC+example 84 and hPCLS+FC+Tranilast). (C) Based on STRING DB analysis Cluster A included functional subnetworks involved in extracellular matrix organization (green), actin cytoskeleton (pink) and interleukin signaling (yellow). (D) List of deregulated proteins within cluster A that were found to form an interacting protein network based on its analysis in the STRING Database.

FIG. 8: 3D assessment of ECM deposition by using IPF patient-derived primary human lung fibroblasts. (A) Primary human lung fibroblasts (phLFs) are derived from explanted IPF lungs, expanded in cell-culture and used for high-throughput drug screening and hit validation. (B) Clinical data of patients from which the phLFs were derived. (C) Graphical representation of the actual workflow used in the ECM deposition assay. (D) Software-based volume rendering of confocal z-stacks of immunostained ECM (collagen I in red) is used for the quantification of ECM volume and automated cell count (Hoechst-stained cell nuclei in blue). Scale bar=500 μm. (E) Orthoview of a confocal z-stack of phLFs (red) depositing collagen V (green) exclusively outside (indicated by white arrows) and on the surface of the cells. Cell nuclei are stained by Hoechst (blue). Scale bar=50 μm and 25 μm. (F) Intricate 3D ECM network of collagen I (red) and collagen V (green) stained fibers, demonstrating areas of co-localizing fibers (white arrows) as well as single fibers (white arrowheads). Cell-nuclei are stained by Hoechst (blue). The confocal z-stack is shown as a maximum intensity projection. Scale bar=50 μm. (G) 4D confocal time-lapse imaging of phLFs for 16 hours showing various dynamic processes occurring during the assembly of single collagen I—ECM fibers (red). White arrows indicate single ECM fibers. Colored asterisks indicate single cell-nuclei (Hoechst in blue) of distinct cells. Scale bar=50 μm

FIG. 9: 3D fibrosis disease model using IPF patient-derived primary human lung fibroblasts. (A) phLFs treated with 1 ng/ml TGFβ1 transdifferentiated to myofibroblasts which incorporated αSMA (green) into actin-stress fibers. Cell nuclei are stained by Hoechst (blue). Scale bar=200 μm. Quantification of the mean fluorescence intensity (MFI) of αSMA expression of three different patient phLFs after TGFβ1 treatment (n=3). (B) Venn-diagram showing an overlap of 17 ECM proteins between the myofibroblast surface proteome (pink) and a published “core matrisome” (blue). (C) Heatmap of protein expression levels of ECM proteins on the surface of myofibroblasts (phLFs+TGFβ1), identifying collagen I and fibulin 1 among the highest upregulated ECM proteins. Red and blue indicate high and low protein expression levels, respectively. (D) 3D confocal immunofluorescence microscopy of phLFs and phLFs+TGFβ1. phLFs+TGFβ1 showed increased ECM deposition of collagen I (red), collagen V (green) and fibulin 1 (yellow). Cell nuclei were stained by Hoechst (blue). The confocal z-stack is shown as a maximum intensity projection. Scale bar=500 μm. (E) Software based quantification of the deposited ECM volume displays a significant increase in the amount of deposited ECM in phLFs+TGFβ1, whereas the amount of cells remains unchanged. n=4 (four different patient phLFs). Statistics: One-way ANOVA with Bonferroni-correction. (F) 3D confocal immunofluorescence microscopy assessing the ECM deposition of phLFs, phLFs+TGFβ1 and phLFs+TGFβ1+EDHB. Ethyl-3,4-dihydroxy-benzoate (EDHB) treatment inhibits the ECM deposition of collagen I (red), collagen V (green) and fibulin 1 (yellow). Cell nuclei were stained by Hoechst (blue). The confocal z-stack is shown as a maximum intensity projection. Scale bar=500 μm. (G) Software based quantification of the deposited ECM volume of data shown in (F). Data are presented as means t SEM. Differences between groups were evaluated with paired t-tests. * p<0.05.

FIG. 10: Fibrotic Pattern Detection by Artificial Intelligence (FANTAIL) using a CNN for hit-identification within ECM deposition screening data of 1509 FDA-approved compounds. (A) Outline of the supervised multilayered deep convolutional neuronal network (CNN) developed for detecting fibrotic and non-fibrotic patterns in images containing deposited ECM derived from 3D confocal microscopy of immunofluorescently labelled collagen I, collagen V and fibulin 1. (B) The training dataset consisted of assay controls and additional samples treated with phLFs+TGFβ1 plus 5% ethanol. The CNN network was exclusively trained to detect inhibitors of ECM deposition as well as false positive hits due to cytotoxic effects. (C) UMAP clustering of predicted hits sorted out false positive hits due to immunofluorescence artefacts. After this final filtering, N-(3′,4′-dimethoxycinnamoyl)-anthranilic-acid (Tranilast) was determined as a promising candidate for repurposing in IPF. (D) Pie chart demonstrating the classification of detected hits into groups of similar molecular functions and biological processes. Tranilast is classified within the GPCR targeting molecules. (E) 3D confocal microscopy of immunofluorescently labelled collagen I (red), collagen V (green) and fibulin 1 (yellow) to validate the inhibitory effect of Tranilast treated phLFs+TGFβ1 on the deposition of ECM in a dose-dependent manner (untreated, vehicle, 75 μM, 150 μM, 300 μM). Cell nuclei were stained by Hoechst (blue). The confocal z-stack is shown as a maximum intensity projection. Scale bar=500 μm. (F) Quantification and statistical analysis of the inhibitory effects of Tranilast on phLFs+TGFβ1 on ECM deposition of collagen I, collagen V and fibulin 1 normalized to the number of cells. ns=not significant. * p<0.05 and ** p<0.01. Statistics: One-way ANOVA with Bonferroni-correction. All quantitative data represent means±SEM. n=3 (three different patient phLFs).

FIG. 11: Genome-wide transcriptomic analysis of N23Ps identifying a novel antifibrotic target network. (A) Experimental outline of transcriptional analysis of the human fibrosis model and treatment with ECM-deposition inhibiting N23Ps. (B) Volcano plots depicting all significantly differentially expressed genes (>2 fold, FDR<10%) in phLFs+TGFβ1 phLFs+TGFβ1+example 84 and phLFs+example 84 highlighting the ten highest (red) and lowest (blue) abundant transcripts. (C) Venn diagram showing 362 overlapping genes between 2076 deregulated genes in phLFs+TGFβ1 and 661 deregulated genes in phLFs+TGFβ1+example 84. (D) Gene-set enrichment analysis (GSEA) of phLFs+TGFβ1+example 84 showing a negative enrichment for profibrotic gene signatures such as collagen formation, extracellular matrix organization and smooth-muscle contraction. (E) UMAP-RPC overlaying each gene in its cluster with color-coded transcript abundances as fold change. Upregulated genes are depicted in red and downregulated genes in blue. Boxed Cluster A (red) designates genes which were mostly found upregulated in phLFs+TGFβ1 only, and boxed Cluster B (green) designates genes which were upregulated in phLFs+TGFβ31+example 84 and phLFs+example 84. (F) Based on STRING DB analysis, Cluster A included functional subnetworks of molecular components involved in the extracellular matrix organization (green) and the actin cytoskeleton (pink). (G) Based on STRING DB analysis cluster B included functional subnetworks of deubiquitination (yellow), laminin interactions (red), Rho GTPase effectors (green), and ECM receptor interactions (blue).

FIG. 12: Inhibition of myofibroblast transdifferentiation and contractility in a SMURF2 dependent manner. (A) Confocal microscopy images of phLFs concomitantly treated with TGFβ1 and active N23Ps, and stained for αSMA (red) and Hoechst (blue) reducing αSMA positive myofibroblasts. Scale bar=500 μm. (B) Quantification of myofibroblasts by mean fluorescence intensities (MFI) depicted in (A) exhibiting a significant inhibition of myofibroblast transdifferentiation. n=3 (three different patient phLFs). (C) 3D collagen gel contractility assay (scale bar=2000 μm) and its (D) quantification demonstrating a significant inhibition of cellular contractility by example 84. n=3 (three different patient phLFs). (E) Depletion of SMURF2 in IPF-phLFs by siRNA knock-down showing a significant reduction in geneexpression of >70%. n=3 (three different patient phLFs). (F) Protein expression analysis by western blotting exhibiting a significant upregulation of αSMA protein expression in IPF-phLFs+TGFβ1+example 84 depleted of SMURF2. n=4 (four different patient phLFs). (G) Confocal microscopy images of IPF-phLFs+TGFβ1+example 84 depleted of SMURF2 and stained for αSMA (red) and Hoechst (blue). Scale bar=200 μm. (H) Diagram depicting a possible mode of action of N23Ps by a SMURF2-inhibited TGFβ1 signaling and prevention of fibroblast-myofibroblast transdifferentiation. ns=not significant. * p<0.05, ** p<0.01 and *** p<0.001. Statistics: One-way ANOVA with Bonferroni-correction. All quantitative data represent means±SEM.

FIG. 13: A human ex vivo fibrosis model of precision cut lung slices (PCLS) confirms antifibrotic effects of N23Ps. (A) Human ex vivo fibrosis model derived from human lung resections. (B) Volcano plots depicting significantly (p<0.05) differentially expressed proteins (pink) in fibrotic cocktail (FC) treated PCLS, as well as their inhibition with example 84 and Tranilast. The ten highest and lowest abundant proteins are highlighted in red and blue, respectively. (C) Heatmap displaying protein abundancies as described in (B) and demonstrating protein deregulation as a consequence of example 84 and Tranilast treatment. (D) UMAP-RPC clustering of 580 differentially expressed proteins (p<0.1) according to their similarities in expression patterns of the various conditions tested, that is fibrotic cocktail (FC), control cocktail (CC), FC+example 84 and FC+Tranilast (TL). Colored boxes show clusters of proteins which display common abundancies between the different conditions tested. (E) Analysis of commonly regulated protein abundancy clusters found in (D). (F) Immunoplotting and quantification showing diminution of secreted pro-fibrotic PAI-1/SERPINE1 by example 84 in living human PCLS. (G) ELISA data demonstrating diminution of pro-fibrotic CXCL8/IL-8 by example 84 in living human PCLS. * p<0.05 and ** p<0.01. Statistics: One-way ANOVA with Bonferroni-correction. All quantitative data represent means±SEM. n=3 (three different patient PCLS).

FIG. 14: Dose-response relationships of N23Ps, live imaging of ECM deposition and cell-morphology switch. (A) Structural formula for Tranilast and N23Ps (example 84 and example 85) indicating 2-butoxy-substitution at R1 and IC50 values. (B) Dose-response curves of collagen V and fibulin 1 ECM deposition for N23Ps (example 84 and example 85) compared to Tranilast for determining IC50 values. n=3 (three different patient phLFs. (C) Tables of IC50s for active N23Ps compared to Tranilast. (C) 3D confocal images displayed as maximum intensity projection of deposited ECM taken as frames from a live-cell experiment demonstrating dynamic ECM deposition in phLFs, phLFs+TGFβ1 and phLFs+TGFβ31+example 84 during 48 hours. Immunostained collagen I and fibulin 1 depicted as one common signal (=ECM deposition) as a gold look-up table (LUT). Cell nuclei in blue (HOECHST). Scale bar=500 μm. (D) Quantification by mean fluorescent intensity (MFI) of dynamic deposition of collagen I and fibulin 1 in phLFs, phLFs+TGFβ1 and phLFs+TGFβ1+example 84 strikingly demonstrating inhibition of ECM deposition by example 84 over time. (E) Confocal images of calcein stained confluent phLFs+TGFβ1 proving viability in untreated and example 84 treated cells, as well as identifying a morphology switch from elongated to round cells. Cell nuclei were stained by Hoechst (blue). Scale bar=50 μm. (F) Confocal images of subconfluent phLFs+TGFβ1 stained for Hoechst (blue), calcein (green) and phalloidin (red) showing round cell morphologies after treatment with example 84 as well as extensive actin cytoskeletal rearrangements (red). Software-based segmentation of the cells in the images by CellProfiler allowed for the statistical analysis which exhibited significant changes in cell-shape and eccentricity towards round cells after example 84 treatment. Scale bar=500 μm. Data are presented as means t SEM. Differences between groups were evaluated with paired t-tests. * p<0.05. n=3 (three different patient phLFs).

FIG. 15: (D) Dose-response curves of collagen V and fibulin 1 ECM deposition for N23Ps (example 86 and example 87) compared to Tranilast for determining IC50 values. n=3 (three different patient phLFs). (E) Dose-response curves of collagen V and fibulin 1 ECM deposition for N23Ps (example 61 and example 88) compared to Tranilast for determining IC50 values. n=3 (three different patient phLFs). (F) Human dermal fibroblasts (n=3) treated with N23Ps at a working concentration of 50 nM demonstrating reduction of ECM deposition. Scale bar=500 μm. Fibulin 1 in red and cell nucleistained by Hoechst (blue). (G) Quantification of 3D deposition of fibulin 1 shown in FIG. 15D normalized to the cell count. ns=not significant. ** p<0.01 and *** p<0.001. Statistics: One-way ANOVA with Bonferroni-correction. All quantitative data represent means±SEM. n=3 (human dermal fibroblasts from three different individuals). (H) Graphical overlay of chemical structures of active N23Ps discovered by “catalogue” SAR all of which were characterized by a 2-butoxy-substitution at R1. (1) Graphical overlay of chemical structures of active, purpose-synthesized N23Ps with 2-butoxy or 2-o-benzyl substitution at R1.

FIG. 16: (A) Fluorescence wide-field microscopy images (10× objective in low magnification and resolution) of phLFs treated with 5 μM DMSO, 50 μM example 84 and 100 μM Tranilast, and stained for Hoechst (blue), α-Tubulin (green) and filamentous actin (red, Phalloidin). As already pointed out if FIG. 14 (F), example 84 treated cells showed a cell-shape switch towards round cell morphologies. phLFs treated with Tranilast or with DMSO only did not show round but elongated cell morphologies with filamentous actin (red) and microtubules fibers (green). Importantly, example 84 treated phLFs displayed depolymerized, that is non-filamentous, microtubules (green), without affecting actin filaments. Scale bar=100 μm. (B) Fluorescence wide-field microscopy images (63× objective in high magnification and resolution) of phLFs treated with either 5 μM DMSO or 50 μM example 84, and stained for Hoechst (blue), a.-Tubulin (green) and filamentous actin (red, Phalloidin). These images clearly show intact microtubules fibers (in green) in DMSO treated phLFs, whereas mostly depolymerized (non-filamentous) (-tubulin staining (green dots) in example 84 treated phLFs. Scale bar=10 μm. In conclusion this advocates that N23Ps (here example 84) interfered with the regulation of microtubules dynamics and/or polymerization, whereas Tranilast-treated phLFs did not show effects on the microtubule cytoskeleton. This further speaks for an exclusive mode-of-action of N23Ps. (C) Bright-field microscopy of human lung organoids derived from human lung progenitor cells, displaying organoid growth when incubated with either cell culture media only or together with DMSO after 14 days in culture. However, treatment of human organoids with 10 μM example 84 inhibited organoid growth. Quantification was done by counting the amount of organoids found in one well divided by the amount of human lung progenitor cells seeded, which resulted in the colony formation efficiency in %. Scale bar=700 μm. iPSC=induced pluripotent stem cells. (D) Confocal fluorescence microscopy images of calcein (in green representing living cells or organoids) and ethidium homodimer Ill (in red representing dead cells or organoids), demonstrating that many lung progenitor cells are still viable after treatment with 10 μM of example 84 and 14 days in culture. Scale bar=350 μm. In conclusion, N23Ps (here example 84) inhibited growth of human lung organoids derived from human lung progenitor cells or stem cells, advocating for an inhibition of stem cell differentiation and/or proliferation. Thus, the compounds of the present invention show similar effects like colchicine on microtubules (41). Therefore, the compounds of the present invention are also for use in the treatment of inflammatory diseases such as arthrolithiasis, familiar mediterranean fever and pericarditis amongst others by the inhibition of polymorphonuclear leukocyte and macrophage motility as well as intracellular vesicular transport mechanisms.

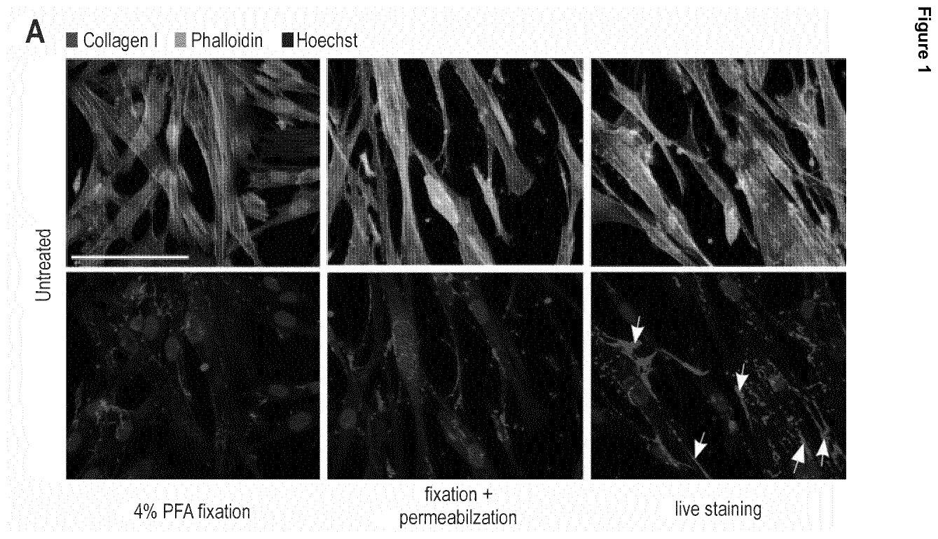

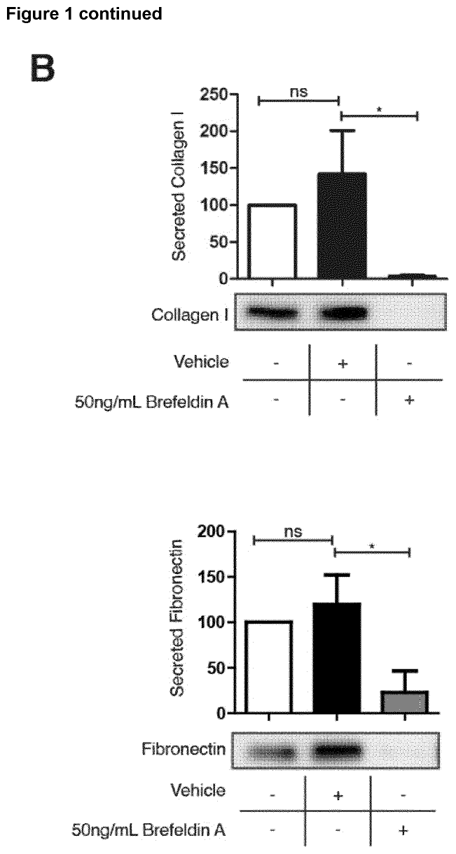

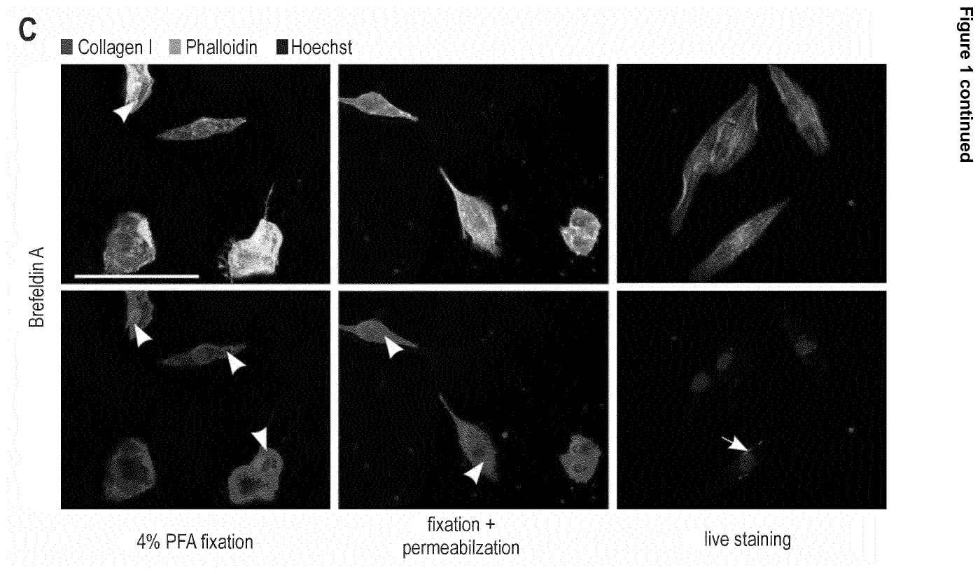

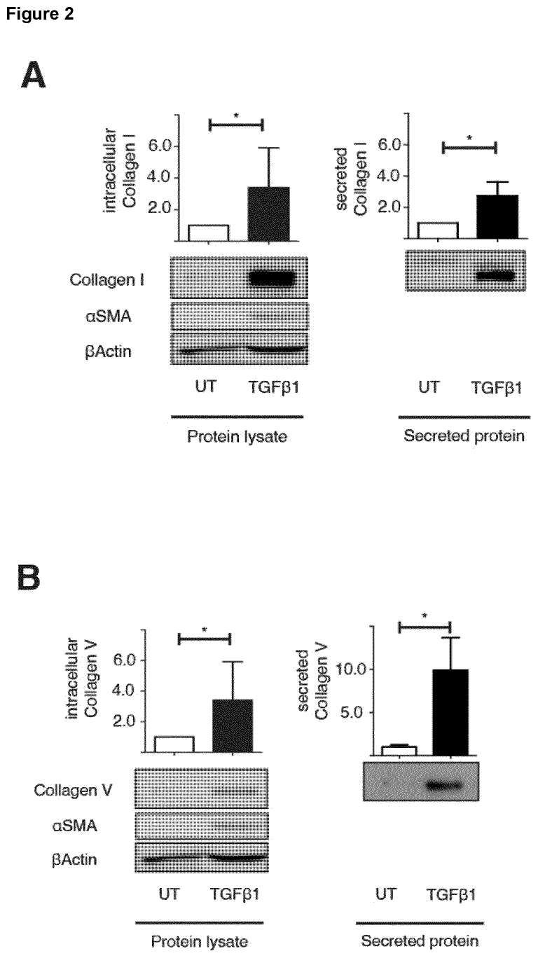

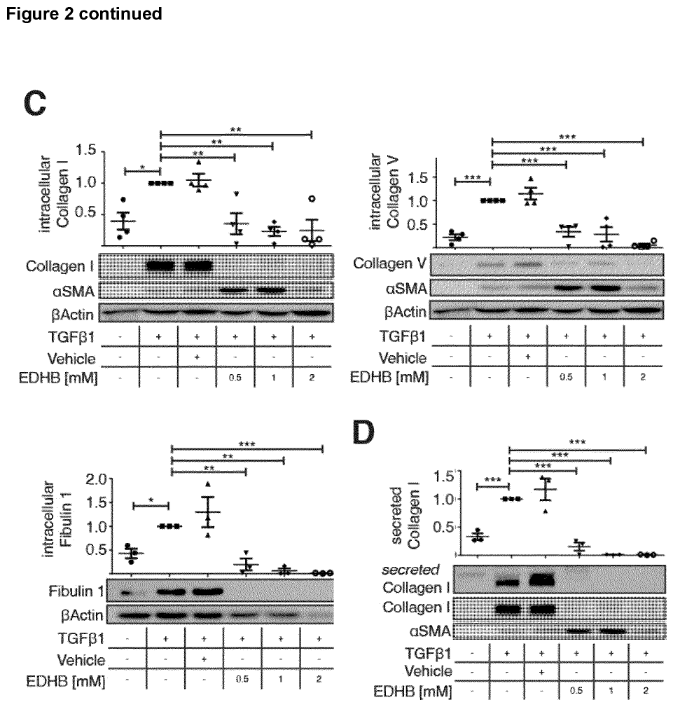

### DETAILED DESCRIPTION OF THE INVENTION

The solution of the present invention is described in the following, exemplified in the appended examples, illustrated in the Figures and reflected in the claims.

### Definitions

The term “alkyl” refers to a monoradical of a saturated straight or branched hydrocarbon. Preferably, the alkyl group comprises from 1 to 10 carbon atoms, i.e., 1, 2, 3, 4, 5, 6, 7, 8, 9, or 10 carbon atoms (such as 1, 2, 3, 4, 5, 6, 7, 8, 9, or 10 carbon atoms), more preferably 1 to 8 carbon atoms, such as 1 to 6 or 1 to 4 carbon atoms. Exemplary alkyl groups include methyl, ethyl, propyl, iso-propyl, butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, and the like.

The term “cycloalkyl” represents cyclic non-aromatic versions of “alkyl” and “alkenyl” with preferably 3 to 10 carbon atoms, i.e., 3, 4, 5, 6, 7, 8, 9, or 10 carbon atoms, more preferably 3 to 8 carbon atoms, even more preferably 3 to 7 carbon atoms. Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl. Preferred examples of cycloalkyl include (C₃-C₈)-cycloalkyl, in particular cyclopropyl, cyclobutyl, cyclopentyl, cyclohexyl, cycloheptyl, cyclooctyl.

In an alkyl group or cycloalkyl group, one or more hydrogen atoms may be replaced by a halogen atom, such as Cl, Br, F, preferably F.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 4, i.e., 1, 2, 3, or 4, carbon-carbon double bonds. Preferably, the alkenyl group comprises from 2 to 10 carbon atoms, i.e., 2, 3, 4, 5, 6, 7, 8, 9, or 10 carbon atoms, more preferably 2 to 8 carbon atoms, such as 2 to 6 carbon atoms or 2 to 4 carbon atoms. Thus, in a preferred embodiment, the alkenyl group comprises from 2 to 10 carbon atoms and 1, 2, 3, 4, or 5 carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, and the like. If an alkenyl group is attached to a nitrogen atom, the double bond cannot be alpha to the nitrogen atom.

The term “alkenylene” refers to a diradical of an unsaturated straight or branched hydrocarbon having at least one carbon-carbon double bond. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 4, i.e., 1, 2, 3, or 4, carbon-carbon double bonds. Preferably, the alkenylene group comprises from 2 to 10 carbon atoms, i.e., 2, 3, 4, 5, 6, 7, 8, 9, or 10 carbon atoms, more preferably 2 to 8 carbon atoms, such as 2 to 6 carbon atoms or 2 to 4 carbon atoms. Thus, in a preferred embodiment, the alkenylene group comprises from 2 to 10 carbon atoms and 1, 2, 3, 4, or 5 carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenylene groups include ethen-1,2-diyl, vinyliden, 1-propen-1,2-diyl, 1-propen-1,3-diyl, 1-propen-2,3-diyl, allyliden, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl, 2-buten-2,3-diyl, 2-buten-2,4-diyl, 2-buten-3,4-diyl, and the like. If an alkenylene group is attached to a nitrogen atom, the double bond cannot be alpha to the nitrogen atom.

The term “alkynyl” refers to a monoradical of an unsaturated straight or branched hydrocarbon having at least one carbon-carbon triple bond. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 4, i.e., 1, 2, 3, or 4, more preferably 1 or 2 carbon-carbon triple bonds. Preferably, the alkynyl group comprises from 2 to 10 carbon atoms, i.e., 2, 3, 4, 5, 6, 7, 8, 9, or 10 carbon atoms, more preferably 2 to 8 carbon atoms, such as 2 to 6 carbon atoms or 2 to 4 carbon atoms. Thus, in a preferred embodiment, the alkynyl group comprises from 2 to 10 carbon atoms and 1, 2, 3, 4, or 5 (preferably 1, 2, or 3) carbon-carbon triple bonds, more preferably it comprises 2 to 8 carbon atoms and 1, 2, 3, or 4 (preferably 1 or 2) carbon-carbon triple bonds, such as 2 to 6 carbon atoms and 1, 2 or 3 carbon-carbon triple bonds or 2 to 4 carbon atoms and 1 or 2 carbon-carbon triple bonds. Exemplary alkynyl groups include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 2-heptynyl, 3-heptynyl, 4-heptynyl, 5-heptynyl, 6-heptynyl, 1-octynyl, 2-octynyl, 3-octynyl, 4-octynyl, 5-octynyl, 6-octynyl, 7-octynyl, 1-nonylyl, 2-nonynyl, 3-nonynyl, 4-nonynyl, 5-nonynyl, 6-nonynyl, 7-nonynyl, 8-nonynyl, 1-decynyl, 2-decynyl, 3-decynyl, 4-decynyl, 5-decynyl, 6-decynyl, 7-decynyl, 8-decynyl, 9-decynyl, and the like. If an alkynyl group is attached to a nitrogen atom, the triple bond cannot be alpha to the nitrogen atom.

The term “heterocyclyl” means a cycloalkyl group as defined above in which from 1, 2, 3, or 4 carbon atoms in the cycloalkyl group are replaced by heteroatoms of O, S, or N. Preferably, in each ring of the heterocyclyl group the maximum number of 0 atoms is 1, the maximum number of S atoms is 1, and the maximum total number of O and S atoms is 2. The term “heterocyclyl” is also meant to encompass partially or completely hydrogenated forms (such as dihydro, tetrahydro or perhydro forms) of the above-mentioned heteroaryl groups. Exemplary heterocyclyl groups include morpholino, isochromanyl, chromanyl, pyrrolidinyl, imidazolidinyl, pyrazolidinyl, piperidinyl, piperazinyl, indolinyl, isoindolinyl, di- and tetrahydrofuranyl, di- and tetrahydrothienyl, di- and tetrahydrooxazolyl, di- and tetrahydroisoxazolyl, di- and tetrahydrooxadiazolyl (1,2,5- and 1,2,3-), dihydropyrrolyl, dihydroimidazolyl, dihydropyrazolyl, di- and tetrahydrotriazolyl (1,2,3- and 1,2,4-), di- and tetrahydrothiazolyl, di- and tetrahydrothiazolyl, di- and tetrahydrothiadiazolyl (1,2,3- and 1,2,5-), di- and tetrahydropyridyl, di- and tetrahydropyrimidinyl, di- and tetrahydropyrazinyl, di- and tetrahydrotriazinyl (1,2,3-, 1,2,4-, and 1,3,5-), di- and tetrahydrobenzofuranyl (1- and 2-), di- and tetrahydroindolyl, di- and tetrahydroisoindolyl, di- and tetrahydrobenzothienyl (1- and 2), di- and tetrahydro-1H-indazolyl, di- and tetrahydrobenzimidazolyl, di- and tetrahydrobenzoxazolyl, di- and tetrahydroindoxazinyl, di- and tetrahydrobenzisoxazolyl, di- and tetrahydrobenzothiazolyl, di- and tetrahydrobenzisothiazolyl, di- and tetrahydrobenzotriazolyl, di- and tetrahydroquinolinyl, di- and tetrahydroisoquinolinyl, di- and tetrahydrobenzodiazinyl, di- and tetrahydroquinoxalinyl, di- and tetrahydroquinazolinyl, di- and tetrahydrobenzotriazinyl (1,2,3- and 1,2,4-), di- and tetrahydropyridazinyl, di- and tetrahydrophenoxazinyl, di- and tetrahydrothiazolopyridinyl (such as 4,5,6-7-tetrahydro[1,3]thiazolo[5,4-c]pyridinyl or 4,5,6-7-tetrahydro[1,3]thiazolo[4,5-c]pyridinyl, e.g., 4,5,6-7-tetrahydro[1,3]thiazolo[5,4-c]pyridin-2-yl or 4,5,6-7-tetrahydro[1,3]thiazolo[4,5-c]pyridin-2-yl), di- and tetrahydropyrrolothiazolyl (such as 5,6-dihydro-4H-pyrrolo[3,4-d][1,3]thiazolyl), di- and tetrahydrophenothiazinyl, di- and tetrahydroisobenzofuranyl, di- and tetrahydrochromenyl, di- and tetrahydroxanthenyl, di- and tetrahydrophenoxathiinyl, di- and tetrahydropyrrolizinyl, di- and tetrahydroindolizinyl, di- and tetrahydroindazolyl, di- and tetrahydropurinyl, di- and tetrahydroquinolizinyl, di- and tetrahydrophthalazinyl, di- and tetrahydronaphthyridinyl (1,5-, 1,6-, 1,7-, 1,8-, and 2,6-), di- and tetrahydrocinnolinyl, di- and tetrahydropteridinyl, di- and tetrahydrocarbazolyl, di- and tetrahydrophenanthridinyl, di- and tetrahydroacridinyl, di- and tetrahydroperimidinyl, di- and tetrahydrophenanthrolinyl (1,7-, 1,8-, 1,10-, 3,8-, and 4,7-), di- and tetrahydrophenazinyl, di- and tetrahydrooxazolopyridinyl, di- and tetrahydroisoxazolopyridinyl, di- and tetrahydropyrrolooxazolyl, and di- and tetrahydropyrrolopyrrolyl. Exemplary 5- or 6-membered heterocyclyl groups include morpholino, pyrrolidinyl, imidazolidinyl, pyrazolidinyl, piperidinyl, piperazinyl, di- and tetrahydrofuranyl, di- and tetrahydrothienyl, di- and tetrahydrooxazolyl, di- and tetrahydroisoxazolyl, di- and tetrahydrooxadiazolyl (1,2,5- and 1,2,3-), dihydropyrrolyl, dihydroimidazolyl, dihydropyrazolyl, di- and tetrahydrotriazolyl (1,2,3- and 1,2,4-), di- and tetrahydrothiazolyl, di- and tetrahydroisothiazolyl, di- and tetrahydrothiadiazolyl (1,2,3- and 1,2,5-), di- and tetrahydropyridyl, di- and tetrahydropyrimidinyl, di- and tetrahydropyrazinyl, di- and tetrahydrotriazinyl (1,2,3-, 1,2,4-, and 1,3,5-), and di- and tetrahydropyridazinyl.

The term “aryl” refers to a monoradical of an aromatic cyclic hydrocarbon. Preferably, the aryl group contains 3 to 10 (e.g., 5 to 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl.

The term “heteroaryl” means an aryl group as defined above in which one or more carbon atoms in the aryl group are replaced by heteroatoms of O, S, or N. Preferably, heteroaryl refers to a five or six-membered aromatic monocyclic ring wherein 1, 2, or 3 carbon atoms are replaced by the same or different heteroatoms of O, N, or S. Alternatively, it means an aromatic bicyclic or tricyclic ring system wherein 1, 2, 3, 4, or 5 carbon atoms are replaced with the same or different heteroatoms of O, N, or S. Preferably, in each ring of the heteroaryl group the maximum number of O atoms is 1, the maximum number of S atoms is 1, and the maximum total number of O and S atoms is 2. Exemplary heteroaryl groups include furanyl, thienyl, oxazolyl, isoxazolyl, oxadiazolyl (1,2,5- and 1,2,3-), pyrrolyl, imidazolyl, pyrazolyl, triazolyl (1,2,3- and 1,2,4-), tetrazolyl, thiazolyl, isothiazolyl, thiadiazolyl (1,2,3- and 1,2,5-), pyridyl, pyrimidinyl, pyrazinyl, triazinyl (1,2,3-, 1,2,4-, and 1,3,5-), benzofuranyl (1- and 2-), indolyl, isoindolyl, benzothienyl (1- and 2-), 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl (1,2,3- and 1,2,4-benzotriazinyl), pyridazinyl, phenoxazinyl, thiazolopyridinyl, pyrrolothiazolyl, phenothiazinyl, isobenzofuranyl, chromenyl, xanthenyl, phenoxathiinyl, pyrrolizinyl, indolizinyl, indazolyl, purinyl, quinolizinyl, phthalazinyl, naphthyridinyl (1,5-, 1,6-, 1,7-, 1,8-, and 2,6-), cinnolinyl, pteridinyl, carbazolyl, phenanthridinyl, acridinyl, perimidinyl, phenanthrolinyl (1,7-, 1,8-, 1,10-, 3,8-, and 4,7-), phenazinyl, oxazolopyridinyl, isoxazolopyridinyl, pyrrolooxazolyl, and pyrrolopyrrolyl. Exemplary 5- or 6-memered heteroaryl groups include furanyl, thienyl, oxazolyl, isoxazolyl, oxadiazolyl (1,2,5- and 1,2,3-), pyrrolyl, imidazolyl, pyrazolyl, triazolyl (1,2,3- and 1,2,4-), thiazolyl, isothiazolyl, thiadiazolyl (1,2,3- and 1,2,5-), pyridyl, pyrimidinyl, pyrazinyl, triazinyl (1,2,3-, 1,2,4-, and 1,3,5-), and pyridazinyl.

The term “azido” means N₃.

In an alkyl group, cycloalkyl group, heterocyclyl, alkenyl group, alkenylene group, aryl group, or heteroaryl group, one or more hydrogen atoms may be replaced by a halogen atom, such as Cl, Br, F, preferably F, —OH, —NH₂, —NHC(O)CH₃, —CN, —N₃, —COOH, and/or —C(O)NH.

The invention comprises a compound according to formula (I)

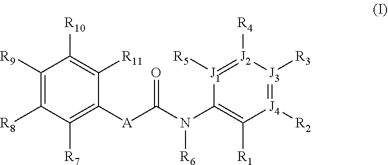

wherein,
R¹ is selected from the group consisting of —OR¹², —O(CH₂)₁(C₃-C₁₀)aryl, —O(CH₂)ᵤ(C₂)alkynyl; —(CH₂)ᵤ(C₃-C₁₀)aryl, —O(CH₂)ᵤ(C₃-C₁₀)cycloalkyl, —(CH₂)ᵤ(C₃-C₁₀)cycloalkyl,

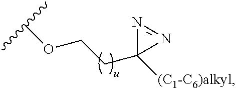

—(CH₂).(C₂)alkynyl.

Preferably, for

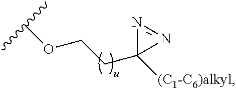

u is 1 and (C₁-C₆)alkyl is methyl.

u is 0 to 6.

R² to R⁵ are independently selected from the group consisting of H, —OR¹², —(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —CHZ₂, —CZ₃—CH₂Z, —OCHZ₂, —OCZ₃, —OCH₂Z—N(R¹³)(R¹⁴), —N(R¹⁶)(OR¹⁶), —S(O)₀₋₂R′⁷, —S(O)₁₋₂OR¹⁸, —OS(O)₁₋₂R¹⁹, —OS(O)₁₋₂OR²⁰, —S(O)₁₋₂N(R²¹)(R²²), —OS(O)₁₋₂N(R²³)(R²⁴), —N(R²⁵)S(O)₁₋₂R²⁶, —NR²⁷S(O)₁₋₂OR²⁸, —NR²⁹S(O)₁₋₂N(R³⁰)(R³¹), —C(═X)R³², —C(═X)XR³³, —XC(═X)R³⁴, and —XC(═X)XR³⁵, —OR³⁶, —O(CH₂)ᵥ(C₃-C₁₀)aryl, —O(CH₂)ᵥ(C₃-C₁₀)cycloalkyl, —O(CH₂)ᵥ(C₂)alkynyl.

R⁶ is selected from the group consisting of H, —(C₁-C₁₀)alkyl, and —(CH₂)₁₋₅(C₃-C₁₀)cycloalkyl; wherein —(C₁-C₁₀)alkyl, benzyl and —(CH₂)₁₋₅(C₃-C₁₀)cycloalkyl optionally are further substituted with at least one substituent selected from the group consisting of Halogen, preferably F.

R⁷ to R¹¹ are independently selected from the group consisting of H, —OR¹², —SR¹², —(C₁-C₁₀)alkyl, halogen, —(C₁-C₁₀)alkylO(C₁-C₁₀)alkyl, cyano, isocyano, cyanato, isocyanato, thiocyanato, isothiocyanato, azido, —(C₂-C₁₀)alkenyl, —(C₂-C₁₀)alkynyl, —O(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —(CH₂)CHZ₂, —CZ₃—CH₂Z, —OCHZ₂, —OCZ₃, —OCH₂Z, —N(R¹³)(R¹⁴), —N(R¹⁵)(OR¹⁶), —S(O)₀₋₂R¹⁷, —S(O)₁₋₂OR¹⁸, —OS(O)₁₋₂R¹⁹, —OS(O)₁₋₂OR²⁰, —S(O)₁₋₂N(R²¹)(R²²), —OS(O)₁₋₂N(R²³)(R²⁴), —N(R²⁵)S(O)₁₋₂R²⁶, —NR²⁷S(O)₁₋₂OR²⁸, —NR²⁹S(O)₁₋₂N(R³⁰)(R³¹), —C(═X)R³², —C(═X)XR³³, —XC(═X)R³⁴, and —XC(═X)XR³⁵, —O(CH₂)ᵥ(C₃-C₁₀)cycloakyl, —O(CH₂)ᵥ(C₁-C₁₀)alkyl and —O(CH₂)ᵥ(C₃-C₁₀)aryl.

Two adjacent rests of R¹ to R⁵ and R⁷ to R¹¹, preferably R⁸ to R¹⁰ optionally may form a ring attached to the underlying aromatic ring of formula (I) according to formula (III) to (XI)

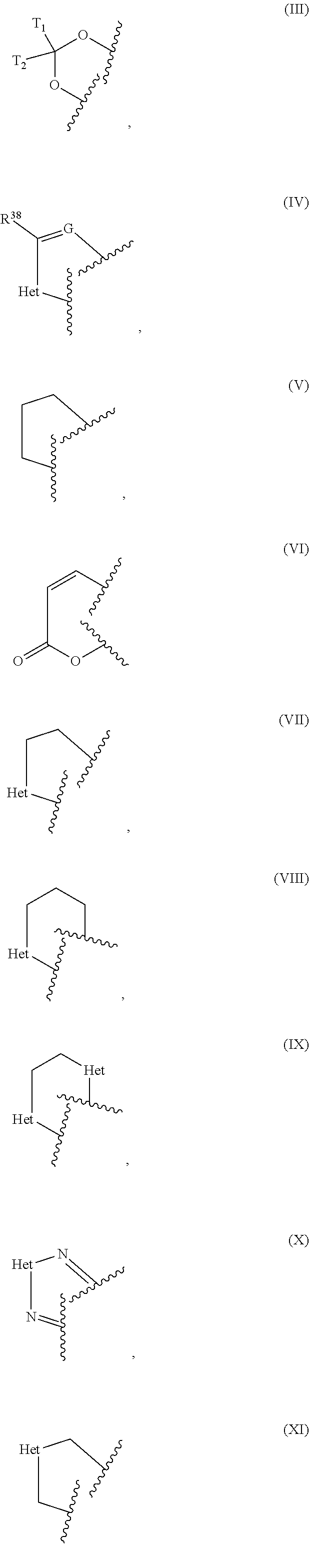

wherein T¹ and T² are independently selected from the group consisting of H, —(C₁-C₁₀)alkyl and halogen;
wherein each hydrogen in formula (III) to (XI) is optionally substituted with halogen, or —(C₃-C₁₀)aryl, preferably F.
R³⁸ is independently selected from the group consisting of H, —(C₁-C₁₀)alkyl;
Het is selected from O, S, NH.
G is selected from CH, NH.

J₁ to J₄ are independently selected from C or N, preferably J₁ to J₄ are C;
wherein if any one of J₁ to J₄ is N, the corresponding R¹ to R⁴ attached to the respective J₁ to J₄ which is (are) N is absent;

R¹² to R³⁶ are independently selected from the group consisting of H, —(C₁-C₁₀)alkyl, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl.

R¹ to R¹¹, independently selected from the group consisting of, —(C₁-C₁₀)alkyl, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —O(CH₂)ᵥ(C₃-C₁₀)cycloalkyl, —O(CH₂)ᵥ(C₁-C₁₀)alkyl and —O(CH₂)(C₃-C₁₀)aryl and R¹² to R³⁵ optionally are further substituted with at least one substituent selected from the group consisting of OR¹², —(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —CHZ₂, —CZ₃—CH₂Z, —OCHZ₂, —OCZ₃, —OCH₂Z, —N(R¹³)(R¹⁴), —N(R¹⁵)(OR¹⁶), —NHC(O)(C₁-C₁₀)alkyl, —S(O)₀₋₂R¹⁷, —S(O)₁₋₂OR¹⁸, —OS(O)₁₋₂R¹⁹, —OS(O)₁₋₂OR²⁰, —S(O)₁₋₂N(R²¹)(R²²), —OS(O)₁₋₂N(R²³)(R²⁴), —N(R²⁵)S(O)₁₂R²⁶, —NR²⁷S(O)₁₋₂OR²⁸, —NR²⁹S(O)₂N(R³⁰)(R³¹), —C(═X)R³², —C(═X)XR³³, —XC(═X)R³⁴, and —XC(═X)XR³⁵, —OR³⁶, and —O(CH₂)ᵥ(C₃-C₁₀)aryl.

v is 0 to 5;

Z is halogen;

X is selected from the group consisting of O, —NH— or S;

A is selected from the group consisting of

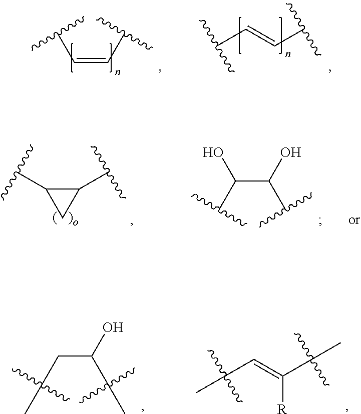

n is 1, 2, or 3, preferably 1;
is 1, 2, or 3, preferably 1;
R is H, (C₁-C₆)alkyl, cyano, —(C₃-C₁₀)cycloalkyl, benzyl or part of a ring wherein R is connected with R⁷ or R¹¹ by

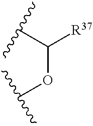

preferably H or benzyl, most preferably H;
with the provision that if n is 2 or 3, A may be

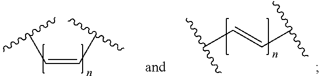

R³ is H or —CF₃;

With the proviso that R^{s} is not —COOH.

In one embodiment,
I) R¹ is selected from the group consisting of —OR¹², —O(CH₂)ᵤ(C₁-C₁₀)aryl, —O(CH₂)ᵤ(C₃-C₁₀)cycloalkyl, —O(CH₂)ᵤ(C₂)alkynyl; preferably of —OR¹²
u is 0 to 5;
R¹² is —(C₁-C₁₀)alkyl, preferably —(C₃-C₅)alkyl, more preferably —(C₄)alkyl and/or
II) A is selected from

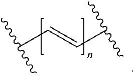

In another embodiment, R² to R⁵ are independently selected from the group consisting of H, —OR¹², —(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —CHZ₂, —CZ₃—CH₂Z, —OCHZ₂, —OCZ₃, —OCH₂Z, —N(R¹³)(R¹⁴), —N(R¹⁵)(OR¹⁶), —C(═X)R³², —C(═X)XR³³, —XC(═X)R³⁴, and —XC(═X)XR³⁵, —O(CH₂)ᵥ(C₃-C₁₀)aryl, —O(CH₂)ᵥ(C₃-C₁₀)cycloalkyl, —O(CH₂)ᵥ(C₂)alkynyl.

In another embodiment, R⁷ to R¹¹ are independently selected from the group consisting of H, —SR¹², —OR¹², —(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —O(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —(CH₂)ᵥCHZ₂, —CZ₃—CH₂Z, —OCHZ₂, —OCZ₃, —OCH₂Z, —N(R¹³)(R¹⁴), —N(R¹⁵)(OR¹⁶), —C(═X)R³², —C(═X)XR³³, —XC(═X)R³⁴, and —XC(═X)XR³⁵, —O(CH₂)ᵥ(C₃-C₁₀)cycloakyl, —O(CH₂)ᵥ(C₁-C₁₀)alkyl and —O(CH₂)ᵥ(C₃-C₁₀)aryl,
wherein two adjacent rests of R¹ to R⁵ and R⁷ to R¹¹ optionally may form a ring attached to the underlying aromatic ring of formula (II) according to

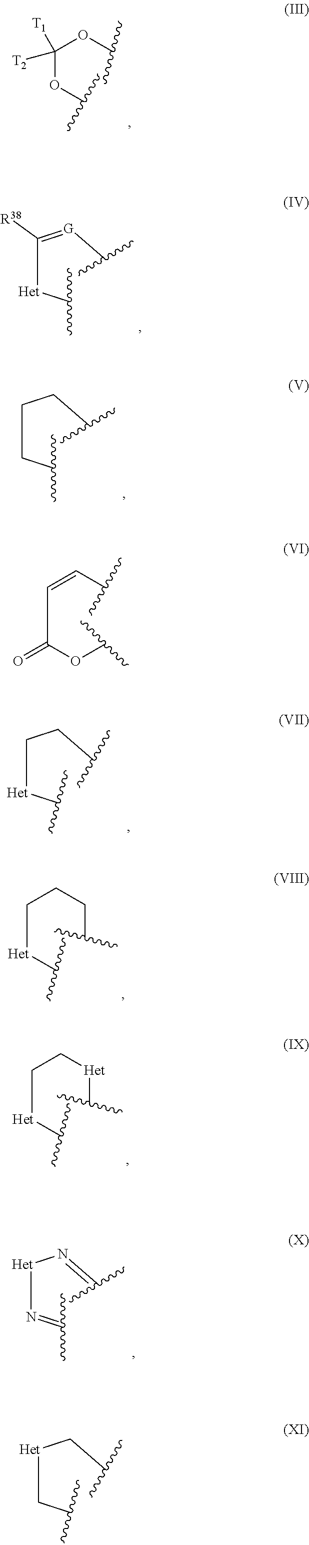

wherein T¹ and T² are independently selected from the group consisting of H, —(C₁-C₁₀)alkyl and halogen;
wherein each hydrogen in formula (III) to (XI) is optionally substituted with halogen, or —(C₃-C₁₀)aryl, preferably F.
Het is selected from O, S, NH.
G is selected from CH, NH.
R³⁸ is independently selected from the group consisting of H, —(C₁-C₁₀)alkyl.

In a further embodiment,
R¹ to R¹¹, selected from the group consisting of —(C₁-C₁₀)alkyl, —(C₂-C₁₀)alkenyl, —(C₂—C)alkynyl, —(C₃-C₁₀)cycloalkyl, —(C₃-C₁₀)heterocyclyl, —(C₃-C₁₀)aryl, —O(CH₂)ᵤ(C₃-C₁₀)cycloakyl, —O(CH₂)ᵥ(C₁-C₁₀)alkyl and —O(CH₂)ᵥ(C₃-C₁₀)aryl and R¹² to R³⁶ optionally are further substituted with a substituent selected from the group consisting of —OR¹²—(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —CHZ₂, —CZ₃—CH₂Z, —OCHZ₂, —OCZ₃, —OCH₂Z, N(R¹³)(R¹⁴), —N(R¹⁵)(OR's), —C(═X)R³², —C(═X)XR³³, —XC(═X)R³⁴, and —XC(═X)XR³⁵, —OR³⁶, and —O(CH₂)ᵥ(C₃-C₁₀)aryl.

If an R¹² is substituted with a further substituent such as —OR¹² then the specific R¹² selected from the group as specified above may be different in the underlying R¹² and in its substituent —OR¹².

In a further embodiment, R⁹ is selected from the group consisting of —OR¹², halogen, —O(C₂-C₁₀)alkynyl, —CZ₃, —OCHZ₂, —OCZ₃, —OCH₂Z, —O(CH₂)ᵥ(C₃-C₁₀)cycloakyl, —O(CH₂)(C₁-C₁₀)alkyl and —O(CH₂)ᵥ(C₃-C₁₀)aryl, wherein R⁹ selected from the group consisting of —O(C₁-C₁₀)alkyl, —OCH₂Z, —O(CH₂)(C₃-C₁₀)cycloakyl, —O(CH₂)ᵥ(C₁-C₁₀)alkyl and —O(CH₂)ᵤ(C₃-C₁₀)aryl optionally is further substituted with at least one substituent selected from the group consisting of Halogen, —OH, —NH₂, —NHC(O)CH₃, —CN, —N₃, and —COOH, —C(O)NH₂.

Further, the invention comprises a compound according to formula (II)

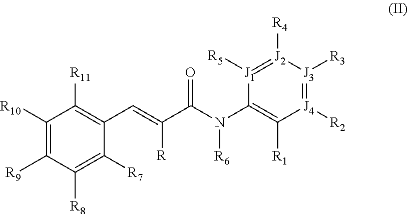

R¹ to R⁴ and R⁶, R⁷, R⁹ and R¹⁰ are independently selected from the group consisting of H, —(C₁-C₁₀)alkyl, —SR¹², halogen, azido, cyano, —O(C₁-C₁₀)alkyl, —(CH₂)ᵤ(C₃-C₁₀)aryl, —O(CH₂)ᵤ(C₃-C₁₀)cycloalkyl, —(CH₂)ᵤ(C₃-C₁₀)cycloalkyl, —(C₂-C₁₀)alkenyl,

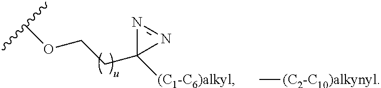

R⁵ is —(C₁-C₁₀)alkyl, —(C₅—C)heteroaryl, —(C₃-C₁₀)aryl, —(CH₂)ᵤ(C₃-C₁₀)cycloalkyl, preferably —(C₄—C)alkyl, or benzyl, most preferably —(C₄)alkyl.

R⁸ is H, —O(C₁-C₁₀)alkyl, —O(CH₂)ᵤ(C₃-C₁₄)aryl, —O(CH₂)ᵤ(C₃-C₁₀)cycloalkyl, —O(C₃-C₁₀)cycloalkyl, or, —O(C₂-C₁₀)alkenyl, preferably —OCH₃.

u is 0 to 6.

R is H, (C₁-C₆)alkyl, cyano, —(C₃-C₁₀)cycloalkyl, benzyl or part of a ring wherein R is connected with R⁷ or R¹¹ by

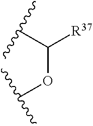

preferably H or benzyl, most preferably H.

R¹¹ is H, —(C₁-C₁₀)alkyl, and —(CH₂)₁₋₅(C₃-C₁₀)cycloalkyl.

R¹ to R¹¹, independently selected from the group consisting of, —(C₁-C₁₀)alkyl, —O(C₁-C₁₀)alkyl, —(C₂-C₁₀)alkenyl, —(C₂-C₁₀)alkynyl, —(C₃-C₁₀)cycloalkyl, —(CH₂)ᵤ(C₃-C₁₄)cycloalkyl, —(C₃-C₁₀)aryl, and —(CH₂)ᵤ(C₃-C₁₀)aryl and R¹² to R³⁵ optionally are further substituted with at least one substituent selected from the group consisting of Halogen, —OH, —NH₂, —NHC(O)CH₃, —CN, —N₃, and —COOH, —C(O)NH₂.

R¹² is —(C₁-C₁₀)alkyl, preferably —(C₃-C₅)alkyl, more preferably —(C₄)alkyl.

Two adjacent rests of R⁸ to R¹⁰ optionally may form a ring attached to the underlying aromatic ring of formula (II) according to

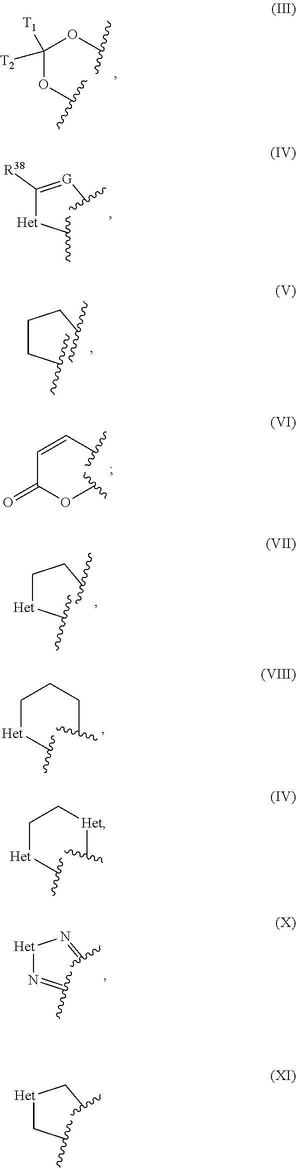

wherein in case of R³, the oxygen where it is attached to may correspond to one of the oxygen/Het atoms shown in the formula (III) to (XI);
wherein T¹ and T² are independently selected from the group consisting of H, —(C₁-C₁₀)alkyl and halogen;
wherein each hydrogen in formula (III) to (XI), optionally is substituted with halogen, or —(C₃-C₁₀)aryl, preferably F.

Het is selected from O.

G is selected from CH, NH.

R³⁸ is independently selected from the group consisting of H, —(C₁-C₁₀)alkyl.

J₁ to J₄ are independently selected from C or N, preferably J₁ to J₄ are C.

Wherein if any one of J₁ to J₄ is N, the corresponding R¹ to R⁴ attached to the respective J₁ to J₄ which is (are) N is absent.

In one embodiment one of J₁ to J₄ is N. In another embodiment two of J₁ to J₄ are N.

With the proviso that
if J₁ to J₄ are C and
I) if R⁵ is —(CH₂)₃CH₃; R⁹ is —OCH₃, —OCH₂CH₃, —O(CH₂)₂CH₃, —OCH₂phenyl or —O(CH₂)₃CH₃; R¹, R², R³, R⁴, R⁷, R¹¹ and R are H; and
a) R⁸ and R¹⁰ are H; or
b) R⁸ is —OCH₃, or —OCH₂CH₃ and R¹⁰ is H; or
c) R¹⁰ is —OCH₃, or —OCH₂CH₃ and R⁸ is of H;
then R is not H;
II) if R⁹ is —OCH₃, —O(CH₂)₂CH₃, —O(2-propyl), —O(CH₂)₄CH₃, —O(CH₂)₅CH₃, —OCH₂(4-chlorophenyl), —O(CH₂)₂CH(CH₃)₂, —OCH₂(2,6-dichlorophenyl), or —OCH₂phenyl; R¹, R², R³, R⁴, R⁷, R¹¹, and R are H; R⁸ is —OCH₃ and R¹⁰ is H, or R¹⁰ is —OCH₃ and R⁸ is H; R⁵ is —(CH₂)₃CH₃; then R⁶ is not H;
III) if R⁹ is —OCH₃; R⁸ is Brand R¹⁰ is H, or R¹⁰ is Br and R⁸ is H; R⁵ is —(CH₂)₃CH₃; R¹, R², R³, R¹, R¹¹ and R are H, then R¹ is not H;
IV) if R⁵ is —(CH₂)₃CH₃, R⁹ is —OCH₃, R⁷ is —OCH₃ and R¹¹ is H or R¹¹ is —OCH₃ and R⁷ is H; R¹, R², R³, R⁴, R⁸, R¹⁰, R¹¹ and R are H; then R⁶ is not H;
V) if R⁹ is —OCH₃, —OCH₂phenyl, —OCH₂(2-fluorophenyl); R⁸ is Brand R¹⁰ is —OCH₃, or R¹⁰ is Br and R⁸ is —OCH₃; R⁵ is —O(CH₂)₃CH₃; R¹, R², R³, R⁴, R⁷, R¹¹, and R are H, then R⁶ is not H;
VI) if R⁹ is —O(CH₂)₃CH₃; R⁸ is —OCH₂CH₃ and R¹⁰ is H, or R¹⁰ is —OCH₂CH₃ and R⁸ is H; R⁵ is —O(CH₂)₃CH₃; R¹, R², R³, R⁴, R⁷, R¹¹, and R are H, then R⁶ is not H;
VII) if R⁹ is —OCH₂(2-chlorophenyl); R⁸ is Br and R¹⁰ is —CH₂CH₃, or R¹⁰ is Br and R⁸ is —OCH₂CH₃; R⁵ is —(CH₂)₃CH₃; R¹, R², R³, R⁴, R⁷, R¹¹, and R, are H, then R⁶ is not H;
VIII) if R⁹ is —O(2-octenyl); R⁸ is Cl and R¹⁰ is H, or R¹⁰ is CI and R⁸ is H; R⁵ is —(CH₂)₃COOH; R¹, R², R³, R⁴, R⁷, R¹¹, and R are H, then is not H;
IX) if R⁹ is —OCH₃; R¹, R², R³, R⁴, R⁷, R⁸, R¹⁰, R¹¹, R are H; and
R⁵ is -(2-fluorophenyl), -phenyl; then R⁶ is not H;
X) if R⁹ is —OCH₂CH₃; R⁵ is —(CH₂)₃CH₃; R¹, R², R³, R⁴, R⁷, R⁸, R¹⁰, R¹¹, and R are H; then R⁶ is not H;
XI) if R⁹ is —OCH₃; R⁸ is —OCH₃ and R¹⁰ is H, or R¹⁰ is —OCH₃ and R⁸ is H; R⁵ is —(CH₂)₃CH₃; R¹, R², R³, R⁴, R⁷, R, and R are H; then R⁶ is not H;
XII) if R⁹ is —OCH₃; R⁵ is —O(CH₂)₃CH₃; R¹, R², R³, R⁴, R⁷, R¹¹, and R are H, and R⁸ is —OCH₂CH₃ and R¹⁰ is H, or R¹⁰ is —OCH₂CH₃ and R⁸ is H; then R⁶ is not H;
XIII) if R⁵ is —(CH₂)₃CH₃; R⁹ is —O(CH₂)₃CH₃, or —OCH₃; R¹, R², R³, R⁴, R⁷, R⁸, R¹⁰, R¹¹, and R are H; then R⁶ is not H;
XIV) if R⁵ is —CH₃, —(CH₂)₂CH₃ or —CH₂CH₃; R⁹ is —OCH₃; R⁸ is —OCH₃ and R¹⁰ is H or R⁸ is H and R¹⁰ is —OCH₃; R¹, R², R³, R⁴, R⁷, R¹¹, and R are H; then R⁶ is not H;
XV) if R⁵ is —(CH₂)₃CH₃; R¹, R², R³, R⁴, R⁷, R⁸, R⁹, R¹⁰ and R¹¹ are H; then R⁶ is not H;
and/or
with the provisio that
if J₁ to J₄ are C the following compounds are not comprised by the compounds according to formula (II):

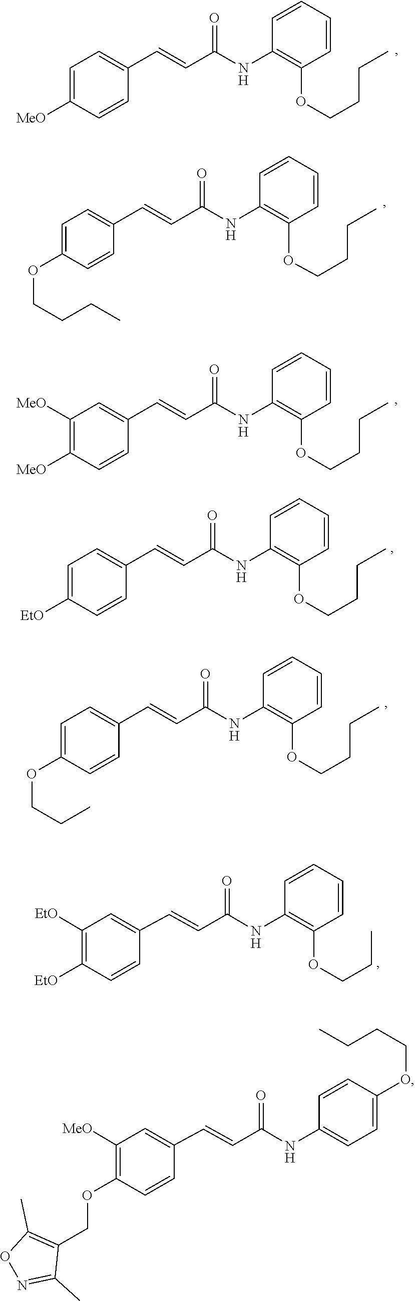

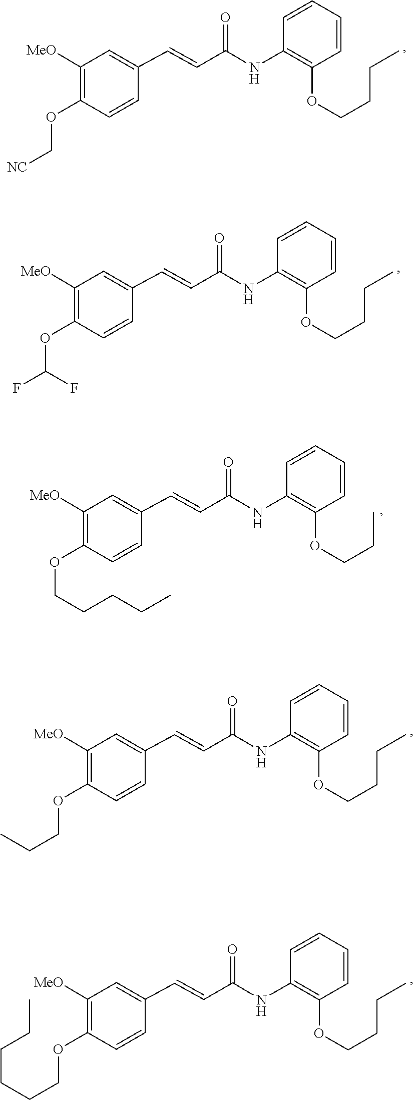

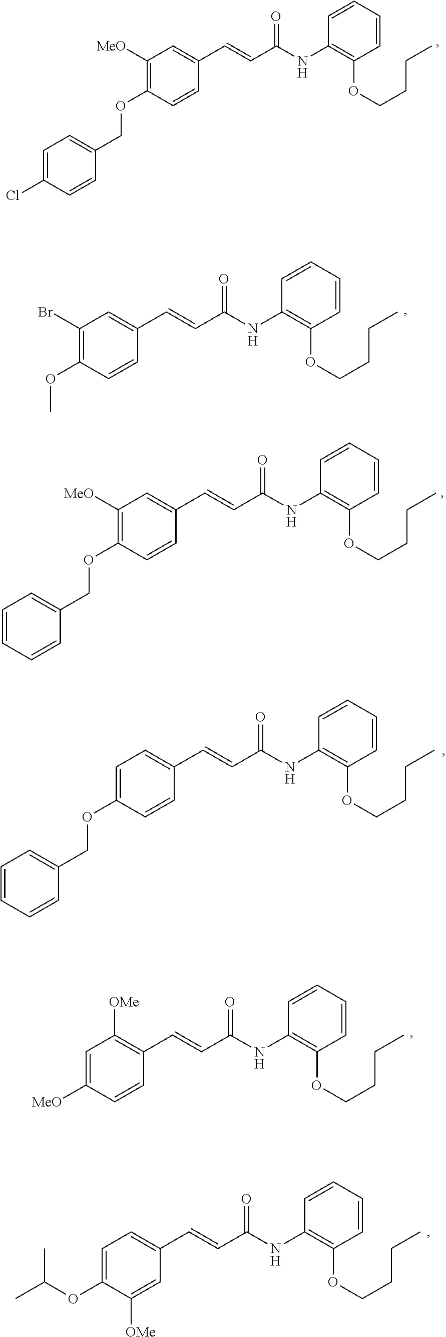

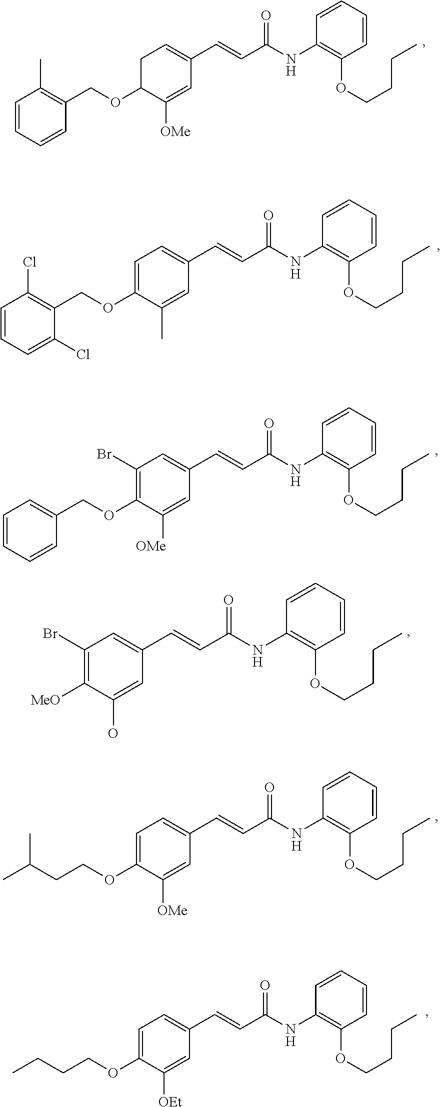

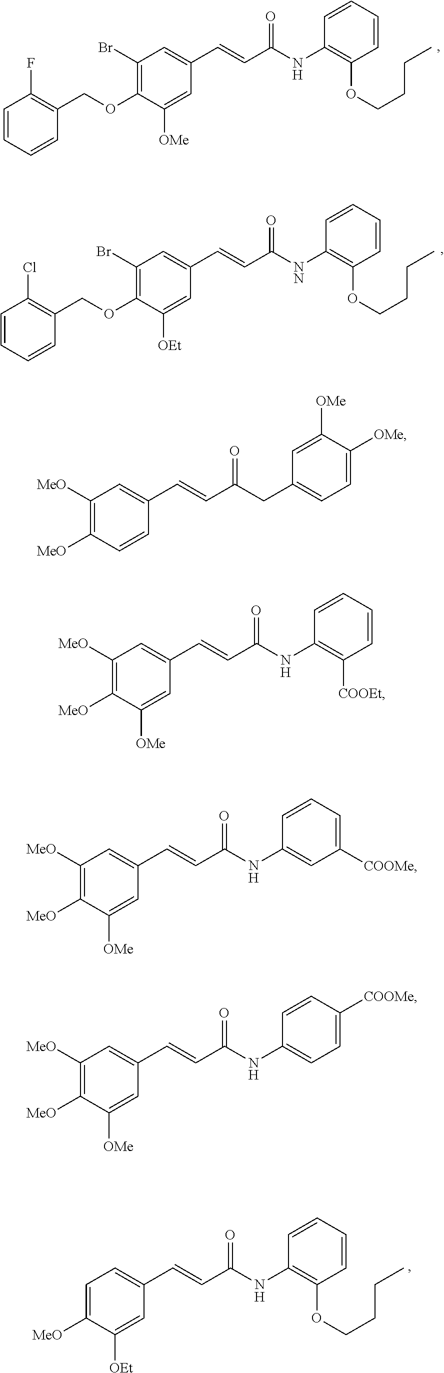

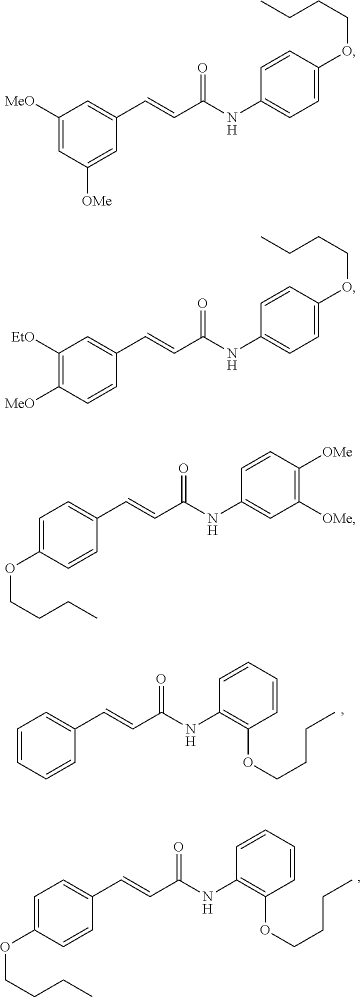 [SMILES: CCCCOc1ccc(NC(=O)/C=C/c2cc(OC)cc(OC)c2)cc1 ; CCCCOc1ccc(NC(=O)/C=C/c2ccc(OC)c(OCC)c2)cc1 ; CCCCOc1ccc(/C=C/C(=O)Nc2ccc(OC)c(OC)c2)cc1 ; CCCCOc1ccccc1NC(=O)/C=C/c1ccccc1 ; CCCCOc1ccc(/C=C/C(=O)Nc2ccccc2OCCCC)cc1]

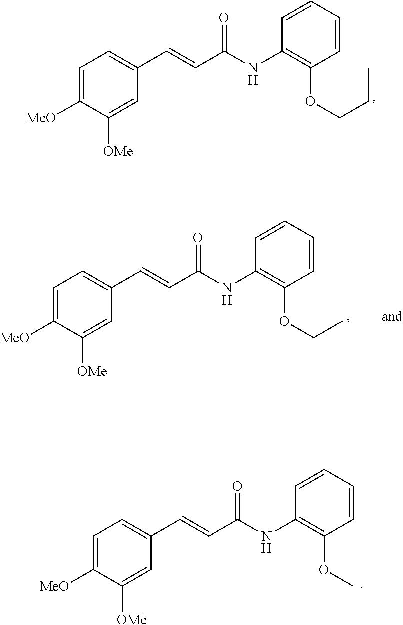 [SMILES: CCCOc1ccccc1NC(=O)/C=C/c1ccc(OC)c(OC)c1 ; CCOc1ccccc1NC(=O)/C=C/c1ccc(OC)c(OC)c1 ; COc1ccccc1NC(=O)/C=C/c1ccc(OC)c(OC)c1]

In a further embodiment, if J, to J₄ are C the following compounds are not or are also not comprised by the compounds according to formula (II):

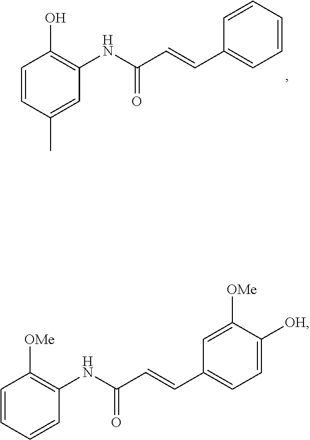 [SMILES: Cc1ccc(O)c(NC(=O)/C=C/c2ccccc2)c1 ; COc1cc(/C=C/C(=O)Nc2ccccc2OC)ccc1O]

In respect to the compounds according to formula (I) or (II), the invention further comprises the following embodiments i) to xxiv):
i) R⁹ is —OR¹²; and R⁸ is —H and R¹⁰ is H or —OR¹²; or
ii) R⁹ is H and R⁸ is H and R¹⁰ is —OR¹²; or
iii) R⁹ and R¹⁰ form a ring, attached to the underlying aromatic ring of formula (II) according to

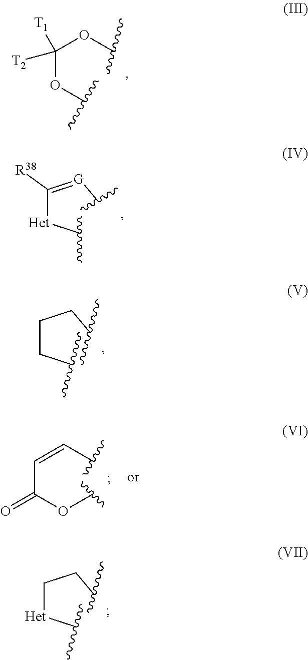

preferably according to formula (III)
wherein T¹ and T² are independently selected from the group consisting of H, —(C₁-C₁₀)alkyl and halogen;
wherein each hydrogen in formula (III) to (VII) is optionally substituted with halogen, or —(C₃-C₁₀)aryl, preferably F;
R³⁸ is independently selected from the group consisting of H, —(C₁-C₁₀)alkyl;
Het is O, S or NH, preferably 0;
G is selected from CH, N. and/or
iv) J₁₋₄ is CH; and/or
v) R² to R⁵, R⁷ or R¹¹ are independently selected from the group consisting of H, —OR¹²—(C₁-C₁₀)alkyl, halogen, cyano, azido, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —CHZ₂, —CZ₃—CH₂Z, —OCHZ₂, —OCZ₃, —OCH₂Z, —OR³⁶, —O(CH₂)ᵥ(C₃-C₁₀)aryl, —O(CH₂)ᵥ(C₃-C₁₀)cycloalkyl, —O(CH₂)ᵥ(C₂)alkynyl; and/or
vi) u=0-3 and/or
vii) R¹² is —O(C₄-C₆)alkyl, —OCH₂(C₃-C₅)cycloalkyl, —OPhenyl or —OCH₂Phenyl; and/or
viii) R⁶═H or C₁-C₄ alkyl or (CH₂)₍₁₋₃₎ alkyl-(C₁-C₆)cycloalkyl; and/or
ix) R² to R⁵, R⁷ or R¹¹ are independently selected from the group consisting of H, —OR¹², —(C₁-C₃)alkyl, halogen, cyano, azido, —CHZ₂, —CZ₃—CH₂Z, —OCHZ₂, —OCZ₃, —OCH₂Z, —O(CH₂)ᵥ(C₃-C₁₀)aryl, —O(CH₂)ᵥ(C₃-C₁₀)cycloalkyl, —O(CH₂)ᵥ(C₂)alkynyl; and/or
x) R¹² is selected from the group consisting of H, —(C₁-C₁₀)alkyl, —(C₂-C₄)alkynyl, —(C₃-C₁₀)cycloalkyl, —(C₃-C₁₀)heterocyclyl, —(C₃-C₁₀)aryl, —(C₃-C₁₀)heteroaryl; and/or;
xi) R² to R⁵, R⁷ or R¹¹ are independently selected from the group consisting of H, —(C₁-C₃)alkyl, halogen, cyano, azido, —CHZ₂, —CZ₃—CH₂Z, —OCHZ₂, —OCZ₃, —OCH₂Z, —(C₃-C₅)cycloalkyl;
xii) R² is H; and/or
xiii) R⁷ is H; and/or
xiv) R¹¹ is H; and/or
xv) R³ is H; and/or
xvi) R⁴ is H; and/or
xvii) R⁵ is H.
xviii) R⁹ is OCH₃ and R¹⁰ is H or R₁₀ is OCH₃ and/or
xix) R⁶ is H; and/or
xx) R¹ is —O(C₁-C₆))alkyl; and/or,
xxi) R¹⁰ is H or —OCH₃; and/or
xxii) R¹ is —O(C₄-C₆))alkyl; and/or
xxiii) R⁹ is —OCH₃ and R¹⁰=H or —OCH₃; and/or
xxiv) R¹⁰ is H.

In respect to the compounds according to formula (II), the invention further comprises the following embodiment(s):
i) R¹ is selected from the group consisting of —O(C₁-C₄)alkyl, —O(C₅-C₁₀)heteroaryl, —O(C₃-C₁₀)aryl, —O(CH₂)ᵤ(C₃-C₁₀)aryl, preferably —Obutyl;
wherein a) (C₅-C₁₀)heteroaryl is preferably selected from the group consisting of

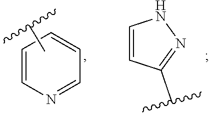

and/or
b) (C₃-C₁₀)aryl is preferably phenyl, optionally substituted with halogen and/or (C₁-C₃)alkyl; and/or
ii) R² is selected from the group consisting of hydrogen, halogen, preferably hydrogen;
wherein halogen is preferably Cl or F;
c) R³ is selected from the group consisting of hydrogen, halogen, preferably hydrogen;
wherein halogen is preferably Cl or F; and/or
iii) R⁴ is selected from the group consisting of hydrogen, halogen, preferably hydrogen; wherein halogen is preferably Cl or F; and/or
iv) R⁵ is selected from the group consisting of hydrogen, halogen, preferably hydrogen;
wherein halogen is preferably Cl or F; and/or
v) R⁶ is H, —(C₁-C₁₀)alkyl, and —(CH₂)₁₋₅cyclopropyl; and/or
vi) R⁷ is H; and/or
vii) R⁸ is H; and/or
viii) R⁹ is H, —O(C₁-C₄)alkyl, —OCH₂CF₃, —O(CH₂)cyclopropyl, —OCF₃, —OCHF₂, or, —O(C₂-C₁₀)alkenyl, —O(CH₂)₃C≡CH; and/or
ix) R¹⁰ is H, halogen, or —O(C₁-C₂)alkyl; and/or
x) wherein two adjacent rests of R⁹ and R¹⁰ form a ring, attached to the underlying aromatic ring of formula (II) according to

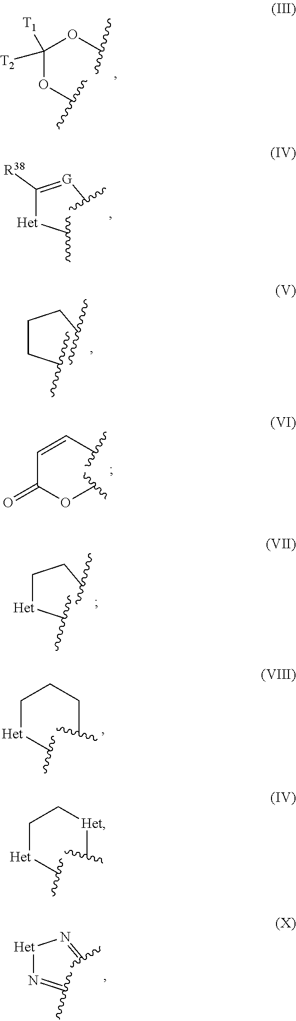

wherein T¹ and T² are independently selected from the group consisting of H, -methyl and F; wherein each hydrogen in formula (III) to (IX) is optionally substituted with methyl;
Het is O;
G is selected from CH;
R³⁸ is independently selected from the group consisting of H, —(C₁-C₁₀)alkyl;
xi) R¹¹ is H or —OCH₃; and/or
xii) R is H, C₁-C₆ alkyl, or benzyl, most preferably H; and/or
xiii) J₁ to J₄ are C or N.

A selection of compounds within the scope of the present invention is listed in the following Table:

TABLE 1

| | |
|---|---|
| 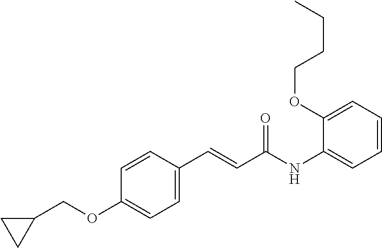 [SMILES: CCCCOc1ccccc1NC(=O)/C=C/c1ccc(OCC2CC2)cc1] | 1 |
| 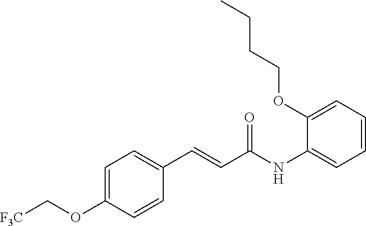 [SMILES: CCCCOc1ccccc1NC(=O)/C=C/c1ccc(OCC(F)(F)F)cc1] | 2 |
| 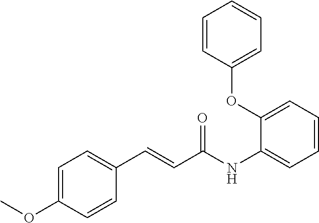 [SMILES: COc1ccc(/C=C/C(=O)Nc2ccccc2Oc2ccccc2)cc1] | 3 |
| 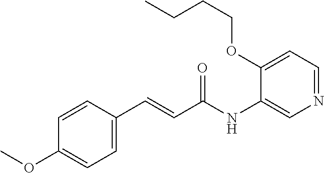 [SMILES: CCCCOc1ccncc1NC(=O)/C=C/c1ccc(OC)cc1] | 4 |
| 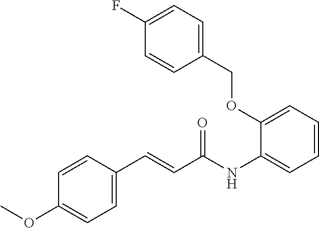 [SMILES: COc1ccc(/C=C/C(=O)Nc2ccccc2OCc2ccc(F)cc2)cc1] | 5 |
| 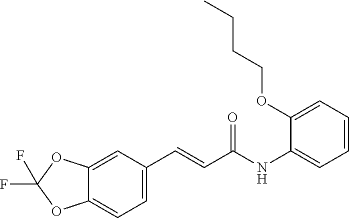 [SMILES: CCCCOc1ccccc1NC(=O)/C=C/c1ccc2c(c1)OC(F)(F)O2] | 6 |
| 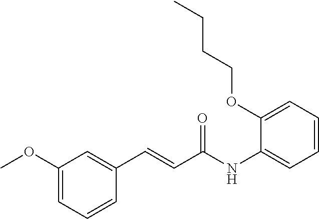 [SMILES: CCCCOc1ccccc1NC(=O)/C=C/c1cccc(OC)c1] | 7 |
| 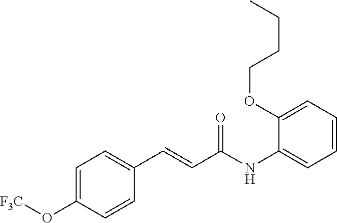 [SMILES: CCCCOc1ccccc1NC(=O)/C=C/c1ccc(OC(F)(F)F)cc1] | 8 |
| 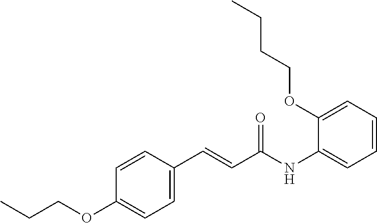 [SMILES: CCCCOc1ccccc1NC(=O)/C=C/c1ccc(OCCC)cc1] | 9 |
| 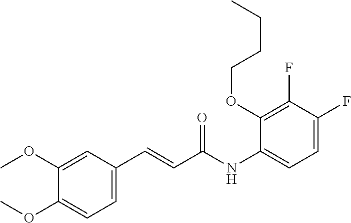 [SMILES: CCCCOc1c(NC(=O)/C=C/c2ccc(OC)c(OC)c2)ccc(F)c1F] | 10 |
| 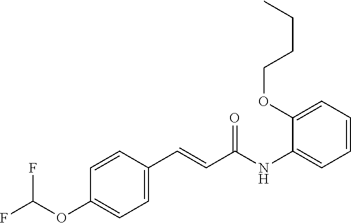 [SMILES: CCCCOc1ccccc1NC(=O)/C=C/c1ccc(OC(F)F)cc1] | 11 |
| 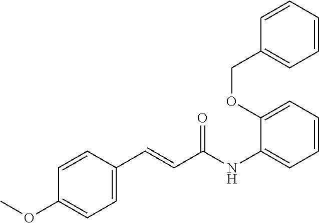 [SMILES: COc1ccc(/C=C/C(=O)Nc2ccccc2OCc2ccccc2)cc1] | 12 |
| 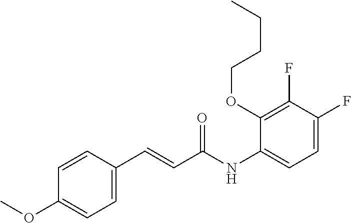 [SMILES: CCCCOc1c(NC(=O)/C=C/c2ccc(OC)cc2)ccc(F)c1F] | 13 |
| 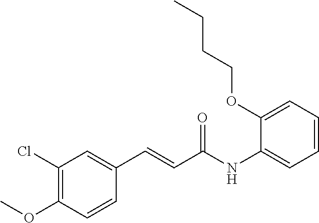 [SMILES: CCCCOc1ccccc1NC(=O)/C=C/c1ccc(OC)c(Cl)c1] | 14 |
| 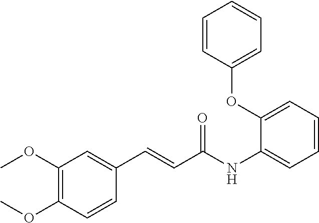 [SMILES: COc1ccc(/C=C/C(=O)Nc2ccccc2Oc2ccccc2)cc1OC] | 15 |
| 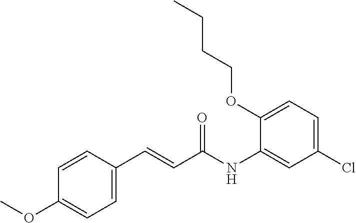 [SMILES: CCCCOc1ccc(Cl)cc1NC(=O)/C=C/c1ccc(OC)cc1] | 16 |
| 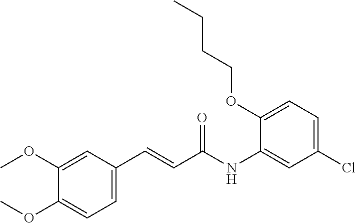 [SMILES: CCCCOc1ccc(Cl)cc1NC(=O)/C=C/c1ccc(OC)c(OC)c1] | 17 |
| 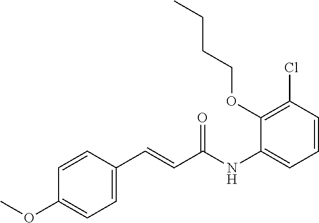 [SMILES: CCCCOc1c(Cl)cccc1NC(=O)/C=C/c1ccc(OC)cc1] | 18 |
| 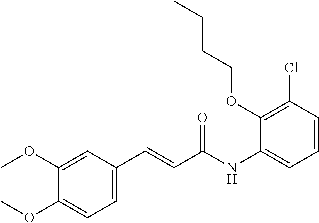 [SMILES: CCCCOc1c(Cl)cccc1NC(=O)/C=C/c1ccc(OC)c(OC)c1] | 19 |
| 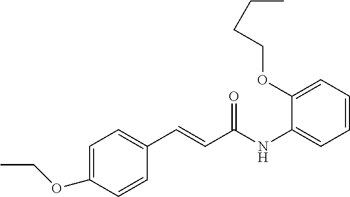 [SMILES: CCCCOc1ccccc1NC(=O)/C=C/c1ccc(OCC)cc1] | 20 |
| 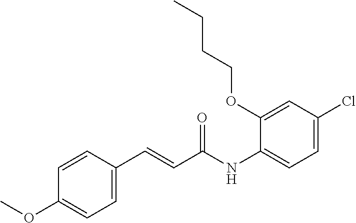 [SMILES: CCCCOc1cc(Cl)ccc1NC(=O)/C=C/c1ccc(OC)cc1] | 21 |
| 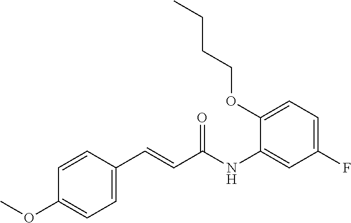 [SMILES: CCCCOc1ccc(F)cc1NC(=O)/C=C/c1ccc(OC)cc1] | 22 |
| 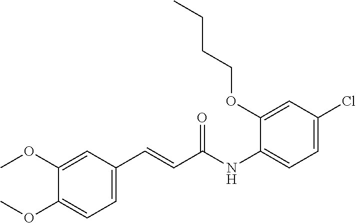 [SMILES: CCCCOc1cc(Cl)ccc1NC(=O)/C=C/c1ccc(OC)c(OC)c1] | 23 |
| 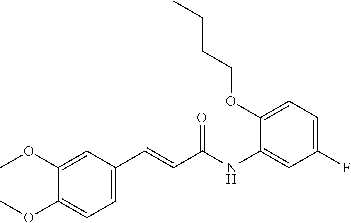 [SMILES: CCCCOc1ccc(F)cc1NC(=O)/C=C/c1ccc(OC)c(OC)c1] | 24 |
| 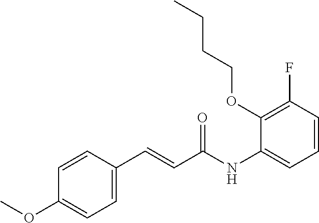 [SMILES: CCCCOc1c(F)cccc1NC(=O)/C=C/c1ccc(OC)cc1] | 25 |
| 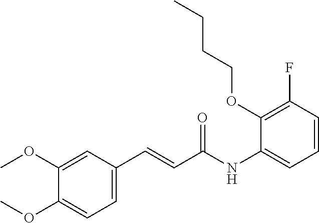 [SMILES: CCCCOc1c(F)cccc1NC(=O)/C=C/c1ccc(OC)c(OC)c1] | 26 |
| 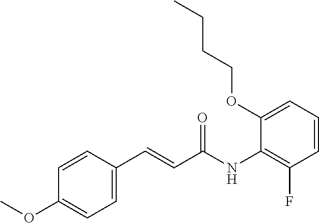 [SMILES: CCCCOc1cccc(F)c1NC(=O)/C=C/c1ccc(OC)cc1] | 27 |
| 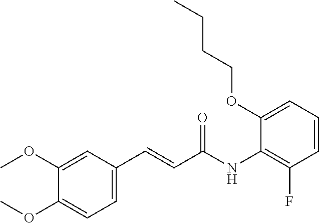 [SMILES: CCCCOc1cccc(F)c1NC(=O)/C=C/c1ccc(OC)c(OC)c1] | 28 |
| 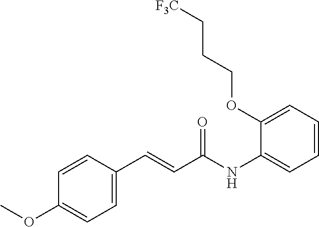 [SMILES: COc1ccc(/C=C/C(=O)Nc2ccccc2OCCCC(F)(F)F)cc1] | 29 |
| 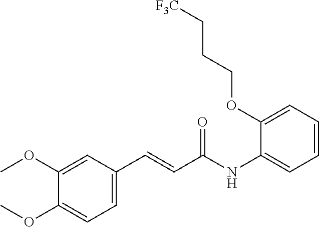 [SMILES: COc1ccc(/C=C/C(=O)Nc2ccccc2OCCCC(F)(F)F)cc1OC] | 30 |
| 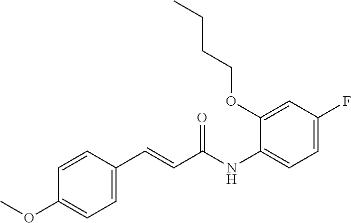 [SMILES: CCCCOc1cc(F)ccc1NC(=O)/C=C/c1ccc(OC)cc1] | 31 |
| 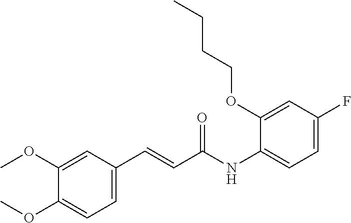 [SMILES: CCCCOc1cc(F)ccc1NC(=O)/C=C/c1ccc(OC)c(OC)c1] | 32 |
| 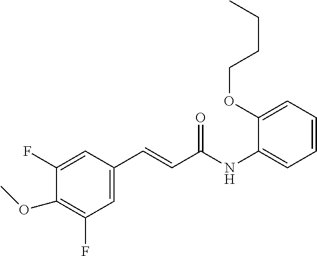 [SMILES: CCCCOc1ccccc1NC(=O)/C=C/c1cc(F)c(OC)c(F)c1] | 33 |
| 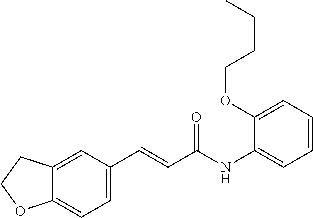 [SMILES: CCCCOc1ccccc1NC(=O)/C=C/c1ccc2c(c1)CCO2] | 34 |
| 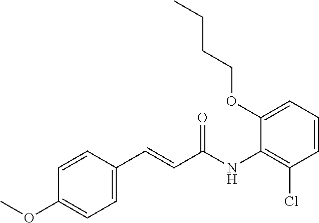 [SMILES: CCCCOc1cccc(Cl)c1NC(=O)/C=C/c1ccc(OC)cc1] | 35 |
| 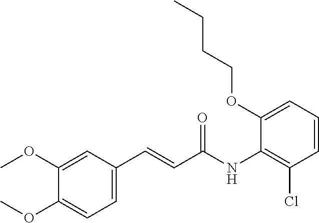 [SMILES: CCCCOc1cccc(Cl)c1NC(=O)/C=C/c1ccc(OC)c(OC)c1] | 36 |
| 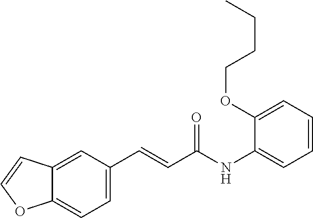 [SMILES: CCCCOc1ccccc1NC(=O)/C=C/c1ccc2occc2c1] | 37 |
| 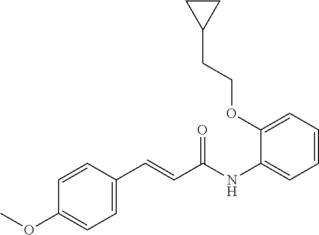 [SMILES: COc1ccc(/C=C/C(=O)Nc2ccccc2OCCC2CC2)cc1] | 38 |
| 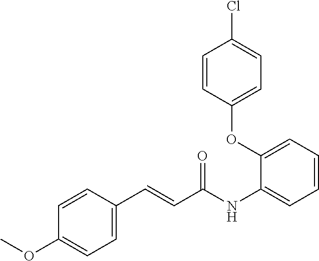 [SMILES: COc1ccc(/C=C/C(=O)Nc2ccccc2Oc2ccc(Cl)cc2)cc1] | 39 |
| 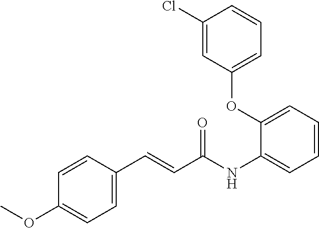 [SMILES: COc1ccc(/C=C/C(=O)Nc2ccccc2Oc2cccc(Cl)c2)cc1] | 40 |
| 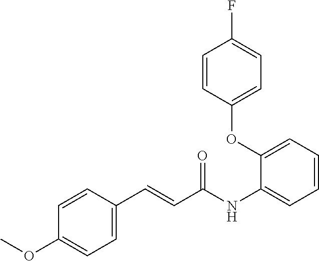 [SMILES: COc1ccc(/C=C/C(=O)Nc2ccccc2Oc2ccc(F)cc2)cc1] | 41 |
| 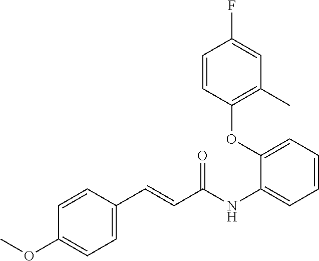 [SMILES: COc1ccc(/C=C/C(=O)Nc2ccccc2Oc2ccc(F)cc2C)cc1] | 42 |
| 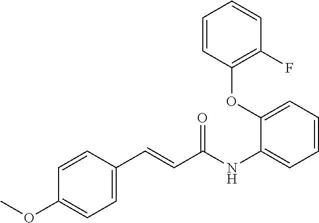 [SMILES: COc1ccc(/C=C/C(=O)Nc2ccccc2Oc2ccccc2F)cc1] | 43 |
| 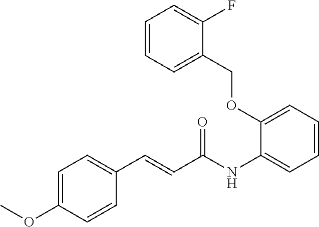 [SMILES: COc1ccc(/C=C/C(=O)Nc2ccccc2OCc2ccccc2F)cc1] | 44 |
| 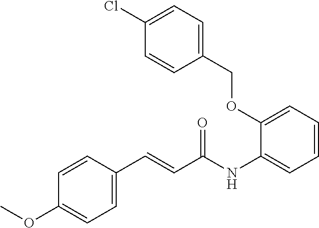 [SMILES: COc1ccc(/C=C/C(=O)Nc2ccccc2OCc2ccc(Cl)cc2)cc1] | 45 |
| 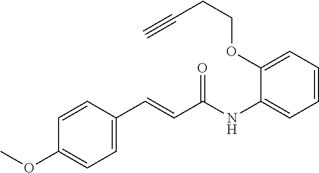 [SMILES: C#CCCOc1ccccc1NC(=O)/C=C/c1ccc(OC)cc1] | 46 |
| 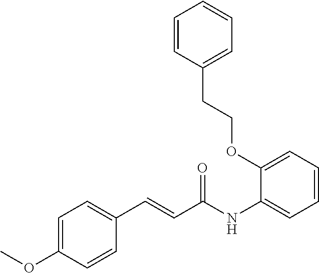 [SMILES: COc1ccc(/C=C/C(=O)Nc2ccccc2OCCc2ccccc2)cc1] | 47 |
| 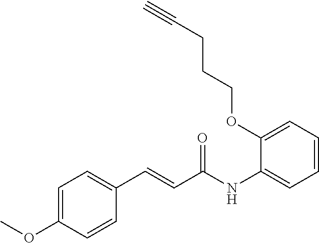 [SMILES: C#CCCCOc1ccccc1NC(=O)/C=C/c1ccc(OC)cc1] | 48 |
| 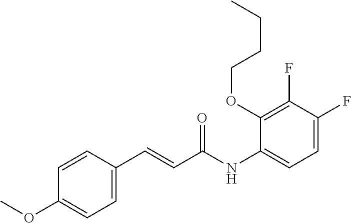 [SMILES: CCCCOc1c(NC(=O)/C=C/c2ccc(OC)cc2)ccc(F)c1F] | 49 |
| 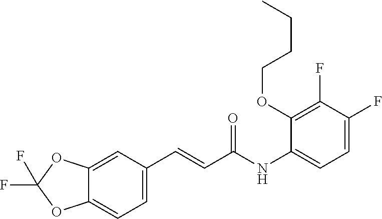 [SMILES: CCCCOc1c(NC(=O)/C=C/c2ccc3c(c2)OC(F)(F)O3)ccc(F)c1F] | 50 |
| 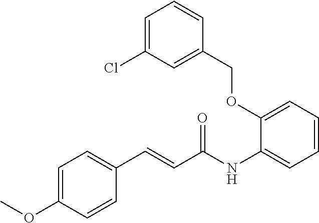 [SMILES: COc1ccc(/C=C/C(=O)Nc2ccccc2OCc2cccc(Cl)c2)cc1] | 51 |
| 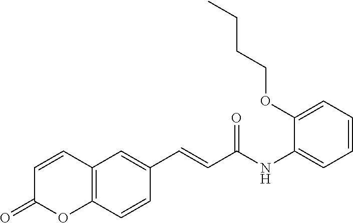 [SMILES: CCCCOc1ccccc1NC(=O)/C=C/c1ccc2oc(=O)ccc2c1] | 52 |
| 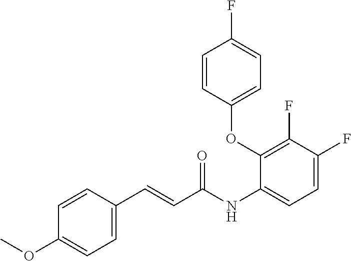 [SMILES: COc1ccc(/C=C/C(=O)Nc2ccc(F)c(F)c2Oc2ccc(F)cc2)cc1] | 53 |
| 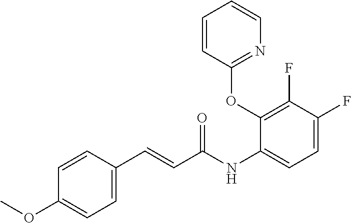 [SMILES: COc1ccc(/C=C/C(=O)Nc2ccc(F)c(F)c2Oc2ccccn2)cc1] | 54 |
| 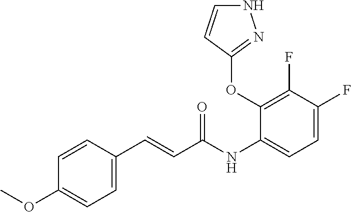 [SMILES: COc1ccc(/C=C/C(=O)Nc2ccc(F)c(F)c2Oc2cc[nH]n2)cc1] | 55 |
| 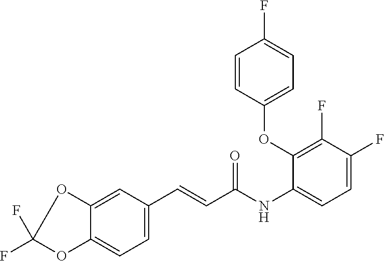 [SMILES: O=C(/C=C/c1ccc2c(c1)OC(F)(F)O2)Nc1ccc(F)c(F)c1Oc1ccc(F)cc1] | 56 |
| 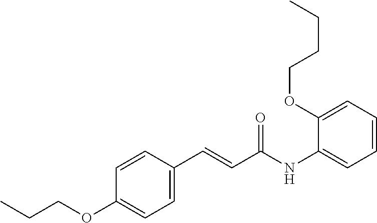 [SMILES: CCCCOc1ccccc1NC(=O)/C=C/c1ccc(OCCC)cc1] | 57 |
| 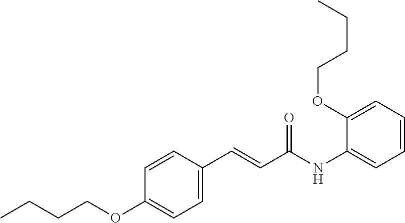 [SMILES: CCCCOc1ccc(/C=C/C(=O)Nc2ccccc2OCCCC)cc1] | 58 |
| 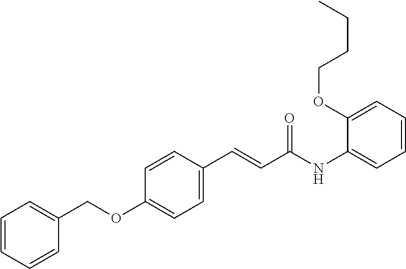 [SMILES: CCCCOc1ccccc1NC(=O)/C=C/c1ccc(OCc2ccccc2)cc1] | 59 |
| 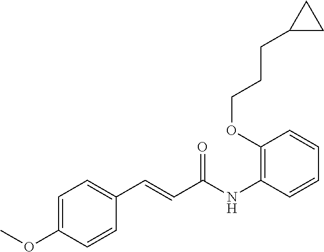 [SMILES: COc1ccc(/C=C/C(=O)Nc2ccccc2OCCCC2CC2)cc1] | 60 |
| 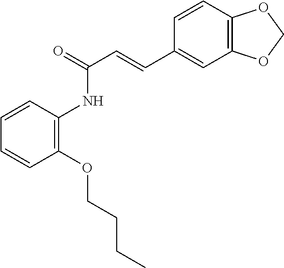 [SMILES: CCCCOc1ccccc1NC(=O)/C=C/c1ccc2c(c1)OCO2] | 61 |
| 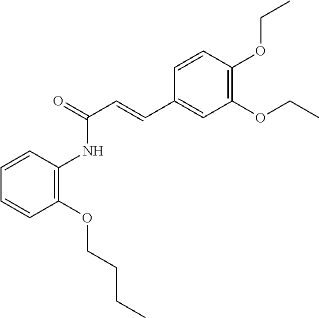 [SMILES: CCCCOc1ccccc1NC(=O)/C=C/c1ccc(OCC)c(OCC)c1] | 62 |
| 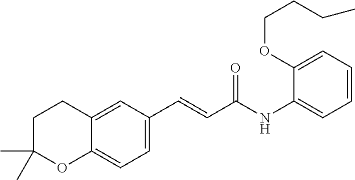 [SMILES: CCCCOc1ccccc1NC(=O)/C=C/c1ccc2c(c1)CCC(C)(C)O2] | 63 |
| 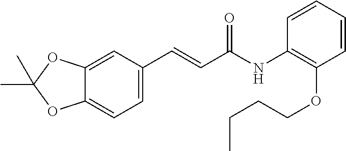 [SMILES: CCCCOc1ccccc1NC(=O)/C=C/c1ccc2c(c1)OC(C)(C)O2] | 64 |
| 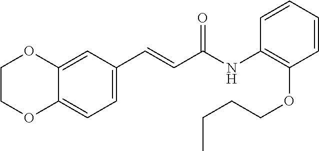 [SMILES: CCCCOc1ccccc1NC(=O)/C=C/c1ccc2c(c1)OCCO2] | 65 |
| 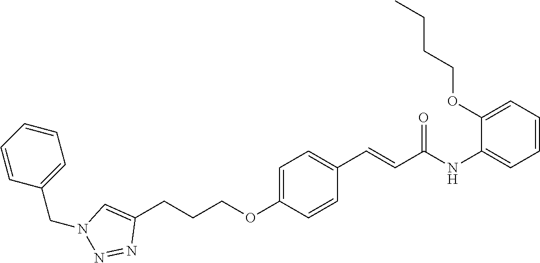 [SMILES: CCCCOc1ccccc1NC(=O)/C=C/c1ccc(OCCCc2cn(Cc3ccccc3)nn2)cc1] | 66 |
| 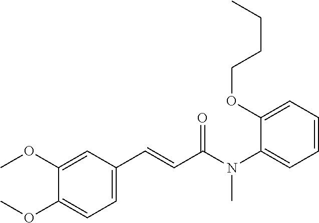 [SMILES: CCCCOc1ccccc1N(C)C(=O)/C=C/c1ccc(OC)c(OC)c1] | 67 |
| 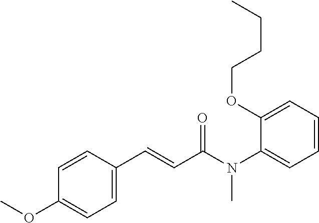 [SMILES: CCCCOc1ccccc1N(C)C(=O)/C=C/c1ccc(OC)cc1] | 68 |
| 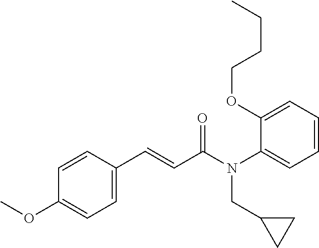 [SMILES: CCCCOc1ccccc1N(CC1CC1)C(=O)/C=C/c1ccc(OC)cc1] | 69 |
| 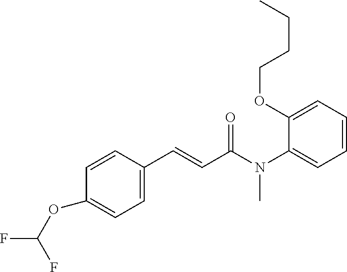 [SMILES: CCCCOc1ccccc1N(C)C(=O)/C=C/c1ccc(OC(F)F)cc1] | 70 |
| 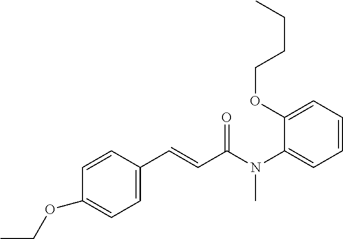 [SMILES: CCCCOc1ccccc1N(C)C(=O)/C=C/c1ccc(OCC)cc1] | 71 |
| 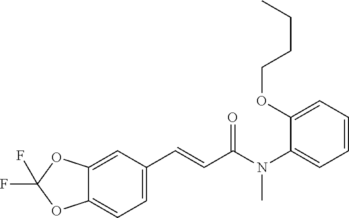 [SMILES: CCCCOc1ccccc1N(C)C(=O)/C=C/c1ccc2c(c1)OC(F)(F)O2] | 72 |
| 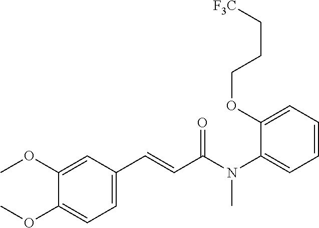 [SMILES: COc1ccc(/C=C/C(=O)N(C)c2ccccc2OCCCC(F)(F)F)cc1OC] | 73 |
| 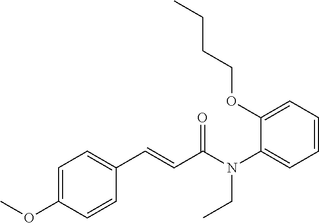 [SMILES: CCCCOc1ccccc1N(CC)C(=O)/C=C/c1ccc(OC)cc1] | 74 |
| 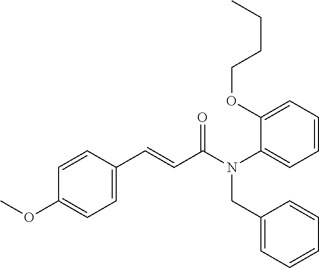 [SMILES: CCCCOc1ccccc1N(Cc1ccccc1)C(=O)/C=C/c1ccc(OC)cc1] | 75 |
| 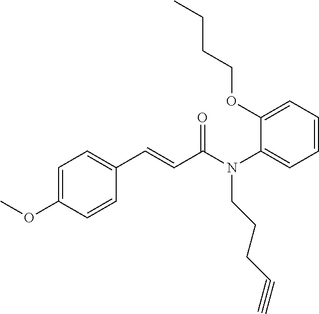 [SMILES: C#CCCCN(C(=O)/C=C/c1ccc(OC)cc1)c1ccccc1OCCCC] | 76 |
| 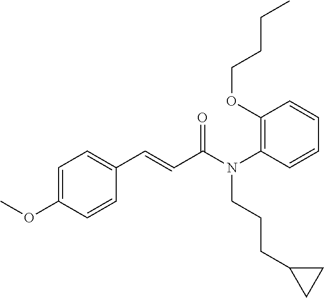 [SMILES: CCCCOc1ccccc1N(CCCC1CC1)C(=O)/C=C/c1ccc(OC)cc1] | 77 |
| 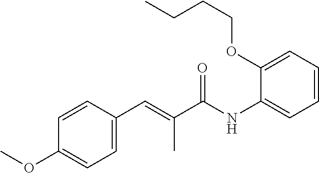 [SMILES: CCCCOc1ccccc1NC(=O)/C(C)=C/c1ccc(OC)cc1] | 78 |
| 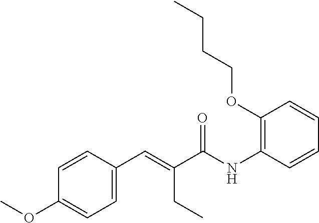 [SMILES: CCCCOc1ccccc1NC(=O)/C(=C/c1ccc(OC)cc1)CC] | 79 |
| 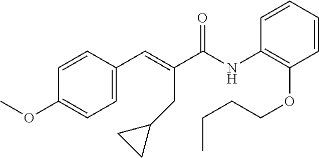 [SMILES: CCCCOc1ccccc1NC(=O)/C(=C/c1ccc(OC)cc1)CC1CC1] | 80 |
| 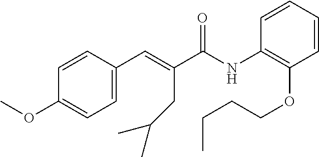 [SMILES: CCCCOc1ccccc1NC(=O)/C(=C/c1ccc(OC)cc1)CC(C)C] | 81 |
| 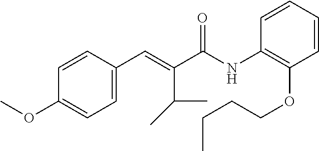 [SMILES: CCCCOc1ccccc1NC(=O)/C(=C/c1ccc(OC)cc1)C(C)C] | 82 |
| 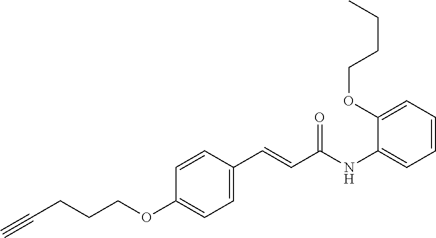 [SMILES: C#CCCCOc1ccc(/C=C/C(=O)Nc2ccccc2OCCCC)cc1] | 83 |
| 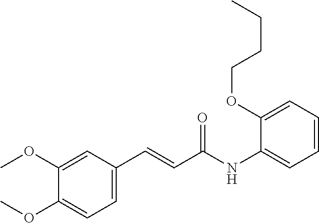 [SMILES: CCCCOc1ccccc1NC(=O)/C=C/c1ccc(OC)c(OC)c1] | 84 |
| 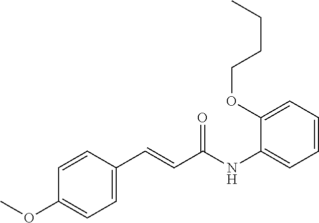 [SMILES: CCCCOc1ccccc1NC(=O)/C=C/c1ccc(OC)cc1] | 85 |
| 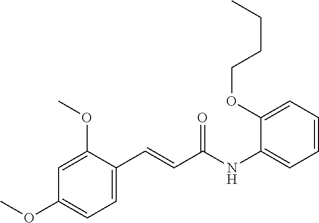 [SMILES: CCCCOc1ccccc1NC(=O)/C=C/c1ccc(OC)cc1OC] | 86 |
| 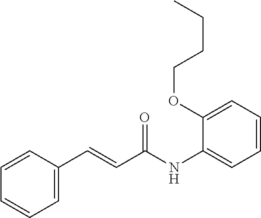 [SMILES: CCCCOc1ccccc1NC(=O)/C=C/c1ccccc1] | 87 |
| 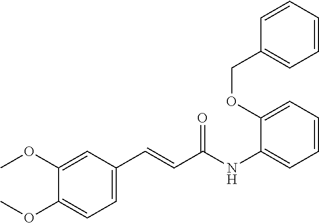 [SMILES: COc1ccc(/C=C/C(=O)Nc2ccccc2OCc2ccccc2)cc1OC] | 88 |
| 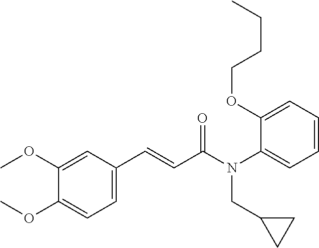 [SMILES: CCCCOc1ccccc1N(CC1CC1)C(=O)/C=C/c1ccc(OC)c(OC)c1] | 89 |
| 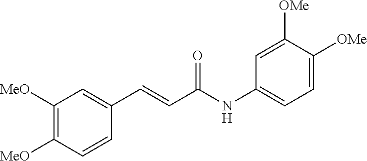 [SMILES: COc1ccc(/C=C/C(=O)Nc2ccc(OC)c(OC)c2)cc1OC] | 90 |
| 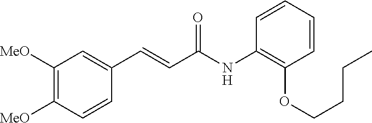 [SMILES: CCCCOc1ccccc1NC(=O)/C=C/c1ccc(OC)c(OC)c1] | 91 |
| 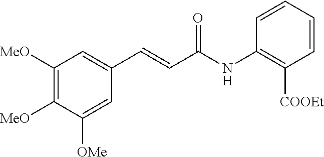 [SMILES: CCOC(=O)c1ccccc1NC(=O)/C=C/c1cc(OC)c(OC)c(OC)c1] | 92 |
| 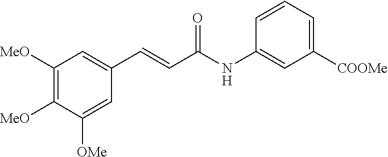 [SMILES: COC(=O)c1cccc(NC(=O)/C=C/c2cc(OC)c(OC)c(OC)c2)c1] | 93 |
| 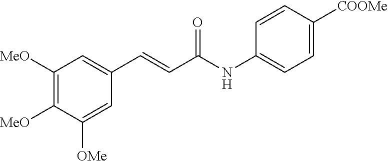 [SMILES: COC(=O)c1ccc(NC(=O)/C=C/c2cc(OC)c(OC)c(OC)c2)cc1] | 94 |
| 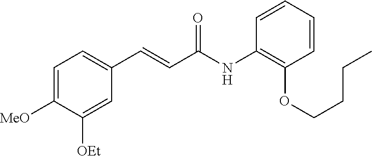 [SMILES: CCCCOc1ccccc1NC(=O)/C=C/c1ccc(OC)c(OCC)c1] | 95 |
| 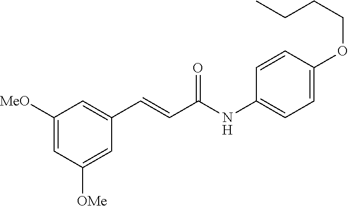 [SMILES: CCCCOc1ccc(NC(=O)/C=C/c2cc(OC)cc(OC)c2)cc1] | 96 |
| 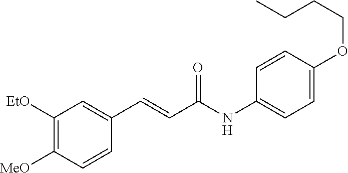 [SMILES: CCCCOc1ccc(NC(=O)/C=C/c2ccc(OC)c(OCC)c2)cc1] | 97 |
| 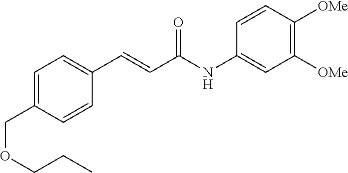 [SMILES: CCCOCc1ccc(/C=C/C(=O)Nc2ccc(OC)c(OC)c2)cc1] | 98 |
| 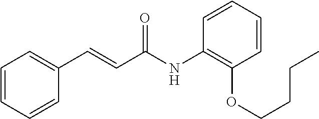 [SMILES: CCCCOc1ccccc1NC(=O)/C=C/c1ccccc1] | 99 |
| 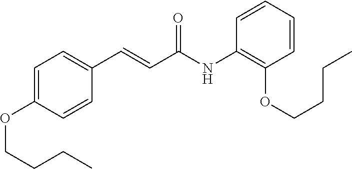 [SMILES: CCCCOc1ccc(/C=C/C(=O)Nc2ccccc2OCCCC)cc1] | 100 |
| 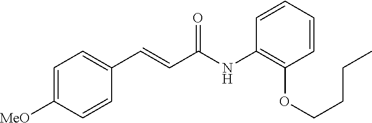 [SMILES: CCCCOc1ccccc1NC(=O)/C=C/c1ccc(OC)cc1] | 101 |
| 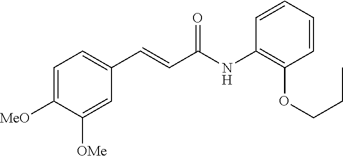 [SMILES: CCCOc1ccccc1NC(=O)/C=C/c1ccc(OC)c(OC)c1] | 102 |
| 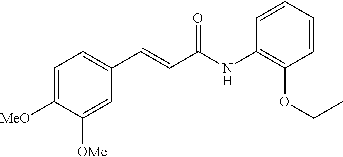 [SMILES: CCOc1ccccc1NC(=O)/C=C/c1ccc(OC)c(OC)c1] | 103 |
| 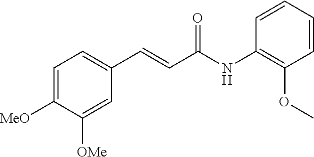 [SMILES: COc1ccccc1NC(=O)/C=C/c1ccc(OC)c(OC)c1] | 104 |
| 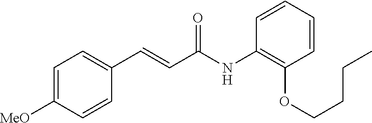 [SMILES: CCCCOc1ccccc1NC(=O)/C=C/c1ccc(OC)cc1] | 105 |
| 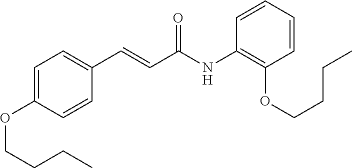 [SMILES: CCCCOc1ccc(/C=C/C(=O)Nc2ccccc2OCCCC)cc1] | 106 |
| 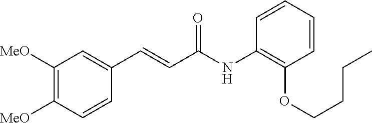 [SMILES: CCCCOc1ccccc1NC(=O)/C=C/c1ccc(OC)c(OC)c1] | 107 |
| 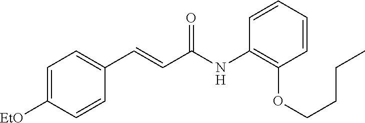 [SMILES: CCCCOc1ccccc1NC(=O)/C=C/c1ccc(OCC)cc1] | 108 |
| 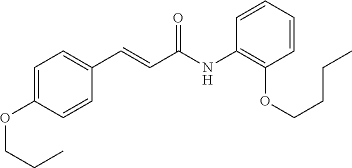 [SMILES: CCCCOc1ccccc1NC(=O)/C=C/c1ccc(OCCC)cc1] | 109 |
| 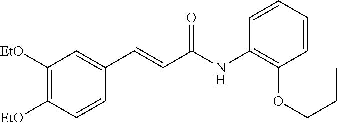 [SMILES: CCCOc1ccccc1NC(=O)/C=C/c1ccc(OCC)c(OCC)c1] | 110 |
| 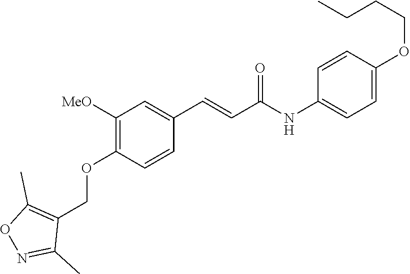 [SMILES: CCCCOc1ccc(NC(=O)/C=C/c2ccc(OCc3c(C)noc3C)c(OC)c2)cc1] | 111 |
| 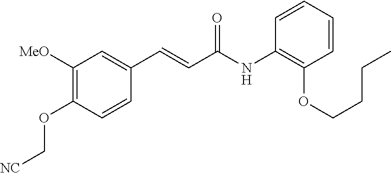 [SMILES: CCCCOc1ccccc1NC(=O)/C=C/c1ccc(OCC#N)c(OC)c1] | 112 |
| 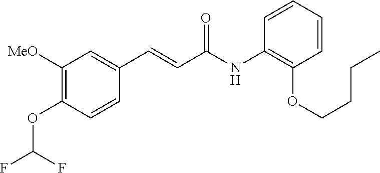 [SMILES: CCCCOc1ccccc1NC(=O)/C=C/c1ccc(OC(F)F)c(OC)c1] | 113 |
| 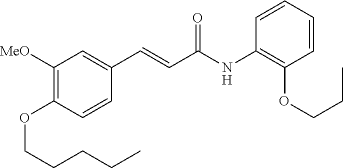 [SMILES: CCCCCOc1ccc(/C=C/C(=O)Nc2ccccc2OCCC)cc1OC] | 114 |
| 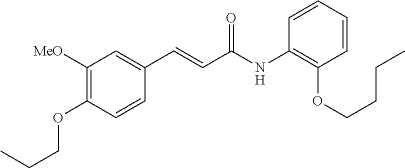 [SMILES: CCCCOc1ccccc1NC(=O)/C=C/c1ccc(OCCC)c(OC)c1] | 115 |
| 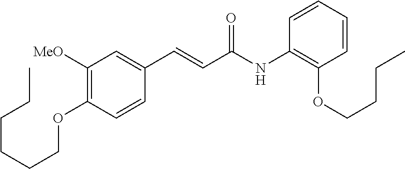 [SMILES: CCCCCCOc1ccc(/C=C/C(=O)Nc2ccccc2OCCCC)cc1OC] | 116 |
| 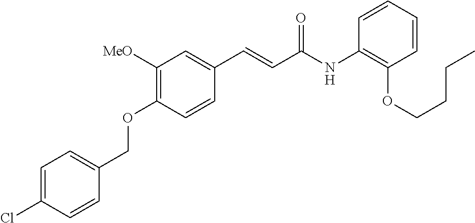 [SMILES: CCCCOc1ccccc1NC(=O)/C=C/c1ccc(OCc2ccc(Cl)cc2)c(OC)c1] | 117 |
| 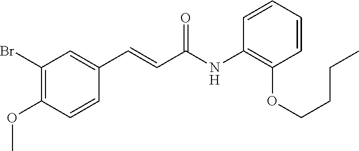 [SMILES: CCCCOc1ccccc1NC(=O)/C=C/c1ccc(OC)c(Br)c1] | 118 |
| 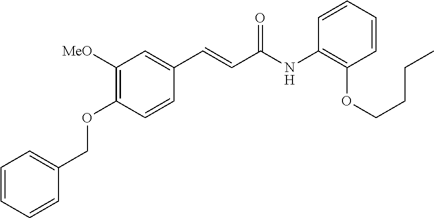 [SMILES: CCCCOc1ccccc1NC(=O)/C=C/c1ccc(OCc2ccccc2)c(OC)c1] | 119 |
| 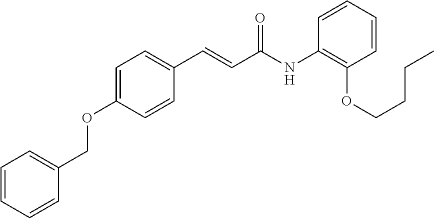 [SMILES: CCCCOc1ccccc1NC(=O)/C=C/c1ccc(OCc2ccccc2)cc1] | 120 |
| 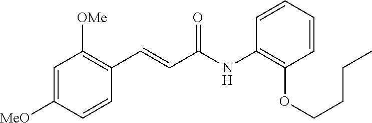 [SMILES: CCCCOc1ccccc1NC(=O)/C=C/c1ccc(OC)cc1OC] | 121 |
| 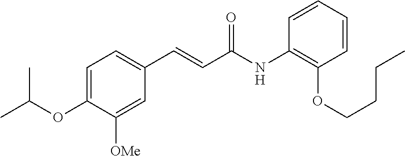 [SMILES: CCCCOc1ccccc1NC(=O)/C=C/c1ccc(OC(C)C)c(OC)c1] | 122 |
| 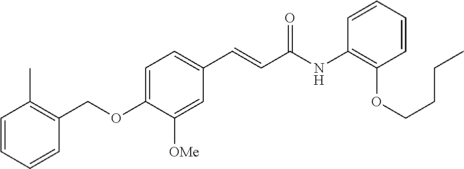 [SMILES: CCCCOc1ccccc1NC(=O)/C=C/c1ccc(OCc2ccccc2C)c(OC)c1] | 123 |
| 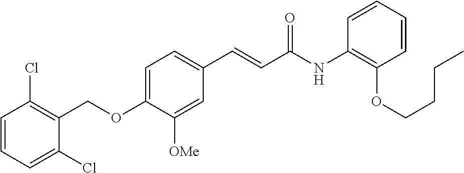 [SMILES: CCCCOc1ccccc1NC(=O)/C=C/c1ccc(OCc2c(Cl)cccc2Cl)c(OC)c1] | 124 |
| 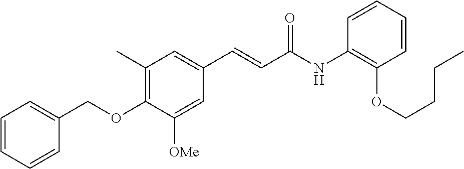 [SMILES: CCCCOc1ccccc1NC(=O)/C=C/c1cc(C)c(OCc2ccccc2)c(OC)c1] | 125 |
| 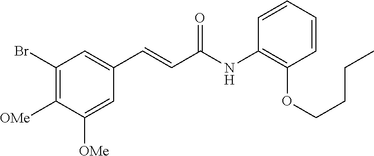 [SMILES: CCCCOc1ccccc1NC(=O)/C=C/c1cc(Br)c(OC)c(OC)c1] | 126 |
| 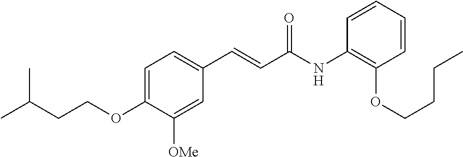 [SMILES: CCCCOc1ccccc1NC(=O)/C=C/c1ccc(OCCC(C)C)c(OC)c1] | 127 |
| 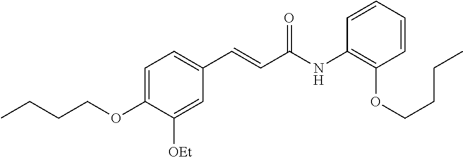 [SMILES: CCCCOc1ccccc1NC(=O)/C=C/c1ccc(OCCCC)c(OCC)c1] | 128 |
| 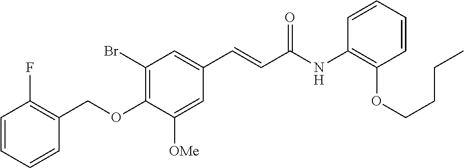 [SMILES: CCCCOc1ccccc1NC(=O)/C=C/c1cc(Br)c(OCc2ccccc2F)c(OC)c1] | 129 |
| 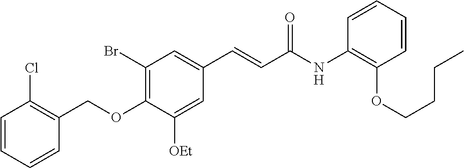 [SMILES: CCCCOc1ccccc1NC(=O)/C=C/c1cc(Br)c(OCc2ccccc2Cl)c(OCC)c1] | 130 |
| 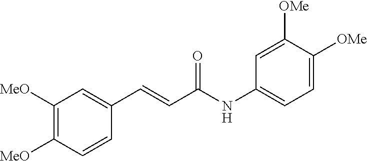 [SMILES: COc1ccc(/C=C/C(=O)Nc2ccc(OC)c(OC)c2)cc1OC] | 131 |
| 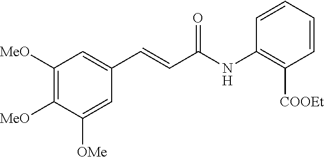 [SMILES: CCOC(=O)c1ccccc1NC(=O)/C=C/c1cc(OC)c(OC)c(OC)c1] | 132 |
| 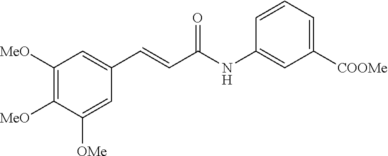 [SMILES: COC(=O)c1cccc(NC(=O)/C=C/c2cc(OC)c(OC)c(OC)c2)c1] | 133 |
| 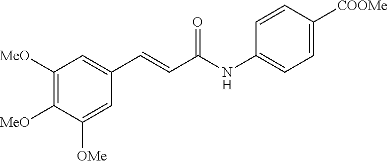 [SMILES: COC(=O)c1ccc(NC(=O)/C=C/c2cc(OC)c(OC)c(OC)c2)cc1] | 134 |
| 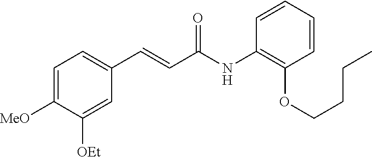 [SMILES: CCCCOc1ccccc1NC(=O)/C=C/c1ccc(OC)c(OCC)c1] | 135 |
| 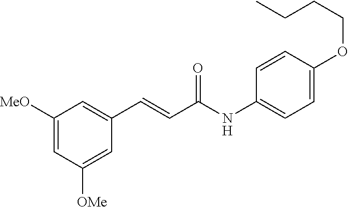 [SMILES: CCCCOc1ccc(NC(=O)/C=C/c2cc(OC)cc(OC)c2)cc1] | 136 |
| 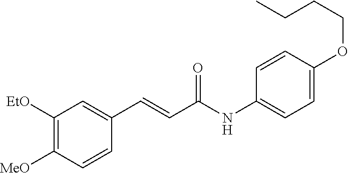 [SMILES: CCCCOc1ccc(NC(=O)/C=C/c2ccc(OC)c(OCC)c2)cc1] | 137 |
| 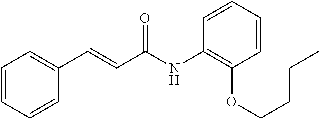 [SMILES: CCCCOc1ccccc1NC(=O)/C=C/c1ccccc1] | 138 |
| 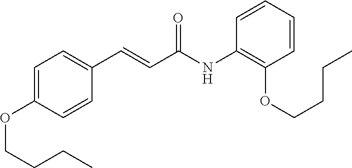 [SMILES: CCCCOc1ccc(/C=C/C(=O)Nc2ccccc2OCCCC)cc1] | 139 |
| 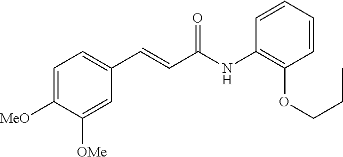 [SMILES: CCCOc1ccccc1NC(=O)/C=C/c1ccc(OC)c(OC)c1] | 139 |
| 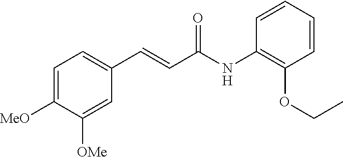 [SMILES: CCOc1ccccc1NC(=O)/C=C/c1ccc(OC)c(OC)c1] | 140 |
| 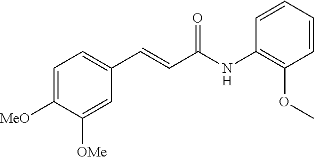 [SMILES: COc1ccccc1NC(=O)/C=C/c1ccc(OC)c(OC)c1] | 141 |
| 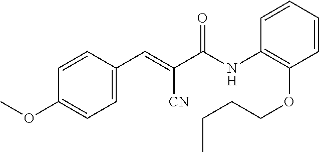 [SMILES: CCCCOc1ccccc1NC(=O)/C(C#N)=C/c1ccc(OC)cc1] | 142 |
| 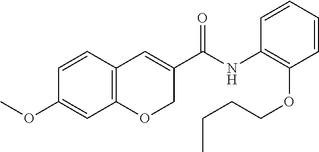 [SMILES: CCCCOc1ccccc1NC(=O)C1=Cc2ccc(OC)cc2OC1] | 143 |
| 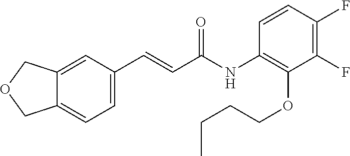 [SMILES: CCCCOc1c(NC(=O)/C=C/c2ccc3c(c2)COC3)ccc(F)c1F] | 144 |
| 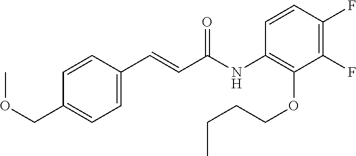 [SMILES: CCCCOc1c(NC(=O)/C=C/c2ccc(COC)cc2)ccc(F)c1F] | 145 |
| 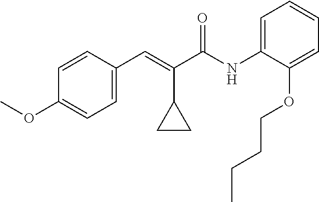 [SMILES: CCCCOc1ccccc1NC(=O)/C(=C/c1ccc(OC)cc1)C1CC1] | 146 |
| 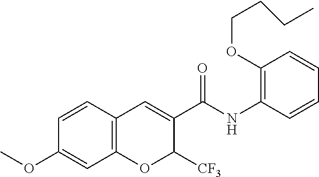 [SMILES: CCCCOc1ccccc1NC(=O)C1=Cc2ccc(OC)cc2OC1C(F)(F)F] | 147 |
| 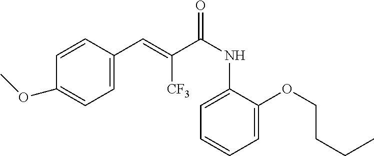 [SMILES: CCCCOc1ccccc1NC(=O)/C(=C/c1ccc(OC)cc1)C(F)(F)F] | 148 |
| 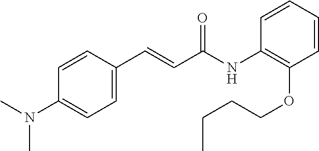 [SMILES: CCCCOc1ccccc1NC(=O)/C=C/c1ccc(N(C)C)cc1] | 149 |
| 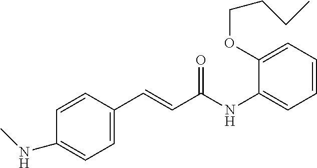 [SMILES: CCCCOc1ccccc1NC(=O)/C=C/c1ccc(NC)cc1] | 150 |
| 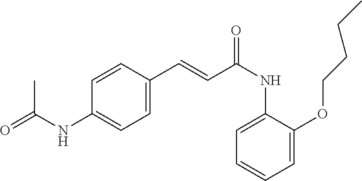 [SMILES: CCCCOc1ccccc1NC(=O)/C=C/c1ccc(NC(C)=O)cc1] | 151 |
| 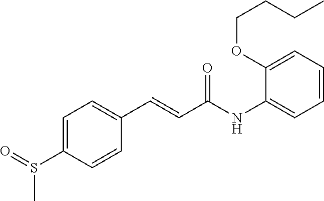 [SMILES: CCCCOc1ccccc1NC(=O)/C=C/c1ccc(S(C)=O)cc1] | 152 |
| 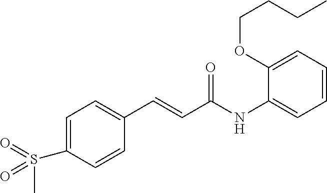 [SMILES: CCCCOc1ccccc1NC(=O)/C=C/c1ccc(S(C)(=O)=O)cc1] | 153 |
| 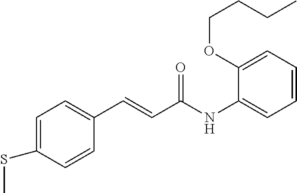 [SMILES: CCCCOc1ccccc1NC(=O)/C=C/c1ccc(SC)cc1] | 154 |
| 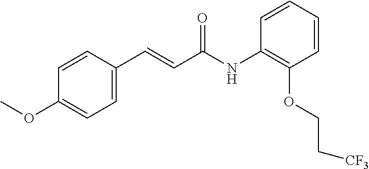 [SMILES: COc1ccc(/C=C/C(=O)Nc2ccccc2OCCC(F)(F)F)cc1] | 155 |
| 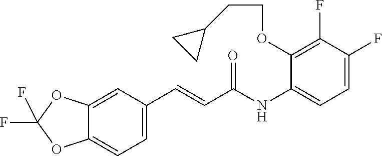 [SMILES: O=C(/C=C/c1ccc2c(c1)OC(F)(F)O2)Nc1ccc(F)c(F)c1OCCC1CC1] | 156 |
| 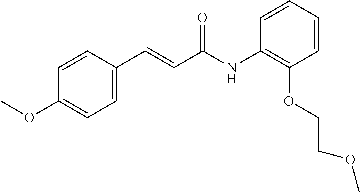 | 157 |
| 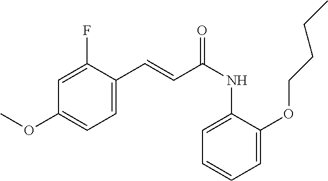 [SMILES: CCCCOc1ccccc1NC(=O)/C=C/c1ccc(OC)cc1F] | 158 |
| | 159 |
| | 160 |
| | 161 |
| | 162 |
| | 163 |
| | 164 |
| 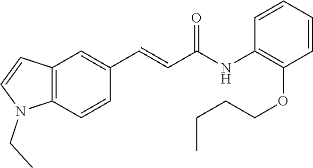 [SMILES: CCCCOc1ccccc1NC(=O)/C=C/c1ccc2c(ccn2CC)c1] | 165 |
| 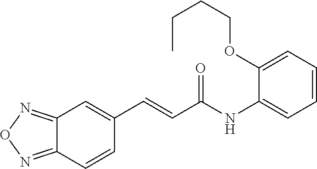 [SMILES: CCCCOc1ccccc1NC(=O)/C=C/c1ccc2nonc2c1] | 166 |
| 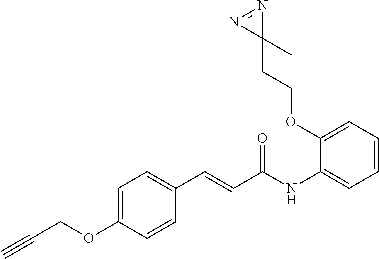 [SMILES: C#CCOc1ccc(/C=C/C(=O)Nc2ccccc2OCCC2(C)N=N2)cc1] | 167 |
| 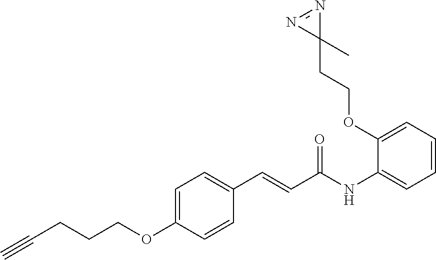 [SMILES: C#CCCCOc1ccc(/C=C/C(=O)Nc2ccccc2OCCC2(C)N=N2)cc1] | 168 |

The invention is further directed to the compound according to formula (I), (II) or according to table 1 for use in medicine.

The invention is further directed to the compound according to formula (I), (II) or according to table 1 for use in the treatment of fibrosis and neoplasia, preferably a fibrosis or neoplasia located in the heart, the lung, the renal tract, the liver, in the skin, in the pleura and retroperitoneum, more preferably the fibrosis is selected from pleural fibrosis, retroperitoneal fibrosis, atrial fibrillation, myocardial interstitial fibrosis, idiopathic pulmonary fibrosis (IPF), interstitial lung diseases, chronic kidney disease, non-alcoholic fat liver disease, skin scars, keloids, tumour-associated desmoplastic reaction.

The invention is further directed to the compound according to formula (I), (II) or according to table 1 use in the treatment of inflammatory diseases, such as arthrolithiasis, familiar mediterranean fever and pericarditis.

Compounds of the invention which contain a basic functionality may form salts with a variety of inorganic or organic acids. Exemplary inorganic and organic acids/bases as well as exemplary acid/base addition salts of the compounds of the present invention are given in the definition of “pharmaceutically acceptable salt” in the section “Pharmaceutical composition”, below. The compounds of the invention which contain an acidic functionality may form salts with a variety of inorganic or organic bases. The compounds of the invention which contain both basic and acidic functionalities may be converted into either base or acid addition salt. The neutral forms of the compounds of the invention may be regenerated by contacting the salt with a base or acid and isolating the parent compound in the conventional manner.

### Synthesis of Compounds of the Present Invention

Many compounds of the present invention are commercially available. Compounds which are not commercially available are in general obtainable as follows and specific examples for the preparation of compounds of the present invention are described in the example part.

As shown in Scheme 1, the compounds of the present invention may be synthesized by an amide formation between a respective aromatic acid and an aniline.

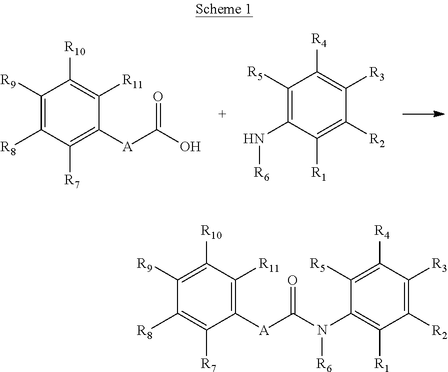

Several reagents and methods for amide formation from acids and amins are known.

Examplary Methods and/or Reagents are:
a) Carbodiimide based reagents for example such as N,N′-Dicyclohexylcarbodiimide (DCC), N,N′-Diisopropylcarbodiimid (DIC), 1-Ethyl-3-(3-dimethylaminopropyl)carbodiimid (EDC) optionally in combination with a weak organic base for example such as triethylamine, ethyldiisopropylamine;
b) benzotriazol-1-yloxytripyrrolidinophosphonium hexafluorophosphate optionally in combination with a weak organic base for example such as triethylamine, ethyldiisopropylamine;
c) converting in a first step the carboxylic acid with oxalylchloride, optionally in the presence of dimethylformamide, into the corresponding acid chloride, followed by a second step, converting the acid chloride with the aniline into the desired amide.

### Pharmaceutical Compositions

Moreover, the invention is directed to a pharmaceutical composition comprising the compound as described above and at least one carrier.

“Pharmaceutical composition” refers to one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

“Carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

A composition of the present invention can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978.

To administer a compound of the invention by certain routes of administration, it may be necessary to coat the compound with, or co-administer the compound with, a material to prevent its inactivation. For example, the compound may be administered to an individual in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7: 27 (1984)).

Pharmaceutically acceptable carriers include sterile aqueous solutions or dispersions and sterile powders for the extemporaneous preparation of sterile injectable solutions or dispersions. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration.

Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the individuals to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulphate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic/pharmaceutical formulations, compositions of the present invention include those suitable for enteral administration (such as oral or rectal) or parenteral administration (such as nasal, topical (including vaginal, buccal and sublingual)). The compositions may conveniently be presented in unit dosage form and may be prepared by any methods known in the art of pharmacy. The amount of active ingredient (in particular, the amount of a compound of the present invention) which can be combined with a carrier material to produce a pharmaceutical composition (such as a single dosage form) will vary depending upon the individual being treated, and the particular mode of administration. The amount of active ingredient which can be combined with a carrier material to produce a single dosage form will generally be that amount of the composition which produces a therapeutic effect.

Generally, out of 100% (for the pharmaceutical formulations/compositions), the amount of active ingredient (in particular, the amount of the compound of the present invention, optionally together with other therapeutically active agents, if present in the pharmaceutical formulations/compositions) will range from about 0.01% to about 99%, preferably from about 0.1% to about 70%, most preferably from about 1% to about 30%, wherein the reminder is preferably composed of the one or more pharmaceutically acceptable excipients.

The amount of active ingredient, e.g., a compound of the invention, in a unit dosage form and/or when administered to an individual or used in therapy, may range from about 0.1 mg to about 1000 mg (for example, from about 1 mg to about 500 mg, such as from about 10 mg to about 200 mg) per unit, administration or therapy. In certain embodiments, a suitable amount of such active ingredient may be calculated using the mass or body surface area of the individual, including amounts of between about 1 mg/Kg and 10 mg/Kg (such as between about 2 mg/Kg and 5 mg/Kg), or between about 1 mg/m² and about 400 mg/m² (such as between about 3 mg/M² and about 350 mg/m² or between about 10 mg/m² and about 200 mg/m2).

Compositions of the present invention which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The expressions “enteral administration” and “administered enterally” as used herein mean that the drug administered is taken up by the stomach and/or the intestine.

Examples of enteral administration include oral and rectal administration. The expressions “parenteral administration” and “administered parenterally” as used herein mean modes of administration other than enteral administration, usually by injection or topical application, and include, without limitation, intravenous, intramuscular, intraarterial, intrathecal, intracapsular, intraosseous, intraorbital, intracardiac, intradermal, intraperitoneal, transtracheal, subcutaneous, subcuticular, intraarticular, subcapsular, intracerebral, intracerebroventricular, subarachnoid, intraspinal, epidural and intrasternal administration (such as by injection and/or infusion) as well as topical administration (e.g., epicutaneous, inhalational, or through mucous membranes (such as buccal, sublingual or vaginal)).

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, pH buffering agents, and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art (cf., e.g., Remington, “The Science and Practice of Pharmacy” edited by Allen, Loyd V., Jr., 22^{nd} edition, Pharmaceutical Sciences, September 2012; Ansel et al., “Pharmaceutical Dosage Forms and Drug Delivery Systems”, 7^{th} edition, Lippincott Williams & Wilkins Publishers, 1999.).

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start with doses of the compounds of the invention employed in the pharmaceutical composition at levels lower than that required in order to achieve the desired therapeutic effect and gradually increase the dosage until the desired effect is achieved.

In general, a suitable daily dose of a composition of the invention will be that amount of the compound which is the lowest dose effective to produce a therapeutic effect. Such an effective dose will generally depend upon the factors described above. It is preferred that administration be oral, intravenous, intramuscular, intraperitoneal, or subcutaneous, preferably administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation/composition.

In one embodiment, the compounds or compositions of the invention may be administered by infusion, preferably slow continuous infusion over a long period, such as more than 24 hours, in order to reduce toxic side effects. The administration may also be performed by continuous infusion over a period of from 2 to 24 hours, such as of from 2 to 12 hours. Such regimen may be repeated one or more times as necessary, for example, after 6 months or 12 months.

In yet another embodiment, the compounds or compositions of the invention are administered by maintenance therapy, such as, e.g., once a week for a period of 6 months or more.

For oral administration, the pharmaceutical composition of the invention can take the form of, for example, tablets or capsules prepared by conventional means with pharmaceutical acceptable excipients such as binding agents (e.g., pregelatinised maize starch, polyvinylpyrrolidone, hydroxypropyl methylcellulose), fillers (e.g., lactose, microcrystalline cellulose, calcium hydrogen phosphate), lubricants (e.g., magnesium stearate, talc, silica), disintegrants (e.g., potato starch, sodium starch glycolate), or wetting agents (e.g., sodium lauryl sulphate). Liquid preparations for oral administration can be in the form of, for example, solutions, syrups, or suspensions, or can be presented as a dry product for constitution with water or other suitable vehicle before use. Such liquid preparation can be prepared by conventional means with pharmaceutically acceptable additives such as suspending agents (e.g., sorbitol, syrup, cellulose derivatives, hydrogenated edible fats), emulsifying agents (e.g., lecithin, acacia), non-aqueous vehicles (e.g., almond oil, oily esters, ethyl alcohol, fractionated vegetable oils), preservatives (e.g., methyl or propyl-p-hydroxycarbonates, sorbic acids). The preparations can also contain buffer salts, flavouring, coloring and sweetening agents as deemed appropriate. Preparations for oral administration can be suitably formulated to give controlled release of the pharmaceutical composition of the invention.

The pharmaceutical composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

For administration by inhalation, the pharmaceutical composition of the invention is conveniently delivered in the form of an aerosol spray presentation from a pressurised pack or a nebulizer, with the use of a suitable propellant (e.g., dichlorodifluoromethane, trichlorofluoromethane, dichlorotetrafluoroethane, carbon dioxide, nitrogen, or other suitable gas). In the case of a pressurised aerosol, the dosage unit can be determined by providing a valve to deliver a metered amount. Capsules and cartridges of, for example, gelatine, for use in an inhaler or insufflator can be formulated containing a powder mix of the pharmaceutical composition of the invention and a suitable powder base such as lactose or starch.

The pharmaceutical composition of the invention can be formulated for parenteral administration by injection, for example, by bolus injection or continuous infusion. Formulations for injection can be presented in units dosage form (e.g., in phial, in multi-dose container), and with an added preservative. The pharmaceutical composition of the invention can take such forms as suspensions, solutions or emulsions in oily or aqueous vehicles, and can contain formulatory agents such as suspending, stabilizing, or dispersing agents. Alternatively, the agent can be in powder form for constitution with a suitable vehicle (e.g., sterile pyrogen-free water) before use. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilised powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Therapeutic/pharmaceutical compositions can be administered with medical devices known in the art. For example, in a preferred embodiment, a therapeutic/pharmaceutical composition of the invention can be administered with a needleless hypodermic injection device, such as the devices disclosed in U.S. Pat. Nos. 5,399,163; 5,383,851; 5,312,335; 5,064,413; 4,941,880; 4,790,824; or U.S. Pat. No. 4,596,556. Examples of well-known implants and modules useful in the present invention include those described in: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system.

Many other such implants, delivery systems, and modules are known to those skilled in the art. In certain embodiments, the compounds of the invention can be formulated to ensure proper distribution in vivo. For example, the blood-brain barrier (BBB) excludes many highly hydrophilic compounds. To ensure that the compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, and thus enhance targeted drug delivery (see, e.g., V. V. Ranade (1989) J. Clin. Pharmacol. 29: 685). Exemplary targeting moieties include folate or biotin (see, e.g., U.S. Pat. No. 5,416,016 to Low et al.); mannosides (Umezawa et al., (1988) Biochem. Biophys. Res. Commun. 153: 1038); antibodies (P. G. Bloeman et al. (1995) FEBS Lett. 357: 140; M. Owais et al. (1995) Antimicrob. Agents Chemother. 39: 180); and surfactant protein A receptor (Briscoe et al. (1995) Am. J. Physiol. 1233: 134).

In one embodiment of the invention, the compounds of the invention are formulated in liposomes. In a more preferred embodiment, the liposomes include a targeting moiety. In a most preferred embodiment, the compounds in the liposomes are delivered by bolus injection to a site proximal to the desired area. Such liposome-based composition should be fluid to the extent that easy syringability exists, should be stable under the conditions of manufacture and storage and should be preserved against the contaminating action of microorganisms such as bacteria and fungi.

A “therapeutically effective dosage” for therapy/treatment can be measured by objective responses which can either be complete or partial. A complete response (CR) is defined as no clinical, radiological or other evidence of a condition, disorder or disease. A partial response (PR) results from a reduction in disease of greater than 50%. Median time to progression is a measure that characterizes the durability of the objective response.

A “therapeutically effective dosage” for therapy/treatment can also be measured by its ability to stabilize the progression of a condition, disorder or disease. The ability of a compound to inhibit, reduce or ameliorate non-apoptotic regulated cell-death and/or to reduce oxidative stress can be evaluated in appropriate animal model systems as such as one or more of those set fourth below. Alternatively, these properties of a compound of the present invention can be evaluated by examining the ability of the compound using in vitro assays known to the skilled practitioner such as one or more of those set fourth below. A therapeutically effective amount of a compound of the present invention can cure, heal, alleviate, relieve, alter, remedy, ameliorate, improve or affect the condition, disorder or disease or the symptoms of the condition, disorder or disease or the predisposition toward the condition, disorder or disease in an individual. One of ordinary skill in the art would be able to determine such amounts based on such factors as the individual's size, the severity of the individual's symptoms, and the particular composition or route of administration selected.

An injectable composition should be sterile and fluid to the extent that the composition is deliverable by syringe. In addition to water, the carrier can be an isotonic buffered saline solution, ethanol, polyol (for example, glycerol, propylene glycol, and liquid polyethylene glycol, and the like), and suitable mixtures thereof.

The pharmaceutical composition of the invention can also, if desired, be presented in a pack, or dispenser device which can contain one or more unit dosage forms containing the said agent. The pack can for example comprise metal or plastic foil, such as blister pack. The pack or dispenser device can be accompanied with instruction for administration.

The pharmaceutical composition of the invention can be administered as sole active agent or can be administered in combination with other therapeutically and/or cosmetically active agents.

Further, the invention is directed to a screening assay, comprising the steps: a) culturing adherent cells which deposit at least one protein in the presence of at least one test compound; b) staining of at least one protein deposited by the adherent cells; c) fixation of adherent cells and the at least one protein; d) microscopic detection of a signal of the at least one stained deposited protein; e) data analysis of signals detected in step d) comprising quantification of the amount of the at least one protein deposited in the presence of the at least one test compound. wherein step b) is carried out before step c).

In step a), cells are cultured in an adherent cell culture. The cells may be cultured in an adherent cell culture applying any conditions known to the person skilled in the art suitable to culture the respective cell type. Optionally, the cells are starved before application in step a), preferably for 5 to 48 h, more preferably 10 to 30 h, most preferably for 20 to 26 h.

Preferably, the adherent cells are primary cells. In one embodiment, the adherent cells are primary patient derived human cells most preferably human lung fibroblasts. In another embodiment the cells are primary animal derived cells or any adherent immortalized cells.

Preferably, the at least one protein in step a) is an extracellular matrix protein.

More preferably, the at least one extracellular matrix protein is selected from the group consisting of collagen type 5, collagen type 1, and fibulin 1. In a further embodiment more than one protein is deposited. For example at least two, at least three or at least four proteins are deposited.

Preferably, step a) is carried out for at least 24 h, more preferably 60 to 90 hours, particular preferred 65 to 80 hours, most preferably for 70 to 75 hours.

The at least one test compound is preferably a compound which is expected to inhibit deposition of the at least one protein. It is the purpose of the assay to identify potential compounds which inhibit the deposition of the at least one protein and to quantify the extend of the inhibition by the respected test compound. Optionally, the at least one test compound in step a) is a small molecule; and/or oligonucleotides, peptides, proteins, protacs, anticalins, antibodies, or CRISPRs.

Optionally, in step a) at least one growth factor, preferably at least one Transforming Growth Factor β (TGF β), more preferably, TGF β1 is present.

In one embodiment, staining in step b), comprises the binding of at least one antibody to the at least one protein or at least one probe binding to the at least one protein.

Optionally, the antibody or probe comprises at least one detectable label that is directly conjugated to the antibody and wherein optionally the detectable label has fluorescence property, preferably the detectable lable is a fluorophore selected from AlexaFluor 488, AlexaFluor 555, AlexaFluor 637; AlexaFluor 647, AlexaFluor 568, AlexaFluor 568 and/or Qdots.

In a further embodiment, staining in step b) comprises the binding of at least one first antibody (FA) to at least one protein and the subsequent binding of at least one first antibody (FA) with at least one secondary antibody (SA), wherein the at least one second antibody (SA) comprises at least one detectable label that is conjugated to the antibody and wherein optionally the detectable label has fluorescence property, preferably the detectable label is a fluorophore selected from AlexaFluor 488, AlexaFluor 555, AlexaFluor 637; AlexaFluor 647, AlexaFluor 568, AlexaFluor 568, and/or Qdot.

Optionally, in step b) at least one further co-staining is present and selected from the group consisting of cell-nuclei staining, live-dead staining, myofibroblast markers (e.g. αSMA-staining), apoptosis markers (e.g. Caspase3/7 staining).

In step c) fixation may be carried out with any reagents which are suitable for the purpose and known to the person skilled in the art. Respective conditions are known in the art.

Examplary conditions used in certain embodiments of the inventions are 4% PFA for 30 min at 37° C. or 100% methanol for 2 min at −20° C. Staining in step b) is carried out before fixation in step b).

Preferably, in step d) 2D, 3D or 4D imaging is carried out. More preferably, step d) is carried out with a conventional or confocal imaging apparatus.

Data analysis in step e) may comprise using a machine learning model, such as neural networks.

### EXAMPLES OF THE INVENTION

### 1. Synthesis of Compounds

### Example 1: (E)-N-(2-butoxyphenyl)-3-(4-(cyclopropylmethoxy)phenyl)acrylamide

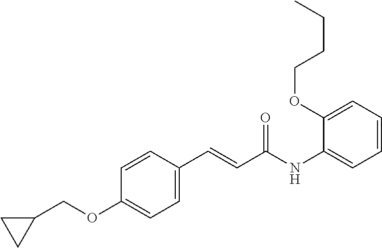 [SMILES: CCCCOc1ccccc1NC(=O)/C=C/c1ccc(OCC2CC2)cc1]

### a) (E)-N-(2-butoxyphenyl)-3-(4-hydroxyphenyl)acrylamide

(E)-3-(4-hydroxyphenyl)acrylic acid (120 mg, 0.73 mmol) was dissolved in 2 mL of dry DMF. The solution was cooled in an ice bath and 2-butoxyaniline (0.87 mmol, 1.2 eq) was added followed by a solution of benzotriazol-1-yloxytripyrrolidinophosphonium hexafluorophosphate (1.1 mmol, 1.5 eq) in 2 mL of dichloromethane. Subsequently, triethylamine (1.46 mmol, 2 eq) was added. The mixture was stirred at 0° C. for 30 min and then at room temperature overnight. The mixture was diluted by dichloromethane and washed with 1M aqueous hydrochloric acid, saturated sodium carbonate and brine. The organic layer was dried over magnesium sulphate and concentrated in vacuo. Purification by silica gel chromatography yielded (E)-N-(2-butoxyphenyl)-3-(4-hydroxyphenyl)acrylamide (87 mg).

### b) (E)-N-(2-butoxyphenyl)-3-(4-(cyclopropylmethoxy)phenyl)acrylamide (Example 1)

Diisopropyl azodicarboxylate (2 eq) was added over a period of 10 min at 0° C. to a solution of (E)-N-(2-butoxyphenyl)-3-(4-hydroxyphenyl)acrylamide (1 eq.), cyclopropylmethanol (2 eq.), and triphenyl phosphine (2 eq.) in absolute tetrahydrofurane (15 mL). After 30 minutes the cooling bath was removed, and the solution was stirred at room temperature for 16 h. The solvent was concentrated in vacuo, and the remaining residue was purified by flash chromatography to yield (E)-N-(2-butoxyphenyl)-3-(4-(cyclopropylmethoxy)phenyl)acrylamide in 77% yield.

¹H-NMR (CDCl₃, 400 MHz) 0.36 (2H, q, J=5.11 Hz), 0.64-0.69 (2H, m), 1.03 (3H, t, J=7.40 Hz), 1.25-1.31 (1H, m), 1.54 (2H, sextet, J=7.47 Hz), 1.86 (2H, quintet, J=7.06 Hz), 3.83 (2H, d, J=6.92 Hz), 4.07 (3H, t, J=6.60 Hz), 6.42 (2H, d, J=15.45 Hz), 6.87-6.91 (3H, m), 6.98 (1H, dt, J=1.40, 7.66 Hz), 7.03 (1H, dt, J=1.73, 7.69 Hz), 7.50 (2H, d, J=8.72 Hz), 7.69 (1H, d, J=15.45 Hz), 7.92 (1H, s), 8.51 (1H, d, J=6.52 Hz). ¹³C-NMR (CDCl3, 101 MHz) 3.36, 10.30, 14.04, 19.48, 31.37, 68.55, 73.00, 111.01, 114.99, 118.91, 120.01, 121.17, 123.70, 127.47, 128.26, 129.68, 141.73, 147.38, 160.66, 164.16. HRMS (ESI): m/z [M+Na]⁺ calcd for C₂₃H₂₇NO₃: 388.1889, Found: 388.1894.

### Example 2: (E)-N-(2-butoxyphenyl)-3-(4-(2,2,2-trifluoroethoxy)phenyl)acrylamide

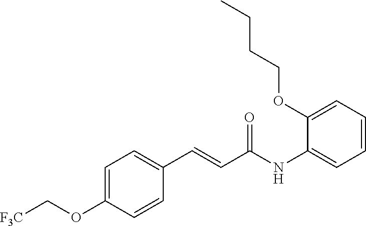 [SMILES: CCCCOc1ccccc1NC(=O)/C=C/c1ccc(OCC(F)(F)F)cc1]

### a) Methyl (E)-3-(4-hydroxyphenyl)acrylate

Concentrated sulfuric acid (0.67 mL) was added to a solution of (E)-3-(4-hydroxyphenyl)acrylic acid (0.8 g) in methanol (40 mL). The solution was heated to reflux for 5 h, cooled to room temperature and then quenched by addition of saturated aqueous sodium bicarbonate solution. The aqueous phase was extracted with ethyl acetate and the combined organic fractions were washed with water, brine, dried over magnesium sulphate and concentrated in vacuo providing methyl (E)-3-(4-hydroxyphenyl)acrylate (800 mg), which was sufficiently pure for further conversion. 1H-NMR (CDCl3 400 MHz) 3.81 (3H, s), 6.02 (1H, s), 6.30 (1H, d, J=15.93 Hz), 6.86 (2H, d, J=8.60 Hz), 7.42 (2H, d, J=8.56 Hz), 7.64 (1H, d, J=15.97 Hz). HLM-01-046, yielding 90%

### b) Methyl (E)-3-(4-(2,2,2-trifluoroethoxy)phenyl)acrylate

A dry flask was charged with NaH (1.2 equiv) under argon. Dry dimethylsulfoxide (4 mL) was added to the reaction flask and stirred at 0° C. for 15 min. A solution of Methyl (E)-3-(4-(2,2,2-trifluoroethoxy)phenyl)acrylate (890 mg, 5 mmol, 1 equiv) in DMSO (2 mL) was slowly added to the suspension over 10 min. The reaction mixture was allowed to stir at 0° C. for 30 min. 2,2,2-trifluoroethyl iodide (1.5 mL, 15 mmol, 3 equiv) was added to the reaction flask. The reaction was then stirred at 80° C. for 24 h. After completion of the reaction, it was quenched by addition of water and extracted with ethyl acetate. The organic phase was evaporated to dryness and purified by silica gel chromatography.

### c) (E)-3-(4-(2,22-trifluoroethoxy)phenyl)acrylic acid

To a solution of methyl (E)-3-(4-(2,2,2-trifluoroethoxy)phenyl)acrylate (1 equiv.) in methanol (4 mL) was added potassium carbonate (5 equiv.) dissolved in water (4 mL). The reaction mixture was refluxed for 3 h after which methanol was removed under reduced pressure. The solution was then cooled to 0° C. and acidified to pH 2 by addition of hydrochloric acid (1M). The mixture was extracted with diethyl ether and the combined organic layer was washed with brine, dried with sodium sulphate, and evaporated in vacuo to give the product as white solid in 90% yield.

### d) (E)-N-(2-butoxyphenyl)-3-(4-(2,2,2-trifluoroethoxy)phenyl)acrylamide

(E)-N-(2-butoxyphenyl)-3-(4-(2,2,2-trifluoroethoxy)phenyl)acrylamide was prepared from (E)-3-(4-(2,2,2-trifluoroethoxy)phenyl)acrylic acid and 2-butoxyaniline in a similar fashion as described for (E)-N-(2-butoxyphenyl)-3-(4-hydroxyphenyl)acrylamide (Example 1, step a). The product was obtained in 63% yield.

¹H-NMR (CDCl3 400 MHz) 1.03 (3H, t, J=7.40 Hz), 1.54 (2H, sextet, J=7.47 Hz), 1.86 (2H, quintet, J=7.06 Hz), 4.07 (2H, t, J=6.60 Hz), 4.38 (2H, q, J=8.04 Hz), 6.46 (1H, d, J=15.49 Hz), 6.89 (2H, dd, J=1.04, 7.96 Hz), 6.94-7.00 (3H, m), 7.04 (1H, dt, J=1.57, 7.70 Hz), 7.53 (2H, d, J=8.68 Hz), 7.69 (1H, d, J=15.49 Hz), 7.95 (1H, s), 8.52 (1H, d, J=6.48 Hz). ¹³C-NMR (CDCl₃) 14.03, 19.47, 31.36, 65.82 (1C, q, J=35.83 Hz), 68.57, 111.04, 115.28, 120.05, 120.23, 121.17, 123.31 (1C, q, J=277.95 Hz), 123.87, 128.13, 129.40, 129.77, 141.08, 147.41, 158.62, 163.80. HRMS (ESI): m/z [M+Na]⁺ calcd for C₂₁H₂₂F₃NO₃: 416.1449, Found: 416.1445.

### Example 3: (E)-3-(4-methoxyphenyl)-N-(2-phenoxyphenyl)acrylamide

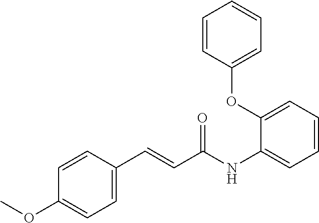

A suspension of (E)-3-(4-methoxyphenyl)acrylic acid (150 mg, 0.84 mmol, 1 eq.) in dry dichloromethane (3 mL) was treated with oxalyl chloride (1.68 mmol, 2 eq.) and a catalytic amount of dimethyl formamide at 0° C. under argon. After 5 min, the solution was allowed to warm and stirred at ambient temperature for one hour. Subsequently the solvent was removed under reduced pressure to give the acid chloride as a yellow solid. A solution of the acid chloride in dry dichloromethane (4 mL) was added to a solution of 2-phenoxyaniline (1.01 mmol, 1.2 eq) and triethyl amine (1.01 mmol 1.2 eq) in dichloromethane (3 mL) at 0° C. The suspension was stirred at room temperature for 16 h and was quenched by addition of water. The mixture was diluted with dichloromethane and washed with aqueous ammonium chloride, saturated sodium bicarbonate, then dried over magnesium sulphate and concentrated in vacuo. The crude product was purified by silica gel chromatography to give 139 mg of (E)-3-(4-methoxyphenyl)-N-(2-phenoxyphenyl)acrylamide in 48% yield (139 mg).

¹H-NMR (CDCl3 400 MHz) 3.82 (1H, s), 6.42 (1H, d, J=15.45 Hz), 6.86 (1H, dd, J=1.30, 8.14 Hz), 6.89 (2H, d, J=8.76 Hz), 7.01 (1H, dt, J=1.53, 7.79 Hz), 7.06 (2H, dd, J=0.96, 8.60 Hz), 7.13-7.18 (2H, m), 7.35-7.39 (2H, m), 7.47 (2H, d, J=8.76 Hz), 7.69 (1H, d, J=15.49 Hz), 7.93 (1H, s), 8.62 (1H, d, J=7.92 Hz). ¹³C-NMR (CDCl₃): 55.45, 114.38, 117.70, 118.65, 118.88, 121.04, 123.97, 124.08, 124.18, 127.43, 129.72, 130.11, 130.24, 142.11, 145.78, 156.55, 161.23, 164.36. HRMS (ESI): m/z [M+Na]⁺ calcd for C₂₂H19NO3: 368.1263, Found: 368.1264.

### Example 4: (E)-N-(4-butoxypyridin-3-yl)-3-(4-methoxyphenyl)acrylamide

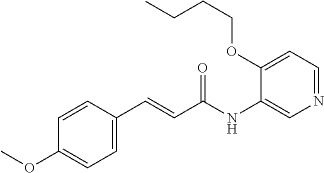

### a) 4-butoxy-3-nitropyridine

To a solution of 3-nitropyridin-4-ol (1.0 g, 7.13 mmol) and triphenyl phosphine (2.8 g, 10.71 mmol, 1.5 eq.) in anhydrous tetrahydrofurane (10 mL) was added diisopropyl azodicarboxylate (2.16 g, 10.71 mmol, 1.5 eq) in tetrahydrofurane (2.5 mL) and 1-butanol (794 mg, 10.71 mmol, 1.5 eq.) in tetrahydrofurane (2.5 mL) at 0° C. simultaneously. The temperature of the reaction mixture was raised slowly to room temperature and stirring continued at room temperature overnight. At the end of this period diluted with ethyl acetate (25 mL) and washed with water (2×50 mL), dried by Na₂SO₄, filtered and the solvent was evaporated. The residue was purified by silica column (dichloromethane/methanol=50/1) to afford 4-butoxy-3-nitropyridine as light yellow solid (400 mg).

¹H-NMR (CDCl3 400 MHz) 0.99 (3H, t, J=7.36 Hz), 1.40 (2H, q, J=7.55 Hz), 1.84 (2H, m, J=7.47 Hz), 3.94 (2H, t, J=7.32 Hz), 6.68 (1H, d, J=7.68 Hz), 7.32 (1H, q, J=3.37 Hz), 8.53 (1H, d, J=2.36 Hz).

### b) 4-butoxypyridin-3-amine

A suspension of 4-butoxy-3-nitropyridine (386 mg, 2.0 mmol) in ethanol (9.2 ml) and acetic acid (0.51 ml) was heated at 60° C. After adding of iron powder (678 mg, 12.0 mmol, 6.0 eq) and iron (III) chloride hexahydrate (55 mg), the mixture was stirred under reflux for 18 hours. After cooling to room temperature, the mixture was diluted with ethyl acetate (100 ml) and filtered through Celite. The filtrate and washings were combined and washed by water. The organic phase was dried by MgSO₄ and then concentrated in vacuum to afford 4-butoxypyridin-3-amine as a light yellow solid (310 mg). The crude product can be used in next step without further purification.

### c) (E)-N-(4-butoxypyridin-3-yl)-3-(4-methoxyphenyl)acrylamide (Example 4)

A suspension of (E)-3-(4-methoxyphenyl)but-2-enoic acid (1.4 mmol, 250 mg) in dry dichloromethane (5 mL) was treated with oxalyl chloride (1.5 eq.) and a catalytic amount of dimethyl formamide (2 drops) at 0° C. under argon atmosphere. After 5 min, the solution was allowed to warm at room temperature and allowed to stir at ambient temperature for 1 h. The solvent was removed under reduced pressure to give the acid chloride as a yellow solid. A solution of the acid chloride in extra dried dichloromethane (7 mL) was added to a solution of 4-butoxypyridin-3-amine (300 mg, 1.2 eq.) and triethylamine (1.2 eq.) in dichloromethane (5 mL) at 0° C. The suspension was stirring at ambient temperature for 4 h and was quenched by water. The mixture was diluted by dichloromethane and washed by aqueous ammonium chloride, saturated sodium bicarbonate, and then dried by MgSO₄. After concentrated in vacuum, the residue was purified by a silica gel column (dichloromethane/methanol 15:1) to afford (E)-N-(4-butoxypyridin-3-yl)-3-(4-methoxyphenyl)acrylamide as a white solid (530 mg).

¹H-NMR (DMSO-d₆, 400 MHz) 0.90 (3H, t, J=7.36 Hz), 1.27 (2H, sextet, J=7.48 Hz), 1.69 (2H, quintet, J=7.30 Hz), 3.79 (3H, s), 3.95 (2H, t, J=7.06 Hz), 6.25 (1H, d, J=7.28 Hz), 6.99 (2H, d, J=8.76 Hz), 7.20 (1H, d, J=15.65 Hz), 7.46 (1H, d, J=15.61 Hz), 7.59 (2H, d, J=8.76 Hz), 7.71 (1H, dd, J=2.26, 7.30 Hz), 8.89 (1H, d, J=2.24 Hz), 9.27 (1H, s). ¹³C-NMR (DMSO-d₆, 100 MHz) 13.41, 18.86, 32.44, 55.28, 56.25, 112.89, 114.37, 119.69, 127.45, 127.66, 129.44, 129.51, 138.60, 140.00, 160.56, 164.27, 169.02.

HRMS (ESI): Found 349.1527. Calc. 349.1528, [M+Na]⁺, M=C₁₉H₂₂N₂O₃

### Example 5: (E)-N-(2-((4-fluorobenzyl)oxy)phenyl)-3-(4-methoxyphenyl)acrylamide

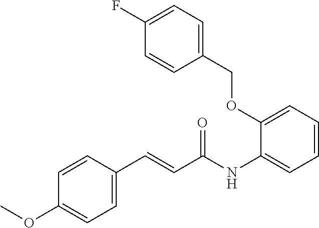

### a) 1-((4-Fluorobenzyl)oxy)-2-nitrobenzene

To a solution of 2-nitrophenol in acetonitrile, anhydrous potassium carbonate was added. The mixture was stirred for 30 minutes at room temperature. Catalytic amount of potassium iodide and 1-(bromomethyl)-4-fluorobenzene were added. The reaction was stirred at 75° C. for 8 h. Water (125 mL) was added to the reaction mixture and the reaction mixture was extracted with ethyl acetate. The combined organic layer was washed with brine and dried over anhydrous sodium sulphate. The solvent was evaporated under reduced pressure and the residue was purified by flash chromatography.

¹H-NMR (CDCl₃): 5.19 (2H, s), 7.08 (4H, m, J=4.53 Hz), 7.44 (2H, q, J=4.65 Hz), 7.51 (1H, m, J=4.36 Hz), 7.86 (1H, q, J=3.21 Hz).

### b) 2-((4-Fluorobenzyl)oxy)aniline

To a solution of 1-((4-fluorobenzyl)oxy)-2-nitrobenzene (1 eq.) in methanol (7.5 mL) and water (7.5 mL) was added ammonium chloride (10 eq.) and iron powder (5 eq.). The reaction mixture was allowed to stir at 90° C. for 16 h. The mixture was filtered, and water (50 mL) was added to the filtrate. The filtrate was extracted with ethyl acetate and the combined organic layer was extracted with brine, dried over magnesium sulphate and concentrated in vacuo to give the crude 2-((4-Fluorobenzyl)oxy)aniline (80% yield), which was used without purification for further conversion.

¹H-NMR (CDCl₃): 1H-NMR (CDCl3, 400 MHz) 3.84 (3H, s), 5.11 (2H, s), 6.36 (1H, d, J=15.45 Hz), 6.90 (2H, d, J=8.72 Hz), 6.93-6.96 (1H, m), 7.00-7.04 (2H, m), 7.12 (2H, t, J=8.62 Hz), 7.42 (1H, dd, J=5.40, 8.44 Hz), 7.48 (2H, d, J=8.68 Hz), 7.67 (1H, d, J=15.45 Hz), 7.87 (1H, s), 8.54 (1H, s). ¹³C-NMR (CDCl₃): 55.48, 70.44, 111.72, 114.40, 115.89 (d, J=21.60 Hz), 119.61 (d, J=157.91 Hz), 121.86, 123.77, 127.48, 128.42, 129.69, 129.78, 132.34 (d, J=3.27 Hz), 141.85, 147.13, 161.22, 161.62, 164.07, 164.19.

### c) (E)-N-(2-((4-fluorobenzyl)oxy)phenyl)-3-(4-methoxyphenyl)acrylamide (Example 5)

(E)-3-(4-methoxyphenyl)acrylic acid and 2-((4-fluorobenzyl)oxy)aniline were reacted in a similar fashion as described for the synthesis of (E)-3-(4-methoxyphenyl)-N-(2-phenoxyphenyl)acrylamide (Example 3) to yield (E)-N-(2-((4-fluorobenzyl)oxy)phenyl)-3-(4-methoxyphenyl)acrylamide in 57% yield.

¹H-NMR (DMSO-d₆, 400 MHz): 3.84 (3H, s), 5.11 (2H, s), 6.36 (1H, d, J=15.45 Hz), 6.90 (2H, d, J=8.72 Hz), 6.95 (1H, m, J=2.37 Hz), 7.03 (2H, t, J=3.96 Hz), 7.12 (2H, t, J=8.62 Hz), 7.42 (2H, q, J=4.61 Hz), 7.48 (2H, d, J=8.68 Hz), 7.67 (1H, d, J=15.45 Hz), 7.87 (1H, s), 8.54 (1H, s). ¹³C-NMR (DMSO-d₆, 100 MHz): δ5.48, 70.44, 111.72, 114.40, 115.89 (d, J=21.60 Hz), 119.61 (d, J=157.91 Hz), 121.86, 123.77, 127.48, 128.42, 129.69, 129.78, 132.34 (d, J=3.27 Hz), 141.85, 147.13, 161.22, 161.62, 164.07, 164.19. HRMS (ESI): Found 400.1310 [M+Na]⁺ calc. 400.1325.

### Example 6: (E)-3-(2,2-difluorobenzo[d][1,3]dioxol-5-yl)-N-(2-methoxyphenyl)acrylamide

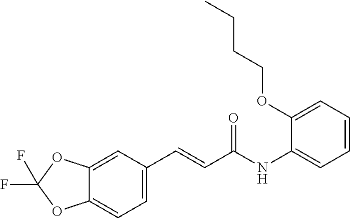 [SMILES: CCCCOc1ccccc1NC(=O)/C=C/c1ccc2c(c1)OC(F)(F)O2]

### a) (E)-3-(2,2-difluorobenzo[d][1,3]dioxol-5-yl)acrylic acid

A mixture of 2.2 g (11.8 mmol) 2,2-difluoro-benzo[1,3]dioxole-5-carbaldehyde, 2.71 g (26.0 mmol) malonic acid, 0.2 g (2.4 mmol) piperidine and 9 ml pyridine was kept at reflux temperature until carbon dioxide development ceased (3 h). After cooling to room temperature the reaction mixture was poured onto 100 g of ice and 30 ml of 6N HCl. The precipitate was isolated, washed with water and dried to afford the desired product HLM1319 (2.53 g, 11.1 mmol, yielding 94%) as white solid, which was used in next step without further purification.

### b) (E)-3-(2,2-difluorobenzo[d][1,3]dioxol-5-yl)-N-(2-methoxyphenyl)acrylamide (Example 6)

By following the synthesis procedure of Example 3 ((E)-3-(4-methoxyphenyl)-N-(2-phenoxyphenyl)acrylamide), (E)-3-(2,2-difluorobenzo[d][1,3]dioxol-5-yl)-N-(2-methoxyphenyl)acrylamide was obtained as brown solid (400 mg, 1.2 mmol, yielding 68%) from (E)-3-(2,2-difluorobenzo[d][1,3]dioxol-5-yl)acrylic acid (400 mg, 1.75 mmol) after purification by silica gel chromatography (PE/EA=8/1).

¹H-NMR (CDCl₃ 400 MHz): 1.03 (3H, t, J=7.40 Hz), 1.53 (2H, sextet, J=7.47 Hz), 1.86 (2H, quintet, J=7.06 Hz), 4.08 (2H, t, J=6.62 Hz), 6.46 (1H, d, J=15.41 Hz), 6.90 (1H, dd, J=0.96, 8.04 Hz), 6.99 (1H, dt, J=1.24, 7.76 Hz), 7.04 (1H, dd, J=1.36, 7.80 Hz), 7.08 (1H, d, J=8.16 Hz), 7.26-7.30 (2H, m), 7.69 (1H, d, J=15.45 Hz), 7.95 (1H, s), 8.51 (1H, d, J=6.84 Hz). ¹³C-NMR (CDCl₃, 100 MHz): 13.92, 19.35, 31.30, 36.55, 68.17, 107.86, 109.85, 111.04, 120.09, 121.16, 121.67, 124.08, 125.07, 127.93, 131.39, 131.76 (1C, t, J=256.34 Hz), 140.69, 144.46, 144.81, 147.41, 163.22. HRMS (ESI): Found: 398.1181 [M+Na] calc. 398.1180.

The following examples, listed in the Table 2 below, were obtained using similar methods as used for synthesis of Examples 1-6.

TABLE 2

| Example | Structure | IUPAC Name | HR-MS |
|---|---|---|---|
| 7 | 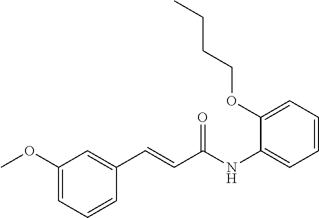 [SMILES: CCCCOc1ccccc1NC(=O)/C=C/c1cccc(OC)c1] | (E)-N-(2- butoxyphenyl)-3- (3- methoxyphenyl) acrylamide | Found: 348.1574 [M + Na]⁺ calc. 348.1576 |
| 8 | 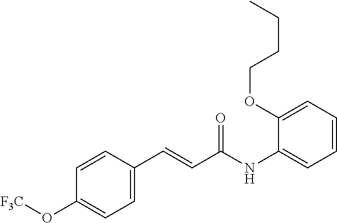 [SMILES: CCCCOc1ccccc1NC(=O)/C=C/c1ccc(OC(F)(F)F)cc1] | (E)-N-(2- butoxyphenyl)-3- (4- (trifluoromethoxy) phenyl)acrylamide | Found: 402.1286 [M + Na]⁺ calc. 402.1293 |
| 9 | 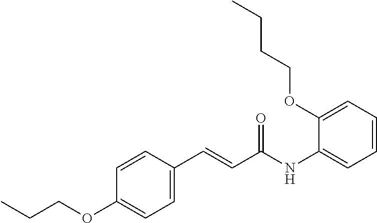 [SMILES: CCCCOc1ccccc1NC(=O)/C=C/c1ccc(OCCC)cc1] | (E)-N-(2- butoxyphenyl)-3- (4- propoxyphenyl) acrylamide | Found: 376.1887 [M + Na]⁺ calc. 376.1889 |
| 10 | 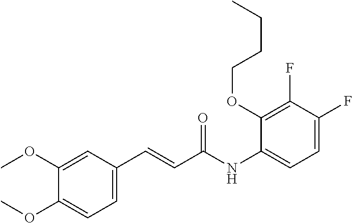 [SMILES: CCCCOc1c(NC(=O)/C=C/c2ccc(OC)c(OC)c2)ccc(F)c1F] | (E)-N-(2-butoxy- 3,4- difluorophenyl)-3- (3,4- dimethoxyphenyl) acrylamide | Found: 414.1493 [M + Na]⁺ calc. 414.1490 |
| 11 | 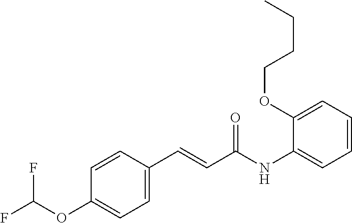 [SMILES: CCCCOc1ccccc1NC(=O)/C=C/c1ccc(OC(F)F)cc1] | (E)-N-(2- butoxyphenyl)-3- (4- (difluoromethoxy) phenyl)acrylamide | Found: 382.1418 [M + Na]⁺ calc. 382.1419 |
| 12 | 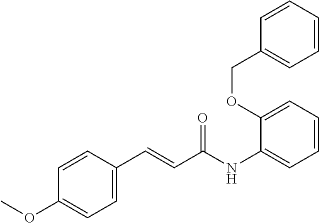 [SMILES: COc1ccc(/C=C/C(=O)Nc2ccccc2OCc2ccccc2)cc1] | (E)-N-(2- (benzyloxy)phenyl)- 3-(4- methoxyphenyl) acrylamide | Found: 382.1418 [M + Na]⁺ calc. 382.1419 |
| 13 | 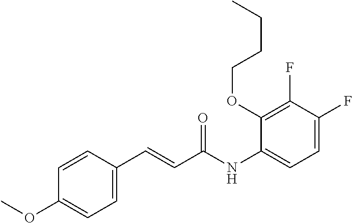 [SMILES: CCCCOc1c(NC(=O)/C=C/c2ccc(OC)cc2)ccc(F)c1F] | E)-N-(2-butoxy- 3,4- difluorophenyl)-3- (4- methoxyphenyl) acrylamide | Found: 384.1386 [M + Na]⁺ calc 384.1387 |
| 14 | 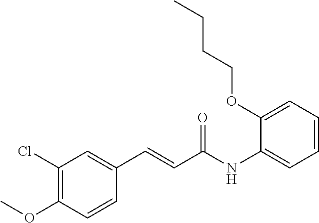 [SMILES: CCCCOc1ccccc1NC(=O)/C=C/c1ccc(OC)c(Cl)c1] | (E)-N-(2- butoxyphenyl)-3- (3-chloro-4- methoxyphenyl) acrylamide | Found: 382.1181 [M + Na]⁺ calc 382.1186 |
| 15 | 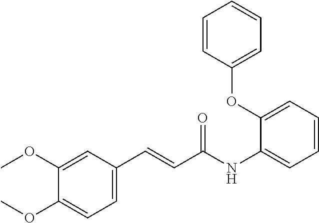 [SMILES: COc1ccc(/C=C/C(=O)Nc2ccccc2Oc2ccccc2)cc1OC] | (E)-3-(3,4- dimethoxyphenyl)- N-(2- phenoxyphenyl) acrylamide | Found: 398.1366 [M + Na]⁺ calc 398.1368 |
| 16 | 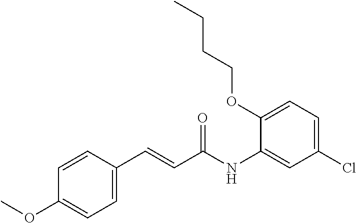 [SMILES: CCCCOc1ccc(Cl)cc1NC(=O)/C=C/c1ccc(OC)cc1] | (E)-N-(2-butoxy-5- chlorophenyl)-3- (4- methoxyphenyl) acrylamide | Found: 382.1187 [M + Na]⁺ calc 382.1186 |
| 17 | 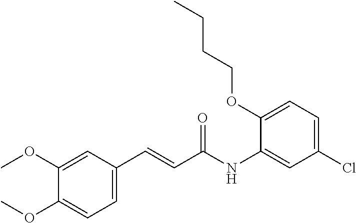 [SMILES: CCCCOc1ccc(Cl)cc1NC(=O)/C=C/c1ccc(OC)c(OC)c1] | (E)-N-(2-butoxy-5- chlorophenyl)-3- (3,4- dimethoxyphenyl) acrylamide | Found: 382.1187 [M + Na]⁺ calc 382.1186 |
| 18 | 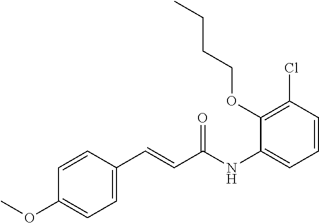 [SMILES: CCCCOc1c(Cl)cccc1NC(=O)/C=C/c1ccc(OC)cc1] | (E)-N-(2-butoxy-6- chlorophenyl)-3- (4- methoxyphenyl) acrylamide | Found: 412.1285 [M + Na]⁺ calc 412.1292 |
| 19 | 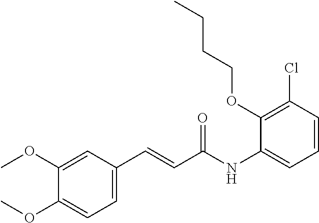 [SMILES: CCCCOc1c(Cl)cccc1NC(=O)/C=C/c1ccc(OC)c(OC)c1] | E)-N-(2-butoxy-3- chlorophenyl)-3- (3,4- dimethoxyphenyl) acrylamide | Found: 382.1183 [M + Na]⁺ calc 382.1186 |
| 20 | 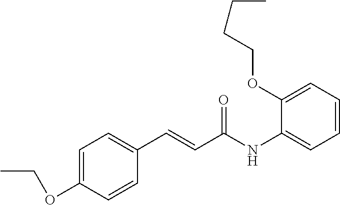 [SMILES: CCCCOc1ccccc1NC(=O)/C=C/c1ccc(OCC)cc1] | (E)-N-(2- butoxyphenyl)-3- (4- ethoxyphenyl) acrylamide | Found: 362.1726 [M + Na]⁺ calc 362.1732 |
| 21 | 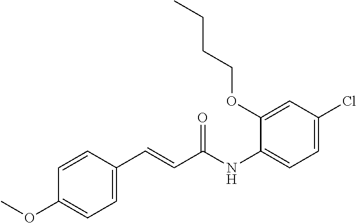 [SMILES: CCCCOc1cc(Cl)ccc1NC(=O)/C=C/c1ccc(OC)cc1] | (E)-N-(2-butoxy-4- chlorophenyl)-3- (4- methoxyphenyl) acrylamide | Found: 382.1191 [M + Na] calc. 382.1186 |
| 22 | 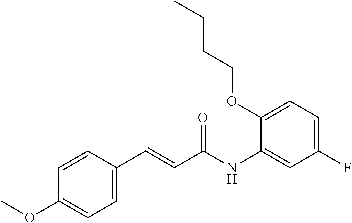 [SMILES: CCCCOc1ccc(F)cc1NC(=O)/C=C/c1ccc(OC)cc1] | (E)-N-(2-butoxy-5- fluorophenyl)-3- (4- methoxyphenyl) acrylamide | Found: 366.1483 [M + Na]⁺ calc. 366.1481 |
| 23 | 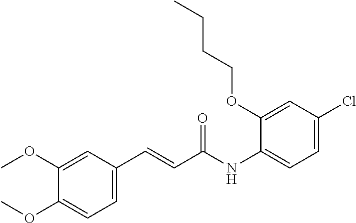 [SMILES: CCCCOc1cc(Cl)ccc1NC(=O)/C=C/c1ccc(OC)c(OC)c1] | (E)-N-(2-butoxy-4- chlorophenyl)-3- (3,4- dimethoxyphenyl) acrylamide | Found: 412.1295 [M + Na]⁺ calc. 412.1292 |
| 24 | 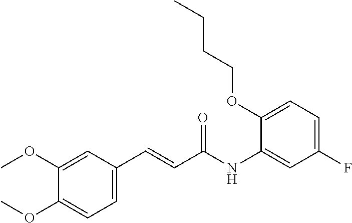 [SMILES: CCCCOc1ccc(F)cc1NC(=O)/C=C/c1ccc(OC)c(OC)c1] | (E)-N-(2-butoxy-5- fluorophenyl)-3- (3,4- dimethoxyphenyl) acrylamide | Found: 396.1588 [M + Na]⁺ calc. 396.1387 |
| 25 | 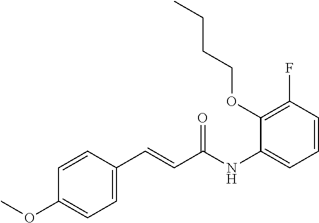 [SMILES: CCCCOc1c(F)cccc1NC(=O)/C=C/c1ccc(OC)cc1] | (E)-N-(2-butoxy-3- fluorophenyl)-3- (4- methoxyphenyl) acrylamide | Found: 366.1478 [M + Na]⁺ calc. 366.1481 |
| 26 | 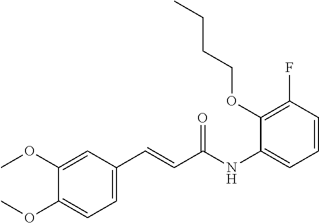 [SMILES: CCCCOc1c(F)cccc1NC(=O)/C=C/c1ccc(OC)c(OC)c1] | (E)-N-(2-butoxy-3- fluorophenyl)-3- (3,4- dimethoxyphenyl) acrylamide | Found: 396.1585 [M + Na] calc. 396.1587 |
| 27 | 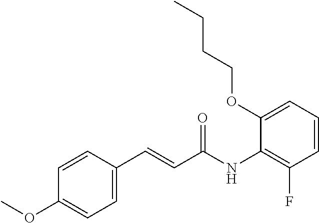 [SMILES: CCCCOc1cccc(F)c1NC(=O)/C=C/c1ccc(OC)cc1] | (E)-N-(2-butoxy-6- fluorophenyl)-3- (4- methoxyphenyl) acrylamide | Found: 366.1479 [M + Na] calc. 366.1481 |
| 28 | 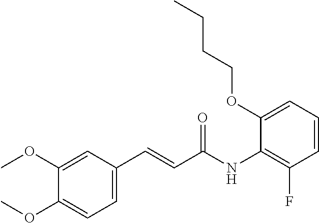 [SMILES: CCCCOc1cccc(F)c1NC(=O)/C=C/c1ccc(OC)c(OC)c1] | (E)-N-(2-butoxy-6- fluorophenyl)-3- (3,4- dimethoxyphenyl) acrylamide | Found: 396.1583 [M + Na] calc. 396.1587 |
| 29 | 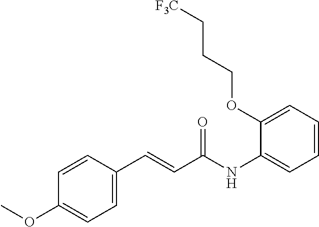 [SMILES: COc1ccc(/C=C/C(=O)Nc2ccccc2OCCCC(F)(F)F)cc1] | (E)-3-(4- methoxyphenyl)- N-(2-(4,4,4- trifluorobutoxy) phenyl)acrylamide | Found: 402.1292 [M + Na]⁺ calc. 402.1293 |
| 30 | 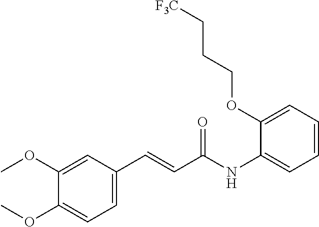 [SMILES: COc1ccc(/C=C/C(=O)Nc2ccccc2OCCCC(F)(F)F)cc1OC] | (E)-3-(3,4- dimethoxyphenyl)- N-(2-(4,4,4- trifluorobutoxy) phenyl)acrylamide | Found: 432.1397 [M + Na]⁺ calc. 432.1399 |
| 31 | 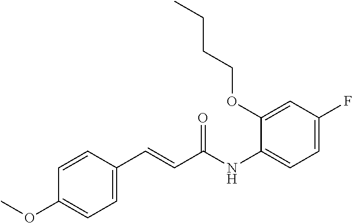 [SMILES: CCCCOc1cc(F)ccc1NC(=O)/C=C/c1ccc(OC)cc1] | (E)-N-(2-butoxy-4- fluorophenyl)-3- (4- methoxyphenyl) acrylamide | Found: 376.1484 [M + Na]⁺ calc. 366.1481 |
| 32 | 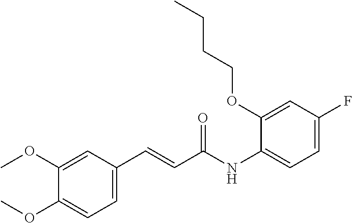 [SMILES: CCCCOc1cc(F)ccc1NC(=O)/C=C/c1ccc(OC)c(OC)c1] | (E)-N-(2-butoxy-4- fluorophenyl)-3- (3,4- dimethoxyphenyl) acrylamide | Found: 396.1589 [M + Na]⁺ calc. 396.1587 |
| 33 | 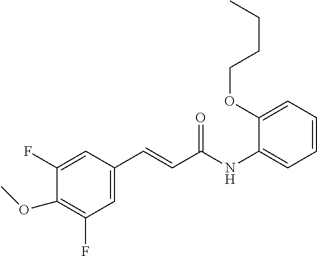 [SMILES: CCCCOc1ccccc1NC(=O)/C=C/c1cc(F)c(OC)c(F)c1] | (E)-N-(2- butoxyphenyl)-3- (3,5-difluoro-4- methoxyphenyl) acrylamide | Found: 360.1572 [M + Na]⁺ calc. 360.1576 |
| 34 | 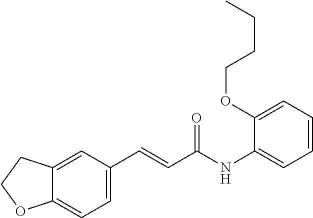 [SMILES: CCCCOc1ccccc1NC(=O)/C=C/c1ccc2c(c1)CCO2] | (E)-N-(2- butoxyphenyl)-3- (2,3- dihydrobenzofuran- 5-yl)acrylamide | Found: 360.1572 [M + Na]⁺ calc. 360.1576 |
| 35 | 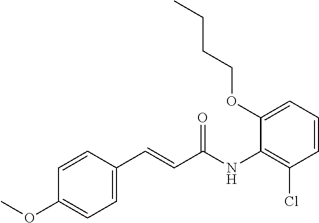 [SMILES: CCCCOc1cccc(Cl)c1NC(=O)/C=C/c1ccc(OC)cc1] | (E)-N-(2-butoxy-6- chlorophenyl)-3- (4-methoxyphenyl) acrylamide | Found: 382.1174 [M + Na]⁺ calc. 382.1186 |
| 36 | 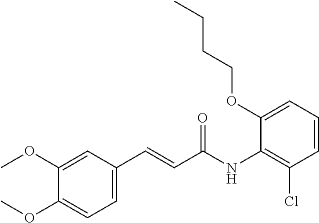 [SMILES: CCCCOc1cccc(Cl)c1NC(=O)/C=C/c1ccc(OC)c(OC)c1] | (E)-N-(2-butoxy-6- chlorophenyl)-3- (3,4- dimethoxyphenyl) acrylamide | Found: 412.1291 [M + Na]⁺ calc. 412.1292 |
| 37 | 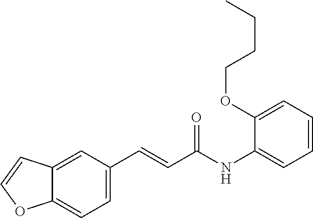 [SMILES: CCCCOc1ccccc1NC(=O)/C=C/c1ccc2occc2c1] | (E)-3- (benzofuran-5-yl)- N-(2- butoxyphenyl) acrylamide | Found: 358.1414 [M + Na]⁺ calc. 358.1419 |
| 38 | 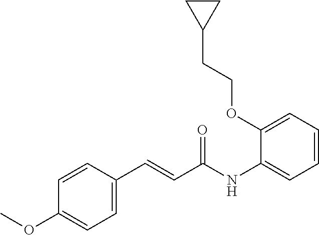 [SMILES: COc1ccc(/C=C/C(=O)Nc2ccccc2OCCC2CC2)cc1] | (E)-N-(2-(2- cyclopropylethoxy) phenyl)-3-(4- methoxyphenyl) acrylamide | Found: 360.1577 [M + Na]⁺ calc. 360.1576 |
| 39 | 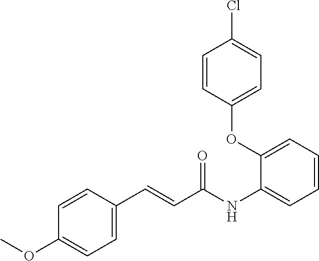 [SMILES: COc1ccc(/C=C/C(=O)Nc2ccccc2Oc2ccc(Cl)cc2)cc1] | (E)-N-(2-(4- chlorophenoxy) phenyl)-3-(4- methoxyphenyl) acrylamide | Found: 402.0875 [M + Na]⁺ calc. 402.0873 |
| 40 | 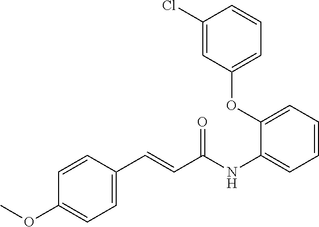 [SMILES: COc1ccc(/C=C/C(=O)Nc2ccccc2Oc2cccc(Cl)c2)cc1] | (E)-N-(2-(3- chlorophenoxy) phenyl)-3-(4- methoxyphenyl) acrylamide | Found: 402.0875 [M + Na]⁺ calc. 402.0873 |
| 41 | 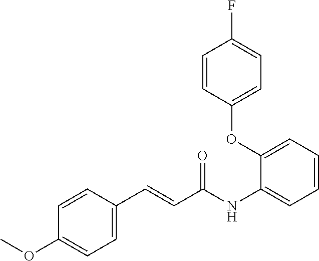 [SMILES: COc1ccc(/C=C/C(=O)Nc2ccccc2Oc2ccc(F)cc2)cc1] | (E)-N-(2-(4- fluorophenoxy) phenyl)-3-(4- methoxyphenyl) acrylamide | Found: 386.1156 [M + Na]⁺ calc. 386.1168 |
| 42 | 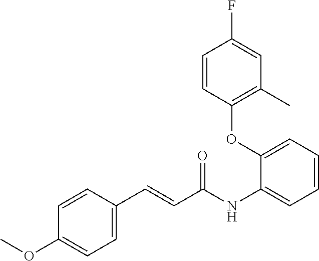 [SMILES: COc1ccc(/C=C/C(=O)Nc2ccccc2Oc2ccc(F)cc2C)cc1] | (E)-N-(2-(4-fluoro- 2- methylphenoxy) phenyl)-3-(4- methoxyphenyl) acrylamide | Found: 400.1328 [M + Na]⁺ calc. 400.1325 |
| 43 | 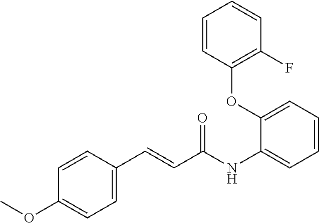 [SMILES: COc1ccc(/C=C/C(=O)Nc2ccccc2Oc2ccccc2F)cc1] | (E)-N-(2-(2- fluorophenoxy) phenyl)-3-(4- methoxyphenyl) acrylamide | Found: 386.1167 [M + Na]⁺ calc. 386.1168 |
| 44 | 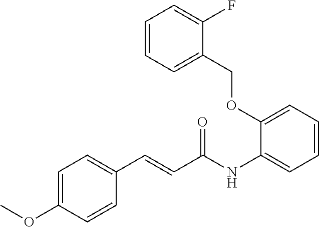 [SMILES: COc1ccc(/C=C/C(=O)Nc2ccccc2OCc2ccccc2F)cc1] | (E)-N-(2-((2- fluorobenzyl)oxy) phenyl)-3-(4- methoxyphenyl) acrylamide | Found: 400.1312 [M + Na]⁺ calc. 400.1325 |
| 45 | 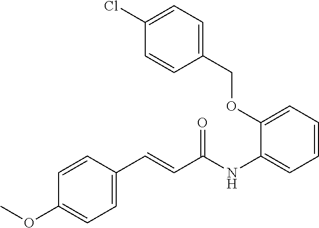 [SMILES: COc1ccc(/C=C/C(=O)Nc2ccccc2OCc2ccc(Cl)cc2)cc1] | (E)-N-(2-((4- chlorobenzyl)oxy) phenyl)-3-(4- methoxyphenyl) acrylamide | Found: 416.1029 [M + Na]⁺ calc. 416.1024 |
| 46 | 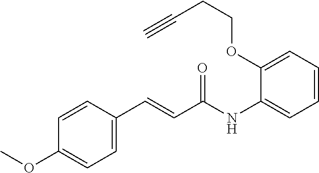 [SMILES: C#CCCOc1ccccc1NC(=O)/C=C/c1ccc(OC)cc1] | (E)-N-(2-(but-3- yn-1- yloxy)phenyl)-3- (4- methoxyphenyl) acrylamide | Found: 344.1247 [M + Na]⁺ calc. 344.1263 |
| 47 | 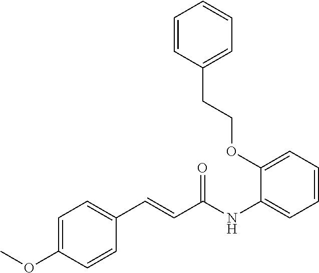 [SMILES: COc1ccc(/C=C/C(=O)Nc2ccccc2OCCc2ccccc2)cc1] | (E)-3-(4- methoxyphenyl)- N-(2- phenethoxyphenyl) acrylamide | Found: 396.1571 [M + Na]⁺ calc. 396.1576 |
| 48 | 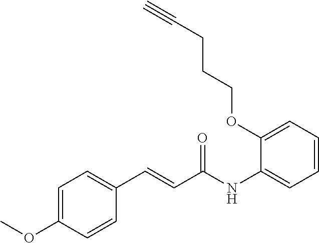 [SMILES: C#CCCCOc1ccccc1NC(=O)/C=C/c1ccc(OC)cc1] | (E)-3-(4- methoxyphenyl)- N-(2-(pent-4-yn-1- yloxy)phenyl) acrylamide | Found: 358.1430 [M + Na]⁺ calc. 358.1419 |
| 49 | 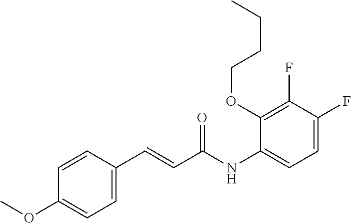 [SMILES: CCCCOc1c(NC(=O)/C=C/c2ccc(OC)cc2)ccc(F)c1F] | (E)-N-(2-butoxy- 3,4- difluorophenyl)-3- (4- methoxyphenyl) acrylamide | Found: 360.1572 [M + Na]⁺ calc. 360.1576 |
| 50 | 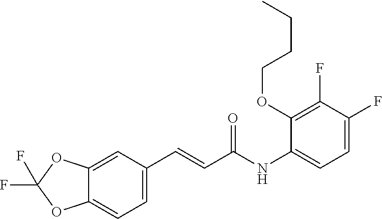 [SMILES: CCCCOc1c(NC(=O)/C=C/c2ccc3c(c2)OC(F)(F)O3)ccc(F)c1F] | (E)-N-(2-butoxy- 3,4- difluorophenyl)-3- (2,2- difluorobenzo[d][1, 3]dioxol-5- yl)acrylamide | Found: 434.0991 [M + Na]⁺ calc. 434.0991 |
| 51 | 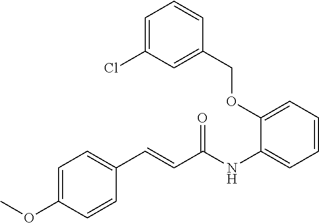 [SMILES: COc1ccc(/C=C/C(=O)Nc2ccccc2OCc2cccc(Cl)c2)cc1] | (E)-N-(2-((3- chlorobenzyl)oxy) phenyl)-3-(4- methoxyphenyl) acrylamide | Found: 416.1031 [M + Na]⁺ calc. 416.1029 |
| 52 | 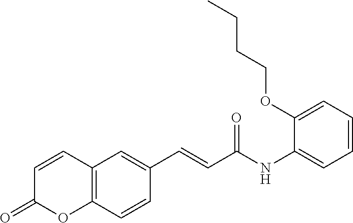 [SMILES: CCCCOc1ccccc1NC(=O)/C=C/c1ccc2oc(=O)ccc2c1] | (E)-N-(2- butoxyphenyl)-3- (2-oxo-2H- chromen-6- yl)acrylamide | Found: 386.1352 [M + Na]⁺ calc. 386.1364 |
| 53 | 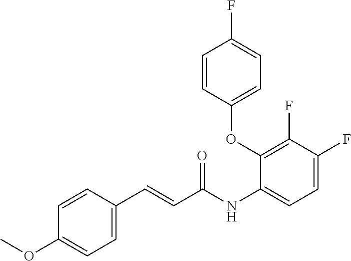 [SMILES: COc1ccc(/C=C/C(=O)Nc2ccc(F)c(F)c2Oc2ccc(F)cc2)cc1] | (E)-N-(3,4- difluoro-2-(4- fluorophenoxy) phenyl)-3-(4- methoxyphenyl) acrylamide | Found: 360.1572 [M + Na]⁺ calc. 360.1576 |
| 54 | 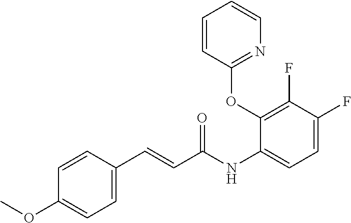 [SMILES: COc1ccc(/C=C/C(=O)Nc2ccc(F)c(F)c2Oc2ccccn2)cc1] | (E)-N-(3,4- difluoro-2-(pyridin- 2-yloxy)phenyl)-3-(4- methoxyphenyl) acrylamide | Found: 360.1572 [M + Na]⁺ calc. 360.1576 |
| 55 | 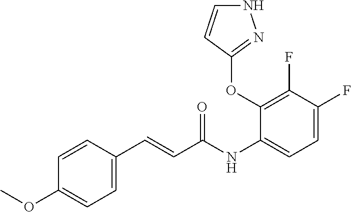 [SMILES: COc1ccc(/C=C/C(=O)Nc2ccc(F)c(F)c2Oc2cc[nH]n2)cc1] | (E)-N-(2-((1H- pyrazol-3-yl)oxy)-3,4- difluorophenyl)-3-(4- methoxyphenyl) acrylamide | Found: 360.1572 [M + Na]⁺ calc. 360.1576 |
| 56 | 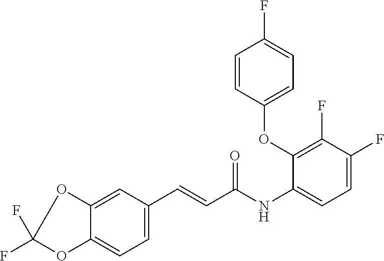 [SMILES: O=C(/C=C/c1ccc2c(c1)OC(F)(F)O2)Nc1ccc(F)c(F)c1Oc1ccc(F)cc1] | (E)-N-(3,4- difluoro-2-(4- fluorophenoxy) phenyl)-3-(2,2- difluorobenzo[d] [1,3]dioxol-5- yl)acrylamide | Found: 360.1572 [M + Na]⁺ calc. 360.1576 |
| 57 | 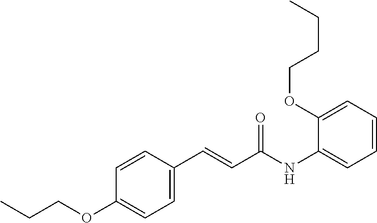 [SMILES: CCCCOc1ccccc1NC(=O)/C=C/c1ccc(OCCC)cc1] | (E)-N-(2- butoxyphenyl)-3- (4- propoxyphenyl) acrylamide | Found: 360.1572 [M + Na]⁺ calc. 360.1576 |
| 58 | 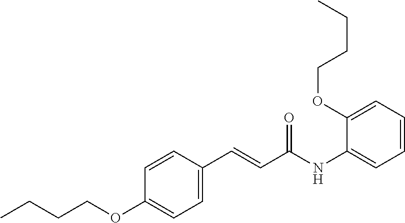 [SMILES: CCCCOc1ccc(/C=C/C(=O)Nc2ccccc2OCCCC)cc1] | (E)-N-(2- butoxyphenyl)-3- (4- butoxyphenyl) acrylamide | Found: 360.1572 [M + Na]⁺ calc. 360.1576 |
| 59 | 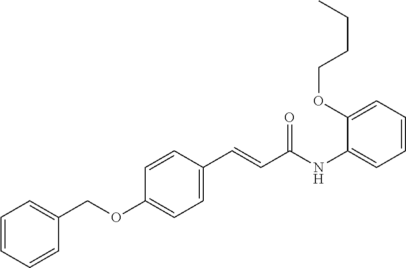 [SMILES: CCCCOc1ccccc1NC(=O)/C=C/c1ccc(OCc2ccccc2)cc1] | (E)-3-(4- (benzyloxy)phenyl)- N-(2- butoxyphenyl) acrylamide | Found: 360.1572 [M + Na]⁺ calc. 360.1576 |
| 60 | 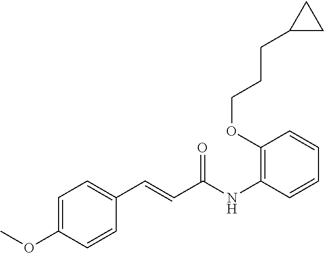 [SMILES: COc1ccc(/C=C/C(=O)Nc2ccccc2OCCCC2CC2)cc1] | (E)-N-(2-(3- cyclopropylpropoxy) phenyl)-3-(4- methoxyphenyl) acrylamide | Found: 374.1727 [M + Na]⁺ calc. 374.1732 |
| 61 | 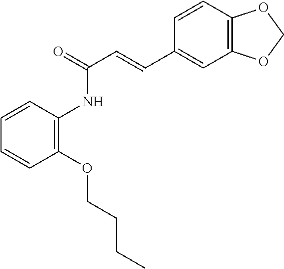 [SMILES: CCCCOc1ccccc1NC(=O)/C=C/c1ccc2c(c1)OCO2] | (E)-3- (benzo[d][1,3]dioxol- 5-yl)-N-(2- butoxyphenyl) acrylamide | 339.14/ 339.91 |
| 62 | 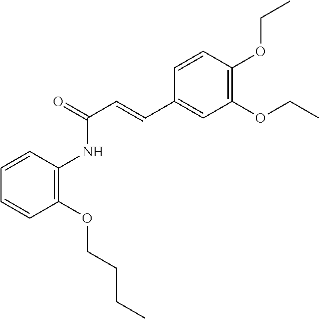 [SMILES: CCCCOc1ccccc1NC(=O)/C=C/c1ccc(OCC)c(OCC)c1] | (E)-N-(2- butoxyphenyl)-3- (3,4- diethoxyphenyl) acrylamide | 383.2/383.95 |
| 63 | 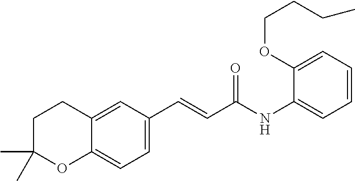 [SMILES: CCCCOc1ccccc1NC(=O)/C=C/c1ccc2c(c1)CCC(C)(C)O2] | (E)-N-(2- butoxyphenyl)-3- 2,2- dimethylchroman- 6-yl)acrylamide | Found: 402.2049 [M + Na]⁺ calc. 402.2045 |
| 64 | 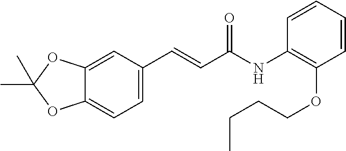 [SMILES: CCCCOc1ccccc1NC(=O)/C=C/c1ccc2c(c1)OC(C)(C)O2] | (E)-N-(2- butoxyphenyl)-3- (2,2- dimethylbenzo[d] [1,3]dioxol-5- yl)acrylamide | Found: 390.1685 [M + Na]⁺ calc. 390.1681 |
| 65 | 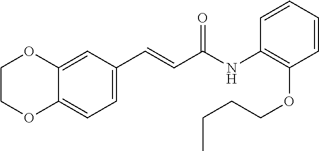 [SMILES: CCCCOc1ccccc1NC(=O)/C=C/c1ccc2c(c1)OCCO2] | (E)-N-(2- butoxyphenyl)-3- (2,3- dihydrobenzo[b][1, 4]dioxin-6- yl)acrylamide | Found: 376.1533 [M + Na]⁺ calc. 376.1525 |
| 83 | 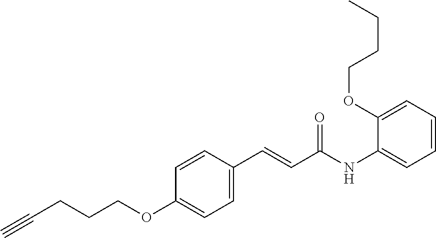 [SMILES: C#CCCCOc1ccc(/C=C/C(=O)Nc2ccccc2OCCCC)cc1] | (E)-N-(2- butoxyphenyl)-3- (4-(pent-4-yn-1- yloxy)phenyl) acrylamide | Found: 400.1893 [M + Na]⁺, calc. 400.1889 |
| 84 | 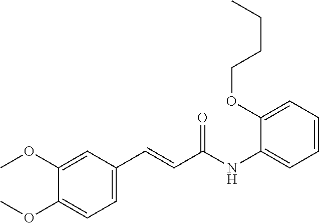 [SMILES: CCCCOc1ccccc1NC(=O)/C=C/c1ccc(OC)c(OC)c1] | (E)-N-(2- butoxyphenyl)-3- (3,4- dimethoxyphenyl) acrylamide | Found: 378.1686 [M + Na]⁺ calc. 378.1681 |
| 85 | 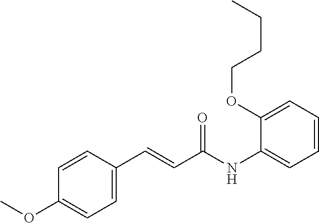 [SMILES: CCCCOc1ccccc1NC(=O)/C=C/c1ccc(OC)cc1] | (E)-N-(2- butoxyphenyl)-3- (4- dimethoxyphenyl) acrylamide | Found: 348.1575 [M + Na]⁺ calc. 348.1576 |
| 86 | 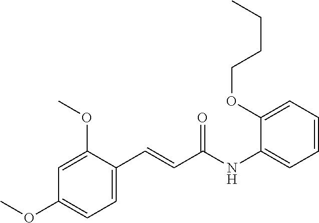 [SMILES: CCCCOc1ccccc1NC(=O)/C=C/c1ccc(OC)cc1OC] | (E)-3-(4- butoxyphenyl)-N- (2,4- dimethoxyphenyl) acrylamide | |
| 87 | 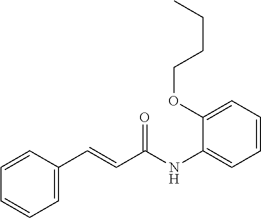 [SMILES: CCCCOc1ccccc1NC(=O)/C=C/c1ccccc1] | (E)- N-(2- Butoxyphenyl)-3- phenylprop-2- enamide | |
| 88 | 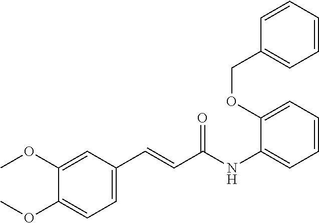 [SMILES: COc1ccc(/C=C/C(=O)Nc2ccccc2OCc2ccccc2)cc1OC] | (E)-N-(2-(benzyl- oxy)phenyl)-3- (3,4-dimethoxy- phenyl)acrylamide | Found: 382.1418 [M + Na] calc. 382.1419 |
| 142 | 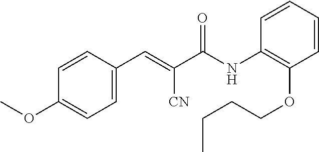 [SMILES: CCCCOc1ccccc1NC(=O)/C(C#N)=C/c1ccc(OC)cc1] | (E)-N-(2- butoxyphenyl)-2- cyano-3-(4- methoxyphenyl) acrylamide | Found: 373.1522 [M + Na] calc. 373.1528 |
| 143 | 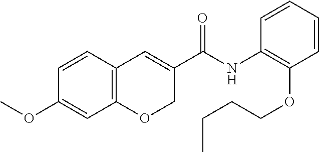 [SMILES: CCCCOc1ccccc1NC(=O)C1=Cc2ccc(OC)cc2OC1] | N-(2- butoxyphenyl)-7- methoxy-2H- chromene-3- carboxamide | Found: 376.1521 [M + Na] calc. 376.1525 |
| 144 | 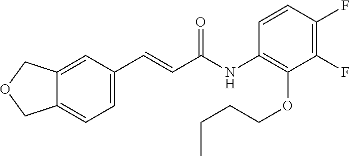 [SMILES: CCCCOc1c(NC(=O)/C=C/c2ccc3c(c2)COC3)ccc(F)c1F] | (E)-N-(2-butoxy- 3,4- difluorophenyl)-3- (1,3- dihydroisobenzofuran- 5-yl)acrylamide | Found: 396.1378 [M + Na] calc. 396.1387 |
| 145 | 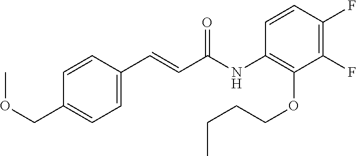 [SMILES: CCCCOc1c(NC(=O)/C=C/c2ccc(COC)cc2)ccc(F)c1F] | (E)-N-(2-butoxy- 3,4- difluorophenyl)-3- (4- (methoxymethyl) phenyl)acrylamide | Found: 398.1548 [M + Na] calc. 398.1544 |
| 147 | 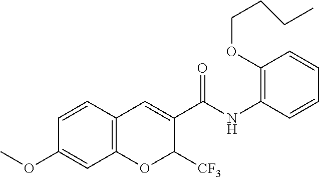 [SMILES: CCCCOc1ccccc1NC(=O)C1=Cc2ccc(OC)cc2OC1C(F)(F)F] | N-(2- butoxyphenyl)-7- methoxy-2- (trifluoromethyl)- 2H-chromene-3- carboxamide | Found: 444.1391 [M + Na] calc. 444.1399 |
| 148 | 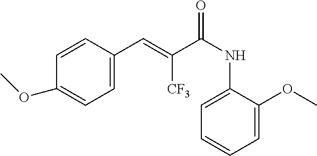 | (Z)-N-(2- butoxyphenyl)-3- (4- methoxyphenyl)- 2- (trifluoromethyl) acrylamide | Found: 416.1447 [M + Na] calc. 416.1449 |
| 149 | 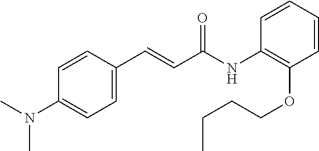 [SMILES: CCCCOc1ccccc1NC(=O)/C=C/c1ccc(N(C)C)cc1] | (E)-N-(2- butoxyphenyl)-3- (4- (dimethylamino) phenyl)acrylamide | Found: 361.1893 [M + Na] calc. 361.1892 |
| 150 | 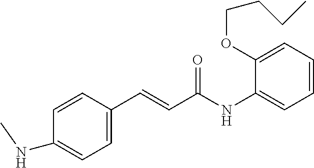 [SMILES: CCCCOc1ccccc1NC(=O)/C=C/c1ccc(NC)cc1] | (E)-N-(2- butoxyphenyl)-3- (4- (methylamino) phenyl)acrylamide | Found: 347.1726 [M + Na] calc. 347.1735 |
| 151 | 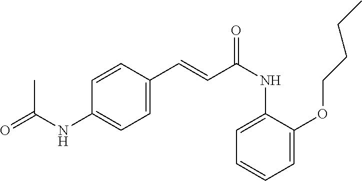 [SMILES: CCCCOc1ccccc1NC(=O)/C=C/c1ccc(NC(C)=O)cc1] | (E)-3-(4- acetamidophenyl)- N-(2- butoxyphenyl) acrylamide | Found: 375.1680 [M + Na] calc. 375.1685 |
| 152 | 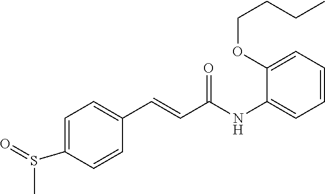 [SMILES: CCCCOc1ccccc1NC(=O)/C=C/c1ccc(S(C)=O)cc1] | (E)-N-(2- butoxyphenyl)-3- (4- (methylsulfinyl) phenyl)acrylamide | Found: 380.1295 [M + Na] calc. 380.1296 |
| 153 | 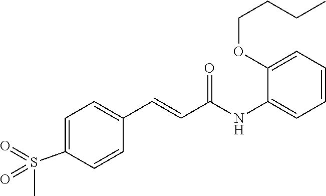 [SMILES: CCCCOc1ccccc1NC(=O)/C=C/c1ccc(S(C)(=O)=O)cc1] | (E)-N-(2- butoxyphenyl)-3- (4- (methylsulfonyl) phenyl)acrylamide | Found: 396.1232 [M + Na] calc. 396.1245 |
| 154 | 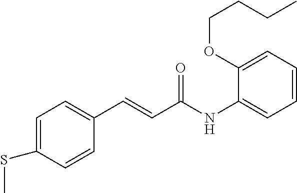 [SMILES: CCCCOc1ccccc1NC(=O)/C=C/c1ccc(SC)cc1] | (E)-N-(2- butoxyphenyl)-3- (4- (methylthio)phenyl) acrylamide | Found: 364.1331 [M + Na] calc. 364.1367 |
| 155 | 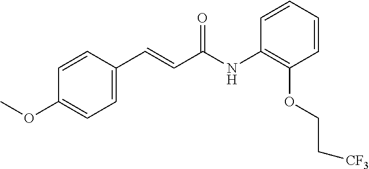 [SMILES: COc1ccc(/C=C/C(=O)Nc2ccccc2OCCC(F)(F)F)cc1] | (E)-3-(4- methoxyphenyl)- N-(2-(3,3,3- trifluoropropoxy) phenyl)acrylamide | Found: 388.1136 [M + Na] calc. 388.1136 |
| 156 | 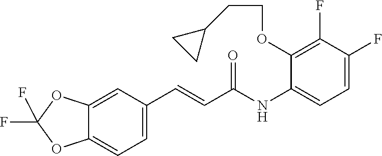 [SMILES: O=C(/C=C/c1ccc2c(c1)OC(F)(F)O2)Nc1ccc(F)c(F)c1OCCC1CC1] | (E)-N-(2-(2- cyclopropylethoxy)- 3,4- difluorophenyl)-3- (2,2- difluorobenzo[d][1, 3]dioxol-5- yl)acrylamide | Found: 446.0993 [M + Na] calc. 446.0991 |
| 157 | 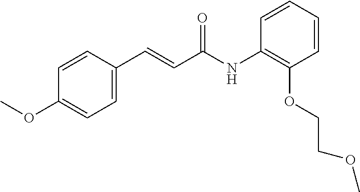 | (E)-N-(2-(2- methoxyethoxy) phenyl)-3-(4- methoxyphenyl) acrylamide | Found: 350.1366 [M + Na] calc. 350.1368 |
| 158 | 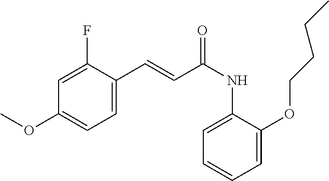 [SMILES: CCCCOc1ccccc1NC(=O)/C=C/c1ccc(OC)cc1F] | (E)-N-(2- butoxyphenyl)-3- (2-fluoro-4- methoxyphenyl) acrylamide | Found: 366.1481 [M + Na] calc. 366.1481 |
| 159 | 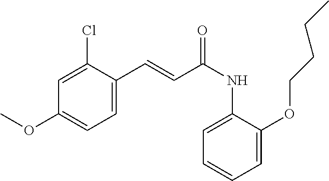 [SMILES: CCCCOc1ccccc1NC(=O)/C=C/c1ccc(OC)cc1Cl] | (E)-N-(2- butoxyphenyl)-3- (2-chloro-4- methoxyphenyl) acrylamide | Found: 382.1185 [M + Na] calc. 382.1186 |
| 160 | 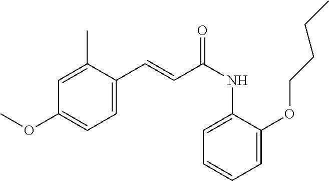 [SMILES: CCCCOc1ccccc1NC(=O)/C=C/c1ccc(OC)cc1C] | (E)-N-(2- butoxyphenyl)-3- (4-methoxy-2- methylphenyl) acrylamide | Found: 362.1741 [M + H] calc. 362.1732 |
| 161 | 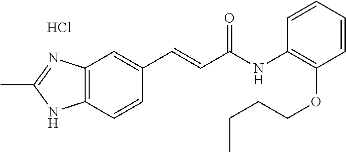 [SMILES: CCCCOc1ccccc1NC(=O)/C=C/c1ccc2[nH]c(C)nc2c1 ; Cl] | (E)-N-(2- butoxyphenyl)-3- (2-methyl-1H- benzo[d]imidazol- 5-yl)acrylamide | Found: 350.1873 [M + H] calc. 350.1869 |
| 162 | 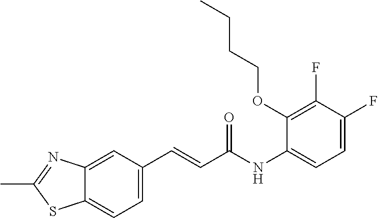 [SMILES: CCCCOc1c(NC(=O)/C=C/c2ccc3sc(C)nc3c2)ccc(F)c1F] | (E)-N-(2-butoxy- 3,4- difluorophenyl)-3- (2- methylbenzo[d] thiazol-5- yl)acrylamide | Found: 425.1113 [M + Na] calc. 425.1111 |
| 163 | 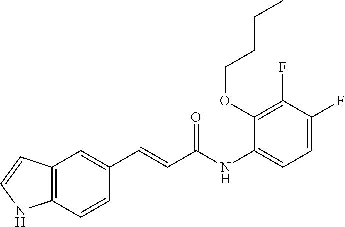 [SMILES: CCCCOc1c(NC(=O)/C=C/c2ccc3[nH]ccc3c2)ccc(F)c1F] | (E)-N-(2-butoxy- 3,4- difluorophenyl)-3- (1H-indol-5- yl)acrylamide | Found: 393.1400 [M + Na] calc. 393.1391 |
| 164 | 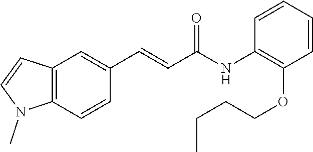 [SMILES: CCCCOc1ccccc1NC(=O)/C=C/c1ccc2c(ccn2C)c1] | (E)-N-(2- butoxyphenyl)-3- (1-methyl-1H- indol-5- yl)acrylamide | Found: 371.1739 [M + Na] calc. 371.1735 |
| 165 | 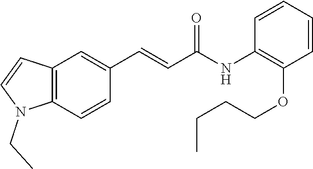 [SMILES: CCCCOc1ccccc1NC(=O)/C=C/c1ccc2c(ccn2CC)c1] | (E)-N-(2- butoxyphenyl)-3- (1-ethyl-1H-indol- 5-yl)acrylamide | Found: 385.1901 [M + Na] calc. 385.1892 |
| 166 | 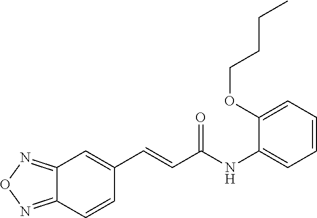 [SMILES: CCCCOc1ccccc1NC(=O)/C=C/c1ccc2nonc2c1] | (E)-3- (benzo[c][1,2,5] oxadiazol-5-yl)-N-(2- butoxyphenyl) acrylamide | Found: 360.1324 [M + Na] calc. 360.1324 |

| Example | ¹H-NMR (CDCl3) | ¹³C-NMR (CDCl3) |
|---|---|---|
| 7 | 0.96 (3H, t, J = 7.38 Hz), 1.48 (2H, sextet, J = 7.48 | 14.05, 19.49, 31.38, 55.47, 68.60, |
| | Hz ), 1.79 (2H, quintet, J = 7.06 Hz), 3.78 (3H, s), | 111.06, 113.37, 115.61, 120.08, |
| | 4.01 (2H, t, J = 6.60 Hz), 6.47 (1H, d, J = 15.49 | 120.69, 121.20, 121.84, 123.93, |
| | Hz), 6.82 (1H, dd, J = 1.46, 7.98 Hz), 6.86 (1H, | 128.09, 129.99, 136.28, 141.94, |
| | ddd, J = 0.71, 2.55, 8.21 Hz), 6.91 (1H, dt, | 147.41, 160.04, 163.67. |
| | J = 1.47, 7.71 Hz), 6.97 (1H, dt, J = 1.77, 7.73 Hz), | |
| | 7.01 (1H, t, J = 1.98 Hz), 7.09 (1H, d, J = 7.68 Hz), | |
| | 7.24 (1H, t, J = 7.92 Hz), 7.63 (1H, d, J = 15.49 | |
| | Hz), 7.90 (1H, s), 8.44 (1H, d, J = 7.08 Hz). | |
| 8 | 1.03 (3H, t, J = 7.38 Hz), 1.55 (2H, sextet, J = 7.47 | 14.05, 19.49, 31.38, 68.61, |
| | Hz), 1.86 (2H, quintet, J = 7.06 Hz), 4.08 (2H, t, | 111.07, 120.11, 120.54 (q), |
| | J = 6.60 Hz), 6.53 (1H, d, J = 15.53 Hz), 6.90 (1H, | 121.22, 121.31, 122.41, 124.08, |
| | m), 6.99 (1H, m), 7.06 (1H, m)6.90 (1H, dd, | 127.97, 129.50, 133.55, 140.42, |
| | J = 1.12, 8.20 Hz), 6.98 (1H, dt, J = 1.24, 11.84 | 147.42, 150.25 (q, J = 1.72 Hz), |
| | Hz), 7.06 (1H, dt, J = 1.44, 7.78 Hz), 7.24 (2H, d, | 163.30. |
| | J = 8.24 Hz), 7.59 (2H, d, J = 8.60 Hz), 7.72 (1H, | |
| | d, J = 15.49 Hz), 7.97 (1H, s), 8.52 (1H, d, J = 7.04 | |
| | Hz). | |
| 9 | 0.95 (3H, t, J = 7.48 Hz),0.97 (3H, t, J = 7.48 Hz), | 10.62, 14.04, 19.48, 22.64, 31.37, |
| | 1.46 (2H, sextet, J = 7.48 Hz), 1.70-1.82 (4H, m), | 68.55, 69.74, 111.01, 114.92, |
| | 3.88 (2H, t, J = 6.56 Hz), 3.99 (2H, t, J = 6.60 Hz), | 118.82, 120.02, 121.17, 123.69, |
| | 6.34 (1H, d, J = 15.45 Hz), 6.80-6.84 (3H, m), | 127.35, 128.27, 129.67, 141.77, |
| | 6.94 (1H, dt, J = 1.77, 11.47 Hz), 7.43 (1H, d, | 147.37, 160.81, 164.18. |
| | J = 8.68 Hz), 7.43 (2H, d, J = 8.68 Hz), 7.61 (1H, | |
| | d, J = 15.45 Hz), 7.85 (1H, s), 8.45 (1H, d, J = 6.40 | |
| | Hz). | |
| 10 | 1.01 (3H, t, J = 7.41 Hz), 1.54 (2H, sextet, J = 7.50 | 13.94, 19.31, 32.24, 56.10, 56.15, |
| | Hz), 1.81 (2H, quintet, J = 7.10 Hz), 3.93 (1H, s), | 74.53 (d, J = 6.28 Hz), 110.06, |
| | 3.94 (1H, s), 4.23 (2H, dt, J = 1.10, 6.62 Hz), 6.37 | 110.79 (d, J = 17.65 Hz), 111.29, |
| | (1H, d, J = 15.43 Hz), 6.86-6.91 (2H, m, J = 3.91 | 114.62, 118.45, 122.46, 127.56, |
| | Hz), 7.07 (1H, d, J = 1.92 Hz), 7.15 (1H, dd, | 128.50 (1C, t, J = 2.86 Hz), 137.10 |
| | J = 1.95, 8.31 Hz), 7.68 (1H, d, J = 15.43 Hz), 7.77 | (d, J = 9.75 Hz), 142.74, 143.75 |
| | (1H, s), 8.22 (1H, t, J = 6.90 Hz). | (dd, J = 14.89, 247.07 Hz), 147.62 |
| | | (dd, J = 11.15, 245.37 Hz), 149.38, |
| | | 151.18, 164.18. |
| 11 | 1.03 (3H, t, J = 7.38 Hz), 1.54 (2H, sextet, J = 7.47 | 14.05, 19.48, 31.36, 68.58, |
| | Hz), 1.86 (2H, quintet, J = 7.04 Hz), 4.08 (3H, t, | 111.04, 117.05 (t, J = 391.00 Hz), |
| | J = 6.60 Hz), 6.50 (1H, d, J = 15.49 Hz), 6.55 (1H, | 119.79, 120.07, 121.19, 121.61, |
| | t, J = 73.48 Hz), 6.90 (1H, d, J = 7.92 Hz), 6.99 | 124.00, 128.02, 129.62, 132.12, |
| | (1H, dt, J = 1.03, 7.77 Hz), 7.05 (1H, dt, J = 1.29, | 140.69, 147.40, 152.29 (1C, t, |
| | 7.76 Hz), 7.14 (2H, d, J = 8.48 Hz), 7.57 (2H, d, | J = 2.82 Hz), 163.48. |
| | J = 8.60 Hz), 7.71 (1H, d, J = 15.53 Hz), 7.97 (1H, | |
| | s), 8.52 (1H, d, J = 6.76 Hz). | |
| 12 | 3.83 (3H, s), 5.15 (2H, s), 6.38 (1H, d, J = 15.49 | 55.50, 71.19, 111.83, 114.40, |
| | Hz), 6.90 (2H, d, J = 8.68 Hz), 6.96-7.04 (3H, m), | 118.97, 121.78, 123.75, 127.56, |
| | 7.39-7.49 (7H, m), 7.67 (1H, d, J = 15.49 Hz), | 127.82, 128.50, 128.52, 128.96, |
| | 7.94 (1H, s), 8.55 (1H, s). | 129.71, 130.99, 136.61, 141.75, |
| | | 147.28, 161.20, 164.21. |
| 13 | 1.01 (3H, t, J = 7.41 Hz), 1.53 (2H, sextet, J = 7.50 | 13.91, 19.27, 32.23, 55.52, 74.50 |
| | Hz), 1.81 (2H, quintet, J = 7.08 Hz), 3.84 (3H, s), | (d, J = 6.26 Hz), 110.73 (d, J = 17.63 |
| | 4.22 (2H, dt, J = 1.02, 6.60 Hz), 6.38 (1H, d, | Hz), 114.50, 114.58, 118.18, |
| | J = 15.43 Hz), 6.87 (1H, q, J = 9.13 Hz), 6.92 (2H, | 127.31, 128.53 (t, J = 2.79 Hz), |
| | d, J = 8.76 Hz), 7.50 (2H, d, J = 8.64 Hz), 7.70 | 129.78, 137.11 (d, J = 9.39 Hz), |
| | (1H, d, J = 15.43 Hz), 7.77 (1H, s), 8.21 (1H, s). | 142.49, 143.73 (dd, J = 14.86, |
| | | 246.98 Hz), 147.59 (dd, J = 11.31, |
| | | 245.33 Hz), 161.43, 164.29. |
| 14 | 1.03 (3H, t, J = 7.40 Hz), 1.53 (2H, sextet, J = 7.48 | 14.03, 19.46, 31.35, 56.38, 68.57, |
| | Hz), 1.86 (2H, quintet, J = 7.08 Hz), 3.93 (3H, s), | 111.02, 112.10, 120.05, 120.34, |
| | 4.07 (2H, t, J = 6.64 Hz), 6.43 (1H, d, J = 15.49 | 121.14, 123.20, 123.89, 128.08, |
| | Hz), 6.89 (1H, dd, J = 1.40, 8.00 Hz), 6.92 (1H, d, | 128.46, 128.58, 129.10, 140.39, |
| | J = 8.52 Hz), 6.97 (1H, dt, J = 1.41, 7.71 Hz), 7.04 | 147.40, 156.32, 163.62. |
| | (1H, dt, J = 1.72, 7.73 Hz), 7.39 (1H, d, J = 2.12 | |
| | Hz), 7.41 (1H, d, J = 2.08 Hz), 7.62 (1H, d, | |
| | J = 2.12 Hz), 7.63 (1H, d, J = 15.41 Hz), 7.94 (1H, | |
| | s), 8.51 (1H, d, J = 6.92 Hz). | |
| 15 | 3.88 (3H, s), 3.88 (3H, s), 6.46 (1H, dd, J = 1.24, | 55.88 , 55.94, 109.63, 111.06, |
| | 15.41 Hz), 6.81-6.84 (2H, m), 6.98-7.05 (4H, m), | 117.58, 118.87, 121.00, 122.51, |
| | 7.07-7.16 (3H, m), 7.33-7.37 (2H, m), 7.66 (1H, | 123.95, 124.01, 124.08, 127.65, |
| | d, J = 15.45 Hz), 8.02 (1H, s), 8.62 (1H, d, J = 8.00 | 130.03, 130.15, 142.25, 145.80, |
| | Hz) | 149.17, 150.85, 156.50, 164.26. |
| 16 | 1.03 (3H, t, J = 7.38 Hz), 1.53 (3H, sextet, J = 7.47 | 14.03, 19.45, 31.27, 55.53, 68.95, |
| | Hz), 1.85 (2H, quintet, J = 7.05 Hz), 3.85 (1H, s), | 111.75, 114.46, 118.50, 119.93, |
| | 4.05 (2H, t, J = 6.60 Hz), 6.39 (1H, d, J = 15.45 | 123.14, 126.23, 127.41, 129.12, |
| | Hz), 6.78 (1H, d, J = 8.68 Hz), 6.92 (1H, d, | 129.80, 142.32, 145.87, 161.35, |
| | J = 8.68 Hz), 6.98 (1H, dd, J = 2.54, 8.62 Hz), 7.52 | 164.13. |
| | (2H, d, J = 8.68 Hz), 7.69 (1H, d, J = 15.45 Hz), | |
| | 7.87 (1H, s), 8.59 (1H, d, J = 1.96 Hz). | |
| 17 | 1.02 (3H, t, J = 7.38 Hz), 1.53 (3H, sextet, J = 7.48 | 14.01, 19.41, 31.23, 56.08, 56.09, |
| | Hz), 1.85 (2H, quintet, J = 7.07 Hz), 3.92 (1H, s), | 68.95, 110.08, 111.21, 111.77, |
| | 3.94 (1H, s), 4.06 (2H, t, J = 6.64 Hz), 6.39 (1H, | 118.76, 119.93, 122.40, 123.16, |
| | d, J = 15.41 Hz), 6.79 (1H, d, J = 8.72 Hz), 6.88 | 126.17, 127.63, 129.06, 142.51, |
| | (1H, d, J = 8.28 Hz), 6.98 (1H, dd, J = 2.54, 8.66 | 145.87, 149.29, 151.04, 164.03. |
| | Hz), 7.08 (1H, d, J = 1.64 Hz), 7.16 (1H, dd, | |
| | J = 1.62, 8.26 Hz), 7.67 (1H, d, J = 15.45 Hz), 7.89 | |
| | (1H, s), 8.59 (1H, d, J = 1.92 Hz). | |
| 18 | 1.03 (3H, t, J = 7.38 Hz), 1.60 (2H, sextet, J = 7.48 | 14.04, 19.47, 32.40, 55.54, 73.73, |
| | Hz), 1.85 (2H, quintet, J = 7.04 Hz), 3.85 (1H, s), | 114.52, 118.23, 119.04, 124.98, |
| | 4.05 (2H, t, J = 6.54 Hz), 6.39 (1H, d, J = 15.45 | 125.22, 127.02, 127.31, 129.80, |
| | Hz), 6.93 (2H, d, J = 8.72 Hz), 7.04-7.10 (2H, m), | 133.70, 142.61, 144.27, 161.45, |
| | 7.51 (2H, d, J = 8.68 Hz), 7.71 (1H, d, J = 15.45 | 164.26. |
| | Hz), 7.93 (1H, s), 8.41 (1H, d, J = 6.08 Hz). | |
| 19 | 1.03 (3H, t, J = 7.38 Hz), 1.60 (2H, m, J = 7.44 | 14.05, 19.50, 32.39, 56.08, 56.14, |
| | Hz), 1.85 (2H, m, J = 7.04 Hz), 3.92 (1H, s), 3.94 | 73.73, 110.02, 111.29, 118.51, |
| | (1H, s), 4.06 (3H, t, J = 6.54 Hz), 6.39 (1H, d, | 119.05, 122.47, 125.03, 125.23, |
| | J = 15.45 Hz), 6.89 (1H, d, J = 8.32 Hz), 7.04-7.10 | 127.04, 127.56, 133.66, 142.82, |
| | (2H, m), 7.15 (1H, dd, J = 1.62, 8.30 Hz), 7.69 | 144.28, 149.37, 151.19, 164.15. |
| | (1H, d, J = 15.41 Hz), 7.95 (1H, s), 8.40 (1H, d, | |
| | J = 6.80 Hz). | |
| 20 | 1.03 (3H, t, J = 7.40 Hz), 1.43 (3H, t, J = 7.00 Hz), | 14.04, 14.87, 19.48, 31.37, 63.72, |
| | 1.54 (2H, sextet, J = 7.48 Hz), 1.86 (2H, quintet, | 68.56, 111.02, 114.89, 118.87, |
| | J = 7.04 Hz), 4.04-4.09 (4H, m), 6.42 (1H, d, | 120.02, 121.17, 123.69, 127.41, |
| | J = 15.45 Hz), 6.88-6.89 (3H, m), 6.98 (1H, dt, | 128.27, 129.68, 141.75, 147.37, |
| | J = 1.29, 7.63 Hz), 7.03 (1H, dt, J = 1.65, 7.66 Hz), | 160.60, 164.17. |
| | 7.50 (2H, d, J = 8.68 Hz), 7.69 (1H, d, J = 15.45 | |
| | Hz), 7.92 (1H, s), 8.52 (1H, d, J = 5.92 Hz). | |
| 21 | 1.03 (3H, t, J = 7.40 Hz), 1.54 (2H, sextet, J = 7.48 | 14.01, 19.43, 31.20, 55.53, 68.96, |
| | Hz), 1.86 (2H, quintet, J = 7.06 Hz), 3.84 (3H, s), | 111.70, 114.46, 118.63, 120.64, |
| | 4.05 (2H, t, J = 6.62 Hz), 6.40 (1H, d, J = 15.41 | 120.99, 126.92, 127.47, 128.55, |
| | Hz), 6.87 (1H, d, J = 2.12 Hz), 6.91 (1H, d, | 129.76, 142.11, 147.92, 161.32, |
| | J = 8.68 Hz), 6.95 (1H, dd, J = 2.16, 8.68 Hz), 7.51 | 164.11. |
| | (2H, d, J = 8.72 Hz), 7.69 (1H, d, J = 15.45 Hz), | |
| | 7.81 (1H, s), 8.47 (1H, d, J = 8.40 Hz). | |
| 22 | 1.03 (3H, t, J = 7.40 Hz), 1.53 (2H, sextet, J = 7.47 | 14.04, 19.48, 31.38, 55.53, 69.23, |
| | Hz), 1.80-1.88 (2H, m), 3.84 (1H, s), 4.04 (2H, t, | 107.68 (d, J = 29.35 Hz), 109.07 (d, |
| | J = 6.62 Hz), 6.40 (1H, d, J = 15.45 Hz), 6.70 (1H, | J = 23.44 Hz), 111.41 (d, J = 9.29 |
| | dt, J = 3.05, 8.44 Hz), 6.79 (1H, dd, J = 4.94, 8.94 | Hz), 114.47, 118.54, 127.42, |
| | Hz), 6.92 (2H, d, J = 8.76 Hz), 7.51 (2H, d, | 129.10 (d, J = 11.93 Hz), 129.79, |
| | J = 8.68 Hz), 7.70 (1H, d, J = 15.45 Hz), 7.94 (1H, | 142.31, 143.47 (d, J = 2.57 Hz), |
| | s), 8.36 (1H, dd, J = 2.84, 10.68 Hz). | 157.11 (d, J = 237.04 Hz), 161.36, |
| | | 164.18. |
| 23 | 1.02 (3H, t, J = 7.38 Hz), 1.52 (2H, sextet, J = 7.47 | 14.01, 19.41, 31.19, 56.11, 56.13, |
| | Hz), 1.86 (2H, quintet, J = 7.07 Hz), 3.91 (3H, s), | 68.99, 110.13, 111.26, 111.74, |
| | 3.93 (3H, s), 4.05 (2H, t, J = 6.62 Hz), 6.40 (1H, | 118.90, 120.72, 121.01, 122.36, |
| | d, J = 15.41 Hz), 6.86-6.88 (2H, m), 6.95 (1H, dd, | 126.88, 127.72, 128.61, 142.36, |
| | J = 2.02, 8.66 Hz), 7.06 (1H, d, J = 1.52 Hz), 7.14 | 147.94, 149.33, 151.05, 164.02. |
| | (1H, dd, J = 1.56, 8.28 Hz), 7.66 (1H, d, J = 15.45 | |
| | Hz), 7.83 (1H, s), 8.46 (1H, d, J = 8.36 Hz). | |
| 24 | 1.02 (3H, t, J = 7.36 Hz), 1.52 (2H, sextet, J = 7.46 | 14.02, 19.44 31.34, 56.08, 56.10, |
| | Hz), 1.84 (2H, quintet, J = 7.03 Hz), 3.92 (3H, s), | 69.22, 107.68 (d, J = 29.84 Hz), |
| | 3.93 (3H, s), 4.04 (2H, t, J = 6.60 Hz), 6.40 (1H, | 109.10 (d, J = 23.34 Hz), 110.08, |
| | d, J = 15.45 Hz), 6.70 (1H, dt, J = 2.95, 8.39 Hz), | 111.22, 111.43 (d, J = 9.34 Hz), |
| | 6.79 (1H, dd, J = 4.90, 8.90 Hz), 6.88 (1H, d, | 118.78, 122.40, 127.64, 129.04 (d, |
| | J = 8.28 Hz), 7.07 (1H, d, J = 1.28 Hz), 7.15 (1H, | J = 11.87 Hz), 142.52, 143.46 (d, |
| | d, J = 8.20 Hz), 7.67 (1H, d, J = 15.41 Hz), 7.96 | J = 2.44 Hz), 149.30, 151.05, |
| | (1H, s), 8.36 (1H, dd, J = 2.58, 10.58 Hz). | 157.06 (d, J = 237.05 Hz), 164.08. |
| 25 | 1.01 (3H, t, J = 7.36 Hz), 1.53 (2H, sextet, J = 7.46 | 13.93, 19.33, 32.28, 55.50, 74.18 |
| | Hz), 1.80 (2H, quintet, J = 7.01 Hz), 3.84 (3H, s), | (d, J = 6.10 Hz), 111.49 (d, J = 18.83 |
| | 4.17 (2H, t, J = 6.48 Hz), 6.40 (1H, d, J = 15.45 | Hz), 114.47, 115.68 (d, J = 2.83 |
| | Hz), 6.81 (1H, t, J = 9.78 Hz), 6.92 (2H, d, J = 8.60 | Hz), 118.38, 123.74 (d, J = 8.53 |
| | Hz), 6.98-7.03 (1H, m), 7.51 (2H, d, J = 8.56 Hz), | Hz), 127.35, 129.75, 133.07 (d, |
| | 7.71 (1H, d, J = 15.45 Hz), 7.96 (1H, s), 8.28 (1H, | J = 4.64 Hz), 135.53 (d, J = 13.03 |
| | d, J = 8.08 Hz). | Hz), 142.38, 154.78 (d, J = 244.90 |
| | | Hz), 161.37, 164.19. |
| 26 | 1.01 (3H, t, J = 7.38 Hz), 1.55 (2H, sextet, J = 7.46 | 13.95, 19.37, 32.30, 55.52, 74.20 |
| | Hz), 1.80 (2H, quintet, J = 7.04 Hz), 3.92 (3H, s), | (d, J = 6.20 Hz), 110.65 (d, |
| | 3.94 (3H, s), 4.17 (2H, t, J = 6.52 Hz), 6.40 (1H, | J = 124.70 Hz), 111.56 (d, J = 18.82 |
| | d, J = 15.41 Hz), 6.82 (1H, t, J = 9.78 Hz), 6.89 | Hz), 115.70 (d, J = 2.71 Hz), |
| | (1H, d, J = 8.28 Hz), 6.98-7.04 (1H, m), 7.07 (1H, | 118.66, 122.43, 123.78 (d, J = 8.53 |
| | d, J = 1.16 Hz), 7.15 (1H, d, J = 8.28 Hz), 7.69 | Hz), 127.62, 133.03 (d, J = 4.63 |
| | (1H, d, J = 15.45 Hz), 7.97 (1H, s), 8.28 (1H, d, | Hz), 135.53 (d, J = 13.09 Hz), |
| | J = 8.20 Hz). | 142.65, 149.35, 151.12, 153.58, |
| | | 156.01, 164.12. |
| 27 | 0.93 (3H, t, J = 7.12 Hz), 1.44 (2H, sextet, J = 7.23 | 13.94, 19.29, 31.24, 55.46, 68.97, |
| | Hz), 1.75 (2H, quintet, J = 6.90 Hz), 3.82 (3H, s), | 107.66 (d, J = 2.89 Hz), 108.59 (d, |
| | 3.98 (2H, t, J = 6.22 Hz), 6.49 (1H, d, J = 15.28 | J = 20.80 Hz), 114.35, 114.52 (d, |
| | Hz), 6.69 (1H, d, J = 8.16 Hz), 6.76 (1H, t, J = 8.76 | J = 14.80 Hz), 117.43, 127.28 (d, |
| | Hz), 6.89 (2H, d, J = 8.04 Hz), 6.99 (1H, s), 7.14 | J = 9.49 Hz), 127.64, 129.68, |
| | (2H, d, J = 6.84 Hz), 7.47 (2H, d, J = 7.76 Hz), | 142.20, 154.47, 158.06 (d, |
| | 7.70 (1H, d, J = 15.53 Hz). | J = 249.67 Hz), 161.14, 165.03. |
| 28 | 0.94 (3H, t, J = 7.34 Hz), 1.45 (2H, sextet, J = 7.43 | 13.94, 19.29, 31.25, 56.02, 56.07, |
| | Hz), 1.75 (2H, quintet, J = 6.96 Hz), 3.90 (6H, s), | 68.98, 107.66 (d, J = 2.90 Hz), |
| | 3.99 (2H, t, J = 6.50 Hz), 6.49 (1H, d, J = 15.49 | 108.62 (d, J = 20.77 Hz), 110.04, |
| | Hz), 6.70 (1H, d, J = 8.32 Hz), 6.76 (1H, t, J = 8.84 | 111.19, 114.46 (d, J = 14.82 Hz), |
| | Hz), 6.86 (1H, d, J = 8.28 Hz), 6.97 (1H, s), 7.05 | 117.65, 122.28, 127.32 (d, |
| | (1H, s), 7.10-7.17 (1H, m), 7.69 (1H, d, J = 15.53 | J = 10.18 Hz), 127.89, 142.46, |
| | Hz). | 149.25, 150.87, 154.43 (d, J = 4.64 |
| | | Hz), 158.06 (d, J = 249.68 Hz), |
| | | 164.80. |
| 29 | 2.13-2.20 (2H, m), 2.29-2.40 (2H, m), 3.85 (1H, | 22.25 (q, J = 2.95 Hz), 30.91 (q, |
| | s), 4.13 (2H, t, J = 6.18 Hz), 6.42 (1H, d, J = 15.45 | J = 29.36 Hz), 55.49, 67.01, |
| | Hz), 6.87 (1H, dd, J = 1.56, 7.60 Hz), 6.92 (2H, d, | 110.95, 114.43, 118.69, 120.46, |
| | J = 8.68 Hz), 6.99-7.06 (2H, m), 7.51 (2H, d, | 121.74, 123.85, 127.11 (q, |
| | J = 8.68 Hz), 7.70 (1H, d, J = 15.45 Hz), 7.82 (1H, | J = 276.18 Hz), 127.47, 128.13, |
| | s), 8.52 (1H, s). | 129.71, 141.97, 146.92, 161.25, |
| | | 164.21. |
| 30 | 2.16 (2H, m), 2.34 (2H, m), 3.91 (3H, s), 3.92 | 22.29 (q, J = 2.91 Hz), 30.97 (9, |
| | (3H, s), 4.12 (2H, t, J = 6.14 Hz), 6.42 (1H, d, | J = 29.41 Hz), 56.02, 56.12, 67.04, |
| | J = 15.41 Hz), 6.87 (2H, d, J = 8.08 Hz), 6.99-7.07 | 109.90, 111.01, 111.25, 119.03, |
| | (3H, m, J = 4.18 Hz), 7.14 (1H, dd, J = 1.50, 8.22 | 120.46, 121.84, 122.40, 123.89, |
| | Hz), 7.67 (1H, d, J = 15.45 Hz), 7.87 (1H, s), 8.50 | 127.15 (q, J = 276.12 Hz), 127.76, |
| | (1H, s). | 128.16, 142.17, 146.90, 149.35, |
| | | 151.01, 164.08. |
| 31 | 1.03 (3H, t, J = 7.36 Hz), 1.53 (3H, sextet, J = 7.47 | 14.00, 19.40, 31.13, 55.49, 68.91, |
| | Hz), 1.86 (2H, quintet, J = 7.05 Hz), 3.85 (1H, s), | 99.58 (d, J = 27.11 Hz), 106.81 (d, |
| | 4.04 (2H, t, J = 6.58 Hz), 6.40 (1H, d, J = 15.45 | J = 21.58 Hz), 114.41, 118.75, |
| | Hz), 6.61-6.70 (2H, m, J = 4.28 Hz), 6.92 (2H, d, | 120.74 (d, J = 8.40 Hz), 124.32 (d, |
| | J = 8.68 Hz), 7.51 (2H, d, J = 8.64 Hz), 7.69 (1H, | J = 2.27 Hz), 127.51, 129.69, |
| | d, J = 15.45 Hz), 7.73 (1H, s), 8.46 (1H, s). | 141.79, 148.42 (d, J = 8.14 Hz), |
| | | 159.17 (d, J = 242.97 Hz), 161.22, |
| | | 164.03. |
| 32 | 1.02 (3H, t, J = 7.38 Hz), 1.53 (2H, sextet, J = 7.48 | 14.00, 19.39, 31.13, 56.08, 56.10, |
| | Hz), 1.86 (1H, quintet, J = 7.07 Hz), 3.92 (1H, s), | 68.94, 99.63 (d, J = 27.16 Hz), |
| | 3.94 (1H, s), 4.05 (2H, t, J = 6.62 Hz), 6.40 (1H, | 106.84 (d, J = 21.63 Hz), 110.07, |
| | d, J = 15.41 Hz), 6.62-6.70 (2H, m), 6.88 (1H, d, | 111.22, 119.02, 120.81 (d, J = 8.86 |
| | J = 8.32 Hz), 7.07 (1H, d, J = 1.56 Hz), 7.15 (1H, | Hz), 122.28, 124.27 (d, J = 2.33 |
| | dd, J = 1.64, 8.28 Hz), 7.67 (1H, d, J = 15.41 Hz), | Hz), 127.77, 142.07, 148.45 (d, |
| | 7.75 (1H, s), 8.45 (1H, s). | J = 8.96 Hz), 149.29, 150.94, |
| | | 159.21 (d, J = 242.86 Hz), 163.97. |
| 33 | 1.03 (3H, t, J = 7.38 Hz), 1.53 (2H, sextet, J = 7.47 | 14.04, 19.47, 31.35, 61.95 (t, |
| | Hz), 1.86 (2H, quintet, J = 7.07 Hz), 4.05 (3H, s), | J = 3.64 Hz), 68.60 , 111.05, 111.79 |
| | 4.08 (2H, t, J = 6.64 Hz), 6.43 (1H, d, J = 15.36 | (dd, J = 6.82, 16.51 Hz) 120.11, |
| | Hz), 6.89 (1H, dd, J = 0.92, 8.04 Hz), 6.98 (1H, | 121.18, 122.49, 124.15, 127.89, |
| | dt, J = 1.08, 7.70 Hz), 7.05 (1H, dt, J = 1.31, 7.71 | 129.75 (t, J = 8.90 Hz), 137.74 (t, |
| | Hz), 7.07-7.13 (2H,m), 7.57 (1H, d, J = 15.41 Hz), | J = 13.91 Hz), 139.62, 147.41, |
| | 7.96 (1H, s), 8.50 (1H, d, J = 7.12 Hz). | 155.67 (dd, J = 6.41, 248.64 Hz), |
| | | 163.01. |
| 34 | 1.03 (3H, t, J = 7.38 Hz), 1.54 (2H, sextet, J = 7.48 | 14.05 , 19.48, 29.48, 31.39, 68.56, |
| | Hz), 1.85 (2H, quintet, J = 7.06 Hz), 3.23 (2H, t, | 71.93, 109.80, 111.02, 118.31, |
| | J = 8.68 Hz), 4.07 (2H, t, J = 6.60 Hz), 4.62 (2H, t, | 120.03, 121.19, 123.67 124.67, |
| | J = 8.70 Hz), 6.40 (1H, d, J = 15.41 Hz), 6.79 (1H, | 127.70, 128.09, 128.30, 129.24, |
| | d, J = 8.24 Hz), 6.88 (1H, dd, J = 1.30, 7.86 Hz), | 142.17, 147.36, 162.06, 164.26. |
| | 6.97 (1H, dt, J = 1.33, 7.62 Hz), 7.03 (1H, dt, | |
| | J = 1.68, 7.66 Hz), 7.35 (1H, d, J = 8.28 Hz), 7.43 | |
| | (1H, s), 7.68 (1H, d, J = 15.41 Hz), 7.92 (1H, s), | |
| | 8.52 (1H, d, J = 6.08 Hz). | |
| 35 | 0.91 (3H, t, J = 7.38 Hz), 1.42 (2H, sextet, J = 7.48 | 13.94, 19.27, 31.25, 55.48, 68.93, |
| | Hz), 1.73 (2H, quintet, J = 7.01 Hz), 3.83 (1H, s), | 110.93, 114.36, 121.72, 124.03, |
| | 3.98 (2H, t, J = 6.52 Hz), 6.44 (1H, d, J = 13.64 | 127.65, 128.16, 129.71, 130.34, |
| | Hz), 6.84 (1H, d, J = 8.28 Hz), 6.89 (2H, d, | |
| | J = 8.60 Hz), 7.04 (1H, d, J = 7.20 Hz), 7.16 (1H, t, | 132.90, 142.30, 155.45, 161.16, |
| | J = 8.20 Hz), 7.46 (2H, d, J = 8.08 Hz), 7.71 (1H, | 174.83. |
| | d, J = 15.57 Hz). | |
| 36 | 0.91 (3H, t, J = 7.34 Hz), 1.42 (2H, sextet, J = 7.43 | 13.93, 19.25, 31.24, 56.00, 56.05, |
| | Hz), 1.73 (2H, quintet, J = 6.96 Hz), 3.90 (6H, s), | 68.91, 110.00, 110.92, 111.15, |
| | 3.97 (2H, t, J = 6.48 Hz), 6.47 (1H, d, J = 14.12 | 117.67, 121.68, 122.26, 123.97, |
| | Hz), 6.84 (2H, t, J = 7.42 Hz), 7.04 (3H, d, J = 4.80 | 127.89, 128.12, 132.83, 142.39, |
| | Hz), 7.09-7.17 (2H, m), 7.68 (1H, d, J = 15.53 | 149.20, 150.82, 155.40, 164.96. |
| | Hz). | |
| 37 | 1.04 (3H, t, J = 7.38 Hz), 1.55 (2H, sextet, J = 7.44 | 14.06, 19.50, 31.39, 68.58, |
| | Hz), 1.87 (2H, quintet, J = 7.03 Hz), 4.08 (2H, t, | 106.92, 111.04, 112.01, 120.06, |
| | J = 6.56 Hz), 6.55 (1H, d, J = 15.45 Hz), 6.80 (1H, | 120.29, 121.21, 121.69, 123.83, |
| | d, J = 1.24 Hz), 6.90 (1H, d, J = 7.80 Hz), 6.99 | 124.22, 128.18, 128.21, 130.00, |
| | (1H, dt, J = 0.92, 7.77 Hz), 7.05 (1H, dt, J = 1.36, | 142.51, 146.12, 147.39, 155.98, |
| | 7.76 Hz), 7.53 (2H, q, J = 8.40 Hz), 7.65 (1H, d, | 163.94. |
| | J = 1.80 Hz), 7.79 (1H, s), 7.85 (1H, d, J = 15.45 | |
| | Hz), 7.98 (1H, s), 8.54 (1H, d, J = 5.84 Hz). | |
| 38 | 0.18 (2H, q, J = 4.83 Hz), 0.54-0.58 (2H, m), | 4.52, 8.09, 34.48, 55.49, 68.87, |
| | 0.82-0.90 (1H, m), 1.77 (2H, q, J = 6.63 Hz), 3.84 | 110.98, 114.41, 118.96, 120.00, |
| | (3H, s), 4.14 (2H, t, J = 6.52 Hz), 6.43 (1H, d, | 121.19, 123.70, 127.57, 128.26, |
| | J = 15.45 Hz), 6.91 (3H, d, J = 8.64 Hz), 6.98 (1H, | 129.66, 141.70, 147.39, 161.18, |
| | dt, J = 1.33, 7.69 Hz), 7.04 (1H, dt, J = 1.60, 7.72 | 164.11. |
| | Hz), 7.51 (2H, d, J = 8.64 Hz), 7.70 (1H, d, | |
| | J = 15.45 Hz), 7.96 (1H, s), 8.53 (1H, d, J = 5.60 | |
| | Hz). | |
| 39 | 3.83 (3H, s), 6.41 (1H, d, J = 15.45 Hz), 6.84 (1H, | 55.50, 114.43, 117.70, 118.48, |
| | dd, J = 1.12, 8.12 Hz), 6.89 (2H, d, J = 8.68 Hz), | 120.14, 121.29, 124.12, 124.63, |
| | 6.97-7.00 (2H,m), 7.02-7.04 (1H, dd, J = 1.20, | 127.39, 129.23, 129.78, 130.14, |
| | 7.88 Hz), 7.16 (1H, dt, J = 1.07, 7.81 Hz), 7.31- | 130.26, 142.39, 145.50, 155.20, |
| | 7.35 (2H, q, J = 2.96 Hz), 7.48 (2H, d, J = 8.72 | 161.33, 164.37. |
| | Hz), 7.69 (1H, d, J = 15.41 Hz), 7.82 (1H, s), 8.60 | |
| | (1H, d, J = 7.88 Hz). | |
| 40 | 3.83 (3H, s), 6.40 (1H, d, J = 15.41 Hz), 6.88- | 55.51, 114.43, 116.64, 118.33, |
| | 6.93 (4H, q, J = 6.72 Hz), 7.03-7.07 (2H, q, | 118.44, 119.05, 121.34, 124.19, |
| | J = 5.26 Hz), 77.13 (1H, dd, J = 0.72, 8.04 Hz), | 124.21, 125.00, 127.38, 129.80, |
| | 7.19 (1H, dt, J = 0.91, 7.77 Hz), 7.28 (1H, t, | 130.47, 130.91, 135.51, 142.43, |
| | J = 8.26 Hz), 7.49 (2H, d, J = 8.68 Hz), 7.69 (1H, | 144.92, 157.52, 161.34, 164.37. |
| | d, J = 15.45 Hz), 7.78 (1H, s), 8.61 (1H, d, J = 7.84 | |
| | Hz). | |
| 41 | 3.83 (3H, s), 6.43 (1H, d, J = 15.45 Hz), 6.78 (1H, | 55.50, 114.43, 116.61, 116.85, |
| | dd, J = 0.96, 8.12 Hz), 6.90 (1H, d, J = 8.56 Hz), | 118.57, 120.66 (d, J = 8.29 Hz), |
| | 7.98-7.09 (5H, m, ), 7.13 (1H, t, J = 7.80 Hz), | 121.12, 124.01, 124.11, 127.42, |
| | 7.49 (1H, d, J = 8.60 Hz), 7.70 (1H, d, J = 15.41 | 129.77, 129.88, 142.29, 146.26, |
| | Hz), 7.90 (1H, s), 8.59 (1H, d, J = 7.76 Hz). | 152.18 (d, J = 2.64 Hz), 159.31 (d, |
| | | J = 242.84 Hz), 161.30, 164.36. |
| 42 | 2.22 (3H, s), 3.83 (3H, s), 6.49 (1H, d, J = 15.41 | 16.41 (d, J = 1.25 Hz), 55.49, |
| | Hz), 6.55 (1H, d, J = 7.96 Hz), 6.89-7.00 (6H, m), | 114.07 (d, J = 23.23 Hz), 114.42, |
| | 7.07 (1H, t, J = 7.56 Hz), 7.50 (1H, d, J = 8.56 Hz), | 114.54, 118.14 (d, J = 22.91 Hz), |
| | 7.73 (1H, d, J = 15.41 Hz), 8.01 (1H, s), 8.60 (1H, | 118.66, 120.90, 121.85 (d, J = 8.84 |
| | d, J = 7.32 Hz). | Hz), 123.16, 123.89, 127.46, |
| | | 128.85, 129.75, 132.40 (d, J = 8.17 |
| | | Hz), 142.21, 146.70, 149.35 (d, |
| | | J = 2.67 Hz), 159.60 (d, J = 243.32 |
| | | Hz), 161.28, 164.36. |
| 43 | 3.82 (3H, s), 6.47 (1H, d, J = 15.45 Hz), 6.77 (1H, | 55.45, 114.38, 115.79, 117.34 (d, |
| | d, J = 8.04 Hz), 6.89 (2H, d, J = 8.60 Hz), 6.99 | J = 18.05 Hz), 118.64, 121.16, |
| | (1H, t, J = 7.22 Hz), 7.06-7.23 (5H, m,), 7.49 (2H, | 121.97, 123.87, 124.09, 125.03 |
| | d, J = 8.64 Hz), 7.71 (1H, d, J = 15.41 Hz), 8.00 | (d, J = 3.95 Hz), 125.61 (d, J = 6.93 |
| | (1H, s), 8.60 (1H, d, J = 7.52 Hz). | Hz), 127.45, 129.41, 129.74, |
| | | 142.18, 143.20 (d, J = 11.47 Hz), |
| | | 146.05, 154.27 (d, J = 248.84 Hz), |
| | | 161.24, 164.39. |
| 44 | 3.83 (3H, s), 5.22 (2H, s), 6.40 (1H, d, J = 15.49 | 55.49, 65.51 (d, J = 4.04 Hz), |
| | Hz), 6.90 (2H, d, J = 8.68 Hz), 6.99-7.06 (3H, m), | 112.30, 114.39, 115.83 (d, |
| | 7.13-7.20 (2H, m), 7.34-7.40 (1H, m), 7.44 (1H, | J = 21.15 Hz), 118.95, 120.35, |
| | dt, J = 1.52, 7.61 Hz), 7.48 (2H, d, J = 8.68 Hz), | 122.15, 123.63 (d, J = 14.30 Hz), |
| | 7.68 (1H, d, J = 15.49 Hz), 7.97 (1H, s), 8.54 (1H, | 123.77, 124.57 (d, J = 3.62 Hz), |
| | s). | 127.56, 128.79, 129.70, 130.28 |
| | | (d, J = 3.89 Hz), 130.51 (d, J = 8.25 |
| | | Hz), 141.75, 146.96, 161.02 (d, |
| | | J = 247.66 Hz), 161.18, 164.24. |
| 45 | 3.84 (3H, s), 5.11 (2H, s), 6.37 (1H, d, J = 15.45 | 55.51, 70.36, 111.70, 114.42, |
| | Hz), 6.89-6.94 (3H, m), 7.00-7.04 (2H, m), 7.36- | 118.80, 120.46, 121.95, 123.79, |
| | 7.42 (4H, m), 7.48 (2H, d, J = 8.68 Hz), 7.67 (1H, | 127.49, 128.41, 129.17, 129.18, |
| | d, J = 15.45 Hz), 7.88 (1H, s), 8.53 (1H, s). | 129.73, 134.41, 135.05 , 141.93, |
| | | 147.06, 161.25, 164.22. |
| 46 | 2.04 (1H, t, J = 2.36 Hz), 2.10 (2H, m, J = 6.47 | 19.88, 55.51, 67.19, 70.35, 80.79, |
| | Hz), 2.45 (2H, m, J = 3.19 Hz), 3.84 (3H, s), 4.18 | 112.15, 114.44, 119.04, 120.28, |
| | (2H, t, J = 6.14 Hz), 6.46 (1H, d, J = 15.45 Hz), | 122.18, 123.71, 127.58, 128.90, |
| | 6.91 (3H, d, J = 8.48 Hz), 7.02 (2H, m, J = 6.35 | 129.65, 141.67, 146.79, 161.21, |
| | Hz), 7.51 (2H, d, J = 8.60 Hz), 7.70 (1H, d, | 164.24. |
| | J = 15.45 Hz), 7.94 (1H, s), 8.52 (1H, s). | |
| 47 | 3.16 (2H, t, J = 6.44 Hz), 3.86 (3H, s), 4.33 (2H, t, | 35.79, 55.52, 69.24, 111.79, |
| | J = 6.48 Hz), 6.18 (1H, d, J = 15.45 Hz), 6.84-6.96 | 114.42, 119.05, 120.09, 121.71, |
| | (3H, m), 6.99-7.04 (2H, m), 7.28 (1H, d, J = 7.20 | 123.66, 126.88, 127.66, 128.68, |
| | Hz), 7.31-7.39 (4H, m), 7.51 (2H, d, J = 8.56 Hz), | 128.85, 129.05, 129.66, 138.28, |
| | 7.64 (1H, d, J = 15.65 Hz), 7.67 (1H, s), 8.49 (1H, | 141.56, 146.97, 161.19, 164.08. |
| | d, J = 6.12 Hz). | |
| 48 | 2.04 (1H, t, J = 2.46 Hz), 2.10 (2H, quintet, | 15.66, 28.05, 55.50, 67.44, 69.49, |
| | J = 6.48 Hz), 2.45 (2H, dt, J = 2.51, 6.77 Hz), 3.84 | 83.46, 111.27, 114.41, 118.94, |
| | (3H, s), 4.19 (2H, t, J = 6.14 Hz), 6.45 (1H, d, | 120.23, 121.53, 123.77, 127.56, |
| | J = 15.45 Hz), 6.91 (3H, d, J = 8.60 Hz), 6.97-7.06 | 128.34, 129.70, 141.80, 147.15, |
| | (2H, m), 7.52 (2H, d, J = 8.64 Hz), 7.70 (1H, d, | 161.20, 164.19. |
| | J = 15.45 Hz), 7.93 (1H, s), 8.52 (1H, s). | |
| 49 | 1.01 (3H, t, J = 7.41 Hz), 1.53 (2H, sextet, J = 7.50 | 13.91, 19.27, 32.23, 55.52, 74.50 |
| | Hz), 1.81 (2H, quintet, J = 7.08 Hz), 3.84 (3H, s), | (d, J = 6.26 Hz), 110.73 (d, J = 17.63 |
| | 4.22 (2H, dt, J = 1.02, 6.60 Hz), 6.38 (1H, d, | Hz), 114.50, 114.58, 118.18, |
| | J = 15.43 Hz), 6.87 (1H, q, J = 9.13 Hz), 6.92 (2H, | 127.31, 128.53 (t, J = 2.79 Hz), |
| | d, J = 8.76 Hz), 7.50 (2H, d, J = 8.64 Hz), 7.70 | 129.78, 137.11 (d, J = 9.39 Hz), |
| | (1H, d, J = 15.43 Hz), 7.77 (1H, s), 8.21 (1H, s). | 142.49, 143.73 (dd, J = 14.86, |
| | | 246.98 Hz), 147.59 (dd, J = 11.31, |
| | | 245.33 Hz), 161.43, 164.29. |
| 50 | 0.94 (3H, t, J = 7.38 Hz), 1.45 (2H, sextet, J = 7.48 | 13.91, 19.25, 32.21, 74.53 (d, |
| | Hz), 1.74 (2H, quintet, J = 7.08 Hz), 4.16 (2H, t, | J = 6.49 Hz), 107.86, 109.94, |
| | J = 6.24 Hz), 6.35 (1H, d, J = 15.41 Hz), 6.81 (1H, | 110.74 (d, J = 17.78 Hz), 114.66, |
| | q, J = 9.12 Hz), 7.01 (1H, d, J = 8.12 Hz), 7.18- | 120.84, 125.22, 128.16 (t, J = 2.86 |
| | 7.21 (2H, m), 7.62 (1H, d, J = 15.41 Hz), 7.74 | Hz), 129.21, 131.08, 131.76, |
| | (1H, s), 8.13 (1H, s). | 134.32, 141.49, 143.67 (dd, |
| | | J = 14.81, 247.32 Hz), 144.77 (d, |
| | | J = 50.98 Hz), 147.80 (dd, J = 11.18, |
| | | 246.09 Hz), 163.40. |
| 51 | 3.84 (1H, s), 5.13 (2H, s), 6.40 (1H, d, J = 15.45 | 55.52, 70.36, 111.77, 114.41, |
| | Hz), 6.89-6.93 (3H, m), 7.00-7.04 (2H, m), 7.32 | 118.84, 120.46, 122.05, 123.81, |
| | (1H, q, J = 4.25 Hz), 7.36 (2H, d, J = 4.92 Hz), | 125.74, 127.51, 127.78, 128.46, |
| | 7.45 (1H, s), 7.50 (2H, d, J = 8.60 Hz), 7.68 (1H, | 128.70, 129.75, 130.30, 134.88, |
| | d, J = 15.45 Hz), 7.89 (1H, s), 8.54 (1H, s). | 138.64, 141.92, 147.01, 161.24, |
| | | 164.23. |
| 52 | 1.03 (3H, t, J = 7.36 Hz), 1.53 (2H, sextet, J = 7.46 | 14.05, 19.47 31.36, 68.60, 111.09, |
| | Hz), 1.86 (1H, quintet, J = 7.04 Hz), 4.08 (2H, t, | 117.54, 117.71, 119.24, 120.12, |
| | J = 6.60 Hz), 6.46 (1H, d, J = 9.56 Hz), 6.56 (1H, | 121.20, 122.28, 124.16, 127.88, |
| | d, J = 15.49 Hz), 6.90 (1H, d, J = 7.96 Hz), 6.99 | 127.91, 130.73, 131.49, 140.09, |
| | (1H, t, J = 7.26 Hz), 7.06 (1H, t, J = 7.08 Hz), 7.35 | 143.17, 147.43, 154.90, 160.32, |
| | (1H, d, J = 8.60 Hz), 7.63 (1H, d, J = 1.40 Hz), | 163.18. |
| | 7.70-7.77 (3H, m), 7.99 (1H, s), 8.51 (1H, d, | |
| | J = 6.64 Hz). | |
| 53 | 3.83 (3H, s), 6.32 (1H, d, J = 15.40 Hz), 6.88- | 55.54, 113.45 (d, J = 17.59 Hz), |
| | 6.90 (2H, m), 6.93-6.96 (2H, m), 7.01-7.05 (2H, | 114.49, 115.59 (t, J = 5.26 Hz), |
| | m), 7.07-7.10 (1H, m), 7.45-7.47 (2H, m), 7.54 | 116.68 (d, J = 23.66 Hz), 116.83 (d, |
| | (1H, s), 7.67 (1H, d, J = 15.40 Hz), 8.35 (1H, m). | J = 8.31 Hz), 117.70, 127.11, |
| | | 129.90, 142.78 (d, J = 14.91 Hz), |
| | | 143.14, 144.78 (d, J = 14.91 Hz), |
| | | 146.50 (d, J = 10.39 Hz), 148.46 (d, |
| | | J = 10.54 Hz), 153.17 (t, J = 1.89 |
| | | Hz), 158.98 (d, J = 242.32 Hz), |
| | | 161.55 , 164.50. |
| 54 | 3.83 (3H, s), 6.32 (1H, d, J = 15.45 Hz), 6.87- | 55.52, 110.95, 113.52 (d, J = 17.62 |
| | 6.90 (2H, m), 7.05-7.13 (3H, m), 7.44-7.47 (2H, | Hz), 114.46, 115.98, 118.01, |
| | m), 7.66 (2H, d, J = 15.41 Hz), 7.77-7.81 (1H, | 120.14, 127.28, 129.38 (d, J = 2.62 |
| | m,), 8.17-8.19 (1H, m), 8.28 (1H, s). | Hz), 129.83, 132.74, 140.34, |
| | | 142.76, 143.83 (dd, J = 14.87, |
| | | 249.87 Hz), 147.44 (dd, J = 10.17, |
| | | 245.86 Hz), 147.92, 161.43, |
| | | 162.13, 164.47. |
| 55 | 1H-NMR (DMSO, 600 MHz) 3.79 (3H, s), 5.83 | 13C-NMR (DMSO 151MHz ) |
| | (1H, t, J = 2.28 Hz), 6.94 (1H, d, J = 15.67 Hz), | 55.29, 90.14, 112.56 (d, J = 17.47 |
| | 6.98-7.00 (2H, m), 7.32 (1H, q, J = 9.33 Hz), 7.50 | Hz), 114.42, 117.67, 119.29, |
| | (1H, d, J = 15.67 Hz), 7.53-7.56 (2H, m), 7.60 | 127.26, 129.29 (d, J = 1.83 Hz), |
| | (1H, t, J = 2.10 Hz), 8.00 (1H, t, J = 6.60 Hz), 9.69 | 129.44, 130.41, 135.45 (d, |
| | (1H, s), 12.15 (1H, s). | J = 10.28 Hz), 140.50 , 142.36 (d, |
| | | J = 14.55 Hz), 144.00 (d, J = 14.55 |
| | | Hz), 143.18 (dd, J = 14.55, 247.97 |
| | | Hz), 146.49 (dd, J = 10.62, 243.36 |
| | | Hz), 160.64, 161.18, 164.43. |
| 56 | 3.83 (3H, s), 6.32 (1H, d, J = 15.40 Hz), 6.88- | 55.54, 113.45 (d, J = 17.59 Hz), |
| | 6.90 (2H, m), 6.93-6.96 (2H, m), 7.01-7.05 (2H, | 114.49, 115.59 (t, J = 5.26 Hz), |
| | m), 7.07-7.10 (1H, m), 7.45-7.47 (2H, m), 7.54 | 116.68 (d, J = 23.66 Hz), 116.83 (d, |
| | (1H, s), 7.67 (1H, d, J = 15.40 Hz), 8.35 (1H, m). | J = 8.31 Hz), 117.70, 127.11, |
| | | 129.90, 142.78 (d, J = 14.91 Hz), |
| | | 143.14, 144.78 (d, J = 14.91 Hz), |
| | | 146.50 (d, J = 10.39 Hz), 148.46 (d, |
| | | J = 10.54 Hz), 153.17 (t, J = 1.89 |
| | | Hz), 158.98 (d, J = 242.32 Hz), |
| | | 161.55 , 164.50. |
| 57 | 0.98 (3H, t, J = 7.40 Hz), 1.03 (3H, t, J = 7.40 Hz), | 10.63, 14.05, 19.49, 22.65, 31.38, |
| | 1.47-1.56 (4H, m), 1.78 (2H, quintet, J = 7.07 | 68.56, 69.75, 111.01, 114.93, |
| | Hz), 1.86 (2H, quintet, J = 7.01 Hz), 4.00 (1H, t, | 118.83, 120.01, 121.18, 123.69, |
| | J = 6.50 Hz), 4.07 (1H, t, J = 6.58 Hz), 6.42 (1H, d, | 127.36, 128.28, 129.68, 141.79, |
| | J = 15.45 Hz), 6.88-6.92 (3H, m), 6.99 (1H, dt, | 147.35, 160.82, 164.18. |
| | J = 1.48, 7.68 Hz), 7.03 (1H, dt, J = 1.57, 7.70 Hz), | |
| | 7.50 (2H, d, J = 8.64 Hz), 7.69 (1H, d, J = 15.45 | |
| | Hz), 7.92 (1H, s), 8.52 (1H, d, J = 6.12 Hz). | |
| 58 | 0.98 (3H, t, J = 7.40 Hz), 1.03 (3H, t, J = 7.40 Hz), | 13.98, 14.06, 19.36, 19.50, 31.36, |
| | 1.47-1.56 (4H, m), 1.78 (2H, quintet, J = 7.07 | 31.39, 67.96, 68.56, 111.02, |
| | Hz), 1.86 (2H, quintet, J = 7.01 Hz), 4.00 (1H, t, | 114.93, 118.83, 120.02, 121.19, |
| | J = 6.50 Hz), 4.07 (1H, t, J = 6.58 Hz), 6.42 (1H, d, | 123.70, 127.35, 128.29, 129.68, |
| | J = 15.45 Hz), 6.88-6.92 (3H, m), 6.99 (1H, dt, | 141.80, 147.35, 160.84, 164.19. |
| | J = 1.48, 7.68 Hz), 7.03 (1H, dt, J = 1.57, 7.70 Hz), | |
| | 7.50 (2H, d, J = 8.64 Hz), 7.69 (1H, d, J = 15.45 | |
| | Hz), 7.92 (1H, s), 8.52 (1H, d, J = 6.12 Hz). | |
| 59 | 1.03 (3H, t, J = 7.40 Hz), 1.54 (2H, sextet, J = 7.48 | 14.06, 19.50, 31.39, 68.57, 70.23, |
| | Hz), 1.86 (2H, quintet, J = 7.04 Hz), 4.07 (2H, t, | 111.02, 115.32, 119.15, 120.03, |
| | J = 6.58 Hz),5.10 (2H, s) 6.43 (1H, d, J = 15.45 | 121.20, 123.74, 127.62, 127.84, |
| | Hz), 6.89 (1H, dd, J = 0.92, 8.00 Hz), 6.96-7.00 | 128.25, 128.28, 128.81, 129.71, |
| | (3H, m), 7.03 (1H, dt, J = 1.60, 7.80 Hz), 7.34 | 136.66, 141.66, 147.38, 160.37, |
| | (1H, t, J = 6.96 Hz), 7.40 (2H, t, J = 7.28 Hz), 7.42- | 164.10. |
| | 7.45 (2H, m), 7.52 (2H, d, J = 8.64 Hz), 7.69 (1H, | |
| | d, J = 15.49 Hz), 7.92 (1H, s), 8.52 (1H, d, J = 5.92 | |
| | Hz). | |
| 60 | 0.06-0.09 (2H, m), 0.46-0.50 (2H, m), 0.71- | 4.75, 10.67, 29.43, 31.31, 55.52, |
| | 0.79(1H, m), 1.42 (2H, q, J = 7.23 Hz), 1.99 | 68.59, 111.05, 114.43, 118.99, |
| | (2H,quintet, J = 7.02 Hz), 3.85 (3H, s), 4.11 (2H, | 120.04, 121.21, 123.76, 127.59, |
| | t, J = 6.66 Hz), 6.42 (1H, d, J = 15.49 Hz), 6.88- | 128.23, 129.70, 141.75, 147.36, |
| | 6.93 (3H, m), 6.98 (1H, dt, J = 1.44, 7.67 Hz), | 161.21, 164.17 |
| | 7.03 (1H, dt, J = 1.69, 7.67 Hz), 7.52 (2H, d, | |
| | J = 8.72 Hz), 7.70 (1H, d, J = 15.49 Hz), 7.93 (1H, | |
| | s), 8.51 (1H, s). | |
| 61 | 8.51 (d, J = 6.4 Hz, 1H), 7.92 (s, 1H), 7.65 (d, J = | 163.94, 149.41, 148.45, 147.40, |
| | 15.4 Hz, 1H), 7.12 - 6.93 (m, 4H), 6.89 (dd, J = | 141.81, 129.33, 128.23, 124.29, |
| | 7.9, 1.4 Hz, 1H), 6.82 (d, J = 8.0 Hz, 1H), 6.38 | 123.82, 121.20, 120.07, 119.49, |
| | (d, J = 15.4 Hz, 1H), 6.00 (s, 2H), 4.07 (t, J = 6.6 | 111.05, 108.73, 106.63, 101.66, |
| | Hz, 2H), 1.86 (dt, J = 14.4, 6.6 Hz, 2H), 1.60 − | 68.60, 31.41, 19.52, 14.09. |
| | 1.46 (m, 2H), 1.03 (t, J = 7.4 Hz, 3H). | |
| 62 | 8.49 (t, J = 14.2 Hz, 1H), 7.92 (s, 1H), 7.65 (d, J = | |
| | 15.5 Hz, 1H), 7.13 (dd, J = 8.3, 2.0 Hz, 2H), | |
| | 7.09 - 6.94 (m, 2H), 6.94 - 6.81 (m, 2H), 6.40 | |
| | (d, J = 15.4 Hz, 1H), 4.20 - 4.02 (m, 6H), 1.85 | |
| | (d, J = 7.8 Hz, 2H), 1.60 - 1.52 (m, 2H), 1.51 (s, | |
| | 6H), 1.03 (t, J = 7.4 Hz, 3H). | |
| 63 | 1.03 (3H, t, J = 7.40 Hz), 1.35 (6H, s), 1.49-1.59 | 14.05, 19.49, 22.51, 27.04, 31.39, |
| | (2H, m), 1.81-1.89 (4H, m), 2.80 (2H, t, J = 6.74 | 32.75, 68.57, 75.15, 111.02, |
| | Hz), 4.07 (2H, t, J = 6.60 Hz), 6.39 (1H, d, | 117.98, 118.35, 120.00, 121.19, |
| | J = 15.45 Hz), 6.79 (1H, d, J = 8.44 Hz), 6.89 (1H, | 121.35, 123.63, 126.59, 127.14, |
| | dd, J = 1.54, 7.90 Hz), 6.98 (1H, dt, J = 1.57, 7.66 | 128.35, 130.15, 142.14, 147.36, |
| | Hz), 7.03 (1H, dt, J = 1.83, 7.68 Hz), 7.27 (1H, d, | 156.10, 164.33. |
| | J = 1.84 Hz), 7.35 (1H, dd, J = 2.00, 8.48 Hz), 7.66 | |
| | (1H, d, J = 15.41 Hz), 7.91 (1H, s), 8.52 (1H, d, | |
| | J = 6.52 Hz). | |
| 64 | 1.03 (3H, t, J = 7.38 Hz), 1.54 (2H, sextet, J = 7.44 | 14.06, 19.50, 26.04, 31.39, 68.57, |
| | Hz), 1.70 (6H, s), 1.86 (2H, quintet, J = 7.00 Hz), | 106.42, 108.55, 111.02, 117.40, |
| | 4.07 (3H, t, J = 6.54 Hz), 6.35 (1H, d, J = 15.41 | 118.93, 120.00, 121.20, 123.73, |
| | Hz), 6.73 (1H, d, J = 7.88 Hz), 6.89 (1H, d, | 123.87, 128.27, 128.70, 142.09, |
| | J = 7.76 Hz), 6.96-7.05 (4H, m), 7.64 (1H, d, | 147.38, 148.25, 149.37, 164.07. |
| | J = 15.37 Hz), 7.91 (1H, s), 8.51 (1H, d, J = 4.00 | |
| | Hz) | |
| 65 | 1.03 (3H, t, J = 6.76 Hz), 1.54 (2H, sextet, J = 7.74 | 14.04, 19.49, 31.38, 64.39, 64.68, |
| | Hz), 1.85 (2H, quintet, J = 6.96 Hz), 4.07 (2H, t, | 68.56, 111.01, 116.63, 117.78, |
| | J = 6.96 Hz), 4.28 (4H, s), 6.39 (1H, d, J = 15.37 | 119.66, 120.00, 121.17, 122.04, |
| | Hz), 6.88 (2H, s), 6.97-7.10 (4H, m), 7.62 (1H, | 123.75, 128.22, 128.57, 141.60, |
| | d, J = 15.33 Hz), 7.92 (1H, s), 8.51 (1H, s). | 143.81, 145.44, 147.37, 163.97. |
| 83 | 1.03 (3H, t, J = 7.38 Hz), 1.54 (2H, sextet, J = 7.46 | 14.04, 15.27, 19.48, 28.18, 31.38, |
| | Hz), 1.86 (2H, quintet, J = 7.03 Hz), 1.97-2.05 | 66.36, 68.56, 69.14, 83.41, |
| | (3H, m), 2.42 (2H, dt, J = 2.52, 6.91 Hz), 4.07 | 111.02, 114.94, 119.00, 120.03, |
| | (2H, t, J = 6.88 Hz), 4.11 (2H, t, J = 6.14 Hz), 6.42 | 121.18, 123.72, 127.62, 128.25, |
| | (1H, d, J = 15.45 Hz), 6.87-6.93 (3H, m), 6.98 | 129.69, 141.69, 147.37, 160.52, |
| | (1H, dt, J = 1.28, 7.69 Hz), 7.03 (1H, dt, J = 1.68, | 164.14. |
| | 7.76 Hz), 7.51 (2H, d, J = 8.60 Hz), 7.69 (1H, d, | |
| | J = 15.45 Hz), 7.92 (1H, s), 8.52 (1H, d, J = 6.00 | |
| | Hz). | |
| 84 | 1.02 (3H, t, J = 7.38 Hz), 1.54 (2H, sextet, J = 7.47 | 14.05, 19.48, 31.37, 56.09, 56.11, |
| | Hz), 1.86 (2H, quint, J = 7.06 Hz), 3.92 (3H, s), | 68.59, 110.07, 111.06, 111.23, |
| | 3.94 (3H, s), 4.08 (2H, t, J = 6.62 Hz), 6.43 (1H, | 119.29, 120.08, 121.19, 122.28, |
| | d, J = 15.45 Hz), 6.87-6.90 (2H, m), 6.98 (1H, dt, | 123.78, 127.85, 128.21, 141.95, |
| | J = 1.28, 7.65 Hz), 7.04 (1H, dt, J = 1.60, 7.69 Hz), | 147.39, 149.29, 150.91, 164.05. |
| | 7.08 (1H, d, J = 1.64 Hz), 7.15 (1H, dd, J = 1.72, | |
| | 8.24 Hz), 7.67 (1H, d, J = 15.41 Hz), 7.95 (1H, s), | |
| | 8.52 (1H, d, J = 6.08 Hz). | |
| 85 | 1.03 (3H, t, J = 7.40 Hz), 1.54 (2H, sextet, J = 7.48 | 14.05, 19.49 , 31.37 , 55.51, |
| | Hz), 1.86 (2H, quintet, J = 7.05 Hz), 3.84 (3H, s), | 68.55, 111.01, 114.41, 119.01, |
| | 4.07 (2H, t, J = 6.60 Hz), 6.43 (1H, d, J = 15.45 | 119.99, 121.17, 123.71, 127.59, |
| | Hz), 6.89 (1H,dd, J = 1.44, 8.00 Hz), 6.90-6.94 | 128.25, 129.69, 141.70, 147.37, |
| | (2H, m), 6.98 (1H, dt, J = 1.49, 7.67 Hz), 7.03 | 161.19, 164.11. |
| | (1H, dt, J = 1.76, 11.52 Hz), 7.50-7.54 (2H, m), | |
| | 7.70 (1H, d, J = 15.49 Hz), 7.93 (1H, s), 8.52 (1H, | |
| | d, J = 5.92 Hz). | |
| 86 | | |
| 87 | | |
| 88 | 3.83 (3H, s), 5.15 (2H, s), 6.38 (1H, d, J = 15.49 | 149.29, 142.00, 136.64, 128.96, |
| | Hz), 6.90 (2H, d, J = 8.68 Hz), 6.96-7.04 (3H, m), | 128.50, 127.83, 122.41, 121.80, |
| | 7.39-7.49 (7H, m), 7.67 (1H, d, J = 15.49 Hz), | 111.85, 111.21, 109.96, 71.21, |
| | 7.94 (1H, s), 8.55 (1H, s). | 56.10 |
| 142 | 1.01 (3H, t, J = 7.34 Hz), 1.59 (2H, sext, J = 7.47 | 13.94, 19.42, 31.38, 55.73, 68.62, |
| | Hz), 1.88 (2H, quint, J = 6.91 Hz), 3.89 (3H, s), | 101.46, 110.96, 114.86, 117.57, |
| | 4.08 (2H, t, J = 6.26 Hz), 6.91 (1H, d, J = 8.00 Hz), | 119.81, 121.03, 124.69, 124.88, |
| | 6.97-7.01 (3H, m), 7.09 (1H, t, J = 7.22 Hz), 8.00 | 127.38, 133.35, 148.06, 152.49, |
| | (2H, d, J = 8.72 Hz), 8.32 (1H, s), 8.44 (1H, d, | 158.50, 163.54. |
| | J = 7.76 Hz), 8.96 (1H, s). | |
| 143 | 1.04 (3H, t, J = 7.38 Hz), 1.56 (2H, sext, J = 7.48 | |
| | Hz), 1.86 (2H, quint, J = 6.97 Hz), 3.81 (1H, s), | |
| | 4.08 (2H, t, J = 6.42 Hz), 5.09 (2H, d, J = 1.04 Hz), | |
| | 6.45 (1H, d, J = 2.28 Hz), 6.52 (1H, dd, J = 2.42, | |
| | 8.38 Hz), 6.90 (1H, dd, J = 1.22, 7.98 Hz), 6.97 | |
| | (1H, dt, J = 1.31, 7.72 Hz), 7.01-7.06 (3H, m), | |
| | 8.29 (1H, s), 8.40 (1H, dd, J = 1.60, 7.92 Hz). | |
| 144 | 1.00 (3H, t, J = 7.38 Hz), 1.52 (2H, sext, J = 7.44 | 13.90 , 19.23, 32.20, 73.36, 73.55, |
| | Hz), 1.80 (2H, quint, J = 7.08 Hz), 4.23 (3H, t, | 74.50 (d, J = 6.39 Hz), 110.72 (d, |
| | J = 6.60 Hz), 5.12 (4H, s), 6.51 (1H, d, J = 15.49 | J = 17.67 Hz), 114.63, 120.43, |
| | Hz), 6.87 (1H, q, J = 9.12 Hz), 7.27 (1H, d, | 120.49 , 121.62, 127.72, 128.30 |
| | J = 6.48 Hz), 7.42 (1H, s), 7.46 (1H, d, J = 8.04 | (t, J = 3.10 Hz), 134.03, 137.13 (d, |
| | Hz), 7.76 (1H, d, J = 15.49 Hz), 7.82 (1H, s), 8.22 | J = 9.61 Hz), 140.33, 141.67, |
| | (1H, s). | 142.54, 143.67 (dd, J = 15.01, |
| | | 246.97 Hz), 147.69 (dd, J = 10.91, |
| | | 245.82 Hz), 163.83. |
| 145 | 1.01 (3H, t, J = 7.40 Hz), 1.53 (2H, sext, J = 7.50 | 13.92, 19.27, 32.22 , 58.45, 74.32, |
| | Hz), 1.81 (2H, quint, J = 7.07 Hz), 3.41 (3H, s), | 74.52 (1C, d, J = 6.36 Hz), 110.75 |
| | 4.23 (2H, dt, J = 0.79, 6.57 Hz), 4.48 (2H, s), 6.51 | (1C, d, J = 17.71 Hz), 114.60 (1C, |
| | (1H, d, J = 15.49 Hz), 6.88 (1H, q, J = 9.14 Hz), | broad), 120.52 , 128.18, 128.22, |
| | 7.37 (2H, d, J = 8.00 Hz), 7.54 (2H, d, J = 8.04 | 128.36 (1C, t, J = 3.09 Hz), 133.92 |
| | Hz), 7.74 (1H, d, J = 15.49 Hz), 7.81 (1H, s), 8.22 | , 137.12 (1C, d, J = 11.32 Hz), |
| | (1H, s). | 140.70, 142.45 (1C, d, J = 9.18 |
| | | Hz), 142.53, 144.93 (1C, d, |
| | | J = 14.73 Hz), 147.68 (1C, dd, |
| | | J = 11.12, 245.36 Hz), 163.91. |
| 147 | 1.07 (3H, t, J = 7.40 Hz), 1.59 (2H, sext, J = 7.48 | 14.06, 19.58, 31.38, 55.73, 68.58, |
| | Hz), 1.89 (2H, quint, J = 6.93 Hz), 3.85 (3H, s), | 70.88 (1C, q, J = 32.88 Hz), |
| | 4.12 (2H, t, J = 6.40 Hz), 5.95 (1H, q, J = 7.06 Hz), | 101.62, 109.29, 111.14, 112.02, |
| | 6.57-6.60 (2H, m), 6.93 (1H, d, J = 7.64 Hz), 6.99 | 119.07, 119.87, 121.26, 123.65 |
| | (1H, t, J = 7.68 Hz), 7.05-7.12 (2H, m), 7.27 (1H, | (1C, q, J = 287.41 Hz), 124.16, |
| | d, J = 6.16 Hz), 8.36 (1H, s), 8.39 (1H, dd, | 127.62, 129.93, 130.26, 147.49, |
| | J = 1.16, 7.96 Hz.). | 154.67, 162.38, 163.53. |
| 148 | 1.01 (3H, t, J = 7.39 Hz), 1.54 (2H, sext, J = 7.52 | 13.94, 19.3, 31.39, 55.52, 68.50, |
| | Hz), 1.84 (2H, quint, J = 6.94 Hz), 3.86 (3H, s), | 110.95, 114.01, 120.18, 121.13, |
| | 4.07 (2H, t, J = 6.32 Hz), 6.89-6.94 (3H, m), 6.99 | 121.89, 124.46, 124.61, 125.10, |
| | (1H, dt, J = 1.05, 7.74 Hz), 7.08 (1H, dt, J = 1.55, | 127.61, 132.03 (1C, q, J = 2.69 |
| | 7.80 Hz), 7.44 (2H, d, J = 8.56 Hz), 7.98 (1H, s), | Hz), 145.94 (1C, q, J = 3.25 Hz), |
| | 8.44 (1H, dd, J = 1.08, 7.88 Hz), 8.50 (1H, s). | 147.81, 160.84, 161.33. |
| 149 | 1.03 (3H, t, J = 7.40 Hz), 1.54 (2H, sext, J = 7.47 | 14.06, 19.50, 31.39, 40.32, 68.53, |
| | Hz), 1.86 (2H, quint, J = 7.04 Hz), 3.02 (3H, s), | 110.98, 112.00, 116.07, 119.94, |
| | 4.07 (2H, t, J = 6.56 Hz), 6.35 (1H, d, J = 15.36 | 121.17, 122.69, 123.39, 128.53, |
| | Hz), 6.69 (2H, d, J = 8.72 Hz), 6.88 (1H, dd, | 129.66, 131.58, 142.42, 151.63, |
| | J = 1.31, 7.49 Hz), 6.95-7.01 (2H, m), 7.47 (2H, | 164.77. |
| | d, J = 8.72 Hz), 7.67 (2H, d, J = 15.41 Hz), 7.89 | |
| | (1H, s), 8.53 (1H, d, J = 6.72 Hz). | |
| 150 | 1.03 (3H, t, J = 7.38 Hz), 1.54 (2H, sext, J = 7.47 | 14.05, 19.49, 30.48, 31.39, 68.54, |
| | Hz), 1.86 (2H, quint, J = 7.04 Hz), 2.88 (3H, s), | 111.00, 112.27, 115.01, 116.21, |
| | 4.07 (2H, t, J = 6.54 Hz), 6.34 (1H, d, J = 15.36 | 119.95, 121.17, 123.44, 123.79, |
| | Hz), 6.59 (2H, d, J = 8.52 Hz), 6.88 (1H, d, | 128.49, 129.86, 142.49, 150.93, |
| | J = 7.80 Hz), 6.95-7.03 (2H, m), 7.42 (2H, d, | 164.74. |
| | J = 8.48 Hz), 7.65 (1H, d, J = 15.41 Hz), 7.88 (1H, | |
| | $), 8.52 (1H, d, J = 5.92 Hz). | |
| 151 | 0.94 (3H, t, J = 7.37 Hz), 1.46 (2H, sext, J = 7.46 | 13.77, 18.67, 24.09, 30.64, 68.08, |
| | Hz), 1.78 (2H, quint, J = 7.06 Hz), 2.07 (3H, s), | 112.16, 118.96, 120.19, 120.85, |
| | 4.05 (2H, t, J = 6.65 Hz), 6.91 (1H, quint, J = 4.15 | 121.71, 124.29, 127.69, 128.54, |
| | Hz), 7.03-7.07 (3H, m), 7.49 (1H, d, J = 15.61 | 129.50, 139.77, 140.68, 148.71, |
| | Hz), 7.62 (4H, dd, J = 8.50, 29.95 Hz), 8.12 (1H, | 163.85, 168.53 |
| | d, J = 7.36 Hz), 9.05 (1H, s), 10.13 (1H, s). | |
| 152 | 1.02 (3H, t, J = 7.40 Hz), 1.53 (2H, sext, J = 7.47 | 14.04, 19.46, 31.34, 44.12, 68.59, |
| | Hz), 1.86 (2H, quint, J = 7.07 Hz), 2.75 (3H, s), | 111.06, 120.12, 121.18, 123.56, |
| | 4.08 (2H, t, J = 6.64 Hz), 6.64 (1H, d, J = 15.49 | 124.20, 127.87, 128.12, 128.72, |
| | Hz), 6.90 (1H, dd, J = 1.11, 8.07 Hz), 6.99 (1H, | 128.84, 137.67, 140.47, 147.42, |
| | dt, J = 1.16, 7.74 Hz), 7.06 (1H, dt, J = 1.21, 7.76 | 163.08. |
| | Hz), 7.69 (4H, q, J = 8.38 Hz), 7.76 (1H, d, | |
| | J = 15.49 Hz), 8.01 (1H, s), 8.51 (1H, d, J = 7.24 | |
| | Hz). | |
| 153 | 1.02 (3H, t, J = 7.20 Hz), 1.53 (2H, sext, J = 7.16 | 14.04, 19.46, 31.33, 44.63, 68.61, |
| | Hz), 1.86 (2H, quint, J = 6.80 Hz), 3.08 (3H, s), | 111.08, 120.15, 121.19, 124.36, |
| | 4.09 (1H, t, J = 6.34 Hz), 6.68 (1H, d, J = 15.49 | 125.17, 127.73, 128.12, 128.73, |
| | Hz), 6.91 (1H, d, J = 7.68 Hz), 6.99 (1H, t, J = 7.34 | 139.72 140.19, 141.17, 147.46, |
| | Hz), 7.06 (1H, d, J = 7.48 Hz), 7.74 (2H, d, | 162.65. |
| | J = 8.32 Hz), 7.79 (1H, s), 7.96 (2H, d, J = 7.80 | |
| | Hz), 8.03 (1H, s), 8.51 (1H, d, J = 6.80 Hz). | |
| 154 | 1.03 (3H, t, J = 7.38 Hz), 1.54 (1H, sext, J = 7.48 | 14.05, 15.40, 19.48, 31.37, 68.57, |
| | Hz), 1.86 (2H, quint, J = 7.06 Hz), 2.51 (3H, s), | 111.02, 120.03, 120.43, 121.18, |
| | 4.08 (2H, t, J = 6.62 Hz), 6.51 (1H, d, J = 15.45 | 123.85, 126.21, 128.14, 128.46, |
| | Hz), 6.89 (1H, dd, J = 1.32, 7.96 Hz), 6.99 (1H, | 131.44, 141.41, 141.47, 147.37, |
| | dt, J = 1.36, 11.50 Hz), 7.03 (1H, dt, J = 1.71, | 163.83. |
| | 11.52 Hz), 7.24 (2H, d, J = 8.40 Hz), 7.48 (2H, d, | |
| | J = 8.36 Hz), 7.69 (1H, d, J = 15.49 Hz), 7.95 (1H, | |
| | s), 8.52 (1H, d, J = 6.56 Hz). | |
| 155 | 2.65-2.76 (2H, m), 3.84 (3H, s), 4.31 (2H, t, | 34.07 (1C, q, J = 28.89 Hz), 55.51, |
| | J = 5.94 Hz), 6.38 (1H, d, J = 15.48 Hz), 6.87-6.93 | 61.80 (1C, q, J = 3.43 Hz), 111.03, |
| | (3H, m), 7.03-7.06 (2H, m), 7.51 (2H, d, J = 8.63 | 114.43, 118.76, 120.38, 122.32, |
| | Hz), 7.68 (1H, d, J = 15.49 Hz), 7.90 (1H, s), 8.54 | 123.69, 126.22 (1C, q, J = 276.90 |
| | (1H, s). | Hz), 127.50, 128.53, 129.71, |
| | | 141.87, 146.36, 161.25, 164.26. |
| 156 | 0.18 (2H, q, J = 4.97 Hz), 0.53-0.56 (2H, m), | 4.52, 7.86, 35.16, 74.78 (1C, d, |
| | 0.82-0.87 (1H, m), 1.73 (2H, q, J = 6.69 Hz), 4.31 | J = 6.67 Hz), 108.91 (1C, d, |
| | (2H, dt, J = 1.14, 6.58 Hz), 6.41 (1H, d, J = 15.40 | J = 264.67 Hz), 110.70 (1C, d, |
| | Hz), 6.88 (1H, q, J = 9.10 Hz), 7.08 (1H, d, | J = 17.69 Hz), 114.66, 120.83, |
| | J = 8.10 Hz), 7.25-7.27 (2H, m), 7.69 (1H, d, | 125.19, 128.14 (1C, d, J = 3.04 |
| | J = 15.40 Hz), 7.85 (1H, s), 8.22 (1H, s). | Hz), 131.09, 131.77 (1C, t, |
| | | J = 256.63 Hz), 137.12 (1C, d, |
| | | J = 8.44 Hz), 141.53, 143.63 (1C, |
| | | dd, J = 15.00, 247.17 Hz), 144.53, |
| | | 145.04 (1C, d, J = 0.92 Hz), 147.80 |
| | | (1C, dd, J = 11.27, 245.84 Hz), |
| | | 163.41. |
| 157 | 3.49 (3H, s), 3.74-3.76 (2H, m), 3.84 (3H, s), | 55.52, 59.24, 70.01, 71.01, |
| | 4.20-4.22 (2H, m), 6.46 (1H, d, J = 15.49 Hz), | 114.44, 114.53, 119.18, 120.39, |
| | 6.92 (2H, d, J = 8.76 Hz), 6.95-6.99 (1H, m), | 122.85, 123.72, 127.70, 129.64, |
| | 7.00-7.07 (2H, m), 7.51 (2H, d, J = 8.68 Hz), 7.70 | 130.00, 141.58, 147.19, 161.16, |
| | (1H, d, J = 15.53 Hz), 8.31 (1H, s), 8.54 (1H, d, | 164.36. |
| | J = 6.00 Hz). | |
| 158 | 1.02 (3H, t, J = 7.34 Hz), 1.53 (2H, sext, J = 7.39 | 14.03, 19.49, 31.36, 55.85, 68.60, |
| | Hz), 1.86 (2H, quint, J = 7.00 Hz), 3.83 (3H, s), | 102.12 (1C, d, J = 25.67 Hz), |
| | 4.07 (2H, t, J = 6.52 Hz), 6.57 (1H, d, J = 15.65 | 110.77 (1C, d, J = 2.91 Hz), 111.04, |
| | Hz), 6.66 (1H, dd, J = 1.72, 12.68 Hz), 6.72 (1H, | 115.57 (1C, d, J = 12.06 Hz), |
| | dd, J = 2.06, 8.50 Hz), 6.89 (1H, d, J = 7.76 Hz), | 120.02, 121.18, 121.68 (1C, d, |
| | 6.96-7.05 (2H, m), 7.47 (1H, t, J = 8.58 Hz), 7.75 | J = 7.18 Hz), 123.79, 128.21, |
| | (1H, d, J = 15.65 Hz), 7.96 (1H, s), 8.52 (1H, d, | 130.73 (1C, d, J = 5.28 Hz), |
| | J = 6.16 Hz). | 134.93, 161.29, 162.20 (1C, d, |
| | | J = 11.53 Hz), 163.81, 164.07. |
| 159 | 1.02 (3H, t, J = 7.38 Hz), 1.53 (2H, sext, J = 7.48 | 14.05, 19.50, 31.39, 55.79, 68.56, |
| | Hz), 1.85 (2H, quint, J = 7.05 Hz), 3.83 (3H, s), | 110.99, 113.96, 115.01, 119.96, |
| | 4.07 (2H, t, J = 6.56 Hz), 6.46 (1H, d, J = 15.57 | 121.19, 121.98, 123.84, 125.56, |
| | Hz), 6.84 (1H, dd, J = 2.48, 8.72 Hz), 6.89 (1H, | 128.12, 128.56, 136.15, 137.24, |
| | dd, J = 1.38, 7.94 Hz), 6.96 (1H, d, J = 2.51 Hz), | 147.37, 161.25, 163.70. |
| | 6.99 (1H, dd, J = 1.44, 7.77 Hz), 7.04 (1H, dt, | |
| | J = 1.72, 7.72 Hz), 7.60 (1H, d, J = 8.76 Hz), 8.01- | |
| | 8.06 (2H, m), 8.53 (1H, d, J = 6.92 Hz). | |
| 160 | 1.26 (3H, t, J = 7.14 Hz), 1.53 (2H, sext, J = 7.44 | 14.04, 19.50, 20.27, 31.39, 55.42, |
| | Hz), 1.85 (2H, quint, J = 7.01 Hz), 2.46 (3H, s), | 68.57, 111.02, 112.27, 115.90, |
| | 3.83 (3H, s), 4.07 (3H, t, J = 6.59 Hz), 6.75-6.78 | 119.95, 120.04, 121.20, 123.72, |
| | (2H, m), 6.89 (1H, dd, J = 1.10, 7.86 Hz), 6.96- | 126.48, 127.81, 139.25, 139.25, |
| | 7.05 (2H, m), 7.57 (1H, d, J = 8.35 Hz), 7.97 (2H, | 139.85, 139.85, 147.34, 160.89, |
| | d, J = 15.26 Hz), 8.53 (1H, d, J = 4.98 Hz). | 160.89, 164.33. |
| 161 | 1.01 (3H, t, J = 7.38 Hz), 1.54 (2H, sext, J = 7.52 | 12.48, 14.25, 20.30, 32.32, 69.68, |
| | Hz), 1.86 (2H, quint, J = 7.13 Hz), 2.87 (1H, s), | 113.03, 114.38, 115.10, 121.43, |
| | 4.11 (2H, t, J = 6.64 Hz), 6.95 (1H, dt, J = 1.01, | 123.62, 124.14, 126.55, 126.64, |
| | 7.68 Hz), 7.04 (1H, dd, J = 0.96, 8.32 Hz), 7.09- | 128.29, 132.99, 133.18, 134.91, |
| | 7.15 (2H, m), 7.76 (1H, d, J = 5.72 Hz), 7.79 (1H, | 141.41, 151.06 , 153.75, 166.15. |
| | d, J = 12.80 Hz), 7.89 (1H, d, J = 8.40 Hz), 7.95 | |
| | (1H, s), 8.07 (1H, d, J = 7.56 Hz). | |
| 162 | 1.02 (3H, t, J = 7.38 Hz), 1.54 (2H, sext, J = 7.46 | 13.92, 19.27, 20.35, 32.24, 74.52 |
| | Hz), 1.82 (2H, quint, J = 7.05 Hz), 2.86 (1H, s), | (1C, d, J = 6.39 Hz), 110.75 (1C, d, |
| | 4.24 (2H, t, J = 6.42 Hz), 6.58 (1H, d, J = 15.45 | J = 17.68 Hz), 114.63, 120.93, |
| | Hz), 6.89 (1H, q, J = 9.14 Hz), 7.54 (1H, d, | 121.88, 121.94, 124.45, 128.32, |
| | J = 8.28 Hz), 7.83 (2H, d, J = 8.28 Hz), 7.86 (1H, | 132.86, 137.15 (1C, d, J = 9.28 |
| | d, J = 15.49 Hz), 8.12 (1H, s), 8.23 (1H, s). | Hz), 137.52, 142.57, 143.70 (1C, |
| | | q, J = 87.35 Hz), 147.73 (1C, q, |
| | | J = 85.67 Hz), 153.90, 163.74, |
| | | 168.59. |
| 163 | 1.02 (3H, t, J = 7.38 Hz), 1.56 (2H, sext, J = 7.47 | 13.94, 19.29, 32.25, 74.52 (1C, d, |
| | Hz), 1.82 (2H, qunit, J = 7.05 Hz), 4.23 (2H, t, | J = 6.20 Hz), 103.64, 110.76 (1C, d, |
| | J = 6.42 Hz), 6.49 (1H, d, J = 15.41 Hz), 6.60 (1H, | J = 17.60 Hz), 111.73, 114.58, |
| | br), 6.89 (1H, q, J = 9.14 Hz), 7.25 (1H, d, J = 2.60 | 117.60, 121.73, 122.36, 125.49, |
| | Hz), 7.43 (1H, q, J = 9.22 Hz), 7.82 (2H, d, | 126.70, 128.37, 128.67 (1C, t, |
| | J = 21.57 Hz), 7.89 (1H, d, J = 15.41 Hz), 8.25 | J = 2.85 Hz), 137.08, 137.09 (1C, |
| | (1H, br), 8.38 (1H, s). | d, J = 11.08 Hz), 143.76 (1C, dd, |
| | | J = 14.78, 246.85 Hz), 144.59, |
| | | 147.54 (1C, dd, J = 11.25, 245.07 |
| | | Hz), 164.66. |
| 164 | 1.04 (3H, t, J = 7.38 Hz), 1.56 (2H, sext, J = 7.47 | 14.07, 19.51, 31.40, 33.13, 68.56, |
| | Hz), 1.88 (2H, quint, J = 7.04 Hz), 3.81 (3H, s), | 102.06, 109.79, 111.01, 118.20, |
| | 4.08 (2H, t, J = 6.56 Hz),6.53 (2H, dd, J = 6.24, | 120.03, 121.19, 121.29, 122.34, |
| | 9.16 Hz), 6.90 (1H, dd, J = 1.42, 7.74 Hz), 7.00- | 123.57, 126.41, 128.42, 128.86, |
| | 7.06 (2H, m), 7.08 (1H, d, J = 3.08 Hz), 7.32 (1H, | 130.06, 137.78, 143.85, 147.37, |
| | d, J = 8.52 Hz), 7.50 (1H, dd, J = 1.26, 8.54 Hz), | 164.54. |
| | 7.83 (1H, s), 7.88 (1H, d, J = 15.41 Hz), 7.96 (1H, | |
| | s), 8.54 (1H, br). | |
| 165 | 1.04 (3H, t, J = 7.36 Hz), 1.48 (3H, t, J = 7.26 Hz), | 14.07, 15.61, 19.51, 31.41, 41.31, |
| | 1.56 (2H, sext, J = 7.47 Hz), 1.88 (2H, quint, | 68.57, 102.20, 109.84, 111.01, |
| | J = 7.04 Hz), 4.08 (2H, t, J = 6.56 Hz), 4.19 (2H, q, | 118.15, 120.01, 121.15, 121.19, |
| | J = 7.28 Hz), 6.52 (1H, d, J = 10.36 Hz), 6.55 (1H, | 122.45, 123.55, 126.37, 128.28, |
| | d, J = 1.40 Hz), 6.90 (1H, d, J = 7.80 Hz), 6.97- | 128.43, 129.02, 136.82, 143.85, |
| | 7.05 (2H, m), 7.15 (1H, d, J = 3.00 Hz), 7.35 (1H, | 147.38, 164.56. |
| | d, J = 8.60 Hz), 7.48 (1H, d, J = 8.56 Hz), 7.84 | |
| | (1H, s), 7.88 (1H, d, J = 15.45 Hz), 7.96 (1H, s), | |
| | 8.55 (1H, s). | |
| 166 | 1.03 (3H, t, J = 7.34 Hz), 1.54 (2H, sext, J = 7.36 | 14.05, 19.47, 31.34, 68.62, |
| | Hz), 1.87 (2H, quint, J = 7.02 Hz), 4.09 (2H, t, | 111.08, 116.93, 117.21, 120.17, |
| | J = 6.56 Hz), 6.69 (1H, d, J = 15.48 Hz), 6.91 (1H, | 121.22, 124.47, 125.58, 127.66, |
| | d, J = 8.01 Hz), 7.00 (1H, t, J = 7.27 Hz), 7.08 (1H, | 130.09, 138.13, 139.61, 147.45, |
| | t, J = 7.06 Hz), 7.66 (1H, d, J = 9.16 Hz), 7.80 (1H, | 149.12, 149.61, 162.41. |
| | d, J = 15.50 Hz), 7.87 (1H, d, J = 9.43 Hz), 7.93 | |
| | (1H, s), 8.06 (1H, s), 8.52 (1H, d, J = 7.20 Hz). | |

### Example 66: (E)-3-(4-(3-(1-benzyl-1H-1,2,3-triazol-4-yl)propoxy)phenyl)-N-(2-butoxyphenyl)acrylamide

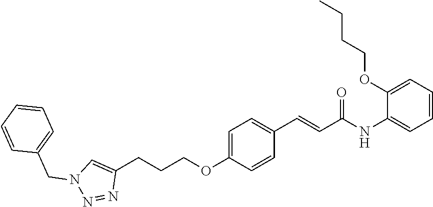 [SMILES: CCCCOc1ccccc1NC(=O)/C=C/c1ccc(OCCCc2cn(Cc3ccccc3)nn2)cc1]

(Azidomethyl)benzene (77 mg, 0.58 mmol) and (E)-N-(2-butoxyphenyl)-3-(4-(pent-4-yn-1-yloxy)phenyl)acrylamide (220 mg, 0.58 mmol) (1.00 equiv.) were dissolved in dichloromethane. To this solution, a freshly prepared solution of copper (11) sulphate pentahydrate (12 mg, 0.047 mmol, 0.08 equiv.) and sodium ascorbate (23 mg, 0.12 mmol, 0.20 equiv.) in water was added, while the mixture was stirred vigorously. The reaction mixture was allowed to stir for 2 h at room temperature. After the reaction was complete, the reaction mixture was extracted with dichloromethane and the organic phase was dried over magnesium sulphate. After the organic layer was evaporated to dryness, the residue was purified by silica gel chromatography (PE/EA=1/1) to afford (E)-3-(4-(3-(1-benzyl-1H-1,2,3-triazol-4-yl)propoxy)phenyl)-N-(2-butoxyphenyl)acrylamide (153 mg, 0.3 mmol, 51%) as white solid. ¹H-NMR (CDCl₃ 400 MHz): 1.03 (3H, t, J=7.40 Hz), 1.54 (2H, sextet, J=7.47 Hz), 1.86 (2H, quintet, J=7.02 Hz), 2.17 (2H, quintet, J=6.80 Hz), 2.90 (2H, t, J=7.46 Hz), 4.02 (2H, t, J=6.16 Hz), 4.08 (2H, t, J=6.60 Hz), 5.49 (2H, s), 6.41 (1H, d, J=15.45 Hz), 6.85-6.90 (3H, m), 6.98 (1H, dt, J=1.15, 7.65 Hz), 7.03 (1H, dt, J=1.61, 7.72 Hz), 7.21-7.25 (3H, m), 7.35-7.37 (3H, m), 7.48 (2H, d, J=8.60 Hz), 7.68 (1H, d, J=15.45 Hz), 7.92 (1H, s), 8.52 (1H, d, J=6.16 Hz). ¹³C-NMR (CDCl₃): 14.04, 19.47, 22.25, 28.84, 31.35, 54.15, 67.05, 68.54, 111.01, 114.88, 118.94, 119.99, 121.01, 121.15, 123.70, 127.50, 128.11, 128.24, 128.78, 129.19, 129.66, 134.95 (1C, s), 141.67, 147.36, 147.65, 160.53, 164.12. HRMS (ESI): Found 360.1572, calc. 360.1576, [M+Na]⁺, M=C₃₁H₃₄N₄O₃

### Example 67: E)-N-(2-butoxyphenyl)-3-(3,4-dimethoxyphenyl)-N-methylacrylamide

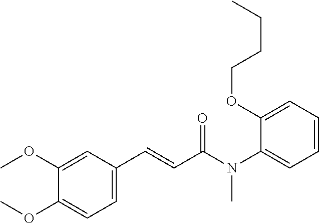 [SMILES: CCCCOc1ccccc1N(C)C(=O)/C=C/c1ccc(OC)c(OC)c1]

(E)-N-(2-butoxyphenyl)-3-(3,4-dimethoxyphenyl)acrylamide (example 84, 1 eq.) was added to a suspension of sodium hydride (1.4 eq) in dry THF (8 mL). The mixture was allowed to stir for 5 min until hydrogen evolution ceased. Methyl iodide (1.4 eq.) was added and stirring was continued for 16 hr. The mixture was poured into ether and washed with brine, dried over magnesium sulphate and evaporated in vacuo to dryness. Purification of the crude material by flash chromatography afforded (E)-N-(2-butoxyphenyl)-3-(3,4-dimethoxyphenyl)-N-methylacrylamide in 76% yield.

¹H-NMR (400 MHz, CDCl₃) 0.89 (3H, t, J=7.40 Hz), 1.42 (2H, sextet, J=7.46 Hz), 1.71 (2H, quintet, J=7.04 Hz), 3.30 (3H, s), 3.80 (3H, s), 3.86 (3H, s), 3.98 (2H, t, J=6.22 Hz), 6.16 (1H, d, J=15.49 Hz), 6.75-6.78 (2H, m), 6.91 (1H, d, J=8.24 Hz), 6.95-6.99 (2H, m), 7.19 (1H, dd, J=1.38, 8.14 Hz), 7.32 (1H, dt, J=1.32, 7.82 Hz), 7.59 (1H, d, J=15.45 Hz). ¹³C-NMR (CDCl₃, 100 MHz) 13.87, 19.32, 31.27, 36.39, 55.96, 56.02, 68.11, 110.58, 111.14, 112.88, 117.00, 120.75, 121.35, 128.64, 129.32, 129.72, 132.29, 141.12, 149.01, 150.34, 154.97, 167.00. HRMS (ESI): m/z [M+Na]⁺ calcd for C₂₂H₂₇NO₄: 392.1838, Found: 392.1823.

The following compounds, as listed in Table 3 below can be prepared in a similar manner as described for Example 67.

TABLE 3

| Example | Structure | IUPAC Name | HR-MS |
|---|---|---|---|
| 68 | 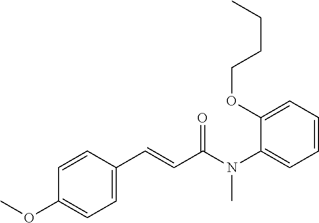 [SMILES: CCCCOc1ccccc1N(C)C(=O)/C=C/c1ccc(OC)cc1] | E)-N-(2-butoxyphenyl)- 3-(4-methoxyphenyl)-N- methylacrylamide | Found: 362.1724 [M + Na]⁺ calc 362.1732 |
| 69 | 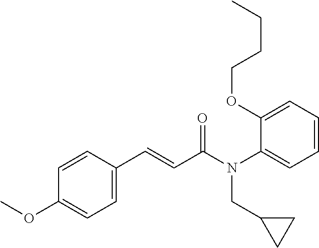 [SMILES: CCCCOc1ccccc1N(CC1CC1)C(=O)/C=C/c1ccc(OC)cc1] | (E)-N-(2-butoxyphenyl)- N-(cyclopropylmethyl)-3-(4- methoxyphenyl)acrylamide | Found: 402.2039 [M + Na]⁺ calc 402.2045 |
| 70 | 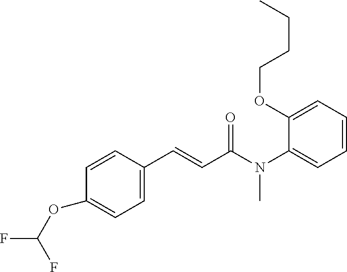 [SMILES: CCCCOc1ccccc1N(C)C(=O)/C=C/c1ccc(OC(F)F)cc1] | (E)-N-(2-butoxyphenyl)- 3-(4- (difluoromethoxy)phenyl)- N-methylacrylamide | Found: 398.1545 [M + Na]⁺ calc. 398.1544 |
| 71 | 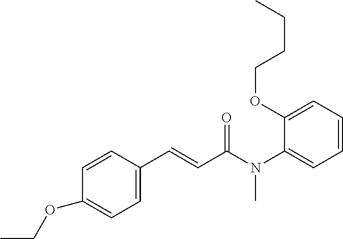 [SMILES: CCCCOc1ccccc1N(C)C(=O)/C=C/c1ccc(OCC)cc1] | (E)-N-(2-butoxyphenyl)- 3-(4-ethoxyphenyl)-N- methylacrylamide | Found: 376.1878 [M + Na]⁺ calc. 376.1889 |
| 72 | 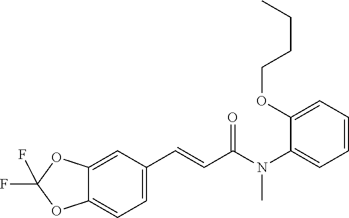 [SMILES: CCCCOc1ccccc1N(C)C(=O)/C=C/c1ccc2c(c1)OC(F)(F)O2] | (E)-N-(2-butoxyphenyl)- 3-(2,2- difluorobenzo[d][1,3] dioxol-5-yl)-N- methylacrylamide | Found: 412.1340 [M + Na]⁺ calc. 412.1336 |
| 73 | 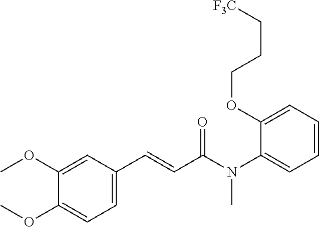 [SMILES: COc1ccc(/C=C/C(=O)N(C)c2ccccc2OCCCC(F)(F)F)cc1OC] | (E)-3-(3,4- dimethoxyphenyl)-N- methyl-N-(2-(4,4,4- trifluorobutoxy)phenyl) acrylamide | Found: 446.1543 [M + Na]⁺ calc. 446.1555 |
| 74 | 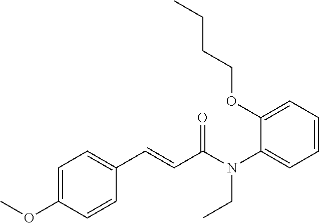 [SMILES: CCCCOc1ccccc1N(CC)C(=O)/C=C/c1ccc(OC)cc1] | (E)-N-(2-butoxyphenyl)- N-ethyl-3-(4- methoxyphenyl)acrylamide | Found: 376.1882 [M + Na]⁺ calc. 376.1889 |
| 75 | 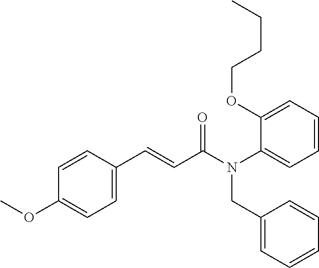 [SMILES: CCCCOc1ccccc1N(Cc1ccccc1)C(=O)/C=C/c1ccc(OC)cc1] | (E)-N-benzyl-N-(2- butoxyphenyl)-3-(4- methoxyphenyl)acrylamide | Found: 438.2043 [M + Na]⁺ calc. 438.2045 |
| 76 | 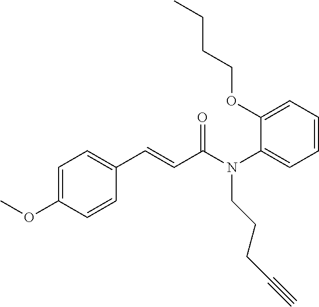 [SMILES: C#CCCCN(C(=O)/C=C/c1ccc(OC)cc1)c1ccccc1OCCCC] | (E)-N-(2-butoxyphenyl)- 3-(4-methoxyphenyl)-N- (pent-4-yn-1- yl)acrylamide | Found: 414.2045 [M + Na]⁺ calc. 414.2042 |
| 77 | 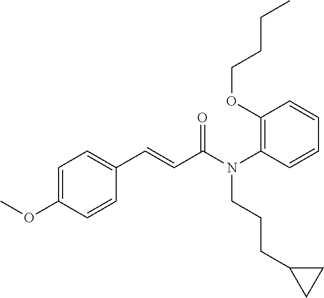 [SMILES: CCCCOc1ccccc1N(CCCC1CC1)C(=O)/C=C/c1ccc(OC)cc1] | (E)-N-(2-butoxyphenyl)- N-(3- cyclopropylpropyl)-3-(4- methoxyphenyl)acrylamide | Found: 430.2358 [M + Na]⁺ calc. 430.2357 |
| 89 | 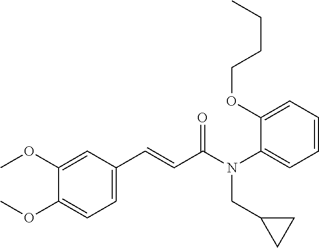 [SMILES: CCCCOc1ccccc1N(CC1CC1)C(=O)/C=C/c1ccc(OC)c(OC)c1] | (E)-N-(2-butoxyphenyl)- N-(cyclopropylmethyl)- 3-(3,4- dimethoxyphenyl)acrylamide | Found: 432.2144 [M + Na] calc432.2151 |

| Example | ¹H-NMR | ¹³C-NMR |
|---|---|---|
| 68 | 0.88 (3H, t, J = 7.38 Hz), 1.42 (2H, | 13.87, 19.32, 31.28, 36.41, |
| | sextet, J = 7.51 Hz), 1.71 (2H, quintet, | 55.42, 68.11, 112.92, 114.14, |
| | J = 6.94 Hz), 3.29 (3H, s), 3.78 (3H, s), | 116.58, 120.80, 128.33, |
| | 3.97 (2H, dt, J = 1.57, 6.30 Hz), 6.16 | 129.33, 129.40, 129.72, |
| | (1H, d, J = 15.53 Hz), 6.79 (2H, d, | 132.32, 140.96, 154.96, |
| | J = 8.68 Hz), 6.95-6.99 (2H, t, J = 7.32 | 160.67, 167.11. |
| | Hz), 7.18 (1H, dd, J = 1.40, 7.96 Hz), | |
| | 7.23-7.26 (2H, d, J = 8.96 Hz), 7.33 | |
| | (1H, dt, J = 1.29, 7.85 Hz), 7.61 (1H, d, | |
| | J = 15.49 Hz). | |
| 69 | 0.06-0.16 (2H, m), 0.35-0.39(2H, m, ), | 3.26, 4.04, 9.95, 13.81, |
| | 0.86 (3H, t, J = 7.38 Hz), 0.94-1.04 (1H, | 19.24, 31.26, 52.52, 55.35, |
| | m), 1.39 (2H, sextet, J = 7.48 Hz), 1.67 | 67.94, 112.59, 114.08, |
| | (2H, quintet, J = 6.94 Hz), 3.31 (1H, dd, | 116.97, 120.49, 128.38, |
| | J = 7.20, 13.85 Hz), 3.76 (3H, s), 3.92- | 129.28, 129.30, 130.89, |
| | 3.98 (3H, m), 6.13 (1H, d, J = 15.53 | 130.96, 140.92, 155.24, |
| | Hz), 6.78 (2H, d, J = 8.76 Hz), 6.94- | 160.58, 166.73. |
| | 6.98 (2H, qm), 7.21-7.24 (3H, m, | |
| | J = 2.33 Hz), 7.32 (1H, dt, J = 1.61, 7.86 | |
| | Hz), 7.61 (1H, d, J = 15.49 Hz). | |
| 70 | 0.88 (3H, t, J = 7.38 Hz), 1.41 (2H, | 13.85, 19.32, 31.26, 36.48 , |
| | sextet, J = 7.50 Hz), 1.70 (2H, quintet, | 68.13, 112.94 , 115.75 (t, |
| | J = 6.93 Hz), 3.30 (1H, s), 3.97 (2H, dt, | J = 260.42 Hz), 119.12, |
| | J = 2.13, 6.30 Hz), 6.23 (1H, d, J = 15.57 | 119.61, 120.86, 129.33, |
| | Hz), 6.47 (1H, t, J = 73.64 Hz), 6.95- | 129.52, 129.60, 132.07, |
| | 7.01 (3H, m), 7.17 (1H, dd, J = 1.46, | 132.86 139.90, 151.81 (t, |
| | 7.98 Hz), 7.29 (2H, d, J = 8.64 Hz), | J = 2.80 Hz), 154.91, 166.59. |
| | 7.34 (1H, dt, J = 1.35, 7.87 Hz), 7.62 | |
| | (1H, d, J = 15.57 Hz). | |
| 71 | 0.88 (3H, t, J = 7.38 Hz), 1.37-1.44 | 13.87, 14.86, 19.33, 31.28, |
| | (5H, m), 1.70 (1H, quintet, J = 6.93 Hz), | 36.42, 63.62, 68.12, 112.92, |
| | 3.29 (1H, s), 3.95-4.03 (4H, m), 6.16 | 114.64, 116.41, 120.80, |
| | (1H, d, J = 15.53 Hz), 6.78 (2H, d, | 128.14, 129.33, 129.41, |
| | J = 8.64 Hz), 6.95-6.99 (2H, m), 7.18 | 129.73, 132.33, 141.06, |
| | (1H, dd, J = 1.56, 8.08 Hz), 7.23 (2H, d, | 154.96, 160.09, 167.18. |
| | J = 8.64 Hz), 7.33 (1H, dt, J = 1.27, 7.87 | |
| | Hz), 7.61 (1H, d, J = 15.49 Hz). | |
| 72 | 0.89 (3H, t, J = 7.38 Hz), 1.42 (2H, | 13.86, 19.33, 31.27, 36.52, |
| | sextet, J = 7.48 Hz), 1.71 (2H, quintet, | 68.14, 107.61, 109.63, |
| | J = 6.92 Hz), 3.30 (3H, s), 3.98 (2H, dt, | 112.97, 119.20, 120.91, |
| | J = 1.25, 6.23 Hz), 6.19 (1H, d, J = 15.49 | 124.76, 129.57, 129.64, |
| | Hz), 6.95-7.00 (4H, m), 7.05 (1H, dd, | 131.69 (t, J = 255.96 Hz), |
| | J = 1.02, 8.34 Hz), 7.17 (1H, q, J = 3.13 | 131.96, 132.10, 139.88, |
| | Hz), 7.35 (1H, dt, J = 1.36, 7.86 Hz), | 144.25 (t, J = 0.91 Hz), |
| | 7.59 (1H, d, J = 15.49 Hz). | 144.38 (t, J = 1.05 Hz), |
| | | 154.89, 166.37. |
| 73 | 1.94-2.01 (2H, m), 2.15-2.25 (2H, m), | 21.13, 22.19 (q, J = 3.00 Hz), |
| | 3.29 (3H, s), 3.78 (3H, s), 3.84 (3H, s), | 30.52 (q, J = 29.20 Hz), |
| | 3.96-4.06 (2H, m, J = 4.70 Hz), 6.13 | 36.49, 55.92, 55.99, 66.49, |
| | (1H, d, J = 15.49 Hz),6.76 (1H, d, | 110.59, 111.14, 112.80, |
| | J = 7.36 Hz), 6.77 (1H, s), 6.90 (1H, q, | 116.61, 121.33, 121.49, |
| | J = 3.36 Hz), 6.95 (1H, q, J = 3.12 Hz), | 127.08 (q, J = 276.24 Hz), |
| | 7.02 (1H, m, J = 3.28 Hz), 7.22 (1H, q, | 128.40, 129.43, 129.77, |
| | J = 3.12 Hz), 7.33 (1H, m, J = 3.47 Hz), | 132.35, 141.52, 149.02, |
| | 7.60 (1H, d, J = 15.49 Hz). | 150.44, 154.36, 166.98. |
| 74 | 0.87 (3H, t, J = 7.38 Hz), 1.13 (1H, t, | 13.85, 19.30, 31.28, 43.39, |
| | J = 7.16 Hz), 1.40 (2H, sextet, J = 7.50 | 55.40, 60.52, 68.03, 112.73, |
| | Hz), 1.65-1.72 (2H, m), 3.61 (1H, | 114.12, 117.08, 120.63, |
| | sextet, J = 6.86 Hz), 3.77 (3H, s), 3.92- | 128.41, 129.35, 130.60, |
| | 4.01 (3H, m), 6.10 (1H, d, J = 15.49 | 130.67, 140.90, 155.32, |
| | Hz), 6.78 (2H, d, J = 8.80 Hz), 6.94- | 160.62, 166.63, 171.29. |
| | 6.99 (2H, m), 7.14 (1H, dd, J = 1.72, | |
| | 7.92 Hz), 7.23 (2H, d, J = 8.76 Hz), | |
| | 7.33 (1H, dt, J = 1.80, 7.86 Hz), 7.61 | |
| | (1H, d, J = 15.49 Hz). | |
| 75 | 0.90 (3H, t, J = 7.36 Hz), 1.42 (2H, | 13.88, 19.31, 31.23, 52.02, |
| | sextet, J = 7.47 Hz), 1.68 (2H, quintet, | 55.42, 68.02, 112.69, 114.16, |
| | J = 6.98 Hz), 3.80 (3H, s), 3.92 (2H, m), | 116.66, 120.51, 127.17, |
| | 4.50 (1H, d, J = 14.36 Hz), 5.45 (1H, d, | 128.23, 128.32, 129.15, |
| | J = 14.36 Hz), 6.18 (1H, d, J = 15.49 | 129.39, 129.44, 130.49, |
| | Hz), 6.81 (2H, d, J = 8.68 Hz), 6.86 | 130.77, 138.09, 141.56, |
| | (1H, t, J = 7.50 Hz), 6.92 (1H, dd, | 155.05, 160.73, 166.99 . |
| | J = 1.50, 7.62 Hz), 6.96 (1H, d, J = 8.00 | |
| | Hz), 7.21-7.33 (8H, m,), 7.71 (1H, d, | |
| | J = 15.49 Hz). | |
| 76 | 0.86 (3H, t, J = 7.38 Hz), 1.40 (2H, | 13.86, 16.46, 19.29, 27.12, |
| | sextet, J = 7.47 Hz), 1.66-1.73 (2H, m), | 31.23, 47.88, 55.43, 68.04, |
| | 1.80 (2H, quintet, J = 7.32 Hz), 1.90 | 68.47, 84.14, 112.73, 114.15, |
| | (1H, t, J = 2.54 Hz), 2.25-2.23 (2H, m), | 116.74, 120.81, 128.32, |
| | 3.74 (1H, q, J = 7.14 Hz), 3.78 (3H, s), | 129.39, 129.47, 130.37, |
| | 3.89-4.00 (3H, m), 6.10 (1H, d, | 130.71, 141.22, 155.29, |
| | J = 15.49 Hz), 6.79 (2H, d, J = 8.68 Hz), | 160.69, 167.01. |
| | 6.96-7.00 (2H, m), 7.16 (1H, dd, | |
| | J = 1.46, 7.98 Hz), 7.22 (2H, d, J = 8.68 | |
| | Hz), 7.34 (1H, m, J = 4.28 Hz), 7.60 | |
| | (1H, d, J = 15.49 Hz). | |
| 77 | −0.02 (2H, q, J = 4.79 Hz), 0.35-0.39 | 4.54, 10.80, 13.86, 19.31, |
| | (2H, m), 0.60-0.67 (1H, m,), 0.87 (3H, | 28.04, 31.28, 32.26, 48.37, |
| | t, J = 7.38 Hz), 1.24 (2H, sextet, J = 7.02 | 55.42, 68.02, 112.76, 114.13, |
| | Hz), 1.40 (2H, quintet, J = 7.50 Hz), | 117.06, 120.64, 128.44, |
| | 1.63-1.72 (4H, m), 3.51-3.58 (1H, m), | 129.30, 129.35, 130.61, |
| | 3.78 (3H, s),3.90-4.02 (3H, m), 6.10 | 130.90, 140.91, 155.29, |
| | (1H, d, J = 15.49 Hz), 6.79 (2H, d, | 160.61, 166.74. |
| | J = 8.68 Hz), 6.95-6.99 (2H,m), 7.16 | |
| | (1H, dd, J = 1.54, 7.86 Hz), 7.22 (2H, d, | |
| | J = 8.72 Hz), 7.34 (1H, dt, J = 1.57, 7.84 | |
| | Hz), 7.60 (1H, d, J = 15.49 Hz). | |
| 89 | 0.05-0.14 (2H, m), 0.34-0.38 (2H, m,), | 3.23, 4.01, 9.92, 13.79, |
| | 0.85 (3H, t, J = 7.38 Hz), 0.94-1.01 (1H, | 19.21, 31.22, 52.46, 55.86, |
| | m), 1.38 (2H, sextet, J = 7.50 Hz), 1.63- | 55.93, 67.91, 110.47, 111.08, |
| | 1.70 (2H, m), 3.31 (1H, dd, J = 7.16, | 112.51, 117.39, 120.43, |
| | 13.85 Hz), 3.76 (1H, s), 3.82 (1H, s), | 121.24, 128.68, 129.24, |
| | 3.89-3.97 (3H, m), 6.11 (1H, d, | 130.87, 130.90 , 141.05, |
| | J = 15.49 Hz), 6.72-6.75 (2H, m), 6.88 | 148.93, 150.21, 155.22, |
| | (1H, dd, J = 1.82, 8.34 Hz), 6.93-6.97 | 166.59. |
| | (2H,m), 7.23 (1H, dd, J = 1.52, 7.84 | |
| | Hz), 7.31 (1H, dt, J = 1.61, 7.87 Hz), | |
| | 7.57 (1H, d, J = 15.45 Hz). | |

### Example 78: (E)-N-(2-butoxyphenyl)-3-(4-methoxyphenyl)-2-methylacrylamide

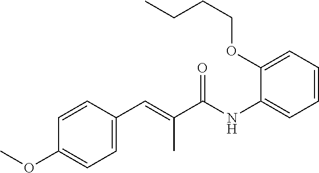 [SMILES: CCCCOc1ccccc1NC(=O)/C(C)=C/c1ccc(OC)cc1]

### a) Ethyl (E)-3-(4-methoxyphenyl)-2-methylacrylate

To a solution of p-anisaldehyde (32 g, 0.24 mol) and ethyl-(2-dimethoxyphosphinyl)-2-propanoate (58.8 g, 0.28 mol) in toluene (160 mL) was added NaOtBu (33.6 g, 0.35 mol) at 0° C. under N2, atmosphere over the course of 30 minutes. The reaction mixture was warmed to room temperature and stirred for 15 min. After completion of the reaction, the reaction mixture was neutralised with 10% aq. HCl The toluene layer was separated, washed with water, dried over sodium sulphate and concentrated in vacuo. The residue was purified by silica gel chromatography to give 22.7 g ethyl (E)-3-(4-methoxyphenyl)-2-methylacrylate.

¹H-NMR (CDCl₃): 1.35 (3H, t, J=7.12 Hz), 2.13 (3H, d, J=1.20 Hz), 3.84 (3H, s), 4.26 (2H, q, J=7.12 Hz), 6.92 (2H, d, J=8.72 Hz), 7.39 (2H, d, J=8.64 Hz), 7.64 (1H, s).

### b) (E)-3-(4-methoxyphenyl)-2-methylacrylic acid

A 50 mL round-bottom flask was charged with ethyl (E)-3-(4-methoxyphenyl)-2-methylacrylate (10 mmol, 1 eq.), LiOH·H2O (50 mmol, 5 eq.) in a mixture of tetrahydrofurane/water (1:1, 0.25 M) The reaction flask was heated to 80° C. and stirred for 3 h. After cooling to room temperature, the mixture was extracted with diethyl ether. The aqueous phase was acidified by addition of 2N hydrochloric acid and extracted with ethyl acetate. The combined organic layers were dried over magnesium sulphate and subsequently evaporated to dryness (18.6 g).

### c) (E)-N-(2-butoxyphenyl)-3-(4-methoxyphenyl)-2-methylacrylamide (Example 78)

By following the synthesis procedure of Example 3 ((E)-3-(4-methoxyphenyl)-N-(2-phenoxyphenyl)acrylamide), (E)-N-(2-butoxyphenyl)-3-(4-methoxyphenyl)-2-methylacrylamide was obtained in 92% yield, employing (E)-3-(4-methoxyphenyl)-2-methylacrylic acid and 2.butoxyaniline. ¹H-NMR (CDCl₃). 1.00 (1H, t, J=7.40 Hz), 1.54 (2H, sextet, J=7.50 Hz), 1.80-1.87 (2H, m), 2.24 (3H, d, J=1.32 Hz), 3.85 (3H, s), 4.07 (2H, t, J=6.38 Hz), 6.89 (1H, dd, J=1.56, 7.88 Hz), 6.93-6.95 (2H, m), 6.99 (1H, dt, J=1.60, 7.70 Hz), 7.04 (1H, dt, J=1.88, 7.67 Hz), 7.37 (2H, d, J=8.68 Hz), 7.47 (1H, s), 8.41 (1H, s), 8.50 (1H, dd, J=1.82, 7.82 Hz). ¹³C-NMR (CDCl₃): 13.99, 14.33, 19.50, 31.45, 55.45, 68.36, 110.90, 114.01, 119.75, 121.22, 123.65, 128.26, 128.75, 130.71, 131.17, 134.60, 147.61, 159.54, 167.39. The examples listed in the following Table 4 were obtained in a similar fashion as described for Example 78.

TABLE 4

| Example | IUPAC | Structure | HR-MS |
|---|---|---|---|
| 79 | (E)-N-(2- butoxyphenyl)-3-(4- methoxyphenyl)-2- methylacrylamide | 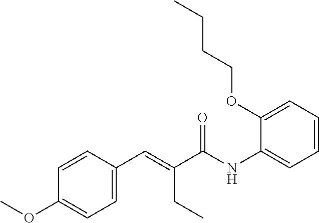 [SMILES: CCCCOc1ccccc1NC(=O)/C(=C/c1ccc(OC)cc1)CC] | Found: 362.1714 [M + Na]⁺ calc. 362.1732 |
| 80 | (E)-N-(2- butoxyphenyl)-2- (cyclopropylmethyl)- 3-(4- methoxyphenyl) acrylamide | 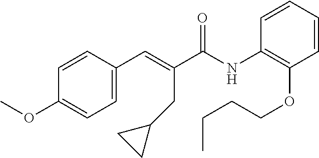 [SMILES: CCCCOc1ccccc1NC(=O)/C(=C/c1ccc(OC)cc1)CC1CC1] | Found: 402.2038 [M + Na]⁺ calc. 402.2045 |
| 81 | (E)-N-(2- butoxyphenyl)-2-(4- methoxybenzylidene)-4- methylpentanamide | 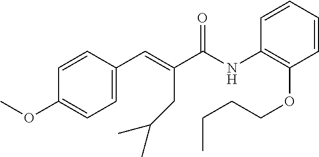 [SMILES: CCCCOc1ccccc1NC(=O)/C(=C/c1ccc(OC)cc1)CC(C)C] | Found: 404.2203 [M + Na]⁺ calc. 404.2202 |
| 82 | (E)-N-(2- butoxyphenyl)-2-(4- methoxybenzylidene)-3- methylbutanamide | 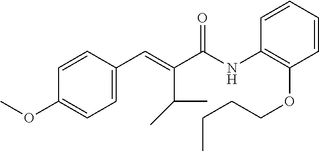 [SMILES: CCCCOc1ccccc1NC(=O)/C(=C/c1ccc(OC)cc1)C(C)C] | Found: 390.2040 [M + Na]⁺ calc. 390.2045 |
| 146 | (E)-N-(2-butoxyphenyl)-2- cyclopropyl-3-(4- methoxyphenyl)acrylamide | 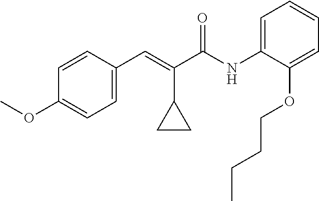 [SMILES: CCCCOc1ccccc1NC(=O)/C(=C/c1ccc(OC)cc1)C1CC1] | Found: 388.1897 [M + Na] calc. 388.1889 |

| Example | ¹H-NMR (CDCl3) | ¹³C-NMR (CDCl3) |
|---|---|---|
| 79 | 0.99 (3H, t, J = 7.40 Hz), 1.28 (3H, t, | 13.75, 13.97, 19.49, 21.18, |
| | J = 7.54 Hz), 1.53 (2H, sextet, J = 7.48 | 31.44, 55.44, 68.36, 110.86, |
| | Hz), 1.83 (2H, quintet, J = 6.93 Hz), 2.68 | 114.09, 119.81, 121.19, 123.65, |
| | (2H, q, J = 7.54 Hz), 3.85 (3H, s), 4.07 | 128.23, 128.62, 130.73, 133.61, |
| | (2H, t, J = 6.34 Hz), 6.89 (1H, dd, | 137.81, 147.59, 159.59, 167.16. |
| | J = 1.22, 7.86 Hz), 6.93-6.95 (2H, m), | |
| | 6.99 (1H, dt, J = 1.31, 7.66 Hz), 7.04 | |
| | (1H, dt, J = 1.71, 7.67 Hz), 7.33-7.37 | |
| | (3H, m), 8.41 (1H, s), 8.50 (1H, dd, | |
| | J = 1.62, 7.78 Hz). | |
| 80 | 0.25 (2H, q, J = 5.00 Hz), 0.50 (2H, q, | 4.80, 10.38, 13.99, 19.51, 31.42, |
| | J = 6.06 Hz), 0.99 (4H, t, J = 7.40 Hz), | 31.64, 55.44, 68.43, 110.98, |
| | 1.53 (2H, sextet, J = 7.47 Hz), 1.83 (2H, | 114.07, 119.99, 121.20, 123.68, |
| | quintet, J = 6.95 Hz), 2.64 (2H, d, J = 6.24 | 128.30, 128.56, 130.81, 133.73, |
| | Hz), 3.85 (3H, s), 4.08 (2H, t, J = 6.40 | 136.48, 147.66, 159.58, 167.98. |
| | Hz), 6.90 (1H, d, J = 7.96 Hz), 6.94 (2H, | |
| | d, J = 8.68 Hz), 6.99 (1H, dt, J = 1.25, | |
| | 7.76 Hz), 7.04 (1H, dt, J = 1.67, 7.79 | |
| | Hz), 7.35-7.39 (3H, m), 8.41 (1H, s), | |
| | 8.49 (1H, dd, J = 1.28, 7.80 Hz). | |
| 81 | 0.95 (6H, d, J = 6.64 Hz), 0.99 (3H, t, | 13.99, 19.54, 22.75, 28.26, |
| | J = 7.40 Hz), 1.53 (2H, sextet, J = 7.48 | 31.44, 36.55, 55.44, 68.39, |
| | Hz), 1.83 (2H, quintet, J = 6.94 Hz), 1.96 | 110.91, 114.03, 119.88, 121.20, |
| | (1H, heptet, J = 6.80 Hz), 2.58 (2H, d, | 123.68, 128.22, 128.73, 130.77, |
| | J = 7.36 Hz), 3.84 (3H, s), 4.07 (2H, t, | 134.18, 137.02, 147.61, 159.42, |
| | J = 6.38 Hz), 6.90 (1H, d, J = 7.96 Hz), | 168.12. |
| | 6.93 (2H, d, J = 8.68 Hz), 6.98 (1H, dt, | |
| | J = 1.28, 7.79 Hz), 7.04 (1H, dt, J = 1.65, | |
| | 7.74 Hz), 7.33 (2H, dd, J = 6.58 Hz),7.34 | |
| | (1H, t, J = 6.58 Hz), 8.34 (1H, s), 8.48 | |
| | (1H, dd, J = 1.38, 7.82 Hz). | |
| 82 | 1.00 (3H, t, J = 7.38 Hz), 1.33 (3H, s), | 14.01, 19.54, 21.71, 28.11, |
| | 1.35 (3H, s), 1.54 (2H, sextet, J = 7.47 | 31.42, 55.44, 68.41, 110.96, |
| | Hz), 1.83 (2H, quintet, J = 6.92 Hz), 3.21 | 114.02, 119.90, 121.15, 123.72, |
| | (1H,quintet, J = 6.95 Hz), 3.84 (3H, s), | 128.16, 128.38, 130.41, 130.50, |
| | 4.07 (2H, t, J = 6.32 Hz), 6.87-7.00 (5H, | 144.36, 147.55, 159.26, 168.40. |
| | m), 7.04 (1H, dt, J = 1.47, 7.69 Hz),7.28 | |
| | (2H, d, J = 8.60 Hz), 8.17 (1H, s), 8.45 | |
| | (1H, dd, J = 1.28, 7.84 Hz). | |
| 146 | 0.50 (2H, q, J = 5.31 Hz), 1.00 (3H, t, | 9.21, 10.21, 13.98, 19.44, 31.59, |
| | J = 7.40 Hz), 1.05-1.10 (2H, m), 1.54 | 55.42, 68.26, 110.66, 113.49, |
| | (2H, sext, J = 7.52 Hz), 1.72-1.76 (1H, | 119.82, 121.15, 123.54, 128.15, |
| | m, J = 3.40 Hz), 1.85 (2H, quint, J = 7.02 | 128.36, 132.20, 132.31, 139.06, |
| | Hz), 3.85 (3H, s), 4.07 (2H, t, J = 6.48 | 147.76, 159.85, 166.00. |
| | Hz), 6.89-6.93 (3H, m), 6.97-7.06 (2H, | |
| | m), 7.57 (2H, d, J = 8.64 Hz), 7.85 (1H, | |
| | s), 8.56 (1H, dd, J = 1.54, 7.78 Hz), 9.26 | |
| | (1H, s). | |

### Example 167. (E)-N-(2-(2-(3-methyl-3H-diazirin-3-yl)ethoxy)phenyl)-3-(4-(prop-2-yn-1-yloxy)phenyl)acrylamide

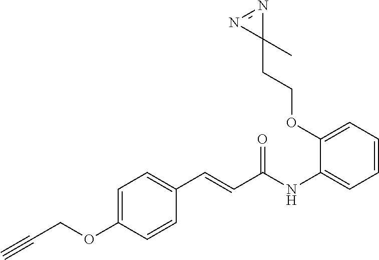 [SMILES: C#CCOc1ccc(/C=C/C(=O)Nc2ccccc2OCCC2(C)N=N2)cc1]

### a) methyl (E)-3-(4-(prop-2-yn-1-yloxy)phenyl)acrylate

To a solution of the methyl (E)-3-(4-hydroxyphenyl)acrylate (2.8 mmol, 500 mg, 1.0 equiv.) and propargyl bromide (3.4 mmol, 401 mg, 1.2 equiv.) in acetone (5.6 mL), K₂CO₃ (3.4 mmol, 465 mg, 1.2 equiv.) was added and the mixture was stirred at 60° C. for 24 h. The reaction mixture was cooled down to room temperature, filtered to remove the solid, and the volatiles removed under reduced pressure. The crude product was purified by column chromatography on silica gel chromatography to afford methyl (E)-3-(4-(prop-2-yn-1-yloxy)phenyl)acrylate (573 mg, 94%).

¹H-NMR (CDCl₃ 400 MHz): 2.54 (1H, t, J=2.38 Hz), 3.79 (3H, s), 4.72 (2H, d, J=2.40 Hz), 6.32 (1H, d, J=15.97 Hz), 6.98 (1H, d, J=8.76 Hz), 7.49 (2H, d, J=8.76 Hz), 7.65 (1H, d, J=15.97 Hz).

¹³C-NMR (CDClₐ 101 Hz): 51.76, 55.94, 76.09, 78.16, 115.36, 115.95, 128.09, 129.79, 144.43, 159.33, 167.81.

### b) (E)-3-(4-(prop-2-yn-1-yloxy)phenyl)acrylic acid

Methyl (E)-3-(4-(prop-2-yn-1-yloxy)phenyl)acrylate (573 mg, 2.7 mmol) was dissolved in methanol (10 ml). 1M sodium hydroxide aqueous solution (10 mL) was added. The reaction mixture was stirred at room temperature for 2 hours. The resultant was acidified (pH=3) with 2N HCl, and the aqueous layer was extracted with ethyl acetate. The combined organic layer was dried over anhydrous sodium sulfate and concentrated under reduced pressure to give (E)-3-(4-(prop-2-yn-1-yloxy)phenyl)acrylic acid 510 mg (yield: 95%, white solid), which was used in next step without purification.

### c) (E)-N-(2-hydroxyphenyl)-3-(4-(prop-2-yn-1-yloxy)phenyl)acrylamide

Added (E)-3-(4-(prop-2-yn-1-yloxy)phenyl)acrylic acid (293 mg, 1 mmol), DIPEA (1 mmol, 174 μL) and HATU (418 mg, 1.1 mmol) in 10 mL DCM at 0° C. and stirred for 30 min. Then 2-aminophenol (2 mmol, 218 mg) was added and stirring was continued overnight. After quenching by 1 M HCl, it was extracted by ethyl acetate and the combined organic phase was dried with Na₂SO₄. After being concentrated in vacuum, the residue was purified by silica column to afford (E)-N-(2-hydroxyphenyl)-3-(4-(prop-2-yn-1-yloxy)phenyl)acrylamide (270 mg, 92%)

¹H-NMR (DMSO 400 MHz): 3.61 (1H, t, J=2.14 Hz), 4.86 (2H, d, J=2.20 Hz), 6.80 (1H, t, J=7.58 Hz), 6.89 (1H, d, J=6.84 Hz), 6.96 (1H, t, J=7.52 Hz), 7.00-7.06 (3H, m), 7.53 (1H, d, J=15.61 Hz), 7.60 (2H, d, J=8.64 Hz), 7.92 (1H, d, J=7.72 Hz), 9.44 (1H, s), 9.99 (1H, s).

HRMS (ESI): Found: 292.0976 [M−H]⁻ calc. 290.0974.

### d) (E)-N-(2-(2-(3-methyl-3H-diazirin-3-yl)ethoxy)phenyl)-3-(4-(prop-2-yn-1-yloxy)phenyl)acrylamide (HLM-01-543)

To a mixture of (E)-N-(2-hydroxyphenyl)-3-(4-(prop-2-yn-1-yloxy)phenyl)acrylamide (0.27 mmol, 80 mg), triphenyl phosphine (1.2 eq, 0.33 mmol, 86 mg), (3-methyl-3H-diazirin-3-yl)ethanol [prepared according to ref. 42] (1.5 eq, 0.41 mmol, 41 mg) in THF (3 ml), diethyl azodicarboxylate (1.2 eq, 0.33 mmol, 51 μL) was added at 0° C. After the solution was stirred overnight at room temperature, the solvent was removed in vacuum, and the residue was purified by silica column to afford (E)-N-(2-(2-(3-methyl-3H-diazirin-3-yl)ethoxy)phenyl)-3-(4-(prop-2-yn-1-yloxy)phenyl)acrylamide (HLM-01-543, 37 mg, 36%).

¹H-NMR (CDCl₃, 400 MHz): 1.14 (3H, s), 1.91 (2H, t, J=5.82 Hz), 2.55 (1H, t, J=2.40 Hz), 4.04 (2H, t, J=5.82 Hz), 4.73 (2H, d, J=2.40 Hz), 6.66 (1H, d, J=15.53 Hz), 6.84-6.87 (1H, m), 6.98-7.03 (4H, m), 7.55 (2H, d, J=8.72 Hz), 7.74 (1H, d, J=15.53 Hz), 8.38 (1H, s), 8.56 (2H, d, J=3.92 Hz).

¹³C-NMR (CDCl₃ 101 MHz): 19.58, 24.76, 34.36, 55.95, 64.19, 76.03, 78.27, 111.19, 115.35, 119.76, 120.18, 121.98, 123.60, 128.57, 128.65, 129.63, 141.37, 146.88, 158.97, 164.38.

HRMS (ESI): Found: 398.1476 [M−H]⁻ calc. 398.1481.

### Example 168: (E)-N-(2-(2-(3-methyl-3H-diazirin-3-yl)ethoxy)phenyl)-3-(4-(pent-4-yn-1-yloxy)phenyl)acrylamide

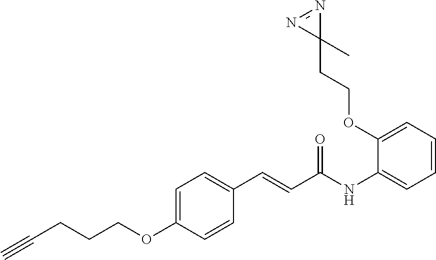 [SMILES: C#CCCCOc1ccc(/C=C/C(=O)Nc2ccccc2OCCC2(C)N=N2)cc1]

By following the synthesis procedure of (E)-N-(2-(2-(3-methyl-3H-diazirin-3-yl)ethoxy)phenyl)-3-(4-(prop-2-yn-1-yloxy)phenyl)acrylamide (HLM-01-543), (E)-N-(2-(2-(3-methyl-3H-diazirin-3-yl)ethoxy)phenyl)-3-(4-(pent-4-yn-1-yloxy)phenyl)acrylamide (HLM-02-544) was obtained as white solid (51 mg, 53%)

¹H-NMR (CDCl₃, 400 MHz). 1.04 (3H, s), 1.81 (2H, t, J=5.82 Hz), 1.87-1.95 (3H, m), 2.32 (1H, dt, J=2.65, 6.95 Hz), 3.94 (2H, t, J=5.82 Hz), 4.01 (2H, t, J=6.10 Hz), 6.54 (1H, d, J=15.53 Hz), 6.75-6.77 (1H, m), 6.82 (1H, d, J=8.76 Hz), 6.91-6.94 (2H, m), 7.43 (2H, d, J=8.64 Hz), 7.64 (1H, d, J=15.53 Hz), 8.26 (1H, s), 8.46 (1H, s).

¹³C-NMR (CDCl₁ 101 MHz). 15.29, 19.60, 24.75, 28.20, 34.38, 64.19, 66.36, 69.14, 83.44, 111.18, 114.97, 119.24, 120.20, 122.00, 123.56, 127.75, 128.71, 129.70, 141.60, 146.87, 160.49, 164.51.

HRMS (ESI): Found: 426.1791 [M−H]⁻ calc. 426.1794.

### 2. Screening Assay

### Material and Methods

### Primary Cell Culture

Primary human lung fibroblasts (phLFs) were isolated by outgrowth from human lung tissue derived from lung explants or tumor-free areas of lung resections as previously described (29, 30). Cells were cultured in Dulbecco's Modified Eagle Medium F-12 with 20% (v/v) special processed fetal bovine serum (PAN Biotech, Cat. No. and 100 International Units Penicillin per mL and 100 μg per mL Streptomycin. Medium was changed every 2-3 days and cells were passaged at 80-90% confluency in a ratio of 1:5 or 1:6. Cells were used for experiments until passage 7. For ECM deposition drug screening, 0.5-1×106 cells were expanded from passage 1 to passage 5, each time in a ratio of 1:6. More than 100×106 cells were trypsinized at passage 5 and cryopreserved in 90% (v/v) fetal bovine serum and 10% (v/v) dimethyl sulfoxide. Cells were frozen slowly by using Mr. Frosty (ThermoFisher Scientific) freezing containers. For reseeding phLFs were thawed in a water bath at 37° C. and the cells were washed with culture medium, prior to plating. After reaching confluency in passage 6, cells were used for the ECM deposition assays. Primary human dermal fibroblast (Cat #DF-F) were purchased from ZenBio Inc. and cultured according to the manufacturer's instructions.

### ECM Deposition Assay

phLFs were cultured in DMEM F-12 medium with 20% fetal bovine serum (FBS) and antibiotic supplement as mentioned above. Cells were seeded with 6000 cells/well in 384-well CellCarrier plates (Perkin Elmer, Cat #6007550). Following overnight incubation, cells were starved in serum-reduced medium (1% FBS with 0.1 mM 2-phosphoascorbate (Sigma, Cat #49752)) for 24 h. Afterwards, cells were treated with TGFβ1 (1 ng/ml) or vehicle, and additionally small molecules or appropriate vehicle controls were added. After 72 h of incubation, medium was changed for starving medium with 1 μg/mL AlexaFluor-488 fluorophore conjugated anti-collagen-type-5 antibodies (SantaCruz, Clone C-5, Cat #sc-166155 AF488), 0.66 μg/mL AlexaFluor-555 fluorophore conjugated anti-collagen-type-1 antibodies (Rockland, Cat #600-401-103-0.1), and 1 μg/mL AlexaFluor-637 fluorophore conjugated anti-fibulin 1 antibodies (SantaCruz, Clone C-5, Cat #sc-25281 AF647) and 1 μg/mL Hoechst H33342 (Sigma). Fluorescenceconjugation of the collagen type 1 antibody was done by using the AlexaFluor-555 Protein Labeling Kit (Invitrogen, Cat #A20174) according to manufacturer's instructions. Labeling efficacy was controlled by photometrical means.

Following four hours of incubation, cells were washed three times with PBS and fixed with paraformaldehyde (PFA). For automated liquid handling in 384 well plates, an INTEGRA Assist Plus (INTEGRA, Zizers, Switzerland) equipped with an INTEGRA Viaflo 1I pipette (INTEGRA, Zizers, Switzerland, Cat #4642), 125 μL GripTips™ pipette tips (INTEGRA, Zizers, Switzerland, Cat #6464) and sterile reagent reservoirs (INTEGRA, Cat #4311) were applied. All automated pipetting steps with the INTEGRA Assist Plus were performed at 9.5 μL/s in order to ensure proper integrity and attachment of the deposited ECM to the culturing surface within the wells of the 384-well plates. During cell seeding the automated liquid handling was performed at 89.3 μL/s. Removal of liquids from the well-plates was done by manually inverting the plates.

Following fixation, the automated imaging was achieved by using aconfocal laser scanning microscope (LSM710, Zeiss) with automated focus detection for three-dimensional image acquisition (1024 px×1024 px×9 px which equals a dimension of 1417 μm×1417 μm×16 μm). For post-acquisition analysis images were imported into IMARIS software (Bitplane) and volume detection or alternatively quantification of the mean fluorescence intensity, as well as Hoechst-stained cell nuclei were automatically counted by using Imaris' spot detection algorithm.

### Human Precision-Cut Lung Slices (PCLS) and Fibrotic Cocktail Treatment

PCLS were prepared as described before (31, 32). Shortly, PCLS were prepared from tumor-free peri-tumor tissue. The lung tissue was inflated with 3% agarose solution and solidified at 4° C. Tissue blocks were cut in μm thick PCLS using a vibration microtome Hyrax V50 (Zeiss). PCLS were cultured in DMEM F-12 medium and treated with a profibrotic cocktail, as described before (31), or vehicle, as well as with small molecules or vehicles for 7 days. After culturing and treatments, supernatants were harvested. PCLS were 500 washed in PBS and protein was extracted as previously described (33). Briefly, PCLS were pooled in an Eppendorf tube and lysed in 500 μl ice-cold RIPA buffer (50 mM Tris-CI pH 7.4, 150 mM NaCl, 1% NP40, 0.25% Na-deoxycholate) containing 1× Roche complete mini protease inhibitor cocktail (Roche, Cat. #11697498001). After an incubation of 2 hours rotating at 4° C., the lung slices were removed from the lysates and the protein content was measured.

### Cytotoxicity Assays

Viability/Cytotoxicity Assay Kit for Animal Live and Dead Cells was obtained from Biotium, Cat. No. 3002). CellEvent™ Caspase 3/7 Green Detection Reagent was acquired from Invitrogen, Cat. No. C₁₀₄₂₃. For MTT-assays, Thiazolyl Blue Tetrazolium was bought from SigmaAldrich (M5655-1G). All these kits and assays were used according to the manufacturer's instructions.

### Antibodies for Immunofluorescence and Dyes

For immunofluorescence microscopy the following antibodies were used: monoclonal mouse anti-collagen type 5 (1 mg/mL) from Sigma Aldrich (Cat #sc-166155), monoclonal mouse anti-collagen type 5 AlexaFluor-488 conjugate from Sigma Aldrich (Cat #sc-166-155AF488), polyclonal rabbit anti-collagen type 1 from Rockland (Cat #600-401-103-0.5), monoclonal mouse anti-fibulin 1 from SantaCruz (Cat #sc-25281), monoclonal mouse anti-fibulin 1 AlexaFluor-647 conjugate (1 mg/mL) from SantaCruz (Cat #sc-25281AF647), and polyclonal rabbit anti-fibronectin (1 mg/mL) from SantaCruz (Cat #sc-9068). Hoecst-33342 was obtained from Sigma (Cat #B2261). The following secondary antibodies were used: AF488 donkey-anti-mouse Ab (Invitrogen, Cat. #A21202), AF568 donkey-anti-mouse Ab (Invitrogen, Cat. #A11004), and AF568 donkey-anti-mouse Ab (Invitrogen, Cat. #A11011). For immunofluorescence stainings of actin-stress fibers Alexa Fluor 568 Phalloidin (Invitrogen, A12380) was used. 4′, 6-diamidino-2-phenylindole (DAPI) was acquired from Sigma-Aldrich (Cat #D9564).

### Immunocytochemistry

For standard immunofluorescence staining, 5000 phLFs were seeded into 96 well imaging plates with a flat bottom (Cat #353376, BD Biosciences). After incubation, cells were fixed with either 4% PFA for 30 min at 37° C. or 100% methanol for 2 min at −20° C. If needed, phLFs were permeabilized with 0.25% (v/v) Triton X-100 in PBS for 15 min. After washing with 100 μL of PBS blocking was done by incubation with 5% (w/v) BSA in PBS for one hour. Primary antibodies were diluted in 1% bovine serum albumin (BSA, Sigma) in PBS, incubated for 16 hours at 4° C. and subsequently washed three times with PBS for 20 minutes each. Secondary antibodies were diluted in 1% bovine serum albumin (BSA, Sigma) in PBS, incubated for one hour at room temperature and subsequently washed three times with PBS for 20 minutes each. 4% paraformaldehyde in phosphate buffered saline (w/v) was prepared from paraformaldehyde from Sigma (Cat #15,812-7). Bovine serum albumin was obtained from Sigma (Cat #A3059). Triton X-100 was obtained from AppliChem (Cat #A1388).

### Confocal 3D and 4D Imaging

Confocal time-lapse microscopy was implemented on an LSM710 system (Carl Zeiss) containing an inverted AxioObserver.Z1 stand equipped with phase-contrast and epi-illumination optics and operated by ZEN2009 software (Carl Zeiss). The following objectives were used for imaging: EC Plan-Neofluar 20×/0.8 NA (Carl Zeiss), LD C-Apochromat 40x/1.1 NA water objective lens (Carl Zeiss) and LCI PLN-NEOF DICIII 63×/1.30 NA water objective lens (Carl Zeiss). For 4D imaging the cells were kept in an incubation chamber (Carl Zeiss) under standard cultivation conditions (37° C. and 5% CO₂). Thickness of single confocal layers within the z-stacks was set according to optimized values suggested by the ZEN2009 software. The confocal data sets were either maximum intensity projected in the ZEN2009 software (Carl Zeiss) and/or imported into Imaris 9.0.0-9.3.1 software (Bitplane) for analysis.

### Protein Isolation, SDS-PAGE, Western Blotting and ELISA

Cells were scraped off the plastic dish directly into 200 μl ice-cold RIPA buffer containing 1× Roche complete mini protease inhibitor cocktail. After incubating the samples for 30 minutes on ice, insoluble material was removed by centrifugation at 14.000 g for 15 minutes at 4° C. and the supernatant was further processed. Samples were mixed with 50 mM Tris-HCl, pH 6.8, 100 mM DTT, 2% SDS, 1% bromphenol blue, and 10% glycerol, and proteins were separated using standard SDS-10% PAGE. For immunoblotting, proteins were transferred to PVDF (Millipore (Billerica, MA, (USA)), 0.45 μm or 0.2 μm) membranes, which were blocked with 5% milk in TBST (0.1% Tween 20 in TBS) and incubated with primary, followed by HRP-conjugated secondary antibodies over night at 4° C. and at room temperature for 1 hour, respectively. For immunoblotting the following primary antibodies were used: monoclonal mouse anticollagen type 5 (1 mg/mL) from Sigma Aldrich (Cat #sc-166155), polyclonal rabbit anti-collagen type 3 (1 mg/mL) from Rockland (Cat #600-401-105), polyclonal rabbit anti-collagen type 1 (1 mg/mL) from Rockland (Cat #600-401-103-0.5), monoclonal mouse anti-fibulin 1 (1 mg/mL) from SantaCruz (Cat #sc-25281), polyclonal rabbit anti-fibronectin (1 mg/mL) from SantaCruz (Cat #sc-9068), and monoclonal mouse anti-p-actin-peroxidase (AC-15, Sigma, 1:10000). Goat anti-rabbit and goat anti-mouse IgG conjugated to horseradish peroxidase (Cell Signaling, 1:10000) were applied as secondary antibodies. CXCL/IL-8 concentrations were determined using Human IL-8/CXCL8 DuoSet ELISA (DY208-05) according to the manufacturer's protocol.

mRNA Isolation, cDNA Synthesis and qRT-PCR

RNA extraction from cultured phLFs was performed using the PeqGold RNA kit (Peqlab) according to the manufacturer's instruction. The concentration of the isolated RNA was assessed spectrophotometrically at a wavelength of 260 nm (NanoDrop 1000). cDNA was synthesized with the GeneAMP PCR kit (Applied Biosystems (Foster City, CA, USA)) utilizing random hexamers using 1 μg of isolated RNA for one 301 reaction. Denaturation was performed in an Eppendorf Mastercycler with the following settings: 302 303 lid=45° C., 70° C. for 10 minutes and 4° C. for 5 minutes. Reverse transcription was performed in an Eppendorf Mastercycler with the following settings: lid=105° C., 20° C. for 10 minutes, 42° C. for 60 minutes and 99° C. for 5 minutes. qRT-PCR reactions were performed in triplicates with SYBR Green I Master in a LightCycler@ 48011 (Roche (Risch, Switzerland)) with standard conditions: 95° C. for 5 min followed by 45 cycles of 95° C. for 5 s (denaturation), 59° C. for 5 s (annealing) and 72° C. for 20 s (elongation). Target genes were normalized to HPRT expression.

### Microarray and UMAP-Regulation Pattern Clustering (UMAP-RPC)

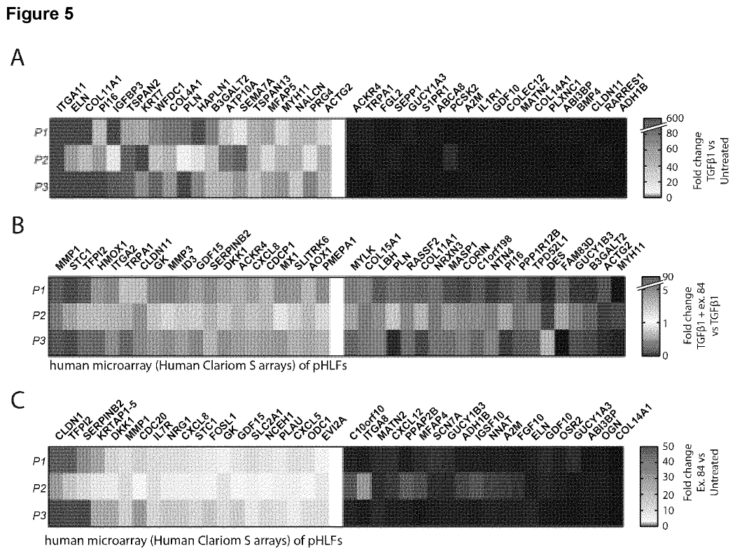

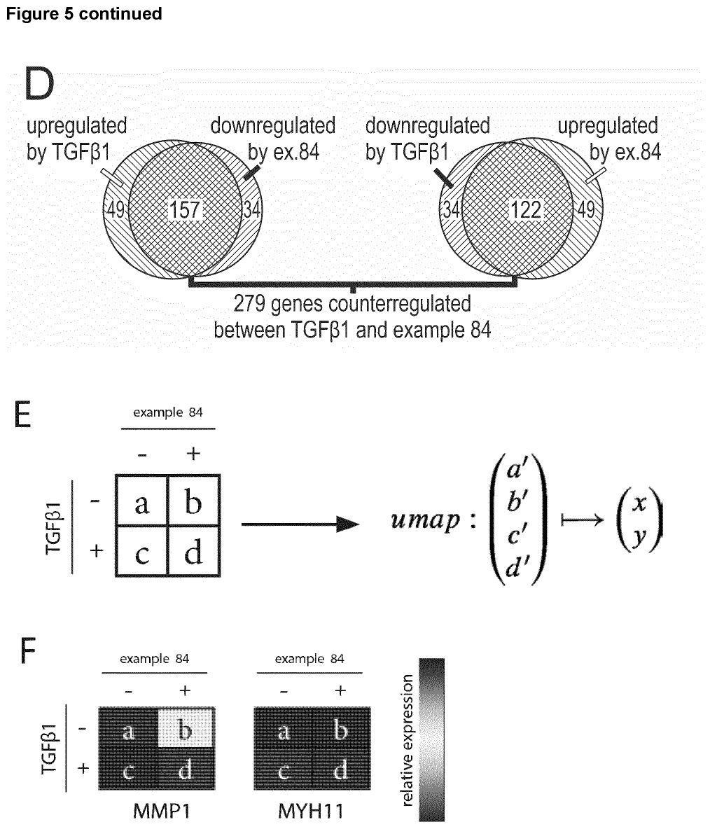

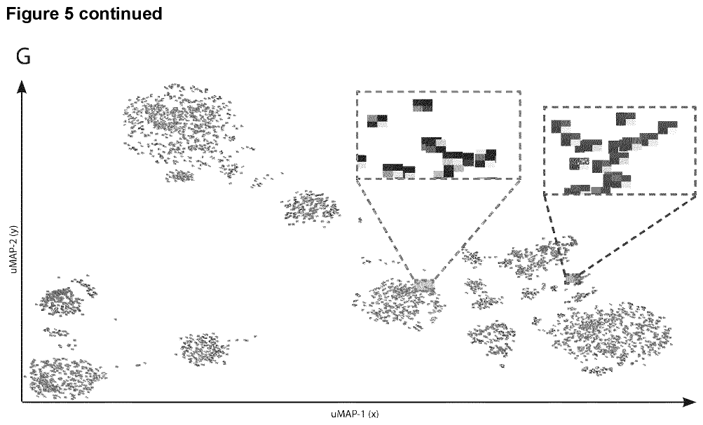

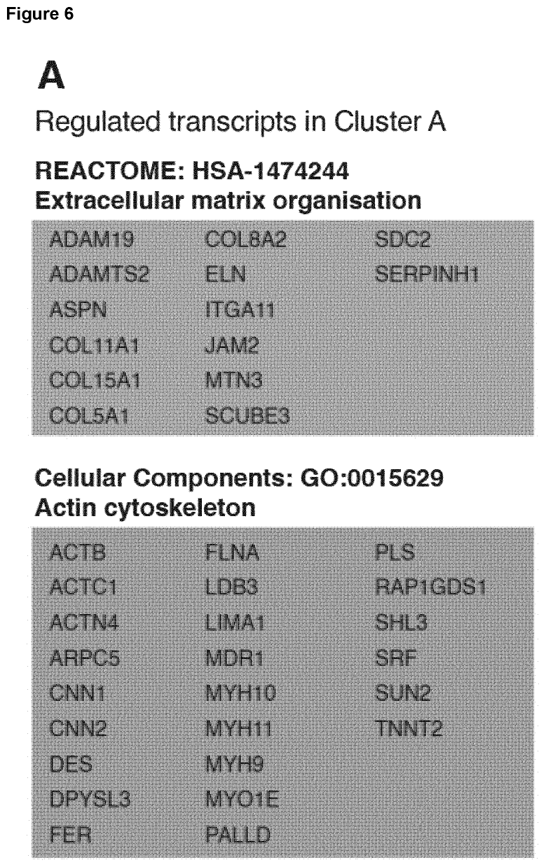

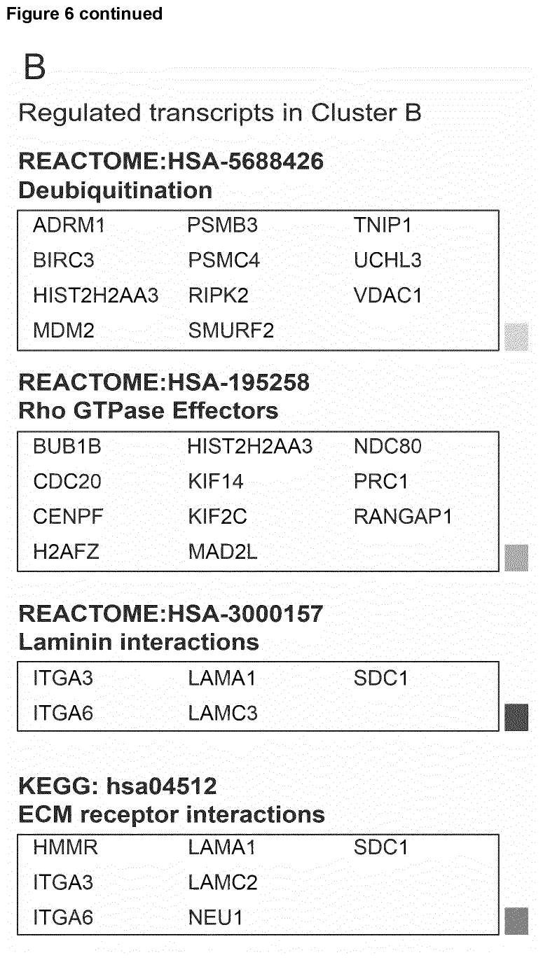

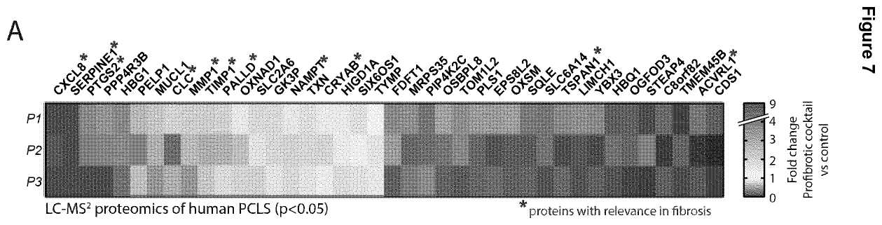

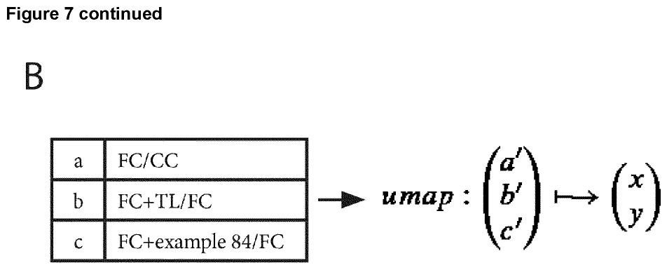

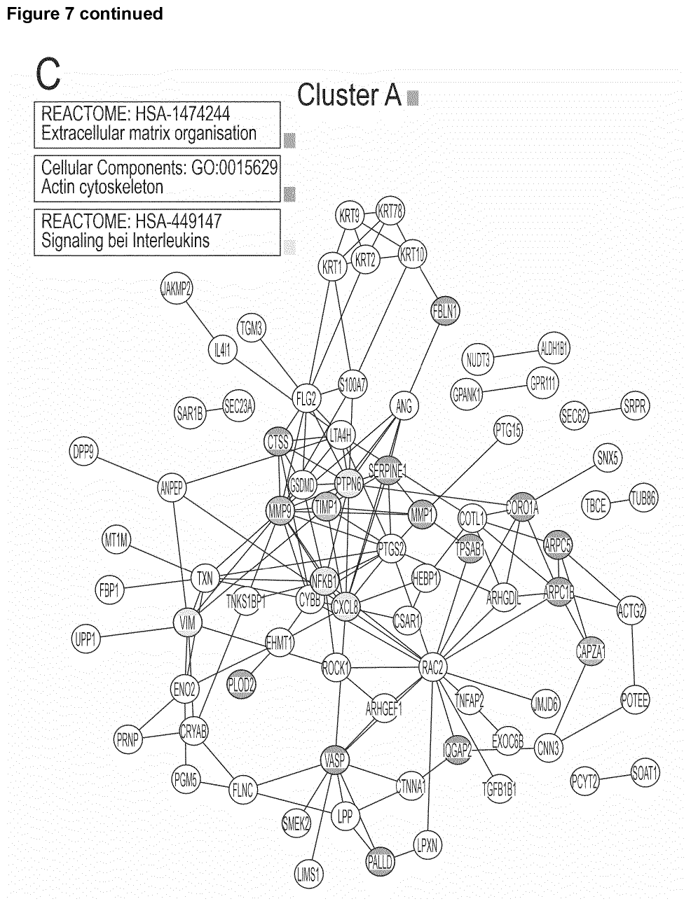

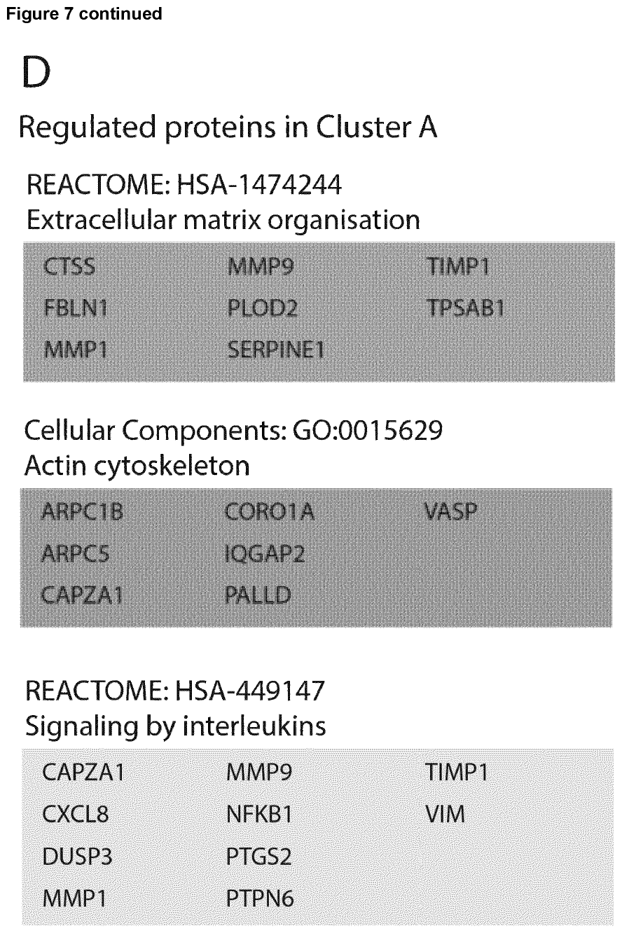

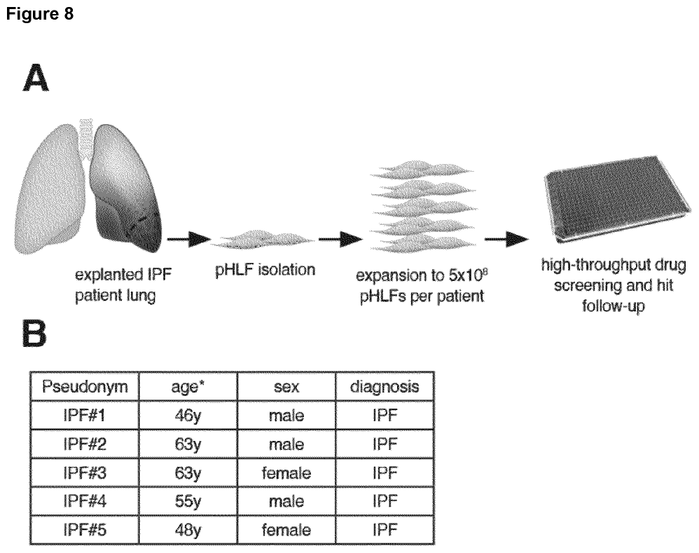

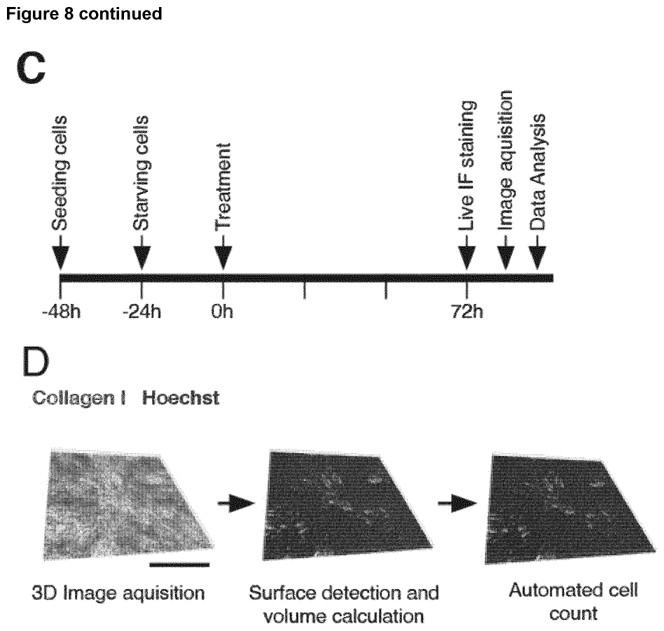

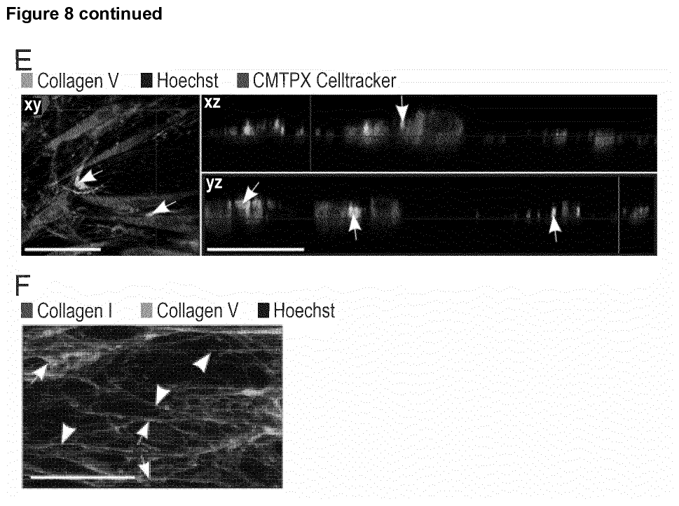

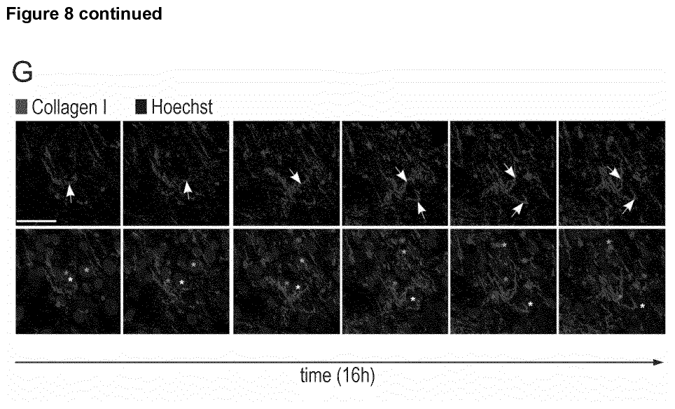

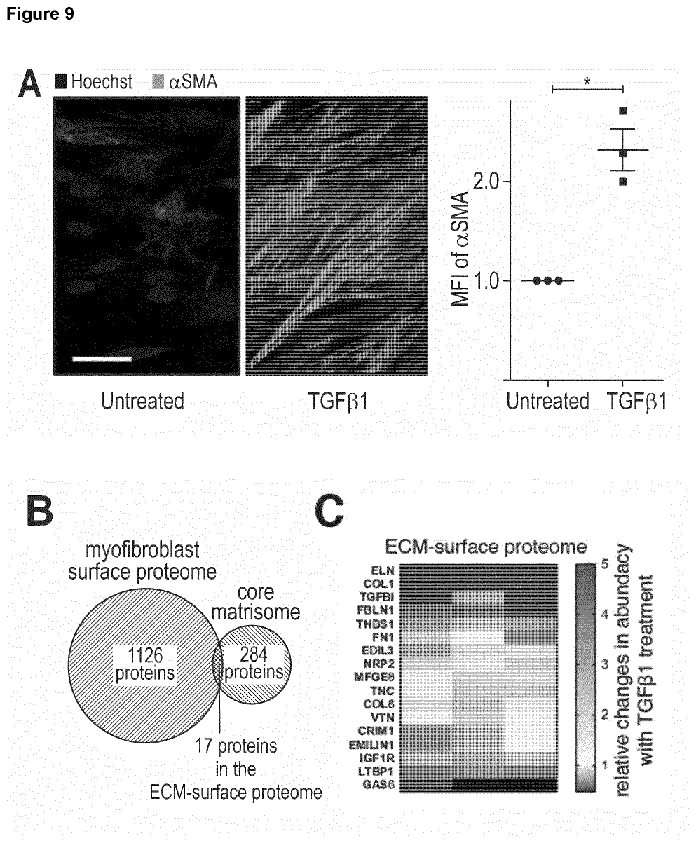

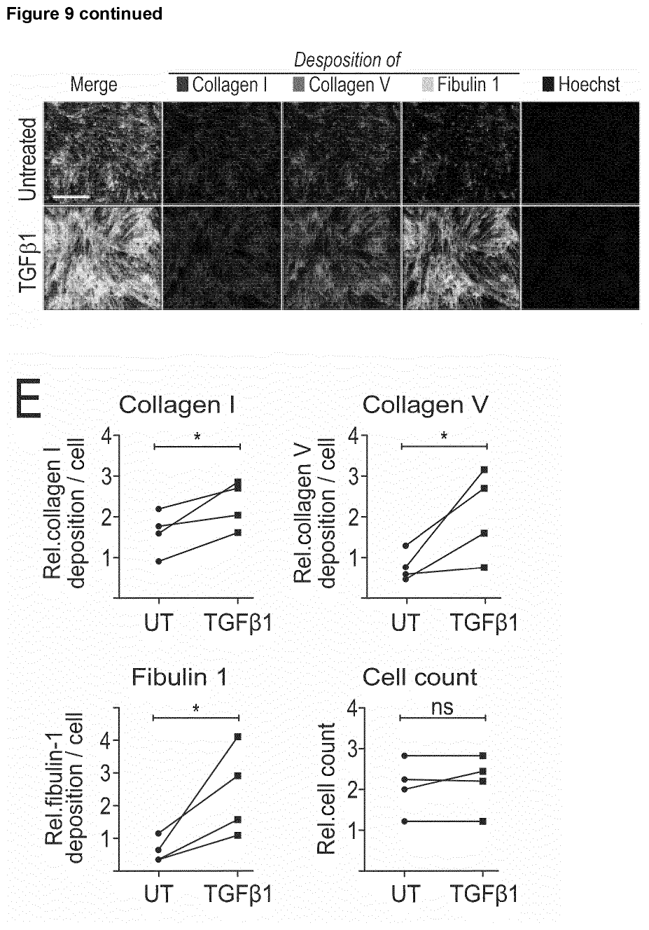

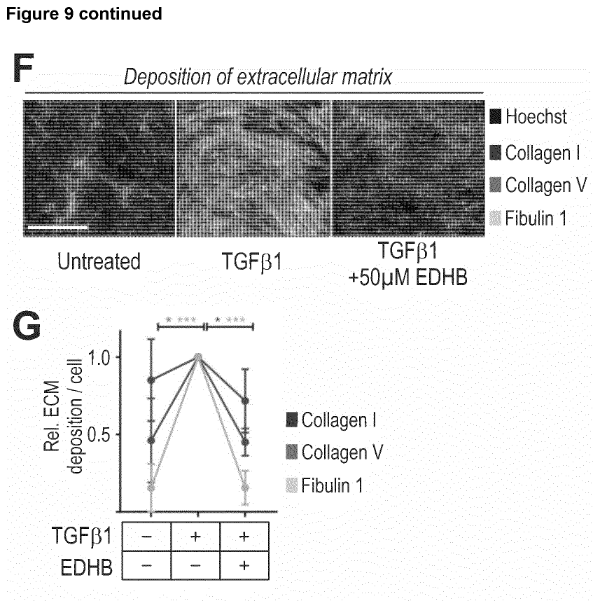

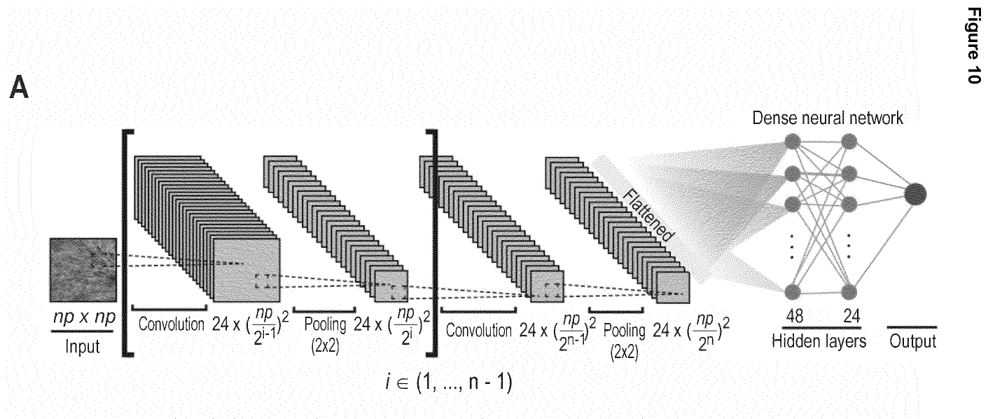

Total RNA was isolated PEQGold Total RNA Kit (PeqLab) according to the manufacturer's instructions including gDNA elimination. The Agilent 2100 Bioanalyzer was used to assess RNA quality and RNA with RIN>7 was used for microarray analysis. Total RNA (150 ng) was amplified using the WT PLUS Reagent Kit (Thermo Fisher Scientific Inc., Waltham, USA). Amplified cDNA was hybridized on Human ClariomS arrays (Thermo Fisher Scientific). Staining and scanning (GeneChip Scanner 3000 7G) was done according to manufacturer's instructions. Transcriptome Analysis Console (TAC; version 4.0.0.25; Thermo Fisher Scientific) was used for quality control and to obtain annotated normalized SST-RMA gene-level data. Statistical analyses were performed by utilizing the statistical programming environment R (R Development Core Team Ref1). Genewise testing for differential expression was done employing the paired limma t-test and Benjamini-Hochberg multiple testing correction (FDR<10%). To reduce background, gene sets were filtered using DABG p-values<0.05 in at least one sample per pair and in at least two of three pairs per analysis. Heatmaps were generated using GraphPad Prism v7, The regulation pattern clustering (RPC) was based on uniform manifold approximation and projection (UMAP) (35). mRNA abundancies from the microarray data were normalized (as seen as an example in FIG. F) and abundancies of all four different conditions summarized in a linear vector (FIG. 5E) that was projected into a bi-dimensional space using UMAP (FIG. 5G). Then, clusters of genes were extracted. Gene/protein interactions were visualized using the String Database (www.stringdb.org).

### Fibrotic Pattern Detection by Artificial Intelligence (FANTAIL)—Inferential Classification and Detection Model for the Inhibition of ECM Deposition

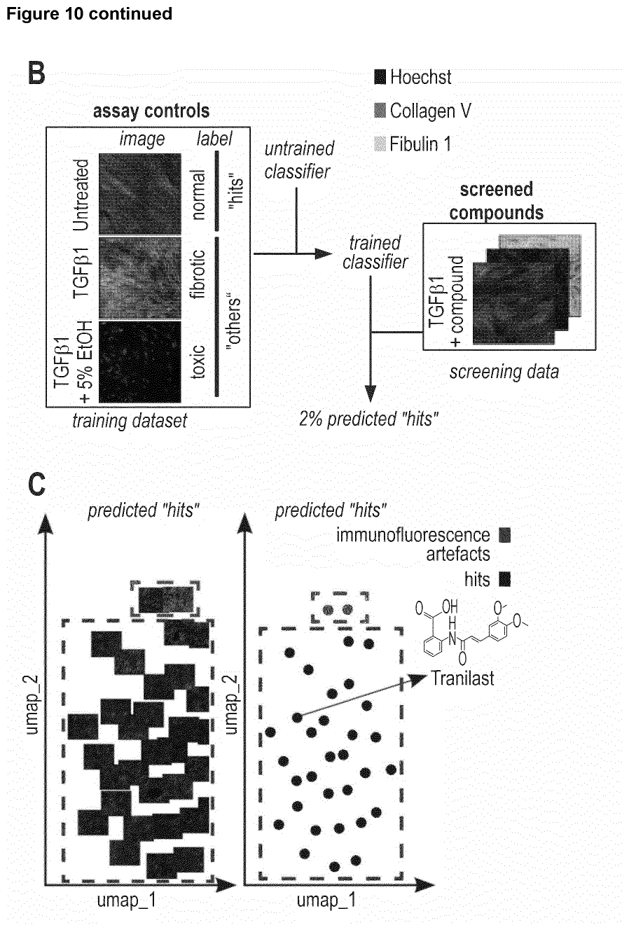

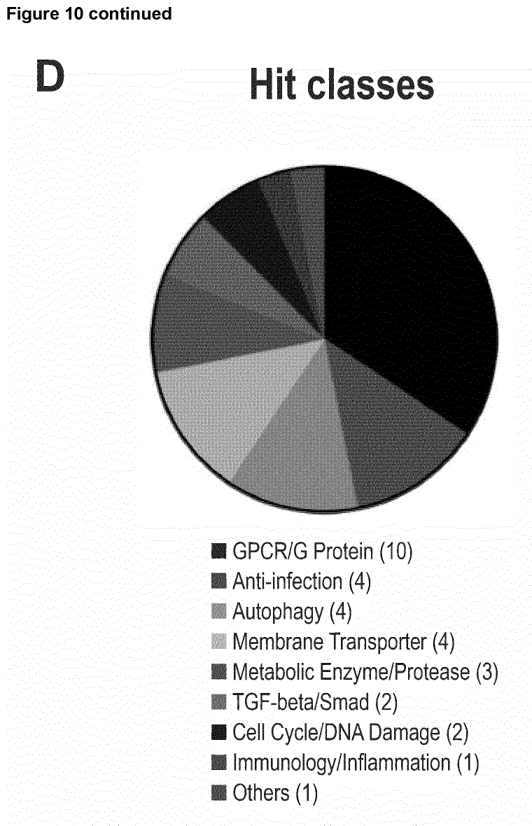

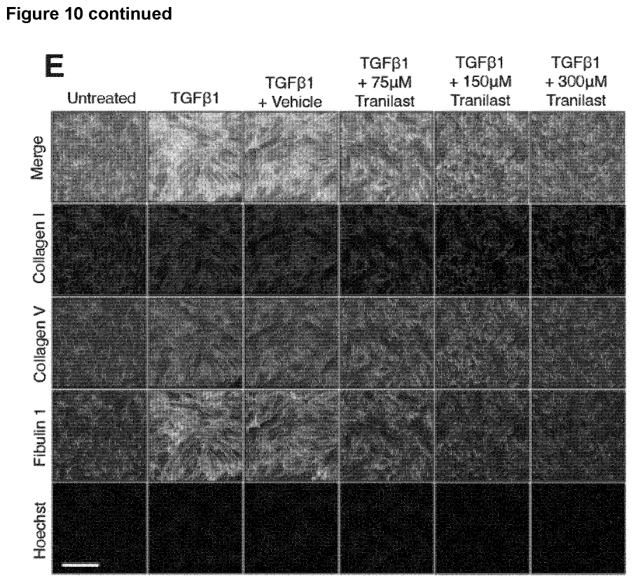

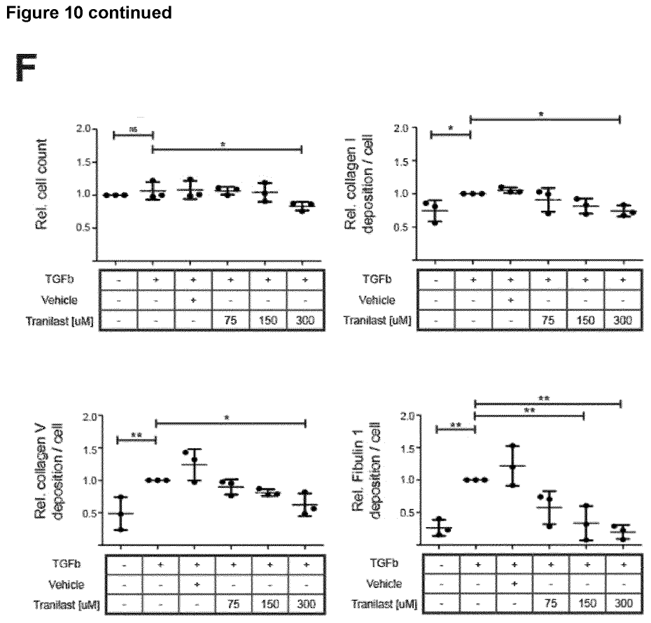

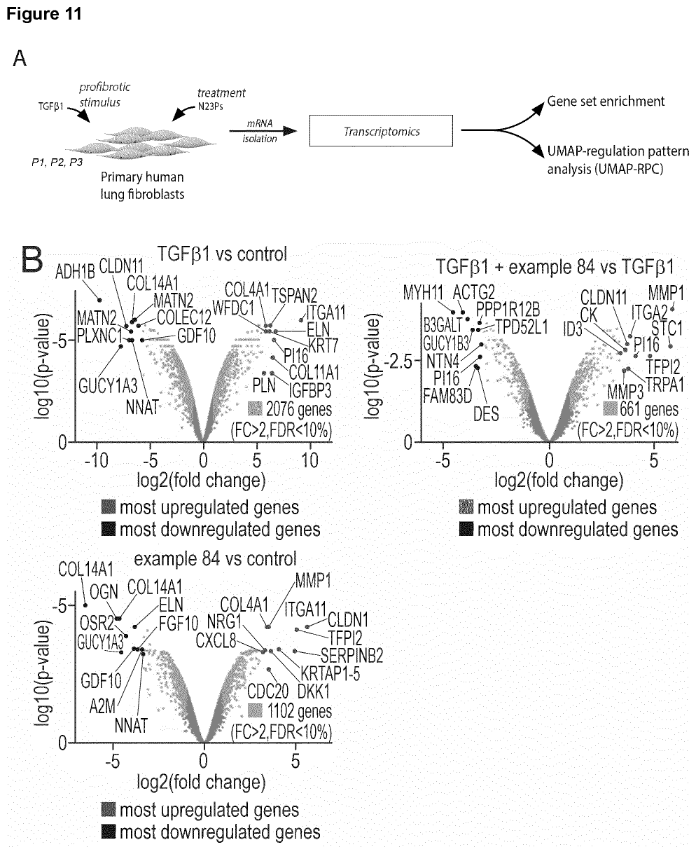

The KERAS high-level API (https github.com/fchciet/keras/) with TensorFlow implementation was used to train Convolutional Neural Network (CNN) on a complex image detection and classification task. The CNN design (FIG. 10A) followed the most accepted guidelines, as in (34). The best convolutional process was reached with 3 convolutional layers convoluting the images with 24 filters per layer and pooling out data with a 2×2 pooling matrix in the convolutional layers. The specific image classification and detection task was based on the detection of interspersed fibrotic and cellular patterns with frequent image edge pattern interruptions. The dimensional orientation of the fibrotic patterns appeared randomly oriented with various shape, size and clustering on images of large dimension (1024×1024×RGB). Hence, the Rectified Linear Unit (ReLU) activation was used as the activation mode to detect pattern edges and the adadelta optimizer was chosen for an efficient CNN learning process. A dataset of image controls was used to train the classifier. This dataset consisted of 295 immunofluorescence images annotated as “toxic” (treated with 5% ethanol), 390 images annotated as “fibrotic” (treated with TGFβ1) and 390 images annotated as “normal” (untreated). Images with the annotation “normal” were labeled as “hits”, while those with the annotations “toxic” and “fibrotic” were combined under the label “others”. Images were randomly assigned to the training dataset (75%) and the validation dataset (25%). As 1024×1024 sized images would be an unusually large input for a CNN, we aimed to test the CNN efficiency of np x np large subsets of each image with np e 128, 256, 512 pixels. Aggressive data augmentation was performed by fragmenting each m×m dimensional image (m=1024) in

$${( ( \frac{m}{np} - 1 ) \times 4 + 1 )}^{2}$$

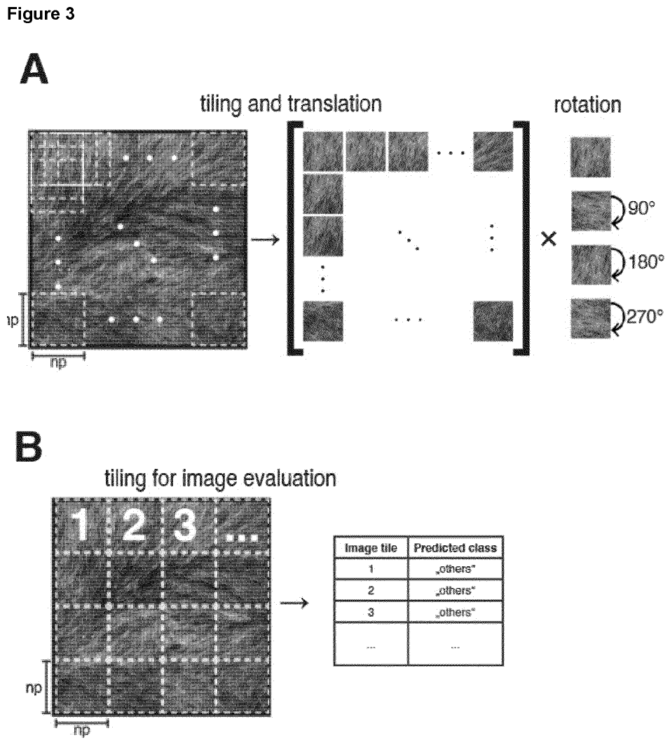

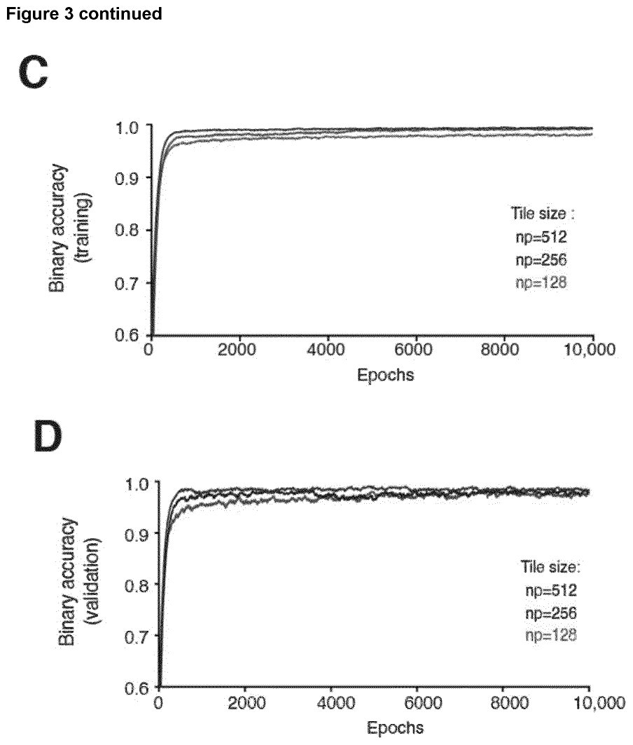

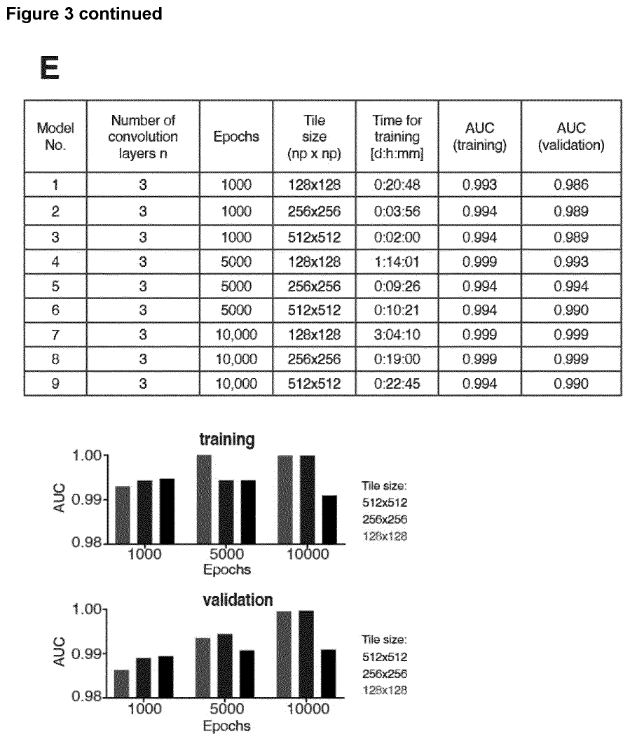

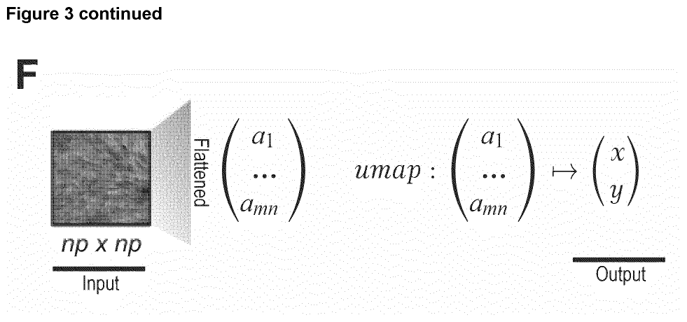

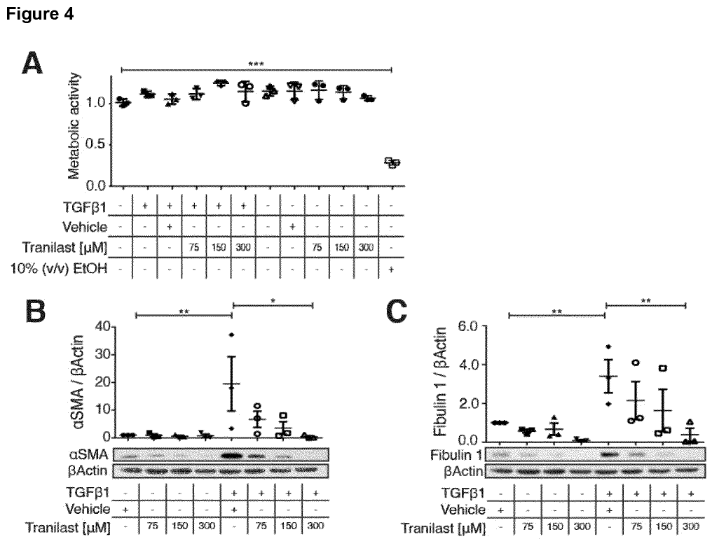

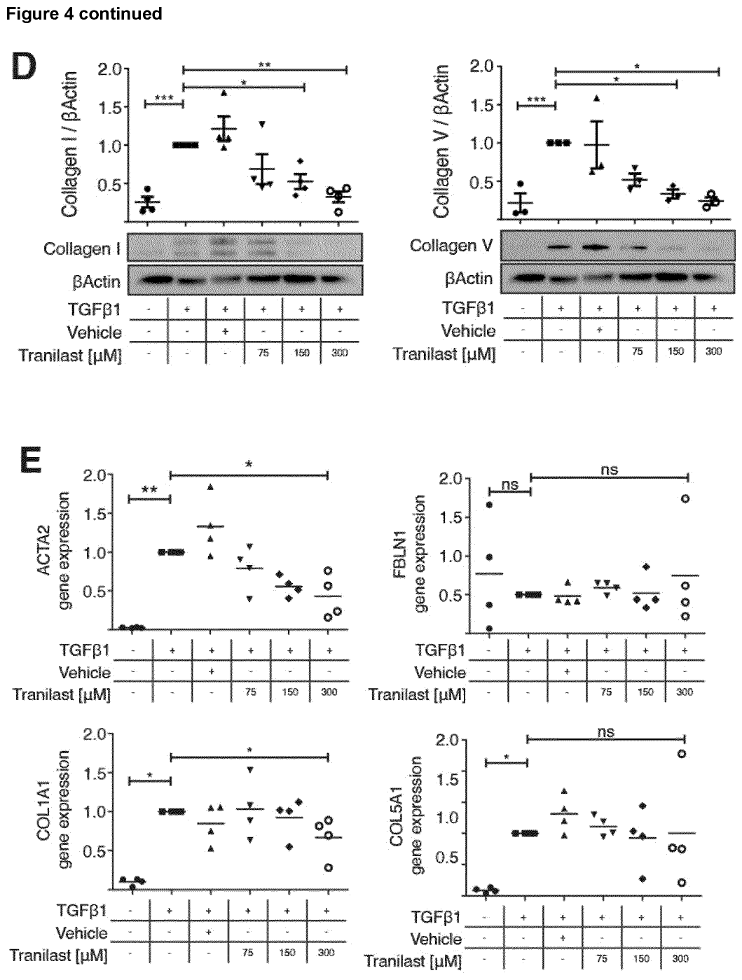

tiles with a 3/4 tile overlap (FIG. 3A) to increase redundancy and pattern fragmentation. For each image M with the dimensions m×m, the np×np sized tiles T were produced as follows:

$$T = M [ {o}_{X} , {o}_{x} + 1 , {o}_{x} + 2 , … , {o}_{x} + np ; {o}_{y} , {o}_{y} + 1 , {o}_{x} + 2 , … , {o}_{y} + np ]$$

$${o}_{x} , {o}_{y} ∈ {⋂}_{k ∈ [ 0 , 1 , … , ( ( \frac{m}{np} - 1 ) \times 4 )} k \times \frac{n}{4}$$

Each data tile T was rotated by θE {0°, 90°, 180°, 270° }, representing different spatial orientations of the ECM (FIG. 3A). Hence,

$$4 \times {( ( \frac{m}{np} - 1 ) \times 4 + 1 )}^{2} $$

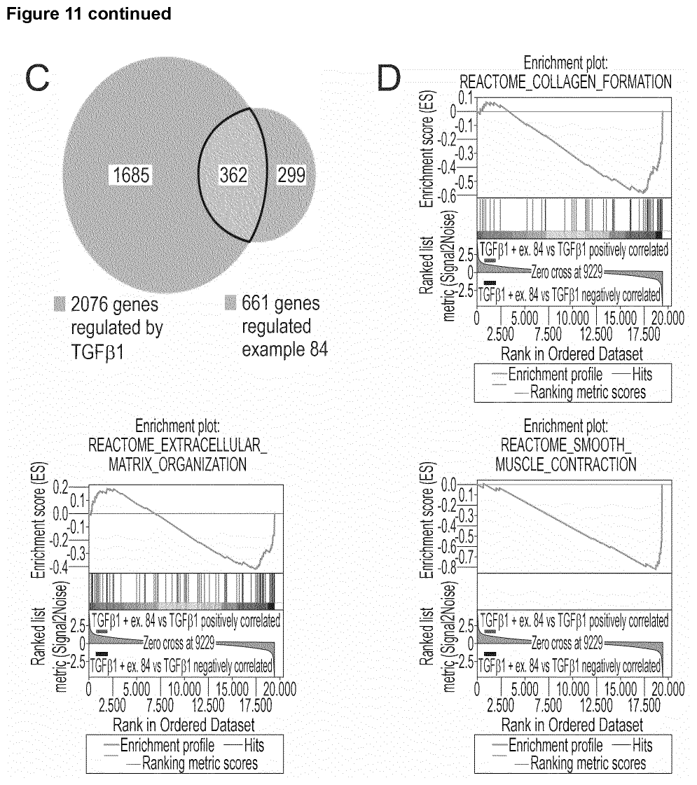

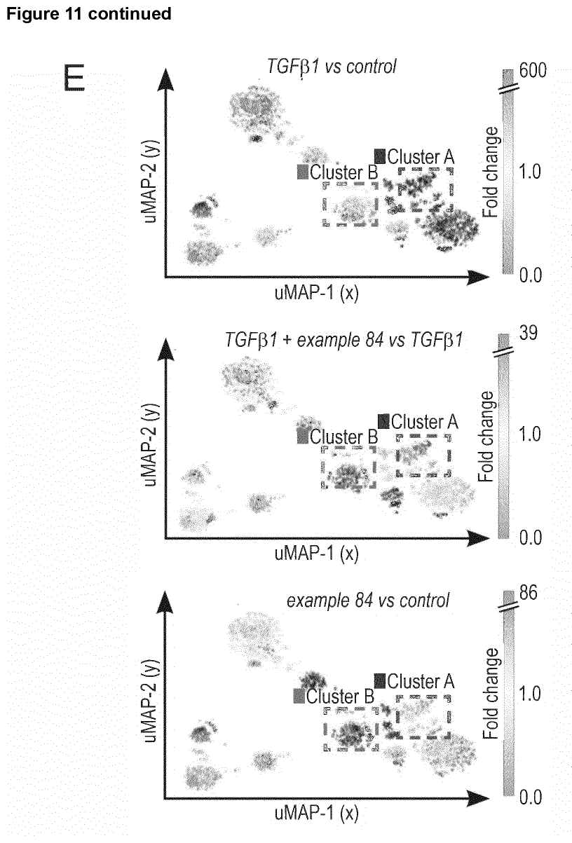

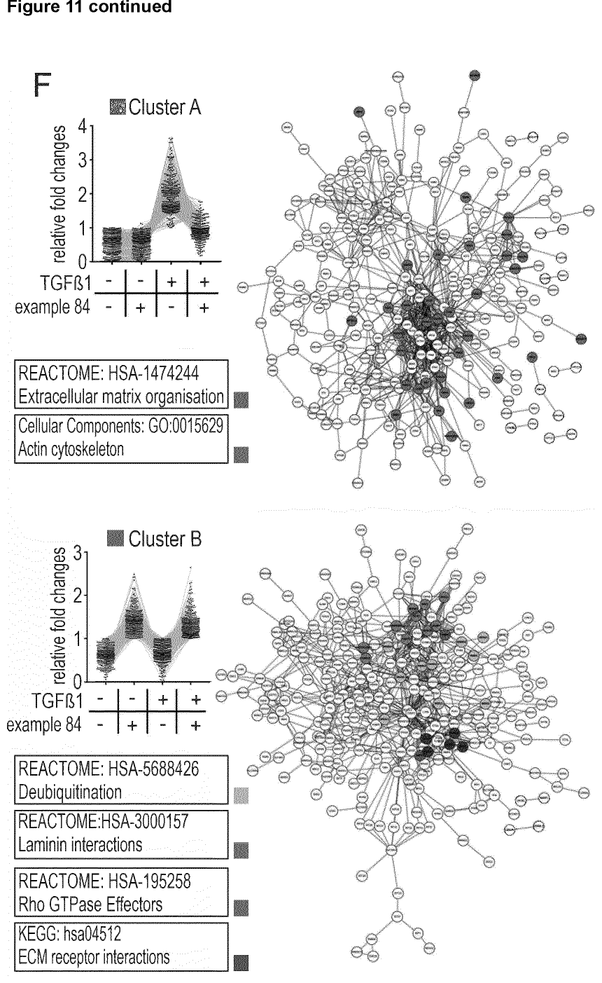

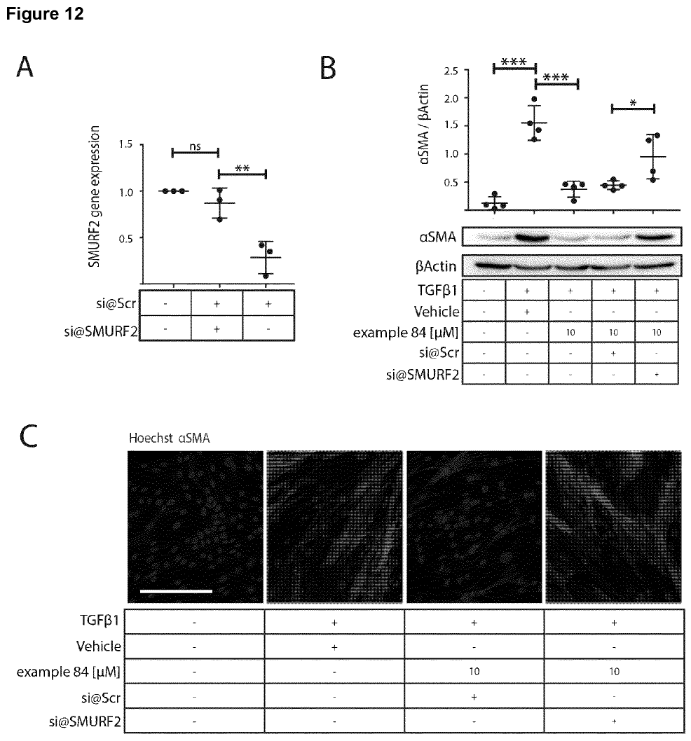

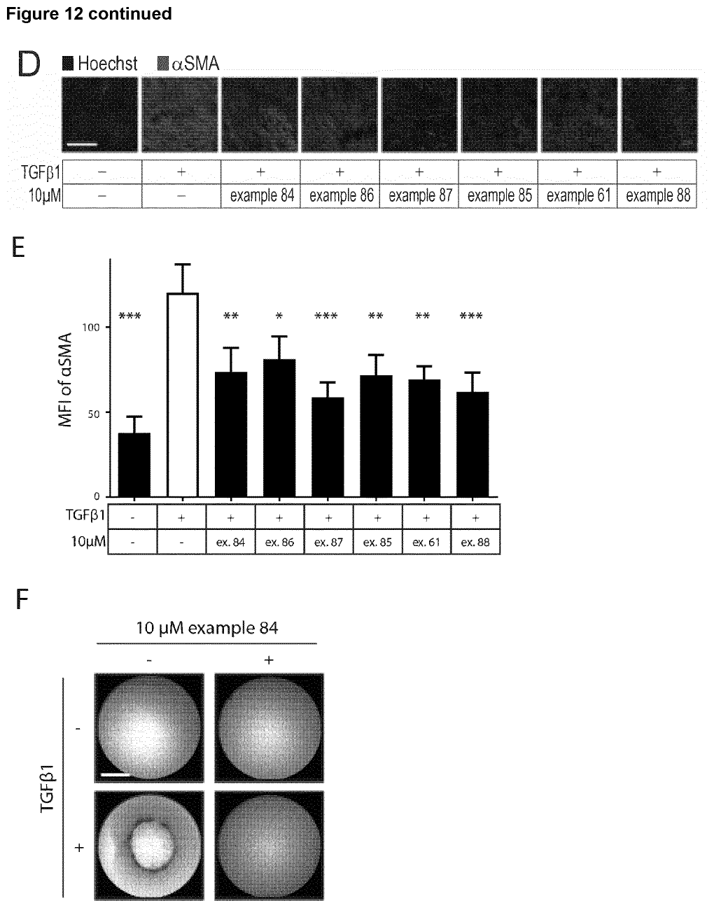

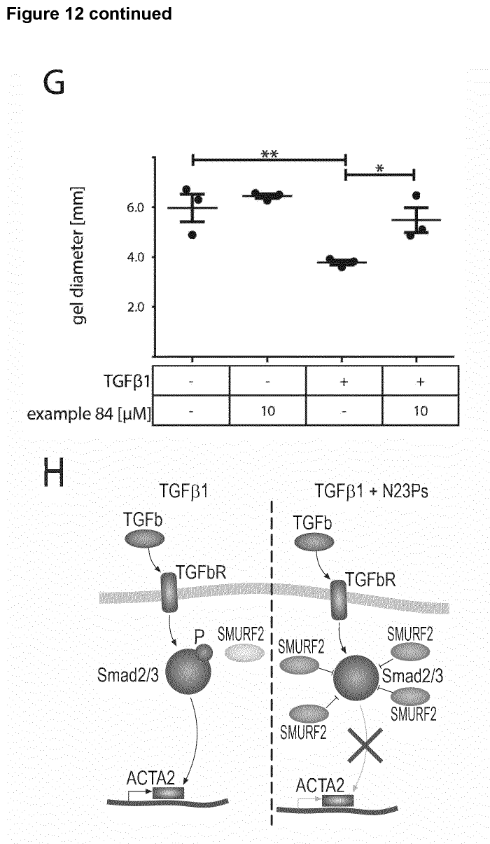

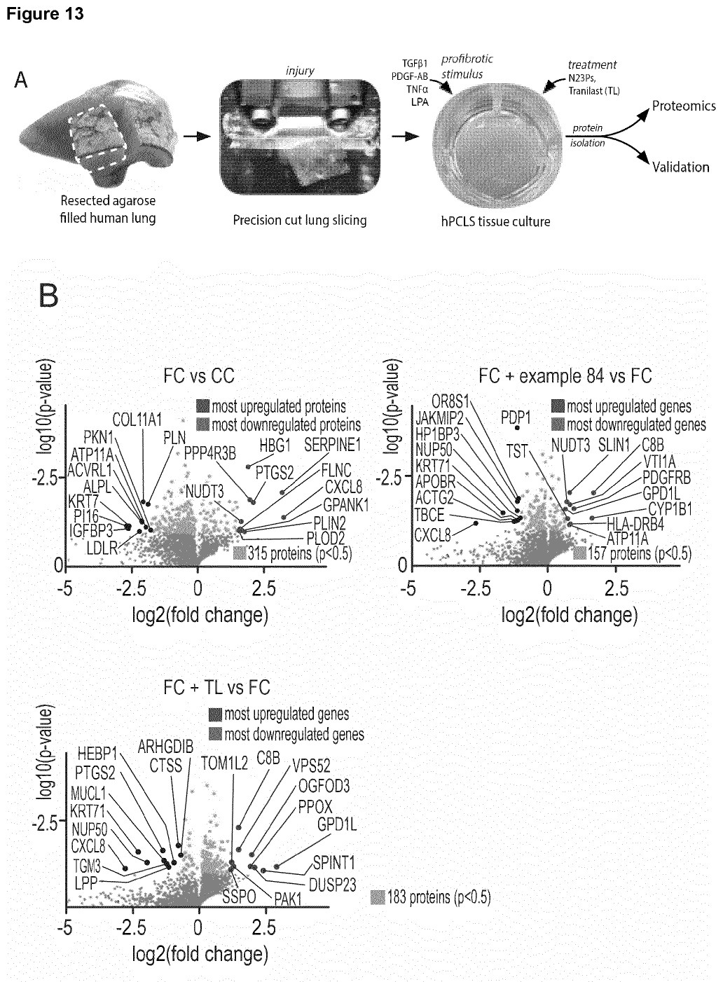

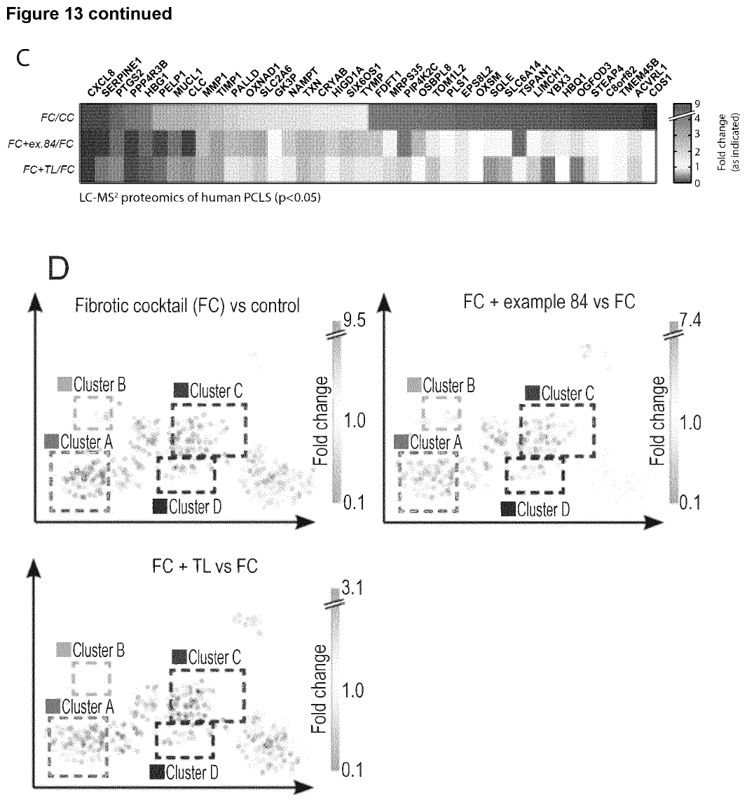

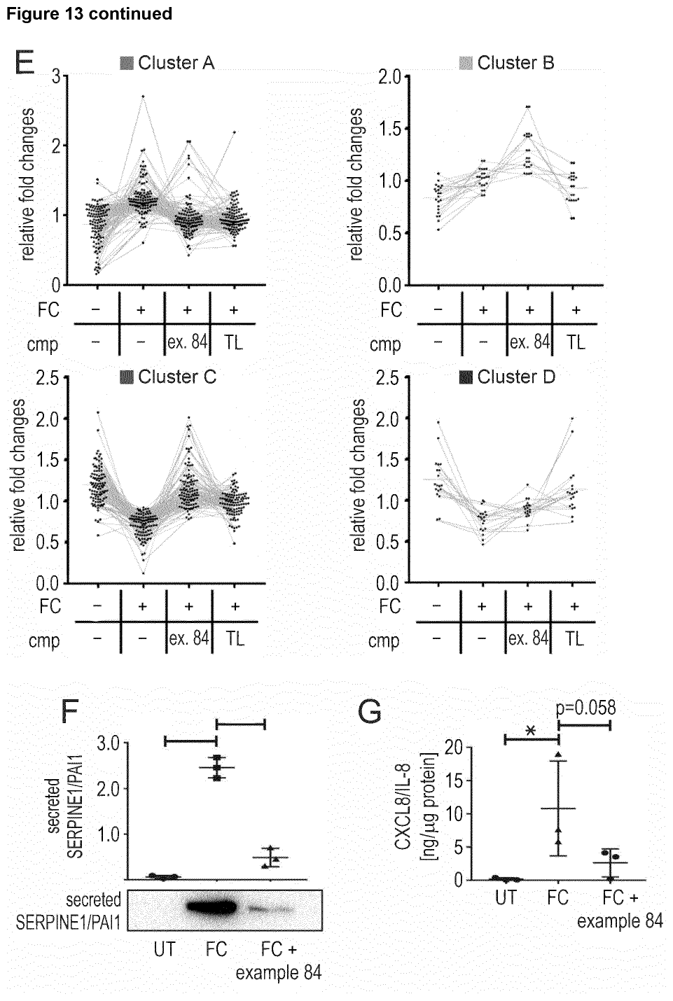

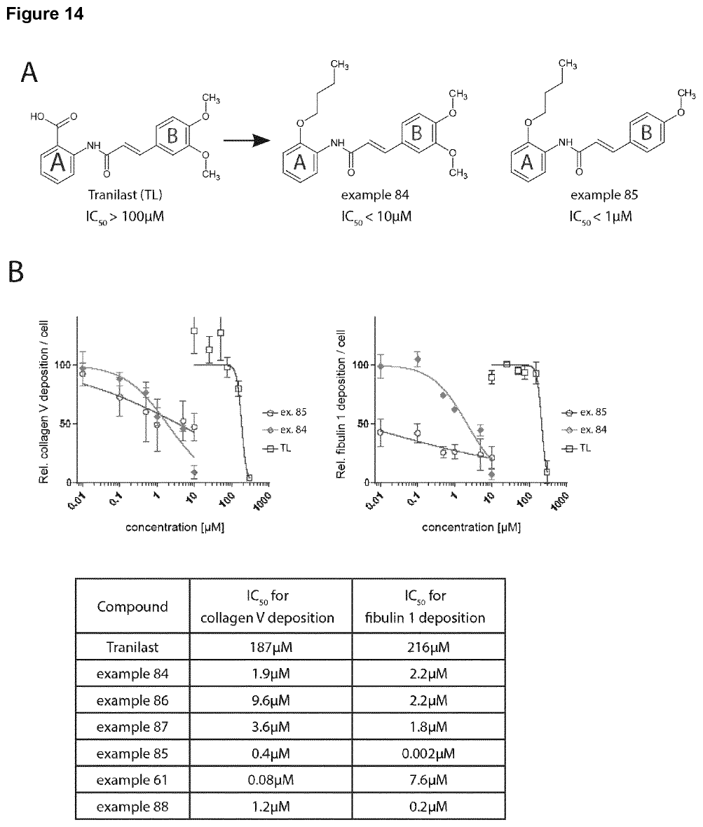

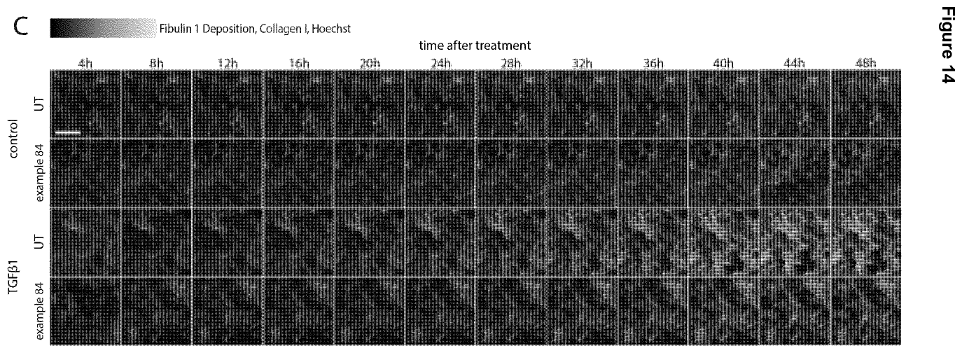

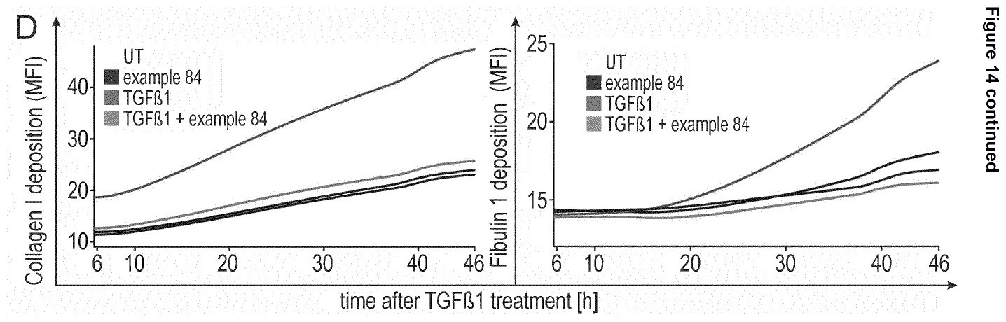

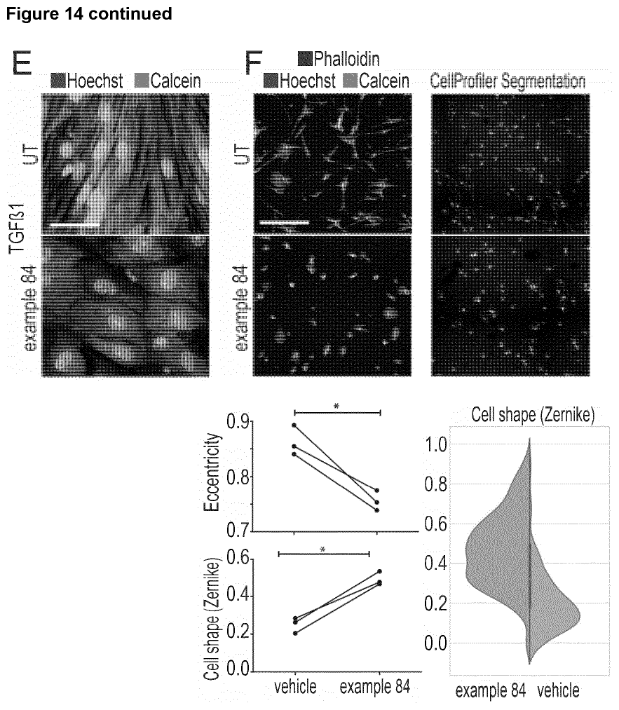

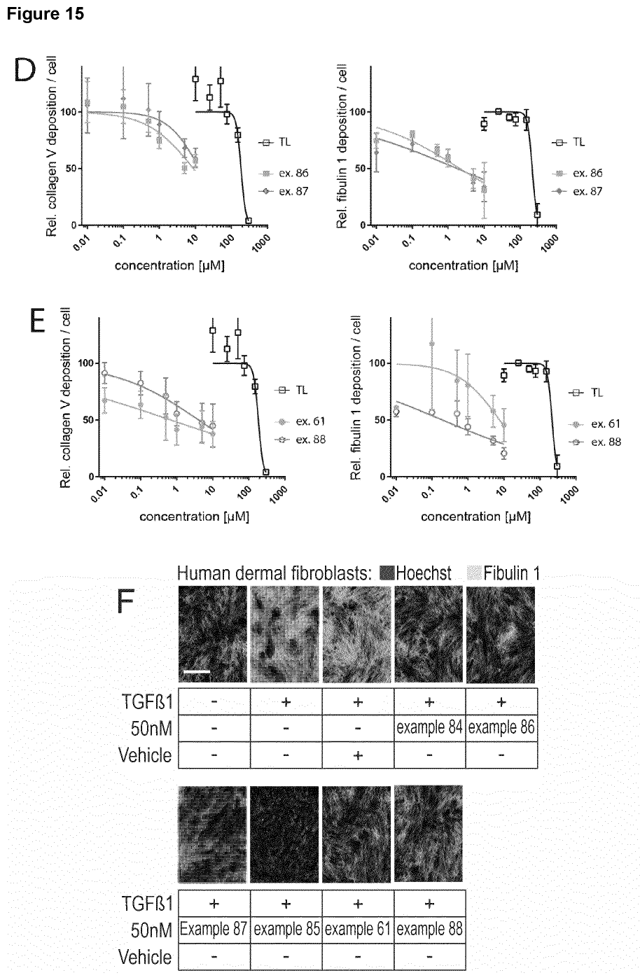

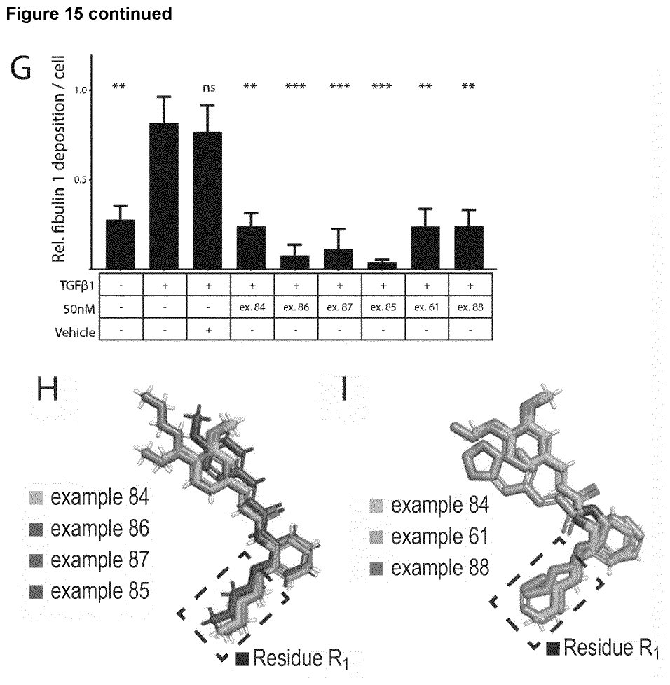

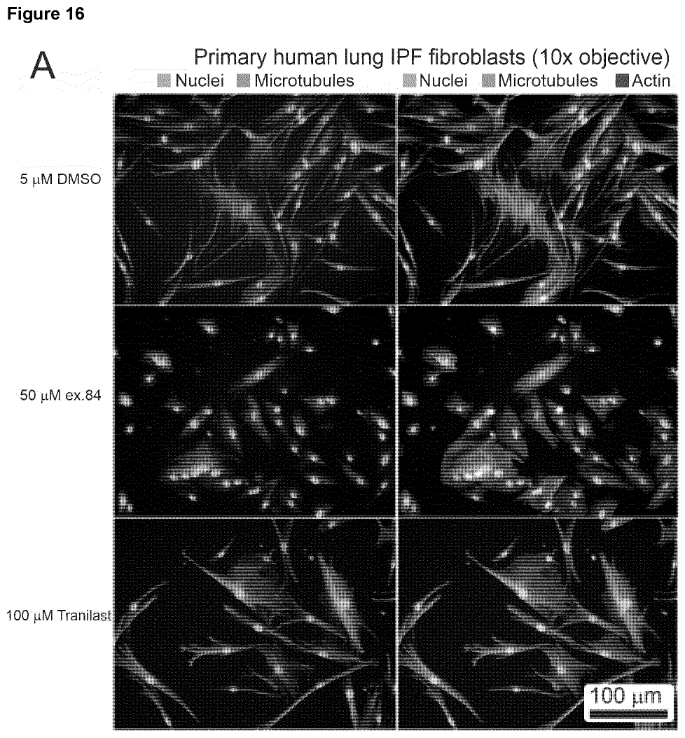

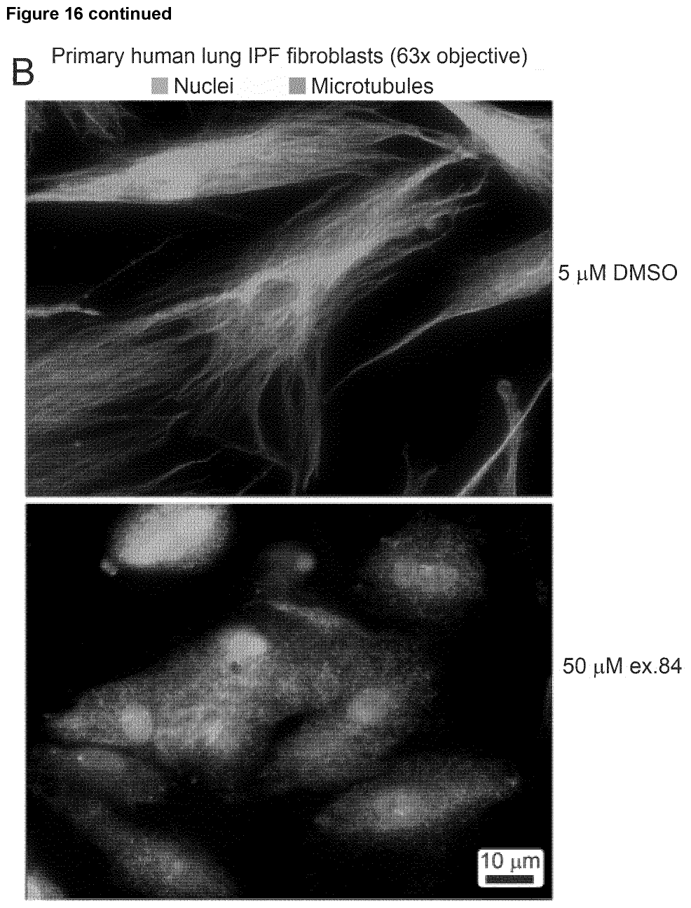

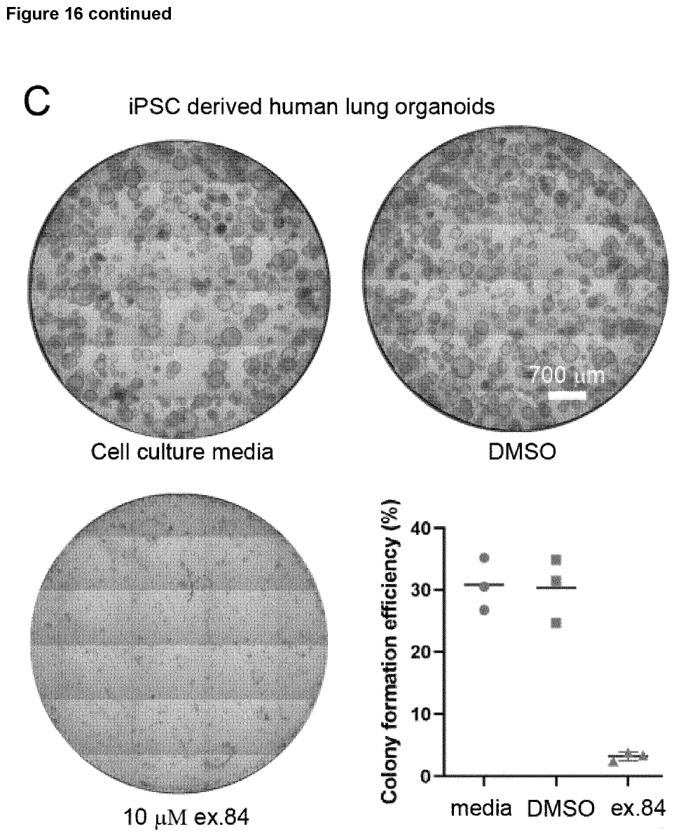

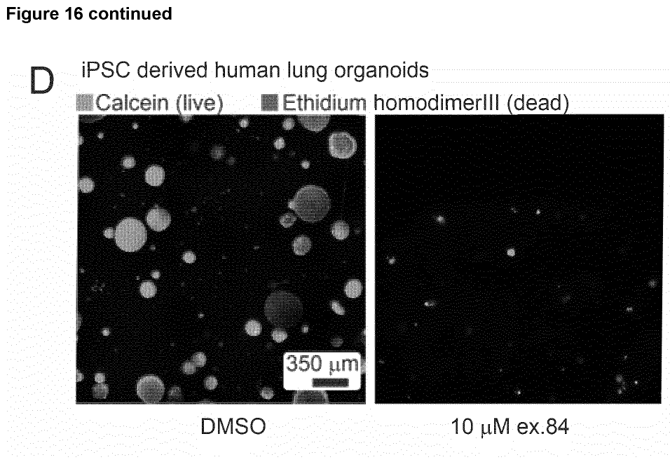

tiles were saved from each original image leading to a significant augmentation of data (100-fold for np=512, 676-fold for np=256 and 3364-fold for np=128). The convolutional neural network as shown in FIG. 11A was trained on all fragments of the training set with fragment size np e 128, 256, 512 and n=3. Code is provided in Code section S1, S2. Learning curves are shown in FIGS. 3C and 3D. Quickest and stable convergence was observed with 512×512 pixels imaging, unsurprising considering the larger amount of information per fragment. Then trained models were evaluated to predict the annotation of an original image of the validation set. Therefore, each original image was fragmented into

$${( \frac{m}{np} )}^{2}$$

not overlapping np×np sized tiles (FIG. 3B) and the share of tiles classified as “hits” was calculated. In order to determine the accuracy of prediction for original images independent of a cut-off value for the share of tiles classified as “hits”, we used Receiver-OperatorCharacteristic analysis (GraphPad Prism v7) and calculated the Area-Under-the-Curve (AUC) for each deep learning model. Different numbers of learning iterations (epochs) and different tile sizes np×np were tested. Generally, accuracy (given as AUC) increased with the number of epochs. After 10,000 epochs models with np=256 and np=128 yielded a similarly high AUC=0.999 (FIG. 3E). Therefore we chose model 7 with np=128, n 3 for further experiments.

### Uniform Manifold Approximation and Projection (UMAP) for Image Clustering

The image clustering chosen was performed using the UMAP (Uniform Manifold Approximation and Projection), a widely used manifold learning technique for dimension reduction. UMAP is constructed from a theoretical framework based in Riemannian geometry and algebraic topology (35). Each m×m dimensional image pixel matrix (m=1024) is flattened as a linear vector (FIG. 3F) and projected using UMAP on a 2-dimensional space (FIG. 10C). Indeed, it was found in this study that the UMAP approach generated 2D data maps that logically respected the AI images classification performed in the present study putting images into a bi-dimensional graph that helped to graphically understand the concept of true positives hits and identify images with artefacts not recognized by the AI.

### Quantification of αSMA Content in Primary Human Lung Fibroblasts

6000 cells/well phLFs were seeded in 384-well CellCarrier plates. Following overnight incubation, cells were starved in serum-reduced medium (1% FBS) for 24 h.

Afterwards, cells were treated with TGFβ1 (1 ng/ml) and different compounds. After 48 h, cells were fixed with 100% ice-cold methanol. Cells were stained for DAPI and αSMA antibody conjugated to Cy3 (Cat. No. C₆₁₉₈-2ML, Sigma). For automated liquid handling in 384 well plates, an INTEGRA Assist Plus was used. Following fixation, the automated imaging was achieved by using a confocal laser scanning microscope (LSM710, Zeiss) with automated focus detection for three-dimensional image acquisition (ECM Deposition Assay). Images were analyzed by measuring the mean fluorescence intensity (MFI) of the αSMA signal in Zen Blue v2.5 (Zeiss).

### Contractility Assay of Primary Human Lung Fibroblasts

In a 96-well imaging plate 50 μl 3D collagen gels were casted per well as described before (30), and 20.000 phLFs per well were seeded on top. Cells were treated with 1 ng/mL TGFβ1 and/or example 84. After 72 h cells were fixed with 4% paraformaldehyde. Collagen gels were imaged using an Axiolmager2 (Zeiss) and the gel diameter was determined using Zen Blue v2.5 (Zeiss).

### LC-MS/MS of Precision Cut Lung Slices (PCLS)

Each 10 μg of protein extract was digested using a modified FASP protocol (36, 37). Briefly, proteins were reduced and alkylated using dithiothreitol and iodoacetamide, and diluted to 4 M urea prior to centrifugation on a 30 kDa filter device (Sartorius). After several washing steps using 8 M urea and 50 mM ammoniumbicarbonate, proteins were digested on the filter by Lys-C and trypsin overnight. Generated peptides were eluted by centrifugation, acidified with TFA and stored at −20° C. Samples were measured on a QExactive HF-X mass spectrometer (Thermo scientific) online coupled to an Ultimate 3000 nano-RSLC (Dionex). Tryptic peptides were automatically loaded on a trap column (300 μm inner diameter (ID)×5 mm, Acclaim PepMap100 C18, 5 μm, 100 Å, LC Packings) prior to C18 reversed phase chromatography on the analytical column (nanoEase MZ HSS T3 Column, 100 Å, 1.8 μm, 75 μm×250 mm, Waters) at 250 nl/min flow rate in a 95 minutes non-linear acetonitrile gradient from 3 to 40% in 0.1% formic acid. Profile precursor spectra from 300 to 1500 m/z were recorded at 60000 resolution with an automatic gain control (AGC) target of 3e6 and a maximum injection time of 30 ms. Subsequently TOP15 fragment spectra of charges 2 to 7 were recorded at 15000 resolution with an AGC target of 1e5, a maximum injection time of 50 ms, an isolation window of 1.6 m/z, a normalized collision energy of 28 and a dynamic exclusion of 30 seconds. Generated raw files were analyzed using Progenesis QI for proteomics (version 4.1, Nonlinear Dynamics, part of Waters) for label-free quantification as described (38, 39). Features of charges 2-7 were used and all MSMS spectra were exported as mgf file. Peptide search was performed using Mascot search engine (version 2.6.2) against the Swissprot human protein database (20237 sequences, 11451954 residues).

Search settings were: 10 ppm precursor tolerance, 0.02 Da fragment tolerance, one missed cleavage allowed, carbamidomethyl on cysteine as fixed modification, deamidation of glutamine and asparagine allowed as variable modification, as well as oxidation of methionine. Applying the percolator algorithm (40) resulted in a peptide false discovery rate (FDR) of 0.46%. Search results were reimported in the Progenesis Q1 software. Proteins were quantified by summing up the abundances of all unique peptides per protein after normalization to identified GAPDH and ACTB peptides. Resulting protein abundances were used for calculation of fold-changes between conditions and repeated-measures ANOVAs within the Progenesis QI software. Proteomics expression data is provided as Table S4.

Smurf2 siRNA-Mediated Silencing

phLFs were reverse transfected with 2 nM or 10 nM Silencer® Pre-designed Smurf2 siRNA (Cat #: AM16708, Ambion, ThermoFisher Scientific, Carlsbad, USA) or 10 nM scrambled Silencer® Negative control No. 1 siRNA (AM4611, Ambion, ThermoFisher Scientific, Carlsbad, USA) in Lipofectamine® RNAiMax transfection reagent (13778-150, ThermoFisher Scientific, Carlsbad, 130 USA) as indicated followed by 1 ng/ml TGFβ1 treatment for 48 h if not indicated differently.

### 3. Assessing N23P Activity in Inhibiting ECM Deposition in IPF-Fibroblasts

Experimental outline: Primary human lung IPF-fibroblasts (phLFs), which were derived from 3 different idiopathic-pulmonary-fibrosis (IPF) patients (n=3), were cultured in DMEM F-12 medium with 20% fetal bovine serum (FBS) and antibiotic supplements. Cells were seeded with 6000 cells/well in 384-well CellCarrier plates. After overnight incubation, cells were starved in serum-reduced medium (1% FBS with 0.1 mM 2-phosphoascorbate for 24 h). Then, phLFs were treated with TGFβ1 (1 ng/ml) or vehicle, and incubated together with synthesized N23Ps (10 μM) or appropriate vehicle controls. After 72 h of incubation, medium was changed for starvation medium with 1 μg/mL AlexaFluor-637 fluorophore conjugated anti-fibulin 1 antibodies (SantaCruz, Clone C-5, Cat #sc-25281 AF647) and 1 μg/mL Hoechst H33342 (Sigma). Following four hours of incubation, cells were washed three times with PBS and fixed with paraformaldehyde (PFA). Following fixation, the automated imaging was achieved by using a confocal laser scanning microscope (LSM710, Zeiss) with automated focus detection for three-dimensional image acquisition (1024 px×1024 px×9 px which equals a dimension of 1417 μm×1417 μm×16 μm). For post-acquisition analysis images were imported into IMARIS software (Bitplane) and quantified for the mean fluorescence intensity, as well as Hoechst-stained cell nuclei were automatically counted by using Imaris' spot detection algorithm.

Assessment of ECM inhibition I: Mean fluorescence intensity (MFI) values represented the degree of fibulin-1 ECM deposition in three different IPF-phLFs (n=3). The inhibitory activity of the tested N23P compounds was graded, whereas we defined a compound as “inactive” if all TGFβ1-induced ECM deposition was preserved (=0% inhibition by the compound). If no increase in TGFβ1-induced ECM deposition was detected, the compound was rated as active (100% inhibition by the compound). Based on this definition three different classes of inhibitory activities have been created: (+++) compounds which displayed >90% inhibition, (++) compounds which displayed 60-90% inhibition, and (+) compounds which displayed 20-60% inhibition of TGFβ1-induced ECM deposition. Compounds which showed an inhibition <20% were classified as inactive. Tranilast exhibits no inhibition at 10 μM.

TABLE 5.1

| Compound | Activity |
|---|---|
| 7 | ++ |
| 11 | ++ |
| 12 | +++ |
| 13 | +++ |
| 67 | +++ |
| 83 | +++ |
| 2 | ++ |
| 3 | +++ |
| 84 | +++ |
| 6 | ++ |
| 20 | ++ |
| 21 | + |
| 29 | + |
| 31 | ++ |
| 33 | ++ |
| 34 | +++ |
| 36 | ++ |
| 37 | +++ |
| 71 | + |
| 5 | +++ |
| 38 | +++ |
| 39 | ++ |
| 43 | ++ |
| 45 | +++ |
| 75 | +++ |
| 79 | ++ |

Assessment of ECM inhibition It: Mean fluorescence intensity (MFI) values represented the degree of fibulin-1 ECM deposition in one or two different IPF-phLFs (n=1-2). The inhibitory activity of the tested N23P compounds was graded, whereas we defined a compound as “inactive” if all TGFβ1-induced ECM deposition was preserved (=0% inhibition by the compound). If no increase in TGFβ1-induced ECM deposition was detected, the compound was rated as active (100% inhibition by the compound). Based on this definition three different classes of inhibitory activities have been created: (+++) compounds which displayed more than 90% inhibition, (++) compounds which displayed 60-90% inhibition, and (+) compounds which displayed 20-60% inhibition of TGFβ1-induced ECM deposition. Compounds which showed an inhibition <20% were classified as inactive.

TABLE 5.2

| Compound | Activity |
|---|---|
| 166 | + |
| 6 | + |
| 162 | ++ |
| 167 | ++ |
| 168 | ++ |
| 82 | + |
| 83 | +++ |
| 155 | ++ |
| 153 | +++ |
| 156 | ++ |
| 161 | +++ |
| 163 | + |
| 60 | +++ |
| 46 | + |
| 63 | + |

### REFERENCES

1. T. A. Wynn, T. R. Ramalingam, Mechanisms of fibrosis: therapeutic translation for fibrotic disease. Nat Med 18, 1028-1040 (2012).
2. T. R. Cox, J. T. Erler, Remodeling and homeostasis of the extracellular matrix: implications for fibroticdiseases and cancer. Dis Model Mech 4, 165-178 (2011).
3. D. C. Rockey, P. D. Bell, J. A. Hill, Fibrosis-A Common Pathway to Organ Injury and Failure. N Engl J Med 373, 96 (2015).
4. S. L. Friedman, D. Sheppard, J. S. Duffield, S. Violette, Therapy for fibrotic diseases: nearing the starting line. Sci Transl Med 5, 167sr161 (2013).
5. L. Richeldi, H. R. Collard, M. G. Jones, Idiopathic pulmonary fibrosis. Lancet 389, 1941-1952 (2017).
6. I. E. Fernandez, O. Eickelberg, New cellular and molecular mechanisms of lung injury and fibrosis in idiopathic pulmonary fibrosis. Lancet 380, 680-688 (2012).
7. G. Sgalla, A. Biffi, L. Richeldi, Idiopathic pulmonary fibrosis: Diagnosis, epidemiology and natural history. Respirology 21, 427-437 (2016).
8. T. E. King, Jr., W. Z. Bradford, S. Castro-Bernardini, E. A. Fagan, I. Glaspole, M. K. Glassberg, E. Gorina, P. M. Hopkins, D. Kardatzke, L. Lancaster, D. J. Lederer, S. D. Nathan, C. A. Pereira, S. A. Sahn, R. Sussman, J. J. Swigris, P. W. Noble, A. S. Group, A phase 3 trial of pirfenidone in patients with idiopathic pulmonary fibrosis. N Engl J Med 370, 2083-2092 (2014).
9. L. Richeldi, R. M. du Bois, G. Raghu, A. Azuma, K. K. Brown, U. Costabel, V. Cottin, K. R. Flaherty, D. M. Hansell, Y. Inoue, D. S. Kim, M. Kolb, A. G. Nicholson, P. W. Noble, M. Selman, H. Taniguchi, M. Brun, F. Le Maulf, M. Girard, S. Stowasser, R. Schlenker-Herceg, B. Disse, H. R. Collard, I. T. Investigators, Efficacy and safety of nintedanib in idiopathic pulmonary fibrosis. N Engl J Med 370, 2071-2082 (2014).
10. K. R. Flaherty, A. U. Wells, V. Cottin, A. Devaraj, S. L. F. Walsh, Y. Inoue, L Richeldi, M. Kolb, K. Tetzlaff, S. Stowasser, C. Coeck, E. Clerisme-Beaty, B. Rosenstock, M. Quaresma, T. Haeufel, R. G. Goeldner, R. Schlenker-Herceg, K. K. Brown, I. T. Investigators, Nintedanib in Progressive Fibrosing Interstitial Lung Diseases. N Engl J Med 381, 1718-1727 (2019).
11. G. Burgstaller, B. Oehrle, M. Gerckens, E. S. White, H. B. Schiller, O. Eickelberg, The instructive extracellular matrix of the lung: basic composition and alterations in chronic lung disease. Eur Respir J 50, (2017).
12. M. Selman, C. Lopez-Otin, A. Pardo, Age-driven developmental drift in the pathogenesis of idiopathicpulmonary fibrosis. Eur Respir J 48, 538-552 (2016).
13. H. B. Schiller, L. E. Fernandez, G. Burgstaller, C. Schaab, R. A. Scheltema, T. Schwarzmayr, T. M. Strom, O. Eickelberg, M. Mann, Time- and compartment-resolved proteome profiling of the extracellular niche in lung injury and repair. Mol Syst Biol 11, 819 (2015).
14. A. Naba, K. R. Clauser, S. Hoersch, H. Liu, S. A. Carr, R. O. Hynes, The matrisome: in silico definition and in vivo characterization by proteomics of normal and tumor extracellular matrices. Mol Cell Proteomics 11, M111 014647 (2012).
15. M. L. Decaris, M. Gatmaitan, S. FlorCruz, F. Luo, K. Li, W. E. Holmes, M. K. Hellerstein, S. M. Turner, C. L. Emson, Proteomic analysis of altered extracellular matrix turnover in bleomycin-induced pulmonary fibrosis. Mol Cell Proteomics 13, 1741-1752 (2014).
16. A. J. Booth, R. Hadley, A. M. Comett, A. A. Dreffs, S. A. Matthes, J. L. Tsui, K. Weiss, J. C. Horowitz, V. F. Fiore, T. H. Barker, B. B. Moore, F. J. Martinez, L. E. Niklason, E. S. White, Acellular normal and fibrotic human lung matrices as a culture system for in vitro investigation. Am J Respir Crit Care Med 186, 866-876 (2012).
17. M. Sato, Y. Muragaki, S. Saika, A. B. Roberts, A. Ooshima, Targeted disruption of TGF-beta1/Smad3 signaling protects against renal tubulointerstitial fibrosis induced by unilateral ureteral obstruction. J Clin Invest 112, 1486-1494 (2003).
18. W. A. Border, N. A. Noble, T. Yamamoto, J. R. Harper, Y. Yamaguchi, M. D. Pierschbacher, E. Ruoslahti, Natural inhibitor of transforming growth factor-beta protects against scarring in experimental kidney disease. Nature 360, 361-364 (1992).
19. D. E. Clouthier, S. A. Comerford, R. E. Hammer, Hepatic fibrosis, glomerulosclerosis, and a lipodystrophy-like syndrome in PEPCK-TGF-beta1 transgenic mice. J Clin Invest 100, 2697-2713 (1997).
20. P. J. Sime, Z. Xing, F. L. Graham, K. G. Csaky, J. Gauldie, Adenovector-mediated gene transfer of active transforming growth factor-beta1 induces prolonged severe fibrosis in rat lung. J Clin Invest 100, 768-776 (1997).
21. U. Bartram, C. P. Speer, The role of transforming growth factor beta in lung development and disease. Chest 125, 754-765 (2004).
22. E. Piek, C. H. Heldin, P. Ten Dijke, Specificity, diversity, and regulation in TGF-beta superfamily signaling. FASEB J 13, 2105-2124 (1999).
23. F. Verrecchia, A. Mauviel, Transforming growth factor-beta signaling through the Smad pathway: role in extracellular matrix gene expression and regulation. J Invest Dermatol 118, 211-215 (2002). 24. K. L. Walton, K. E. Johnson, C. A. Harrison, Targeting TGF-beta Mediated SMAD Signaling for the Prevention of Fibrosis. Front Pharmacol 8, 461 (2017).
25. P. Pakshir, B. Hinz, The big five in fibrosis: Macrophages, myofibroblasts, matrix, mechanics, and miscommunication. Matrix Biol 68-69, 81-93 (2018). 26. R. Rabieian, M. Boshtam, M. Zareei, S. Kouhpayeh, A. Masoudifar, H. Mirzaei, Plasminogen Activator Inhibitor Type-1 as a Regulator of Fibrosis. J Cell Biochem 119, 17-27 (2018).
27. L Yang, J. Herrera, A. Gilbertsen, H. Xia, K. Smith, A. Benyumov, P. B. Bitterman, C. A. Henke, IL-8 mediates idiopathic pulmonary fibrosis mesenchymal progenitor cell fibrogenicity. Am J Physiol Lung Cel/Mol Physiol 314, L127-L136 (2018).
28. a) S. C. Zammita, A. J. Cox, R. M. Gow, Y. Zhang, R. E. Gilbert, H. Krum, D. J. Kelly, S. J. Williams, Bloorganic & Medicinal Chemistry Letters 19 (2009) 7003-7006. b) Isaji, M.; Kikuchi, S.; Miyata, H.; Ajisawa, Y.; Araki-Inazawa, K.; Tsukamoto, Y.; Amano, Y. (2000): Inhibitory effects of tranilast on the proliferation and functions of human pterygium-derived fibroblasts. In Cornea 19 (3), pp. 364-368.
29. C. A. Staab-Weijnitz, I. E. Fernandez, L. Knuppel, J. Maul, K. Heinzelmann, B. M. Juan-Guardela, E.Hennen, G. Preissler, H. Winter, C. Neurohr, R. Hatz, M. Lindner, J. Behr, N. Kaminski, O. Eickelberg, FK506-Binding Protein 10, a Potential Novel Drug Target for Idiopathic Pulmonary Fibrosis. Am J Respir Crit Care Med 192, 455-467 (2015).
30. G. Burgstaller, B. Oehrle, 1. Koch, M. Lindner, O. Eickelberg, Multiplex profiling of cellular invasion in 3D cell culture models. PLoS One 8, e63121 (2013).
31. H. N. Alsafadi, C. A. Staab-Weijnitz, M. Lehmann, M. Lindner, B. Peschel, M. Konigshoff, D. E. Wagner, An ex vivo model to induce early fibrosis-like changes in human precision-cut lung slices. Am J Physiol Lung Cell Mot Physiol 312, L896-L902 (2017).
32. M. Gerckens, H. N. Alsafadi, D. E. Wagner, M. Lindner, G. Burgstaller, M. Konigshoff, Generation of Human 3D Lung Tissue Cultures (3D-LTCs) for Disease Modeling. J Vis Exp, (2019).
33. G. Burgstaller, A. Sengupta, S. Vierkotten, G. Preissler, M. Lindner, J. Behr, M. Konigshoff, O. Eickelberg, Distinct niches within the extracellular matrix dictate fibroblast function in (cell free) 3D lung tissue cultures. Am J Physiol Lung Cell Mot Physiol 314, L708-L723 (2018).
34. C. Scheeder, F. Heigwer, M. Boutros, Machine learning and image-based profiling in drug discovery. Curr Opin Syst Biol10, 43-52 (2018).
35. L. McInnes, J. Healy, J. Melville, UMAP: Uniform Manifold Approximation and Projection for dimension reduction. arXiv, 2018. arXiv preprint arXiv:1802.03426, (2018).
36. J. R. Wisniewski, A. Zougman, N. Nagaraj, M. Mann, Universal sample preparation method for proteome analysis. Nat Methods 6, 359-362 (2009).
37. A. Grosche, A. Hauser, M. F. Lepper, R. Mayo, C. von Toerne, J. Merl-Pham, S. M. Hauck, The Proteome of Native Adult Muller Glial Cells From Murine Retina. Mol Cell Proteomics 15, 462-480 (2016).
38. S. M. Hauck, J. Dietter, R. L. Kramer, F. Hofmaler, J. K. Zipplies, B. Amann, A. Feuchtinger, C. A. Deeg, M. Ueffing, Deciphering membrane-associated molecular processes in target tissue of autoimmune uveitis by label-free quantitative mass spectrometry. Mol Cell Proteomics 9, 2292-2305 (2010).
39. J. Merl, M. Ueffing, S. M. Hauck, C. von Toerne, Direct comparison of MS-based label-free and SILAC quantitative proteome profiling strategies in primary retinal Muller cells. Proteomics 12, 1902-1911 (2012).
40. M. Brosch, L. Yu, T. Hubbard, J. Choudhary, Accurate and sensitive peptide identification with Mascot Percolator. J Proteome Res 8, 3176-3181 (2009).
41. N. Sandbo et al., The Journal of Organic Chemistry, 288, NO. 22, pp. 15466-15473, (2013).
42. Wang, Lei; Tachrim, Zetryana Puteri; Kurokawa, Natsumi; Ohashi, Fumina; Sakihama, Yasuko; et al Molecules (2017), 22(8), 1389/1-1389/8

1. A compound for use in the treatment of fibrosis and neoplasia, preferably a fibrosis or neoplasia located in the heart, the lung, the renal tract, the liver, in the skin, in the pleura and retroperitoneum, more preferably the fibrosis is selected from pleural fibrosis, retroperitoneal fibrosis, atrial fibrillation, myocardial interstitial fibrosis, idiopathic pulmonary fibrosis (IPF), interstitial lung diseases, chronic kidney disease, non-alcoholic fat liver disease, skin scars, keloids, tumor-associated desmoplastic reaction wherein said compound is a compound according to formula (I) 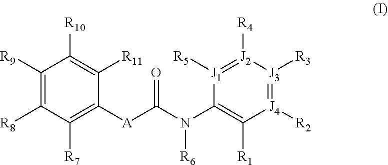
wherein,
R¹ is selected from the group consisting of —OR¹², —O(CH₂)ᵤ(C₃-C₁₀)aryl, —O(CH₂)ᵤ(C₃-C₁₀)cycloalkyl, —O(CH₂)ᵤ(C₂)alkynyl; —(CH₂)₁(C₃-C₁₀)aryl, —O(CH₂)ᵤ(C₃-C₁₀)cycloalkyl, —(CH₂)ᵤ(C₃-C₁₀)cycloalkyl, 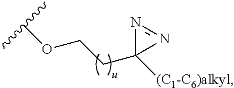
and —(CH₂)ᵤ(C₂)alkynyl;
u is 0 to 6;
R² to R⁵ are independently selected from the group consisting of H, —OR¹², —(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —CHZ₂, —CZ₃—CH₂Z, —OCHZ₂, —OCZ₃, —OCH₂Z—N(R¹³)(R¹⁴), —N(R¹⁵)(OR¹⁶), —S(O)₀₋₂R¹⁷, —S(O)₁₋₂OR¹⁸, —OS(O)₁₋₂R¹⁹—OS(O)₁₋₂OR²⁰, —S(O)₁₋₂N(R²¹)(R²²), —OS(O)₁₋₂N(R²³)(R²⁴), —N(R²⁵)S(O)₁₋₂R²⁶, —NR²⁷S(O)₁₋₂OR²⁸, —NR²⁹S(O)₁₋₂N(R³⁰)(R³¹), —C(═X)R³², —C(═X)XR³³, —XC(═X)R³⁴, and —XC(═X)XR³⁵, —OR³⁶, —O(CH₂(C₃-C₁₀)aryl, —O(CH₂)ᵥ(C₃-C₁₀)cycloalkyl, —O(CH₂)ᵥ(C₂)alkynyl;
R⁶ is selected from the group consisting of H, —(C₁-C₁₀)alkyl, benzyl and —(CH₂)₁₋₅(C₃-C₁₀)cycloalkyl; wherein —(C₁-C₁₀)alkyl, benzyl and —(CH₂)₁₋₅(C₃-C₁₀)cycloalkyl optionally are further substituted with at least one substituent selected from the group consisting of Halogen, preferably F;
R⁷ to R¹¹ are independently selected from the group consisting of H, —OR¹², —SR¹², —(C₁-C₁₀)alkyl, halogen, —(C₁-C₁₀)alkylO(C₁-C₁₀)alkyl, cyano, isocyano, cyanato, isocyanato, thiocyanato, isothiocyanato, azido, —(C₂-C₁₀)alkenyl, —(C₂-C₁₀)alkynyl, —O(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —(CH₂)ᵥCHZ₂, —CZ₃—CH₂Z, —OCHZ₂, —OCZ₃, —OCH₂Z, —N(R¹³)(R¹⁴), —N(R¹⁵)(OR¹⁶), —S(O)₀₋₂R¹⁷, —S(O)₁₋₂OR¹⁸, —OS(O)₁₋₂R¹⁹, —OS(O)₁₋₂OR²⁰, —S(O)₁₋₂N(R²¹)(R²²), —OS(O)₁₋₂N(R²³)(R²⁴), —N(R²⁵)S(O)₁₋₂R²⁶, —NR²⁷S(O)₁₋₂OR²⁸, —NR²⁹S(O)₁₋₂N(R³⁰)(R³¹), —C(═X)R³², —C(═X)XR³³, —XC(═X)R³⁴, and —XC(═X)XR³⁵, —O(CH₂)ᵥ(C₃-C₁₀)cycloakyl, —O(CH₂)ᵥ(C₁-C₁₀)alkyl and —O(CH₂)ᵥ(C₁-C₁₀)aryl,
wherein two adjacent rests of R¹ to R⁵ and R⁷ to R¹¹ optionally may form a ring attached to the underlying aromatic ring of formula (I) according to formula (III) to (XI) 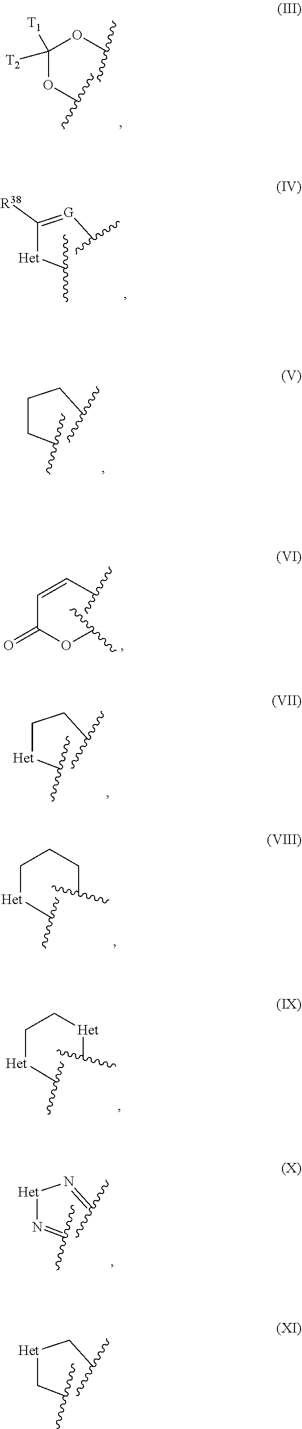
wherein T₁ and T² are independently selected from the group consisting of H, —(C₁-C₁₀)alkyl and halogen;
wherein each hydrogen in formula (III) to (IX) is optionally substituted with halogen, or —(C₃-C₁₀)aryl, —(C₁-C₃)alkyl, preferably F;
Het is selected from O, S, NH, N(C₁-C₁₀)alkyl;
G is selected from CH, N,
J₁ to J₄ are independently selected from C or N, preferably J₁ to J₄ are C;
wherein if any one of J₁ to J₄ is N, the corresponding R¹ to R⁴ attached to the respective J, to J₄ which is (are) N is absent;
R¹² to R³⁶ are independently selected from the group consisting of H, —(C₁-C₁₀)alkyl, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl;
R³⁶ is independently selected from the group consisting of H, —(C₁-C₁₀)alkyl;
R¹ to R¹¹, independently selected from the group consisting of, —(C₁-C₁₀)alkyl, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —O(CH₂)ᵥ(C₃-C₁₀)cycloalkyl, —O(CH₂)ᵥ(C₁-C₁₀)alkyl and —O(CH₂)(C₃-C₁₀)aryl and R¹² to R³⁵ optionally are further substituted with at least one substituent selected from the group consisting of OR¹²—(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —CHZ₂, —CZ₃—CH₂Z, —OCHZ₂, —OCZ₃, —OCH₂Z, —N(R¹³)(R¹⁴), —N(R¹⁵)(OR¹⁶), —NH C(O)(C₁-C₁₀)alkyl, —S(O)₀₋₂R¹⁷, —S(O)₁₋₂OR¹⁸, —OS(O)₁₋₂R¹⁹,—OS(O)₁₋₂OR²⁰, —S(O)₁₋₂N(R²¹)(R²²), —OS(O)₁₋₂N(R²³)(R²⁴), —N(R²⁵)S(O)₁₋₂R²⁶, —NR²⁷S(O)₁₋₂OR²³, —NR²⁹S(O)₁₋₂N(R³⁰)(R³¹), —C(═X)R³², —C(═X)XR³³, —XC(═X)R³⁴, and —XC(═X)XR³⁵, —OR³⁶, and —O(CH₂)ᵥ(C₃-C₁₀)aryl;
v is 0 to 5;
Z is halogen;
X is selected from the group consisting of O, —NH— or S;
A is selected from the group consisting of 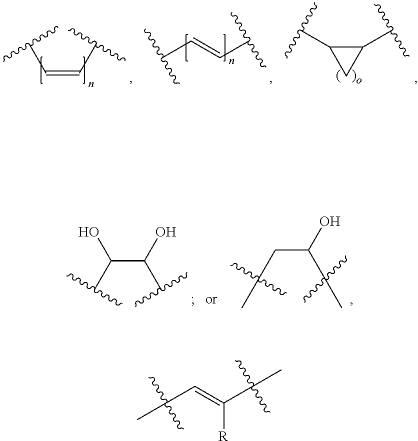
n is 1, 2, or 3, preferably 1;
o is 1, 2, or 3, preferably 1;
R is H, (C₁-C₆)alkyl, cyano, —(C₃-C₁₀)cycloalkyl, benzyl or part of a ring wherein R is connected with R⁷ or R¹¹ by 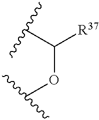
preferably H or benzyl, most preferably H;
R³⁷ is H or —CF₃;
with the provision that if n is 2 or 3, A may be 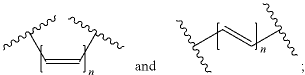
with the proviso that R⁵ is not —COOH.

2. A compound according to formula (II) 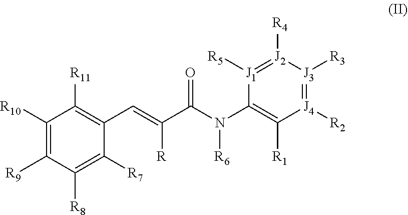
wherein,
R¹ is —OR¹², —O(C₅-C₁₀)heteroaryl, —O(C₃-C₁₀)aryl, —O(CH₂)ᵤ(C₃-C₁a)cycloalkyl, —O(CH₂)ᵤ(C₃-C₁₀)aryl, or 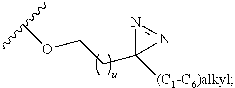
R² to R⁵ and R⁷, and R¹¹ are independently selected from the group consisting of H, —(C₁-C₁₀)alkyl, halogen, azido, cyano, —O(C₁-C₁₀)alkyl, —(CH₂)ᵤ(C₃-C₁₀)aryl, —(CH₂)ᵤ(C₃-C₁₀)cycloalkyl, —(C₂-C₁₀)alkenyl, —(C₂-C₁₀)alkynyl, —(C₃-C₁₀)cycloalkyl, —(C₃-C₁₀)aryl, which optionally are further substituted with at least one substituent selected from the group consisting of Halogen, —OH, —NH₂, —NHC(O)CH₃, —CN, —N₃, and —COOH, —C(O)NH₂;
R⁶ is H, —(C₁-C₁₀)alkyl, benzyl and —(CH₂)₁₋₅(C₃-C₁₀)cycloalkyl; wherein —(C₁-C₁₀)alkyl, benzyl and —(CH₂)₁₋₅(C₃-C₁₀)cycloalkyl optionally are further substituted with at least one substituent selected from the group consisting of Halogen, preferably F;
R⁸, R⁹ and are H, —O(C₁-C₁₀)alkyl, —SR¹², —O(CH₂)ᵤ(C₃-C₁c)aryl, —O(CH₂)ᵤ(C₃-C₁₀)cycloalkyl, —O(C₃-C₁₀)cycloalkyl, or, —O(C₂-C₁₀)alkenyl;
R¹⁰ is H, halogen, —O(C₁-C₁₀)alkyl, —O(CH₂)ᵤ(C₃-C₁₀)aryl, —O(CH₂)ᵤ(C₃-C₁₀)cycloalkyl, —O(C₃-C₁₀)cycloalkyl, or, —O(C₂-C₁₀)alkenyl;
with the proviso that if R₉=H either Rₐ or R₁₀ is —OR¹²
u is 0 to 6;
R is H, (C₁-C₆)alkyl, cyano, —(C₃-C₁₀)cycloalkyl, benzyl or part of a ring wherein R is connected with R⁷ or R¹¹ by 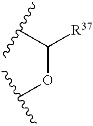
preferably H or benzyl, most preferably H;
R³⁷ is H or —CF₃;
R¹² are independently selected from the group consisting of H, —(C₁-C₁₀)alkyl, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, (CH₂)ᵤ(C₃-C₁₀)aryl, —(CH₂)ᵤ(C₃-C₁₀)heteroaryl —(CH₂)ᵤ(C₃-C₁₀)cycloalkyl; preferably —(C₁-C₁₀)alkyl, more preferably —(C₁-C₄)alkyl;
wherein two adjacent rests of R⁸ to R¹⁰ optionally may form a ring, attached to the underlying aromatic ring of formula (II) according to 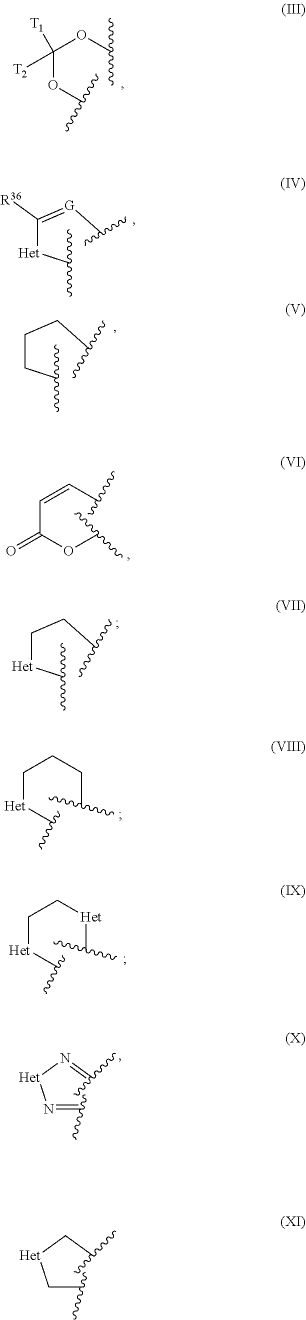
wherein T¹ and T² are independently selected from the group consisting of H, —(C₁-C₁₀)alkyl and halogen;
wherein each hydrogen in formula (III) to (XI) is optionally substituted with halogen, —(C₃-C₁₀)aryl, or —(C₁-C₃)alkyl, preferably F;
Het is O, S, N(C₁-C₁₀)alkyl or NH; preferably O;
R³⁸ is independently selected from the group consisting of H, —(C₁-C₁₀)alkyl;
G is selected from CH, N;
J₁ to J₄ are independently selected from C or N, preferably J₁ to J₄ are C;
wherein if any one of J₁ to J₄ is N, the corresponding R¹ to R⁴ attached to the respective J₁ to J₄ which is (are) N is absent;
with the proviso that
if J₁ to J₄ are C and
I) if R⁵ is —(CH₂)₃CH₃; R⁹ is —OCH₃, —OCH₂CH₃, —O(CH₂)₂CH₃, —OCH₂phenyl or —O(CH₂)₃CH₃; R¹, R², R, R⁴, R, R¹¹ and R are H; and
a) R⁸ and R¹⁰ are H; or
b) R⁸ is —OCH₃, or —OCH₂CH₃ and R¹⁰ is H; or
c) R¹⁰ is —OCH₃, or —OCH₂CH₃ and R⁸ is of H;
then R⁶ is not H;
II) if R⁹ is —OCH₃, —O(CH₂)₂CH₃, —O(2-propyl), —O(CH₂)₄CH₃, —O(CH₂)₅CH₃, —OCH₂(4-chlorophenyl), —O(CH₂)₂CH(CH₃)₂, —OCH₂(2,6-dichlorophenyl), or —OCH₂phenyl; R¹, R², R, R⁴, R⁷, R¹¹, and R are H; R⁸ is —OCH₃ and R¹⁰ is H, or R¹⁰ is —OCH₃ and R⁸ is H; R⁵ is —(CH₂)₃CH₃; then R⁶ is not H;
III) if R⁹ is —OCH₃; R⁸ is Brand R¹⁰ is H, or R¹⁰ is Br and R⁸ is H; R⁵ is —(CH₂)₃CH₃; R¹, R², R³, R⁴, R¹¹ and R are H, then R¹ is not H;
IV) if R⁵ is —(CH₂)₃CH₃, R⁹ is —OCH₃, R⁷ is —OCH₃ and R¹¹ is H or R¹¹ is —OCH₃ and R⁷ is H; R¹, R², R³, R⁴, R⁸, R¹⁰, R¹¹ and R are H; then R⁶ is not H;
V) if R⁹ is —OCH₃, —OCH₂phenyl, or —OCH₂(2-fluorophenyl); R⁸ is Brand R¹⁰ is —OCH₃, or R¹⁰ is Br and R⁸ is —OCH₃; R⁵ is —O(CH₂)₃CH₃; R¹, R², R³, R⁴, R⁷, R¹¹, and R are H, then R⁶ is not H;
VI) if R⁹ is —O(CH₂)₃CH₃; R⁸ is —OCH₂CH₃ and R¹⁰ is H, or R¹⁰ is —OCH₂CH₃ and R⁸ is H; R⁵ is —O(CH₂)₃CH₃; R¹, R², R³, R⁴, R⁷, R¹¹, and R are H, then R⁶ is not H;
VII) if R⁹ is —OCH₂(2-chlorophenyl); R⁸ is Br and R¹⁰ is —CH₂CH₃, or R¹⁰ is Br and R⁸ is —OCH₂CH₃; R⁵ is —(CH₂)₃CH₃; R¹, R², R³, R⁴, R⁷, R¹¹, and R, are H, then R⁶ is not H;
VIII) if R⁹ is —O(2-octenyl); R⁸ is Cl and R¹⁰ is H, or R¹⁰ is CI and R⁸ is H; R is —(CH₂)₃COOH; R¹, R², R³, R⁴, R⁷, R¹¹, and R are H, then is not H;
IX) if R⁹ is —OCH₃; R¹, R², R³, R⁴, R⁷, R⁸, R¹⁰, R¹¹, R are H; and
R⁵ is -(2-fluorophenyl), -phenyl; then R⁶ is not H;
X) if R⁹ is —OCH₂CH₃; R⁵ is —(CH₂)₃CH₃; R¹, R², R³, R⁴, R⁷, R⁸, R¹⁰, R¹¹, and R are H; then R⁶ is not H;
XI) if R⁹ is —OCH₃; R⁸ is —OCH₃ and R¹⁰ is H, or R¹⁰ is —OCH₃ and R⁸ is H; R⁵ is —(CH₂)₃CH₃; R¹, R²R³, R⁴, R⁸, R⁷, and R are H; then R⁶ is not H;
XII) if R⁹ is —OCH₃; R⁵ is —O(CH₂)₃CH₃; R¹, R², R³, R⁴, R⁷, R¹¹, and R are H, and R⁸ is —OCH₂CH₃ and R¹⁰ is H, or R¹⁰ is —OCH₂CH₃ and R⁸ is H; then R⁶ is not H;
XIII) if R⁵ is —(CH₂)₃CH₃; R⁹ is —O(CH₂)₃CH₃, or —OCH₃; R¹, R², R³, R⁴, R⁷, R⁸, R¹⁰, R¹¹, and R are H; then R⁶ is not H;
XIV) if R⁵ is —CH₃, —(CH₂)₂CH₃ or —CH₂CH₃; R⁹ is —OCH₃; R⁸ is —OCH₃ and R¹⁰ is H or R⁸ is H and R¹⁰ is —OCH₃; R¹, R², R³, R⁴, R⁷, R¹¹, and R are H; then R⁶ is not H.
XV) if R⁵ is —(CH₂)₃CH₃; R¹, R², R³, R⁴, R⁷, R⁸, R⁹, R¹⁰ and R¹¹ are H; then R⁶ is not H.

3. The compound for use of claim 1, wherein
I) R¹ is selected from the group consisting of —OR¹², —O(CH₂)₁(C₃-C₁₀)aryl, —O(CH₂)ᵤ(C₃-C₁₀)cycloalkyl, —O(CH₂)ᵤ(C₂)alkynyl; preferably of —OR¹²
u is 0 to 5;
R¹² is —(C₁-C₁₀)alkyl, preferably —(C₃-C₅)alkyl, more preferably —(C₄)alkyl and/or
II) A is selected from 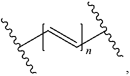
preferably n=1, 2, more preferably n=1 and/or
III) R² to R⁵ are independently selected from the group consisting of H, —OR¹², —(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —CH(Z)₂, —C(Z)₃—CH₂Z, —OCH(Z)₂, —OC(Z)₃, —OCH₂Z, —N(R¹³)(R¹⁴), —N(R¹⁵)(OR¹⁶), —C(═X)R³², —C(═X)XR³³, —XC(═X)R³⁴, and —XC(═X)XR³⁵, O(CH₂)ᵥ(C₃-C₁₀)aryl, —O(CH₂)(C₃-C₁₀)cycloalkyl, —O(CH₂)ᵥ(C₂)alkynyl.

4. The compound for use of claim 1 and wherein
R⁷ to R¹¹ are independently selected from the group consisting of H, —OR¹², —SR¹²—(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —O(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —(CH₂)ᵥCH(Z)₂, —CZ)₃—CH₂Z, —OCH(Z)₂, —OC(Z)₃, —OCH₂Z, —N(R¹³)(R¹⁴), —N(R¹⁵)(OR¹⁶), —C(═X)R³², —C(═X)XR³³, —XC(═X)R³⁴, and —XC(═X)XR³⁵, —O(CH₂)ᵥ(C₃-C₁₀)cycloakyl, —O(CH₂)ᵤ(C₁-C₁₀)alkyl and —O(CH₂)(C₃-C₁₀)aryl,
wherein two adjacent rests of R¹ to R⁶ and R to R¹¹ optionally may form a ring according to 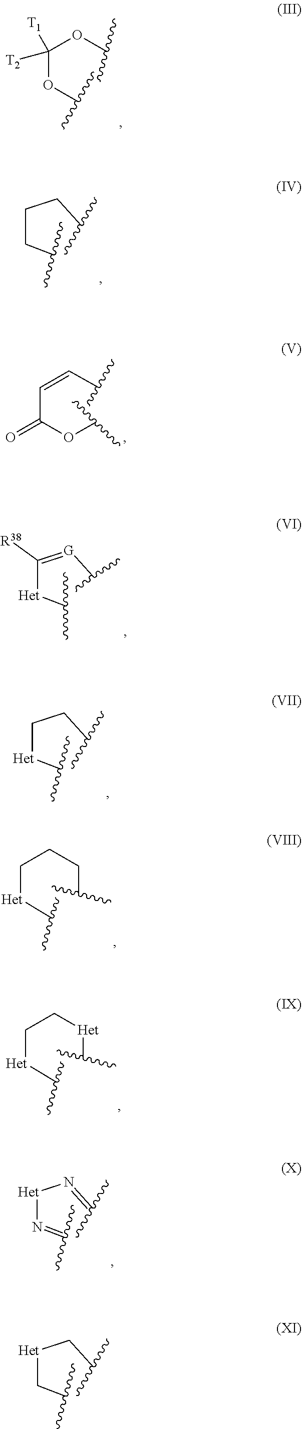
preferably according to formula (III);
wherein T¹ and T² are independently selected from the group consisting of H, —(C₁-C₁₀)alkyl and halogen;
wherein each hydrogen in formula (III) to (XI), optionally is substituted with halogen, or —(C₃-C₁₀)aryl, preferably F;
R³⁸ is independently selected from the group consisting of H, —(C₁-C₁₀)alkyl.

5. The compound for use of claim 1, wherein
R¹ to R¹¹, are selected from the group consisting of, —(C₁-C₁₀)alkyl, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —O(CH₂)(C₃-C₁₀)cycloakyl, —O(CH₂)ᵥ(C₁-C₁₀)alkyl and —O(CH₂)ᵤ(C₃-C₁₀)aryl and R¹² to R³⁵ optionally are further substituted with a substituent selected from the group consisting of OR¹², —(C₁-C₁₀)alkyl, halogen, cyano, isocyano, cyanato, isocyanato, thiocyanato, isothiocyanato, azido, —(C₂-C₁₀)alkenyl, —(C₂-C₁₀)alkynyl, —(C₃-C₁₀)cycloalkyl, —(C₃-C₁₀)heterocyclyl, —(C₁-C₁₀)aryl, —(C₃-C₁₀)heteroaryl, —CHZ₂, —CZ₃—CH₂Z, —OCHZ₂, —OCZ₃, —OCH₂Z, —N(R¹³)(R¹⁴), —N(R¹⁵)(OR¹⁶), —C(═X)R³², —C(═X)XR³³, —XC(═X)R³⁴, and —XC(═X)XR³⁵, —OR³⁶, and —O(CH₂)ᵥ(C₃-C₁₀)aryl.

6. The compound for use of claims 1 and 3 to 5, wherein R⁹ is selected from the group consisting of —OR¹², —SR¹², halogen, —O(C₂-C₁₀)alkynyl, —CZ₃, —OCHZ₂, —OCZ₃, —OCH₂Z, —O(CH₂)ᵥ(C₃-C₁₀)cycloakyl, —O(CH₂)(C₃-C₁₀)alkyl and —O(CH₂)ᵥ(C₃-C₁₀)aryl, wherein R⁹ selected from the group consisting of —O(C₁-C₁₀)alkyl, —OCH₂Z, —O(CH₂)ᵥ(C₃-C₁₀)cycloakyl, —O(CH₂)ᵥ(C₁-C₁₀)alkyl and —O(CH₂)ᵥ(C₃-C₁₀)aryl optionally is further substituted with at least one substituent selected from the group consisting of Halogen, —OH, —NH₂, —NHC(O)CH₃, —CN, —N₃, and —COOH, —C(O)NH₂.

7. The compound of claim 1 or 2, wherein
i) R² to R⁵, R⁷ or R¹¹ are independently selected from the group consisting of H, —OR¹²—(C₁-C₁₀)alkyl, halogen, cyano, azido, —(C₂-C₁₀)alkenyl, —(C₂-C₁₀)alkynyl, —(C₃-C₁₀)cycloalkyl, —(C₃-C₁₀)heterocyclyl, —(C₃-C₁₀)aryl, —(C₃-C₁₀)heteroaryl, —CHZ₂, —CZ₃—CH₂Z, —OCHZ₂, —OCZ₃, —OCH₂Z, —OR³, —O(CH₂)ᵥ(C₃-C₁₀)aryl, —O(CH₂)ᵥ(C₃-C₁₀)cycloalkyl, —O(CH₂)ᵥ(C₂)alkynyl; and/or
ii) R⁰ is —OR¹²; and R⁸ is —H and R¹⁰ is H or —OR¹²; and/or
iii) R¹² is selected from the group consisting of H, —(C₁-C₁₀)alkyl, —(C₂-C₄)alkynyl, —(C₃-C₁₀)cycloalkyl, —(C₃-C₁₀)heterocyclyl, —(C₃-C₁₀)aryl, —(C₃-C₁₀)heteroaryl.

8. The compound of claim 2 or 7, wherein
i) R⁹ is H and R⁸ is H and R¹⁰ is —OR¹²; or
ii) R⁹ and R¹⁰ form a ring, attached to the underlying aromatic ring of formula (II) according to 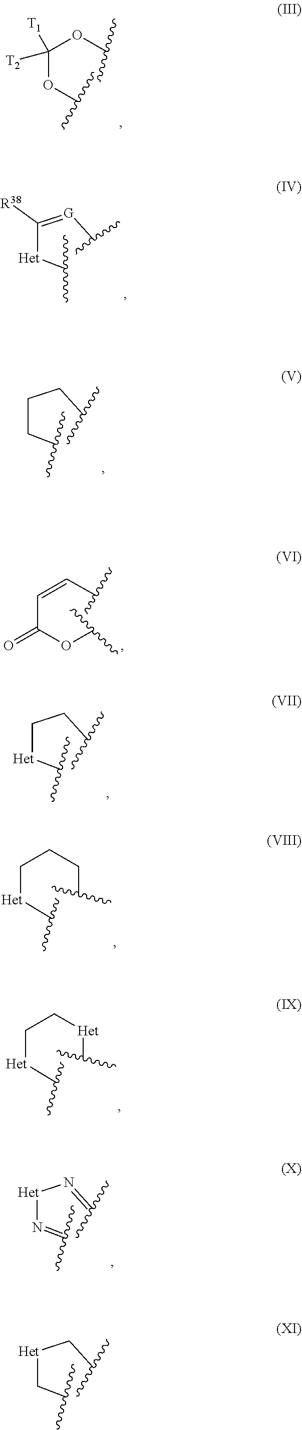
preferably according to formula (III)
wherein T¹ and T² are independently selected from the group consisting of H, —(C₁-C₁₀)alkyl and halogen;
wherein each hydrogen in formula (III) to (X) is optionally substituted with halogen, or —(C₃-C₁₀)aryl, preferably F;
R³⁸ is independently selected from the group consisting of H, —(C₁-C₁₀)alkyl;
Het is O, S or NH, preferably O;
G is selected from CH, N. and/or
iii) J₁₋₄ is CH; and/or
iv) u=0-3 and/or
v) R¹² is —O(C₄-C₆)alkyl, —OCH₂(C₃-C₅)cycloalkyl, —OPhenyl or —OCH₂Phenyl; and/or
vi) R⁶=H or C₁-C₄ alkyl or (CH₂)₍₁₋₃₎ alkyl-(C₁-C₆)cycloalkyl; which optionally are further substituted with at least one substituent selected from the group consisting of Halogen, preferably F; and/or
vii) R² to R⁵, R⁷ or R¹¹ are independently selected from the group consisting of H, —(C₁-C₃)alkyl, halogen, cyano, azido, —CHZ₂, —CZ₃—CH₂Z, —OCHZ₂, —OCZ₃, —OCH₂Z, —(C₃-C₅)cycloalkyl;
viii) R² is H; and/or
ix) R⁷ is H; and/or
x) R¹¹ is H; and/or
xi) R³ is H; and/or
xii) R⁴ is H; and/or
xiii) R⁵ is H and/or
xiv) R⁹ is OCH₃ and R¹⁰ is H or R₁₀ is OCH₃ and/or
xv) R⁶ is H; and/or
xvi) R¹ is —O(C₁-C₆))alkyl; and/or,
xvii) R¹⁰ is H or —OCH₃; and/or
xviii) R¹ is —O(C₄-C₆))alkyl; and/or
xix) R⁹ is —OCH₃ and R¹⁰═H or —OCH₃; and/or
xx) R¹⁰ is H.

9. The compound according to claim 2, 7 and 8 wherein
i) R¹ is selected from the group consisting of —O(C₁-C₄)alkyl, —O(C₅-C₁₀)heteroaryl, —O(C₃-C₁₀)aryl, —O(CH₂)ᵤ(C₃-C₁₀)aryl, preferably —Obutyl;
wherein a) (C₅-C₁₀)heteroaryl is preferably selected from the group consisting of 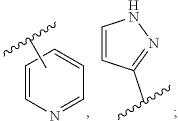
and/or
b) (C₃-C₁₀)aryl is preferably phenyl, optionally substituted with halogen and/or (C₁-C₃)alkyl; and/or
ii) R² is selected from the group consisting of hydrogen, halogen, preferably hydrogen;
wherein halogen is preferably Cl or F;
c) R³ is selected from the group consisting of hydrogen, halogen, preferably hydrogen;
wherein halogen is preferably Cl or F; and/or
iii) R⁴ is selected from the group consisting of hydrogen, halogen, preferably hydrogen;
wherein halogen is preferably Cl or F; and/or
iv) R⁵ is selected from the group consisting of hydrogen, halogen, preferably hydrogen;
wherein halogen is preferably Cl or F; and/or
v) R⁶ is H, —(C₁-C₁₀)alkyl, and —(CH₂)₁₋₅cyclopropyl; and/or
vi) R⁷ is H; and/or
vii) R⁸ is H; and/or
viii) R⁹ is H, —O(C₁-C₄)alkyl, —OCH₂CF₃, —O(CH₂)cyclopropyl, —OCF₃, —OCHF₂, or, —O(C₂-C₁₀)alkenyl, —O(CH₂)₃C≡CH; and/or
ix) R¹⁰ is H, halogen, or —O(C₁-C₂)alkyl; and/or
x) wherein two adjacent rests of R⁹ and R¹⁰ form a ring, attached to the underlying aromatic ring of formula (II) according to 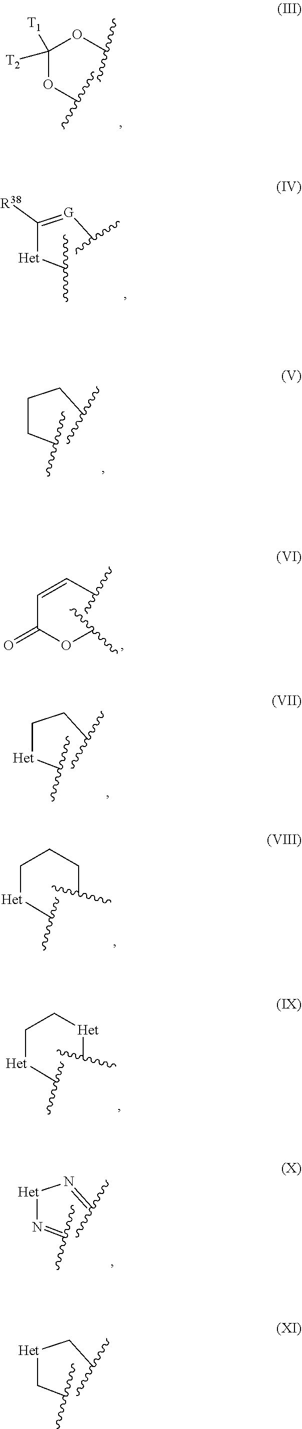
wherein T¹ and T² are independently selected from the group consisting of H, -methyl and F;
wherein each hydrogen in formula (III) to (XI) is optionally substituted with methyl;
R³⁸ is independently selected from the group consisting of H, —(C₁-C₁₀)alkyl;
Het is O;
G is selected from CH;
xi) R¹¹ is H or —OCH₃; and/or
xii) R is H, C₁-C₆ alkyl, or benzyl, most preferably H; and/or
xiii) J₁ to J₄ are C or N.

10. A compound selected from the group consisting of 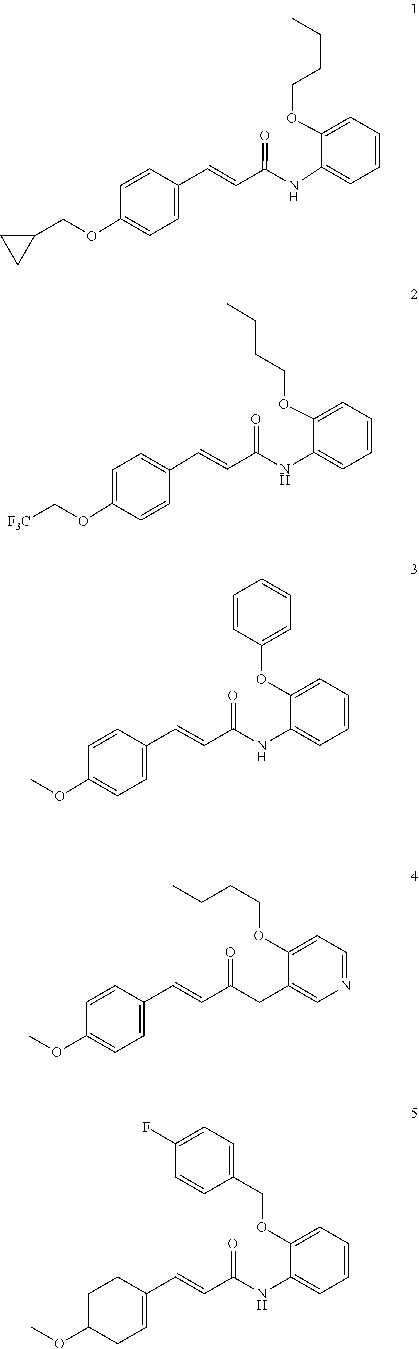 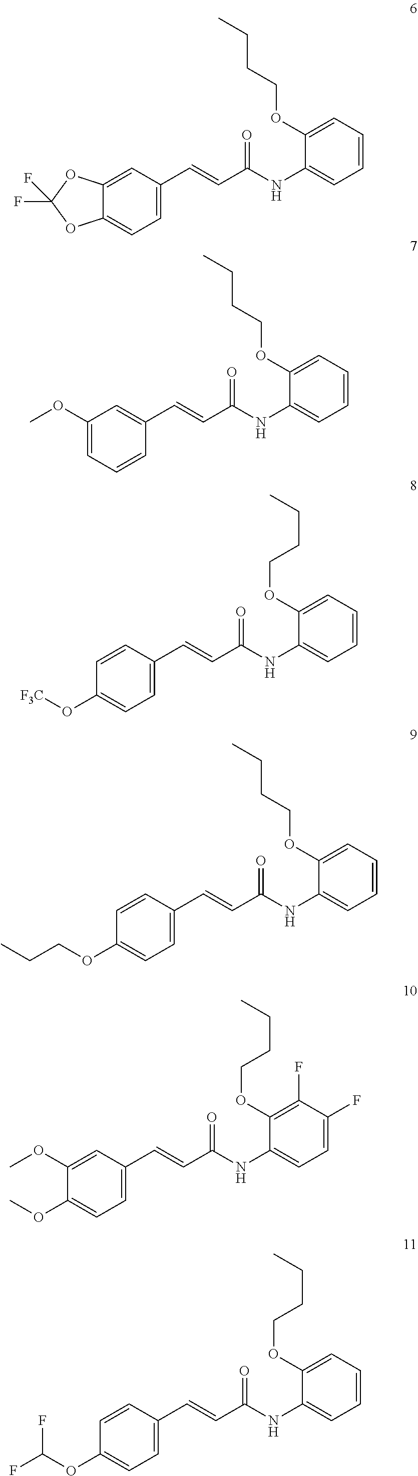 [SMILES: CCCCOc1ccccc1NC(=O)/C=C/c1ccc2c(c1)OC(F)(F)O2 ; CCCCOc1ccccc1NC(=O)/C=C/c1cccc(OC)c1 ; CCCCOc1ccccc1NC(=O)/C=C/c1ccc(OC(F)(F)F)cc1 ; CCCCOc1ccccc1NC(=O)/C=C/c1ccc(OCCC)cc1 ; CCCCOc1c(NC(=O)/C=C/c2ccc(OC)c(OC)c2)ccc(F)c1F ; CCCCOc1ccccc1NC(=O)/C=C/c1ccc(OC(F)F)cc1] 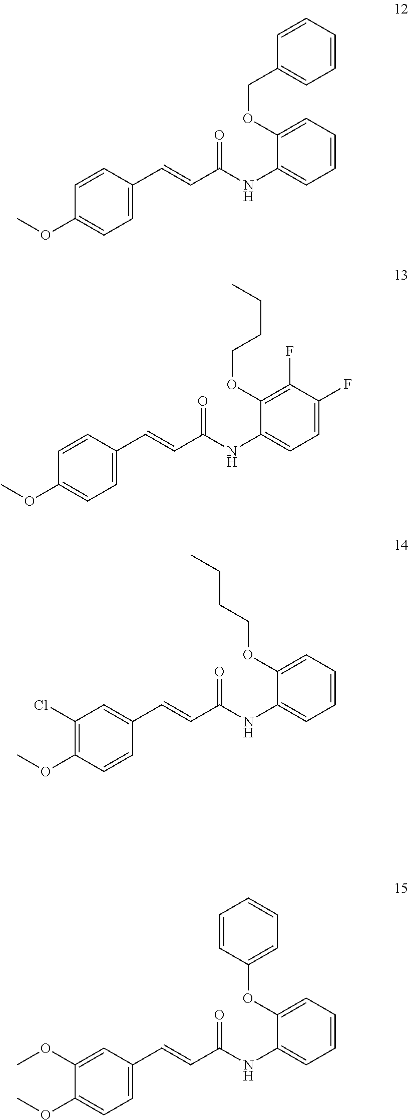 [SMILES: COc1ccc(/C=C/C(=O)Nc2ccccc2OCc2ccccc2)cc1 ; CCCCOc1c(NC(=O)/C=C/c2ccc(OC)cc2)ccc(F)c1F ; CCCCOc1ccccc1NC(=O)/C=C/c1ccc(OC)c(Cl)c1 ; COc1ccc(/C=C/C(=O)Nc2ccccc2Oc2ccccc2)cc1OC] 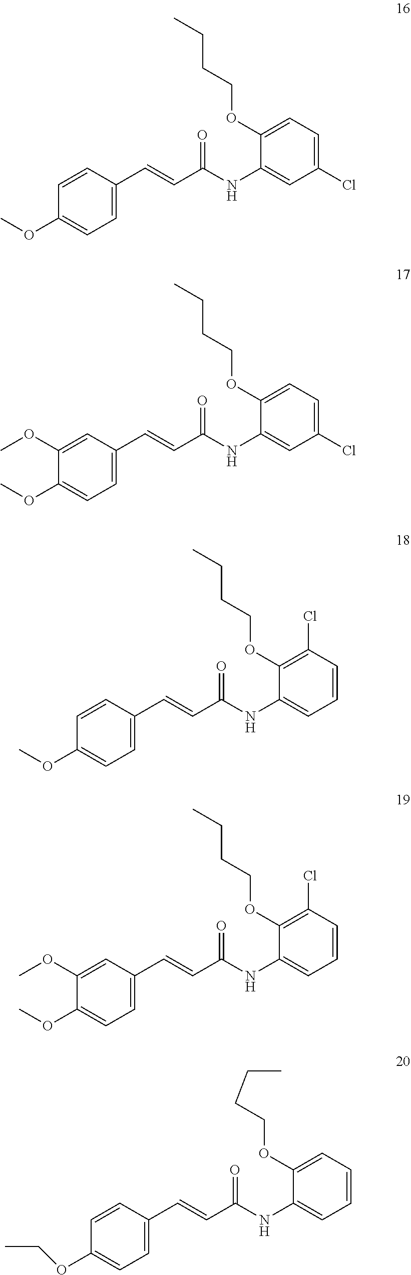 [SMILES: CCCCOc1ccc(Cl)cc1NC(=O)/C=C/c1ccc(OC)cc1 ; CCCCOc1ccc(Cl)cc1NC(=O)/C=C/c1ccc(OC)c(OC)c1 ; CCCCOc1c(Cl)cccc1NC(=O)/C=C/c1ccc(OC)cc1 ; CCCCOc1c(Cl)cccc1NC(=O)/C=C/c1ccc(OC)c(OC)c1 ; CCCCOc1ccccc1NC(=O)/C=C/c1ccc(OCC)cc1] 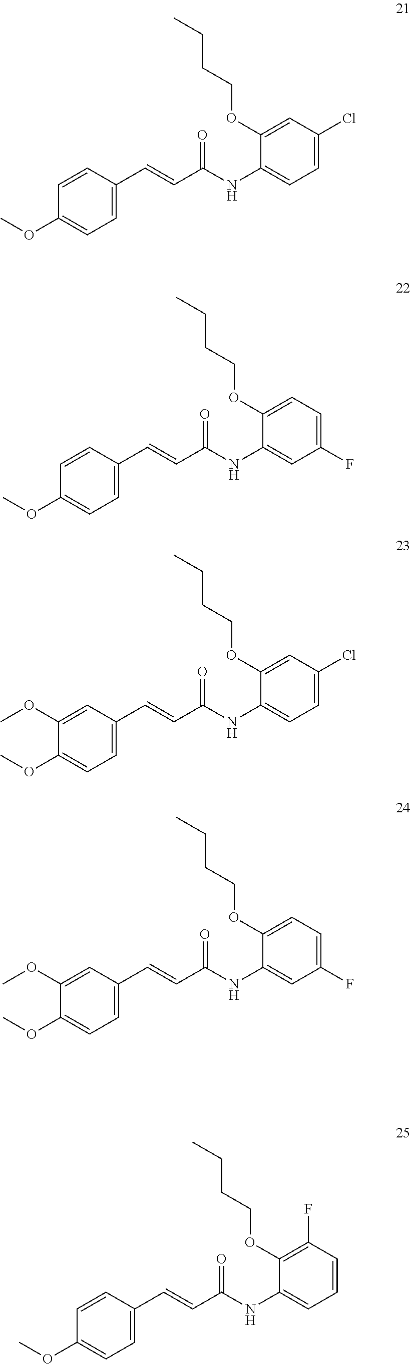 [SMILES: CCCCOc1cc(Cl)ccc1NC(=O)/C=C/c1ccc(OC)cc1 ; CCCCOc1ccc(F)cc1NC(=O)/C=C/c1ccc(OC)cc1 ; CCCCOc1cc(Cl)ccc1NC(=O)/C=C/c1ccc(OC)c(OC)c1 ; CCCCOc1ccc(F)cc1NC(=O)/C=C/c1ccc(OC)c(OC)c1 ; CCCCOc1c(F)cccc1NC(=O)/C=C/c1ccc(OC)cc1] 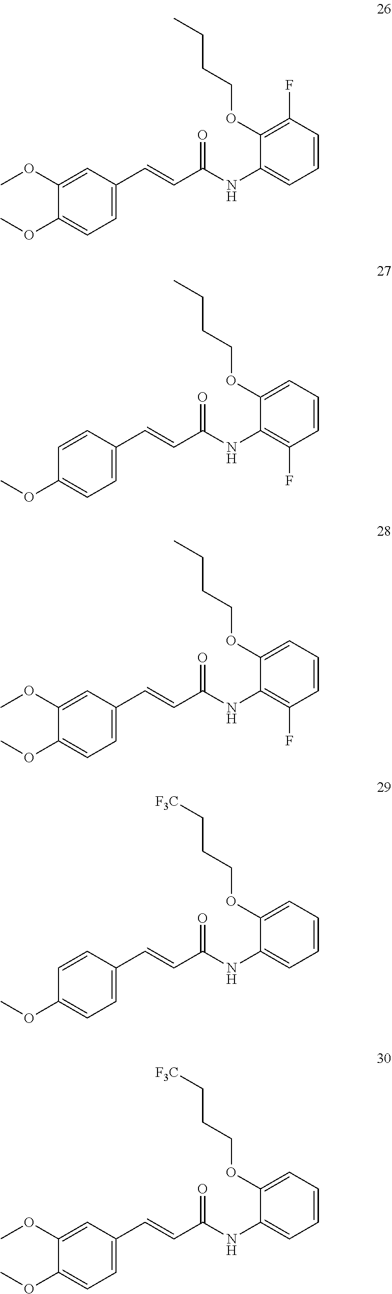 [SMILES: CCCCOc1c(F)cccc1NC(=O)/C=C/c1ccc(OC)c(OC)c1 ; CCCCOc1cccc(F)c1NC(=O)/C=C/c1ccc(OC)cc1 ; CCCCOc1cccc(F)c1NC(=O)/C=C/c1ccc(OC)c(OC)c1 ; COc1ccc(/C=C/C(=O)Nc2ccccc2OCCCC(F)(F)F)cc1 ; COc1ccc(/C=C/C(=O)Nc2ccccc2OCCCC(F)(F)F)cc1OC] 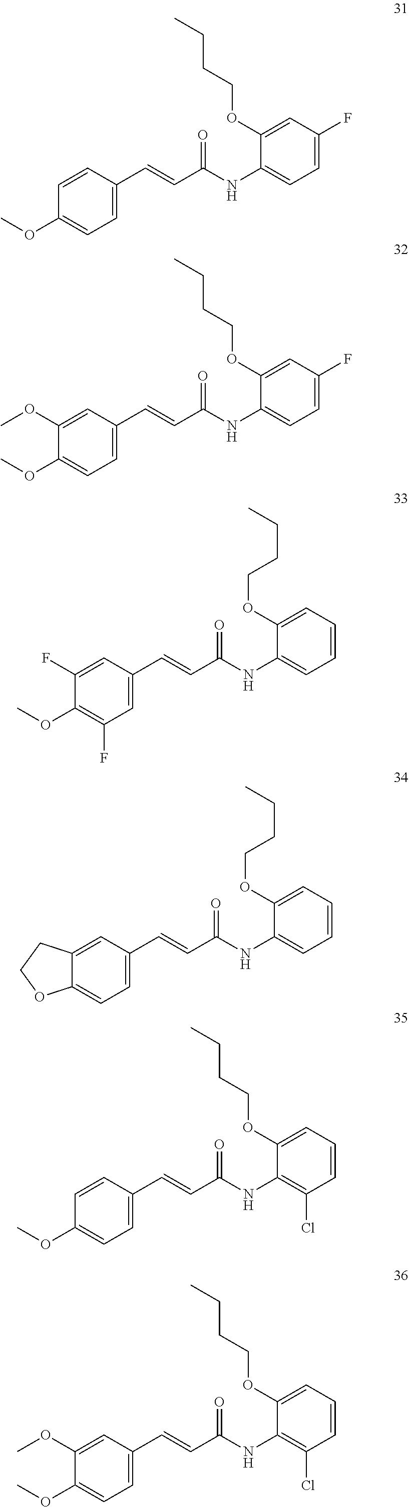 [SMILES: CCCCOc1cc(F)ccc1NC(=O)/C=C/c1ccc(OC)cc1 ; CCCCOc1cc(F)ccc1NC(=O)/C=C/c1ccc(OC)c(OC)c1 ; CCCCOc1ccccc1NC(=O)/C=C/c1cc(F)c(OC)c(F)c1 ; CCCCOc1ccccc1NC(=O)/C=C/c1ccc2c(c1)CCO2 ; CCCCOc1cccc(Cl)c1NC(=O)/C=C/c1ccc(OC)cc1 ; CCCCOc1cccc(Cl)c1NC(=O)/C=C/c1ccc(OC)c(OC)c1] 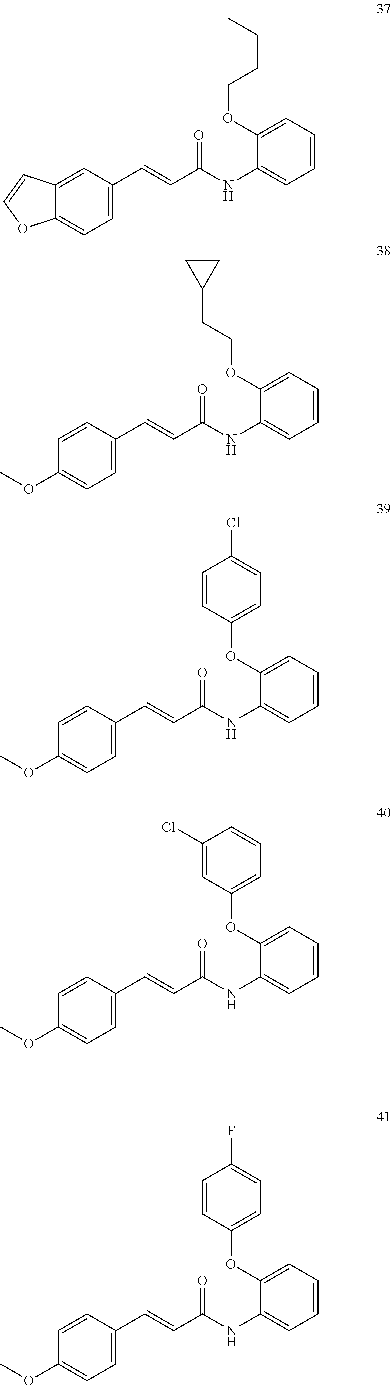 [SMILES: CCCCOc1ccccc1NC(=O)/C=C/c1ccc2occc2c1 ; COc1ccc(/C=C/C(=O)Nc2ccccc2OCCC2CC2)cc1 ; COc1ccc(/C=C/C(=O)Nc2ccccc2Oc2ccc(Cl)cc2)cc1 ; COc1ccc(/C=C/C(=O)Nc2ccccc2Oc2cccc(Cl)c2)cc1 ; COc1ccc(/C=C/C(=O)Nc2ccccc2Oc2ccc(F)cc2)cc1] 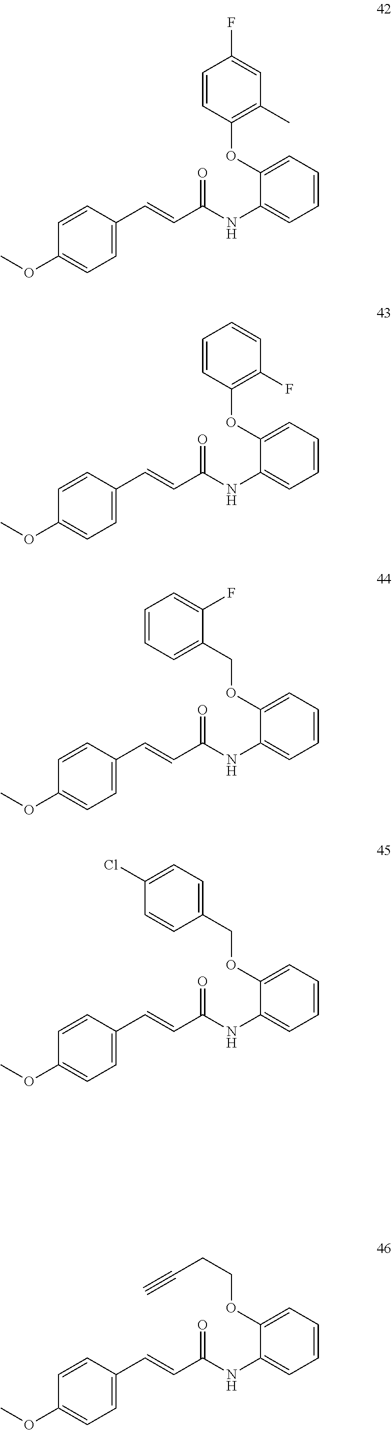 [SMILES: COc1ccc(/C=C/C(=O)Nc2ccccc2Oc2ccc(F)cc2C)cc1 ; COc1ccc(/C=C/C(=O)Nc2ccccc2Oc2ccccc2F)cc1 ; COc1ccc(/C=C/C(=O)Nc2ccccc2OCc2ccccc2F)cc1 ; COc1ccc(/C=C/C(=O)Nc2ccccc2OCc2ccc(Cl)cc2)cc1 ; C#CCCOc1ccccc1NC(=O)/C=C/c1ccc(OC)cc1] 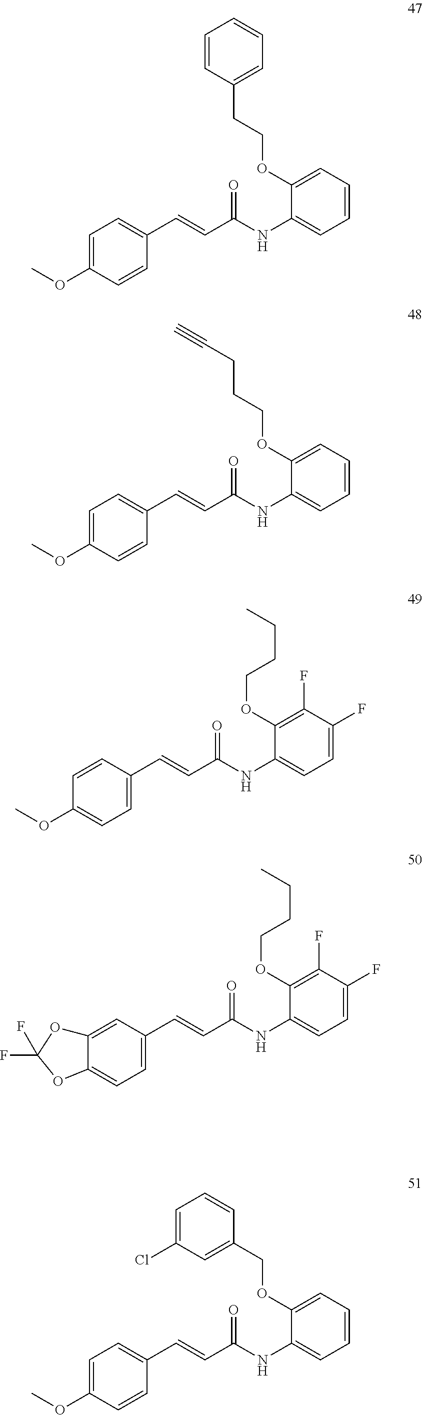 [SMILES: COc1ccc(/C=C/C(=O)Nc2ccccc2OCCc2ccccc2)cc1 ; C#CCCCOc1ccccc1NC(=O)/C=C/c1ccc(OC)cc1 ; CCCCOc1c(NC(=O)/C=C/c2ccc(OC)cc2)ccc(F)c1F ; CCCCOc1c(NC(=O)/C=C/c2ccc3c(c2)OC(F)(F)O3)ccc(F)c1F ; COc1ccc(/C=C/C(=O)Nc2ccccc2OCc2cccc(Cl)c2)cc1] 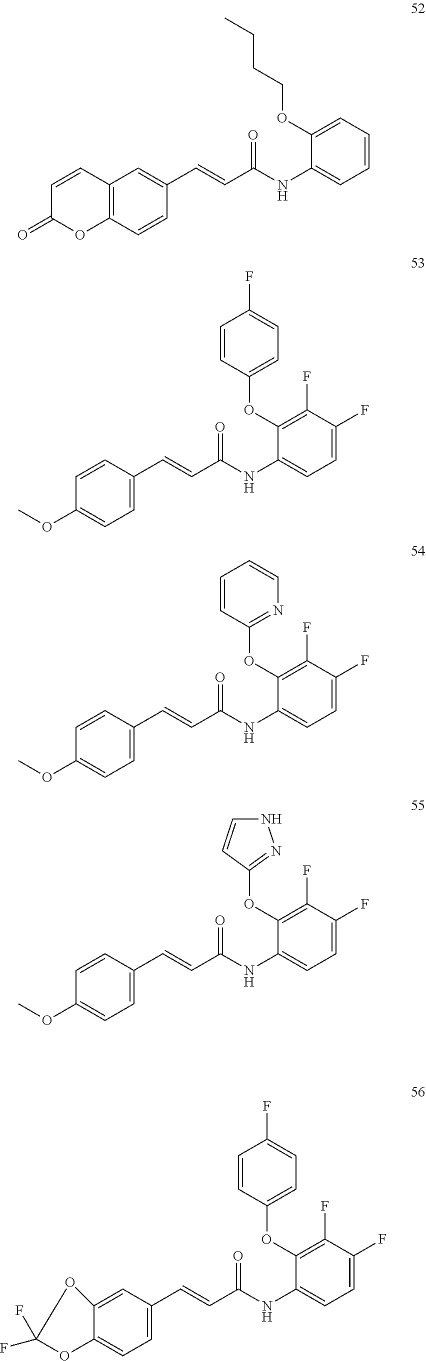 [SMILES: CCCCOc1ccccc1NC(=O)/C=C/c1ccc2oc(=O)ccc2c1 ; COc1ccc(/C=C/C(=O)Nc2ccc(F)c(F)c2Oc2ccc(F)cc2)cc1 ; COc1ccc(/C=C/C(=O)Nc2ccc(F)c(F)c2Oc2ccccn2)cc1 ; COc1ccc(/C=C/C(=O)Nc2ccc(F)c(F)c2Oc2cc[nH]n2)cc1 ; O=C(/C=C/c1ccc2c(c1)OC(F)(F)O2)Nc1ccc(F)c(F)c1Oc1ccc(F)cc1] 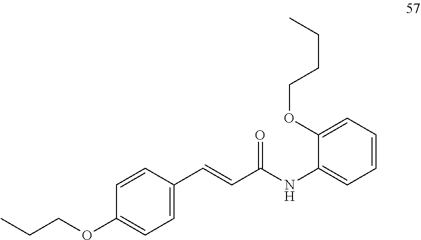 [SMILES: CCCCOc1ccccc1NC(=O)/C=C/c1ccc(OCCC)cc1] 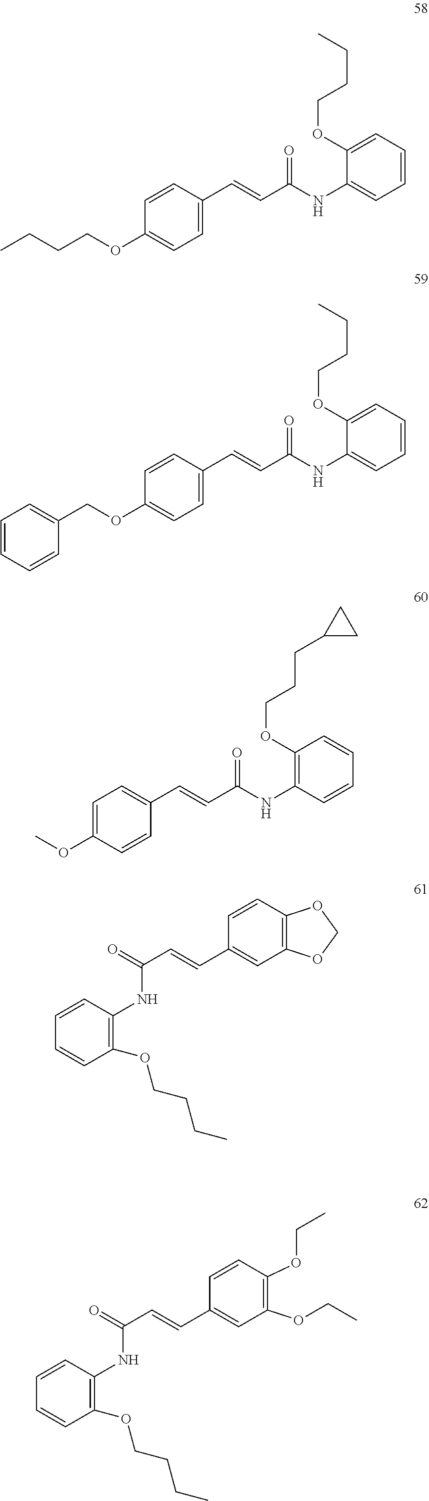 [SMILES: CCCCOc1ccc(/C=C/C(=O)Nc2ccccc2OCCCC)cc1 ; CCCCOc1ccccc1NC(=O)/C=C/c1ccc(OCc2ccccc2)cc1 ; COc1ccc(/C=C/C(=O)Nc2ccccc2OCCCC2CC2)cc1 ; CCCCOc1ccccc1NC(=O)/C=C/c1ccc2c(c1)OCO2 ; CCCCOc1ccccc1NC(=O)/C=C/c1ccc(OCC)c(OCC)c1] 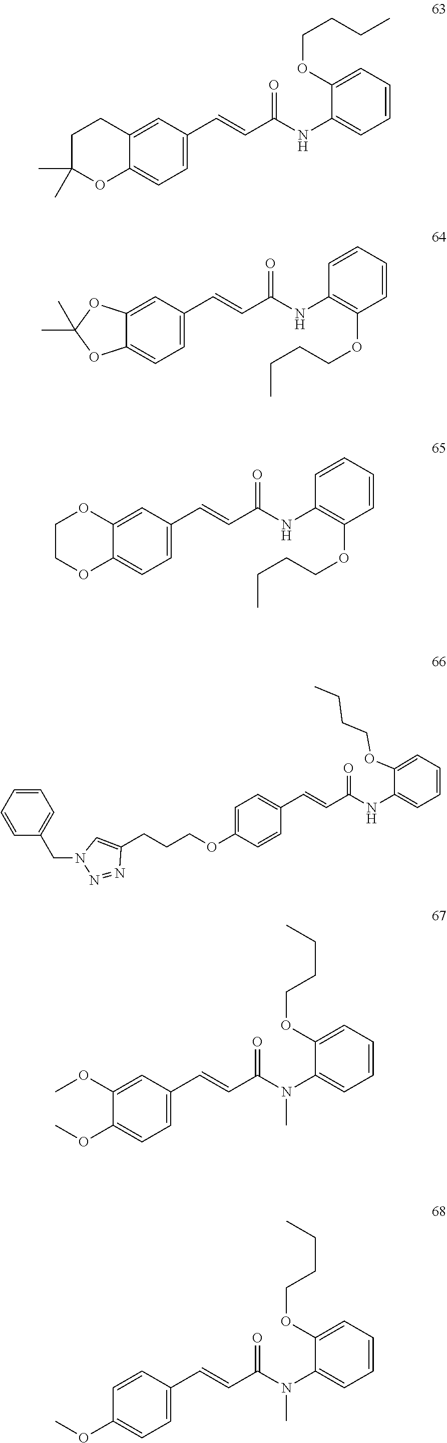 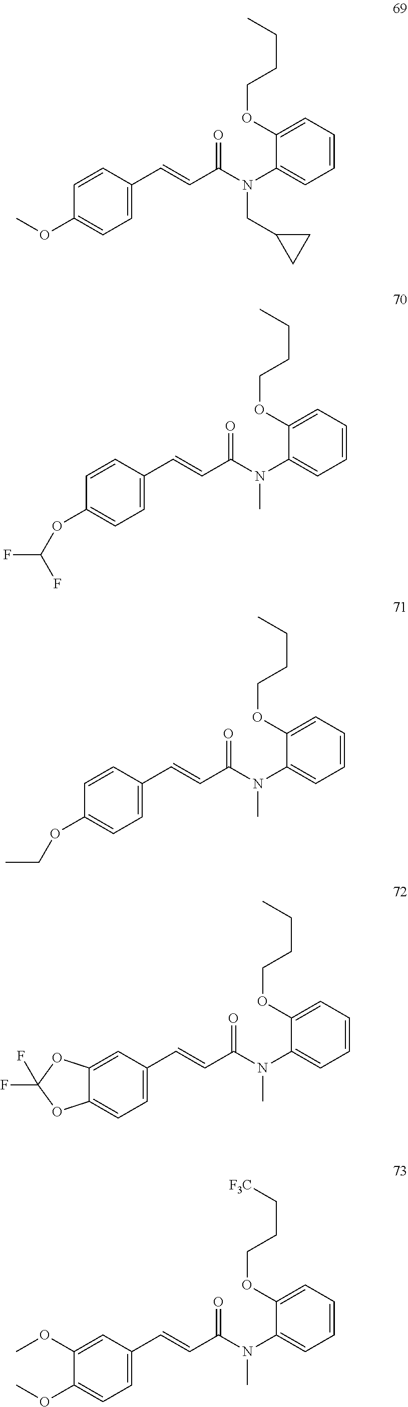 [SMILES: CCCCOc1ccccc1N(CC1CC1)C(=O)/C=C/c1ccc(OC)cc1 ; CCCCOc1ccccc1N(C)C(=O)/C=C/c1ccc(OC(F)F)cc1 ; CCCCOc1ccccc1N(C)C(=O)/C=C/c1ccc(OCC)cc1 ; CCCCOc1ccccc1N(C)C(=O)/C=C/c1ccc2c(c1)OC(F)(F)O2 ; COc1ccc(/C=C/C(=O)N(C)c2ccccc2OCCCC(F)(F)F)cc1OC] 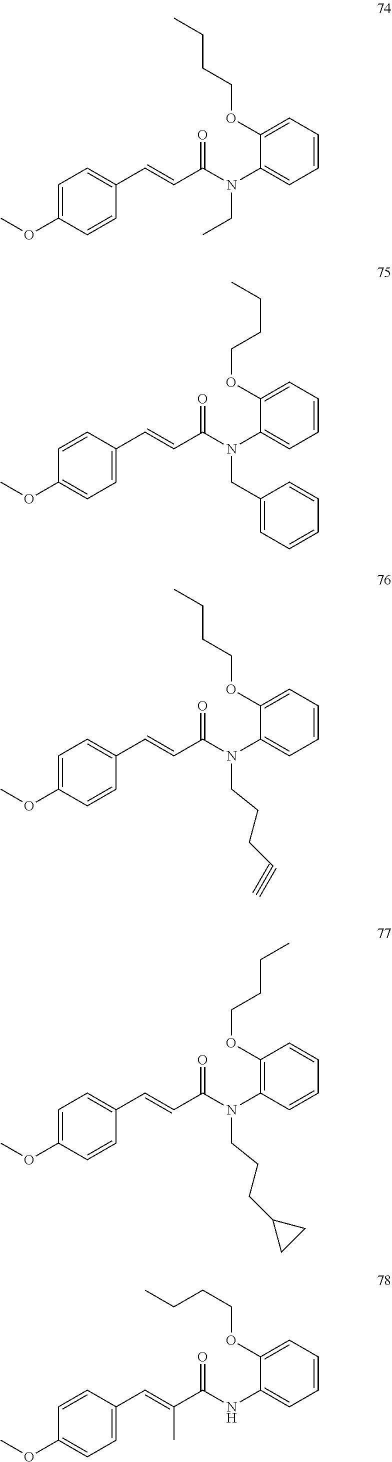 [SMILES: CCCCOc1ccccc1N(CC)C(=O)/C=C/c1ccc(OC)cc1 ; CCCCOc1ccccc1N(Cc1ccccc1)C(=O)/C=C/c1ccc(OC)cc1 ; C#CCCCN(C(=O)/C=C/c1ccc(OC)cc1)c1ccccc1OCCCC ; CCCCOc1ccccc1N(CCCC1CC1)C(=O)/C=C/c1ccc(OC)cc1 ; CCCCOc1ccccc1NC(=O)/C(C)=C/c1ccc(OC)cc1] 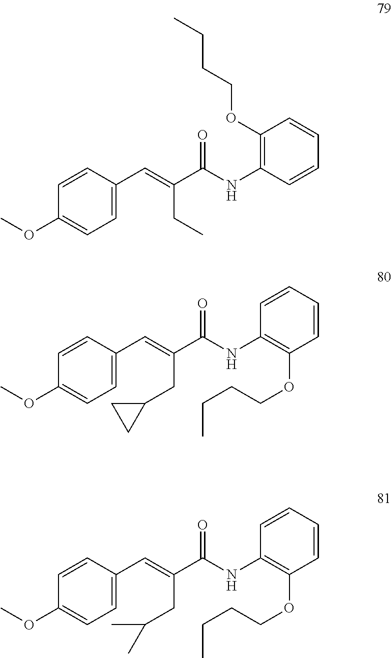 [SMILES: CCCCOc1ccccc1NC(=O)/C(=C/c1ccc(OC)cc1)CC ; CCCCOc1ccccc1NC(=O)/C(=C/c1ccc(OC)cc1)CC1CC1 ; CCCCOc1ccccc1NC(=O)/C(=C/c1ccc(OC)cc1)CC(C)C] 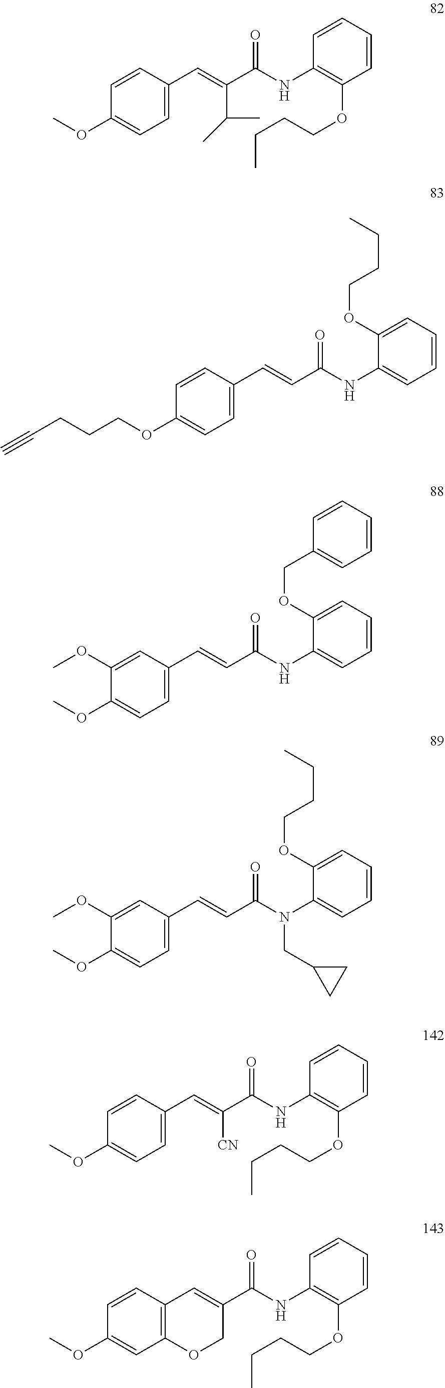 [SMILES: CCCCOc1ccccc1NC(=O)/C(=C/c1ccc(OC)cc1)C(C)C ; C#CCCCOc1ccc(/C=C/C(=O)Nc2ccccc2OCCCC)cc1 ; COc1ccc(/C=C/C(=O)Nc2ccccc2OCc2ccccc2)cc1OC ; CCCCOc1ccccc1N(CC1CC1)C(=O)/C=C/c1ccc(OC)c(OC)c1 ; CCCCOc1ccccc1NC(=O)/C(C#N)=C/c1ccc(OC)cc1 ; CCCCOc1ccccc1NC(=O)C1=Cc2ccc(OC)cc2OC1] 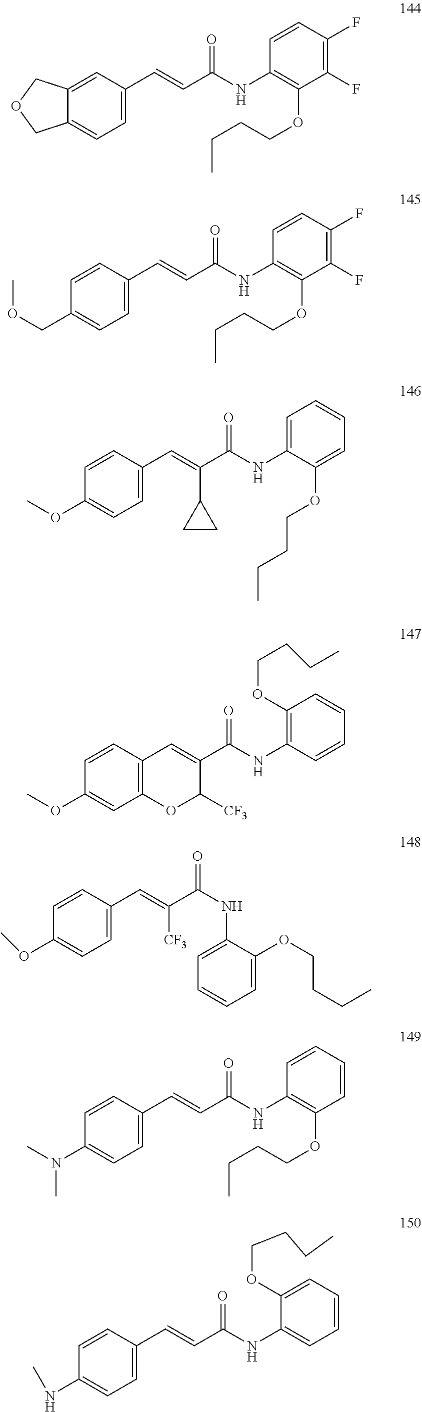 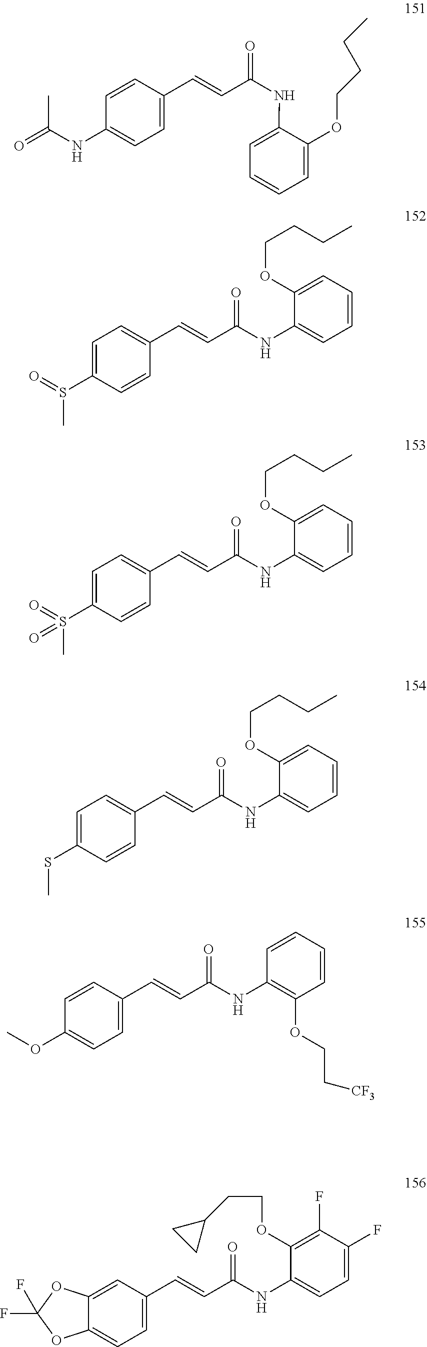 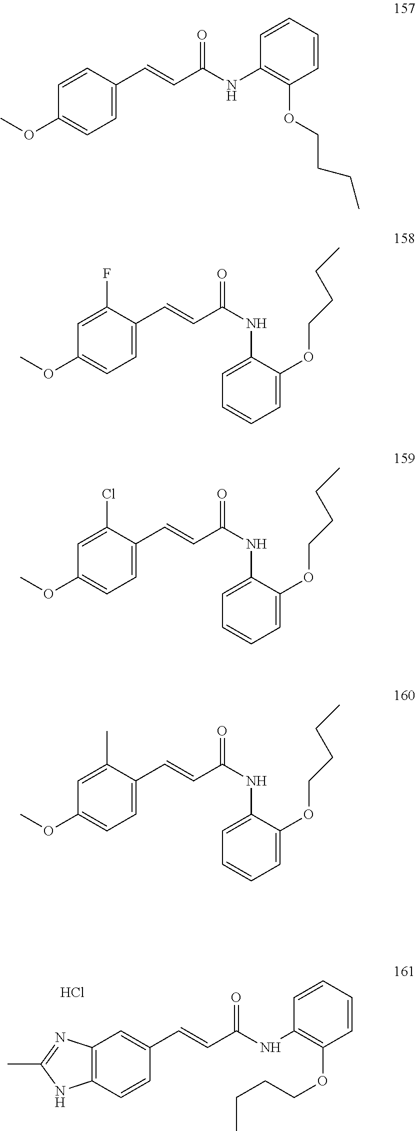 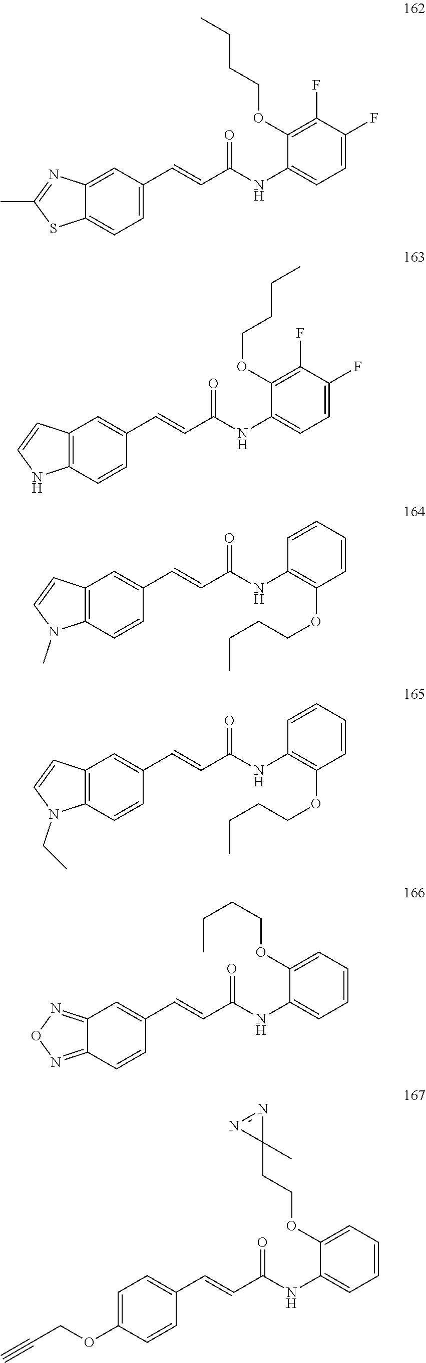 [SMILES: CCCCOc1c(NC(=O)/C=C/c2ccc3sc(C)nc3c2)ccc(F)c1F ; CCCCOc1c(NC(=O)/C=C/c2ccc3[nH]ccc3c2)ccc(F)c1F ; CCCCOc1ccccc1NC(=O)/C=C/c1ccc2c(ccn2C)c1 ; CCCCOc1ccccc1NC(=O)/C=C/c1ccc2c(ccn2CC)c1 ; CCCCOc1ccccc1NC(=O)/C=C/c1ccc2nonc2c1 ; C#CCOc1ccc(/C=C/C(=O)Nc2ccccc2OCCC2(C)N=N2)cc1] 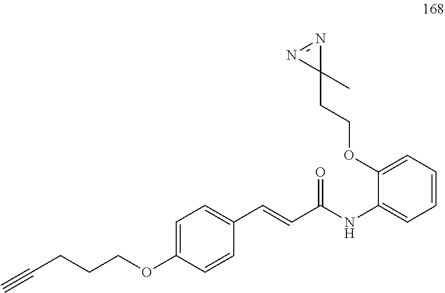 [SMILES: C#CCCCOc1ccc(/C=C/C(=O)Nc2ccccc2OCCC2(C)N=N2)cc1]
Preferably, the compound is selected from the group consisting of

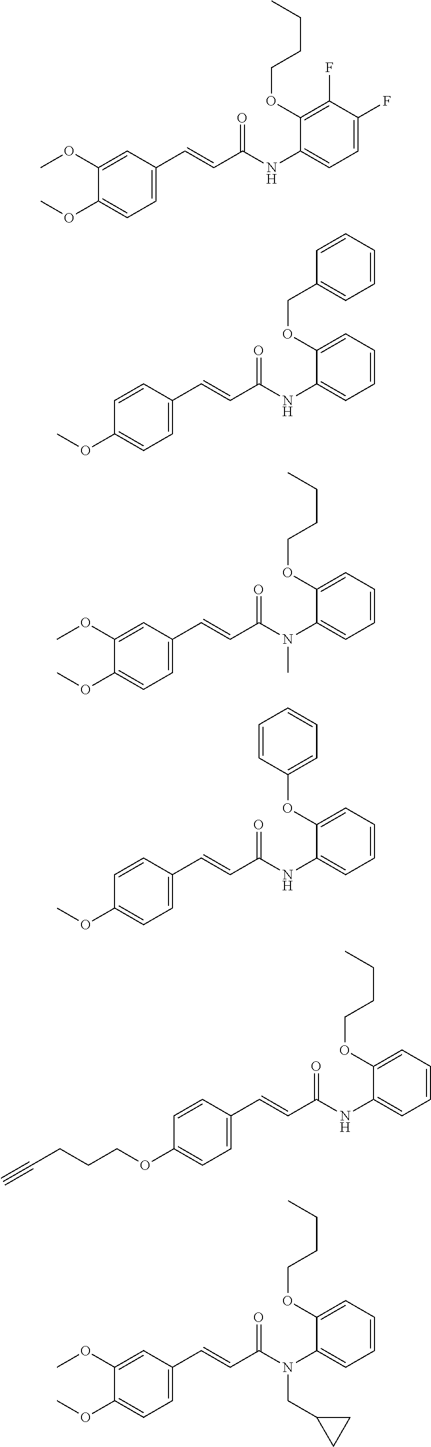

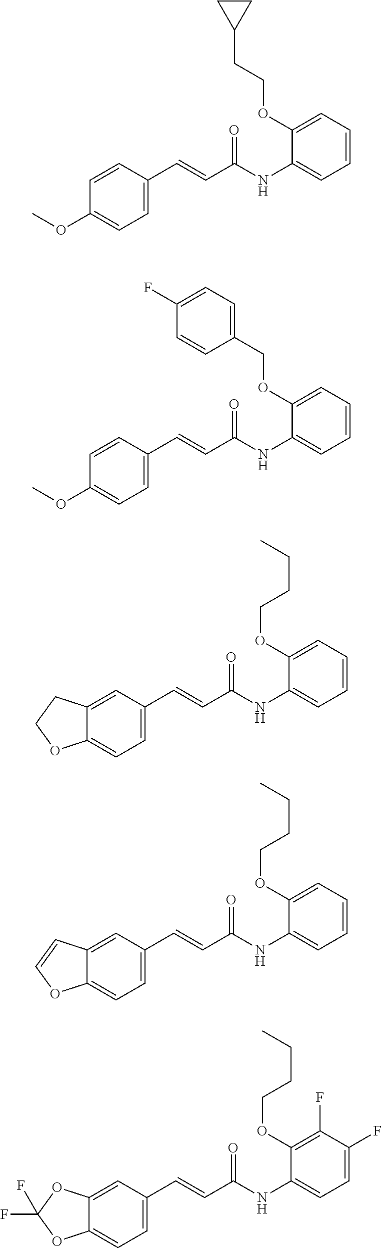 [SMILES: COc1ccc(/C=C/C(=O)Nc2ccccc2OCCC2CC2)cc1 ; COc1ccc(/C=C/C(=O)Nc2ccccc2OCc2ccc(F)cc2)cc1 ; CCCCOc1ccccc1NC(=O)/C=C/c1ccc2c(c1)CCO2 ; CCCCOc1ccccc1NC(=O)/C=C/c1ccc2occc2c1 ; CCCCOc1c(NC(=O)/C=C/c2ccc3c(c2)OC(F)(F)O3)ccc(F)c1F]

11. A compound for use in the treatment of fibrosis and neoplasia, preferably a fibrosis or neoplasia located in the heart, the lung, the renal tract, the liver, in the skin, in the pleura and retroperitoneum, more preferably the fibrosis is selected from pleural fibrosis, retroperitoneal fibrosis, atrial fibrillation, myocardial interstitial fibrosis, idiopathic pulmonary fibrosis (IPF), interstitial lung diseases, chronic kidney disease, non-alcoholic fat liver disease, skin scars, keloids, tumour-associated desmoplastic reaction, wherein said compound is selected from the group consisting of

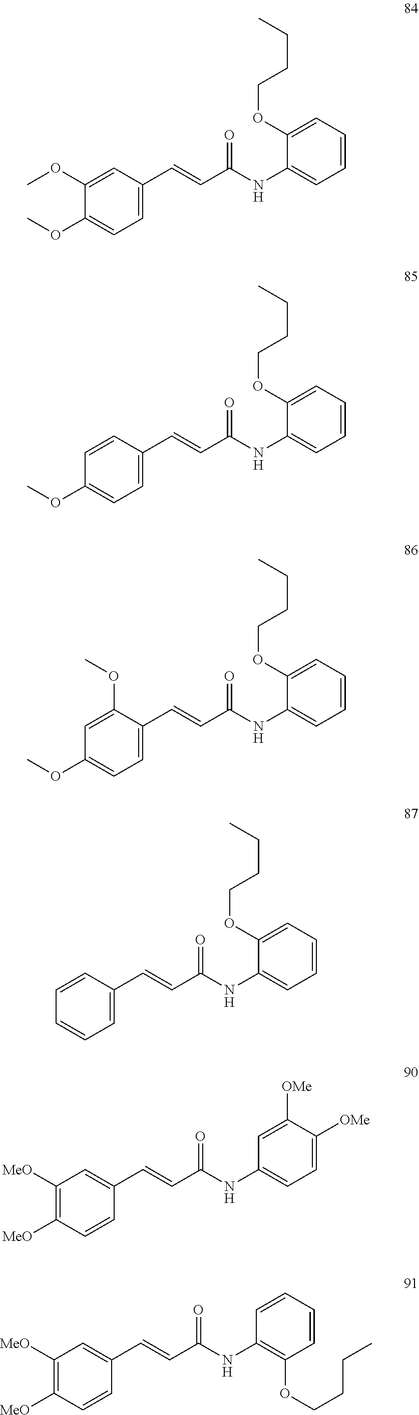 [SMILES: CCCCOc1ccccc1NC(=O)/C=C/c1ccc(OC)c(OC)c1 ; CCCCOc1ccccc1NC(=O)/C=C/c1ccc(OC)cc1 ; CCCCOc1ccccc1NC(=O)/C=C/c1ccc(OC)cc1OC ; CCCCOc1ccccc1NC(=O)/C=C/c1ccccc1 ; COc1ccc(/C=C/C(=O)Nc2ccc(OC)c(OC)c2)cc1OC ; CCCCOc1ccccc1NC(=O)/C=C/c1ccc(OC)c(OC)c1]

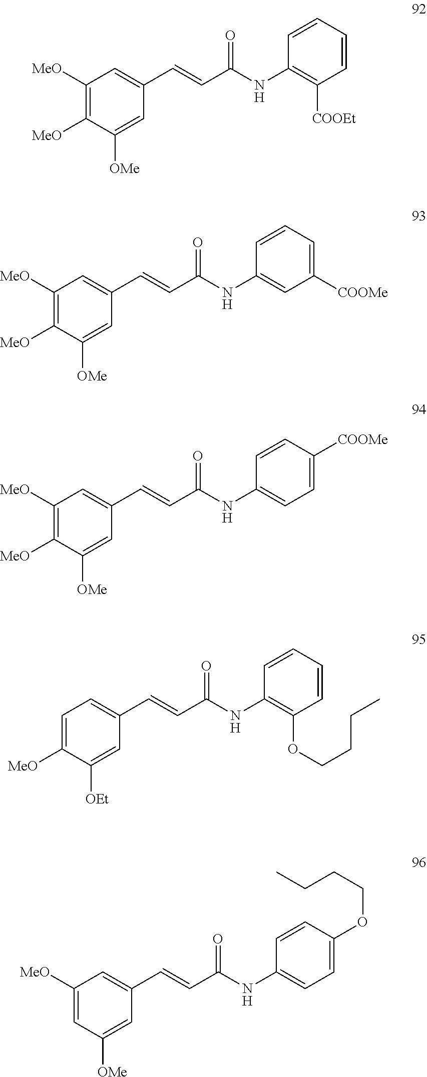 [SMILES: CCOC(=O)c1ccccc1NC(=O)/C=C/c1cc(OC)c(OC)c(OC)c1 ; COC(=O)c1cccc(NC(=O)/C=C/c2cc(OC)c(OC)c(OC)c2)c1 ; COC(=O)c1ccc(NC(=O)/C=C/c2cc(OC)c(OC)c(OC)c2)cc1 ; CCCCOc1ccccc1NC(=O)/C=C/c1ccc(OC)c(OCC)c1 ; CCCCOc1ccc(NC(=O)/C=C/c2cc(OC)cc(OC)c2)cc1]

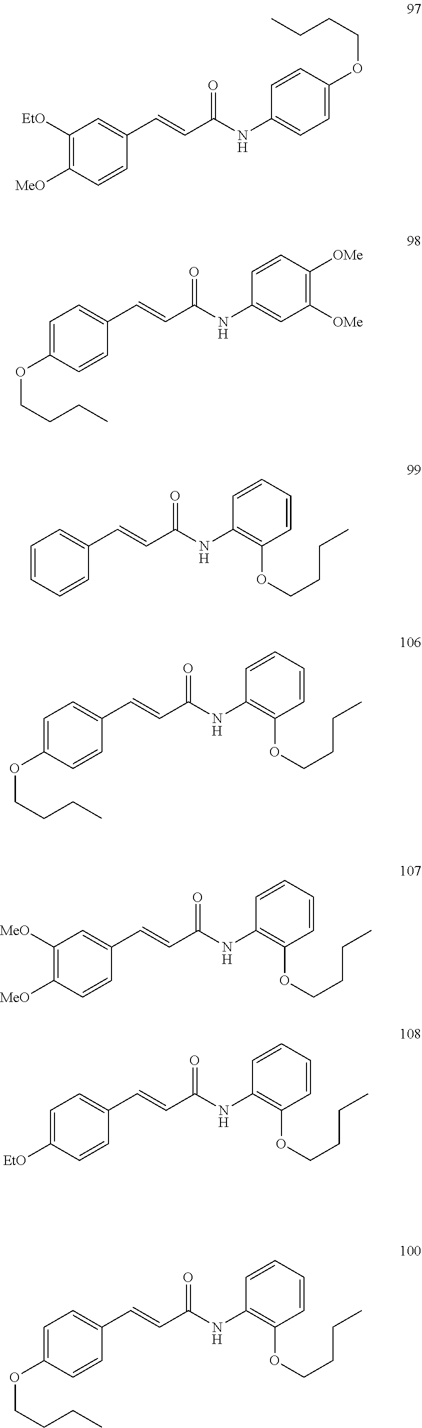

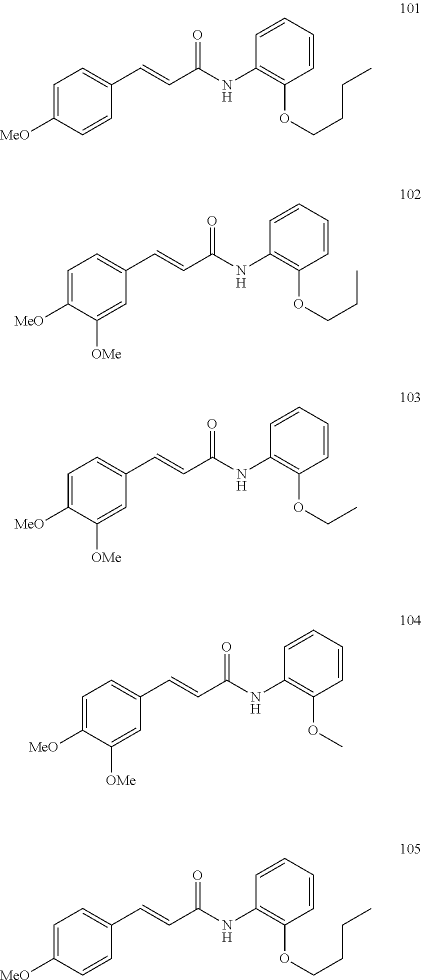 [SMILES: CCCCOc1ccccc1NC(=O)/C=C/c1ccc(OC)cc1 ; CCCOc1ccccc1NC(=O)/C=C/c1ccc(OC)c(OC)c1 ; CCOc1ccccc1NC(=O)/C=C/c1ccc(OC)c(OC)c1 ; COc1ccccc1NC(=O)/C=C/c1ccc(OC)c(OC)c1 ; CCCCOc1ccccc1NC(=O)/C=C/c1ccc(OC)cc1]

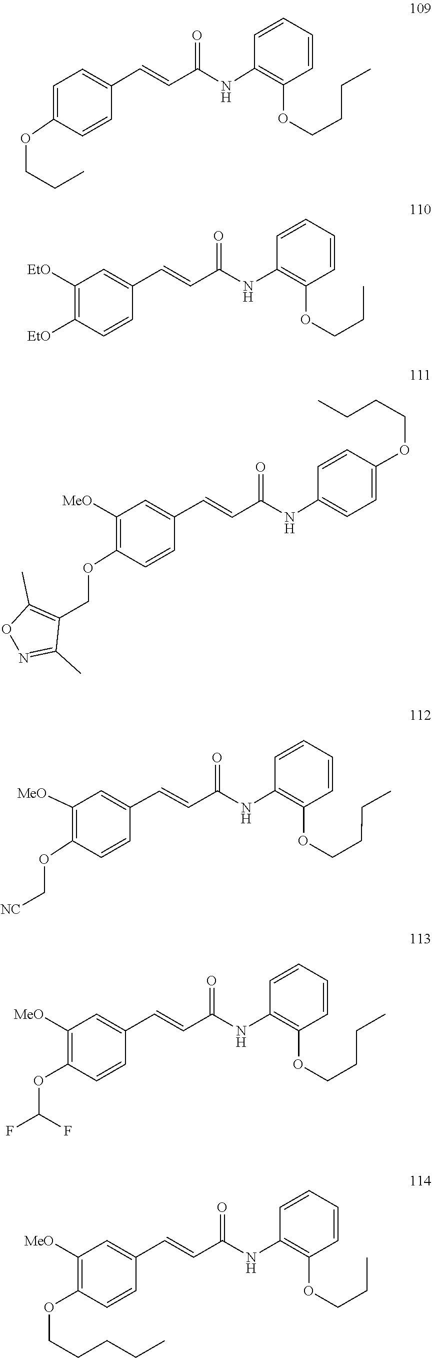 [SMILES: CCCCOc1ccccc1NC(=O)/C=C/c1ccc(OCCC)cc1 ; CCCOc1ccccc1NC(=O)/C=C/c1ccc(OCC)c(OCC)c1 ; CCCCOc1ccc(NC(=O)/C=C/c2ccc(OCc3c(C)noc3C)c(OC)c2)cc1 ; CCCCOc1ccccc1NC(=O)/C=C/c1ccc(OCC#N)c(OC)c1 ; CCCCOc1ccccc1NC(=O)/C=C/c1ccc(OC(F)F)c(OC)c1 ; CCCCCOc1ccc(/C=C/C(=O)Nc2ccccc2OCCC)cc1OC]

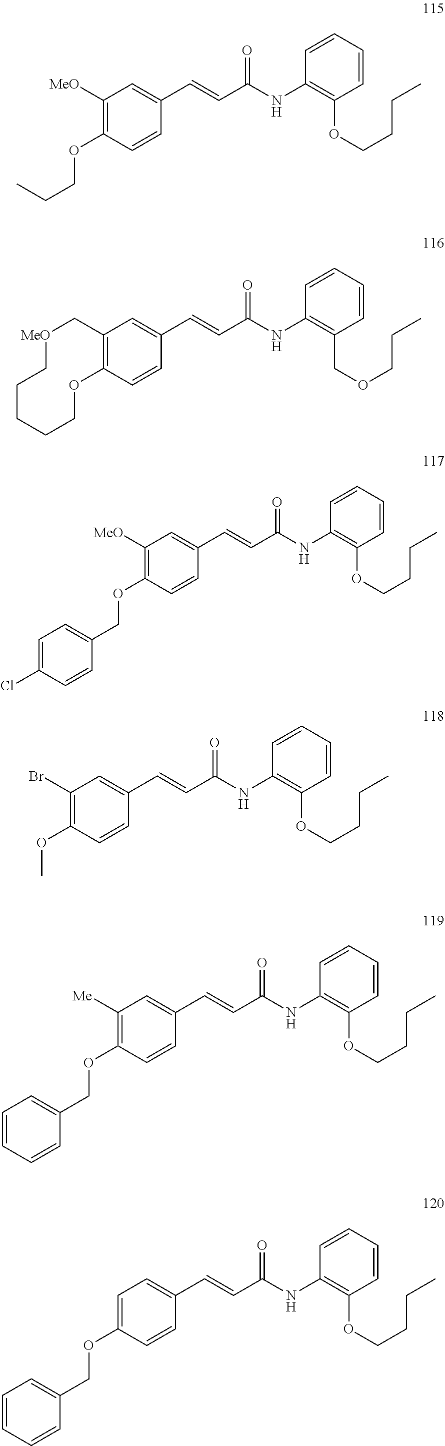

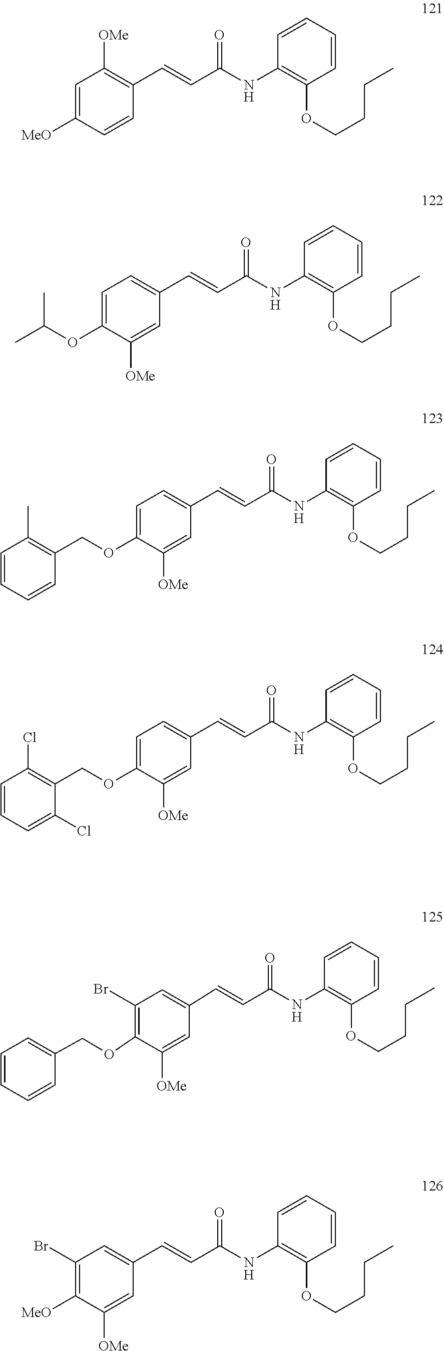

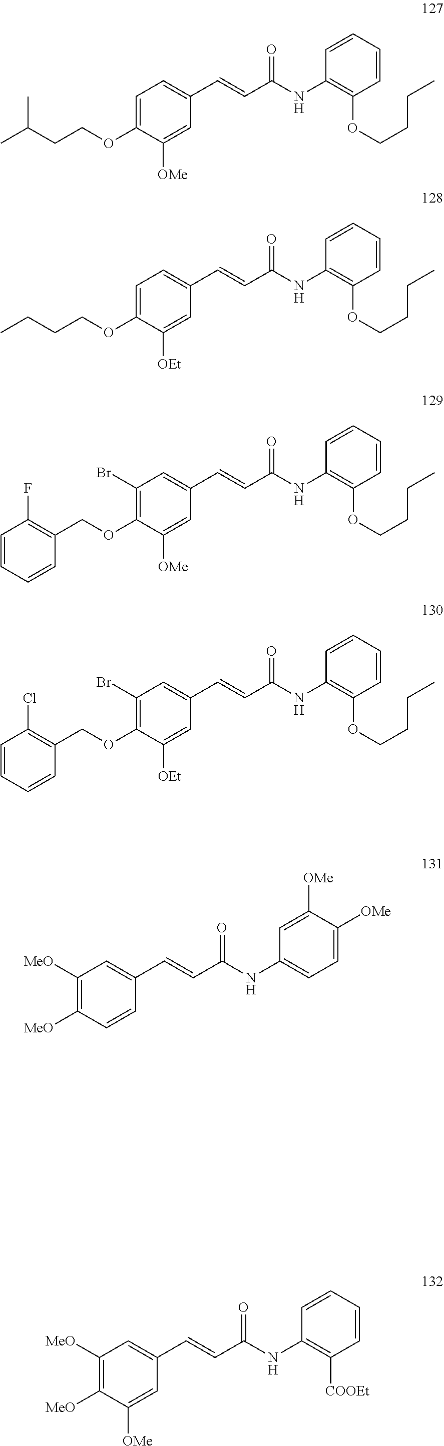

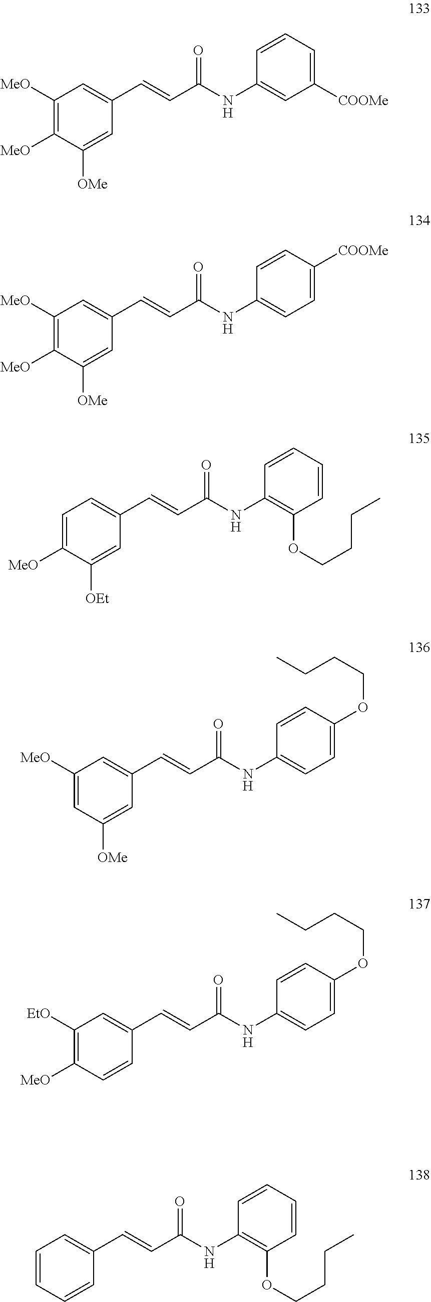 [SMILES: COC(=O)c1cccc(NC(=O)/C=C/c2cc(OC)c(OC)c(OC)c2)c1 ; COC(=O)c1ccc(NC(=O)/C=C/c2cc(OC)c(OC)c(OC)c2)cc1 ; CCCCOc1ccccc1NC(=O)/C=C/c1ccc(OC)c(OCC)c1 ; CCCCOc1ccc(NC(=O)/C=C/c2cc(OC)cc(OC)c2)cc1 ; CCCCOc1ccc(NC(=O)/C=C/c2ccc(OC)c(OCC)c2)cc1 ; CCCCOc1ccccc1NC(=O)/C=C/c1ccccc1]

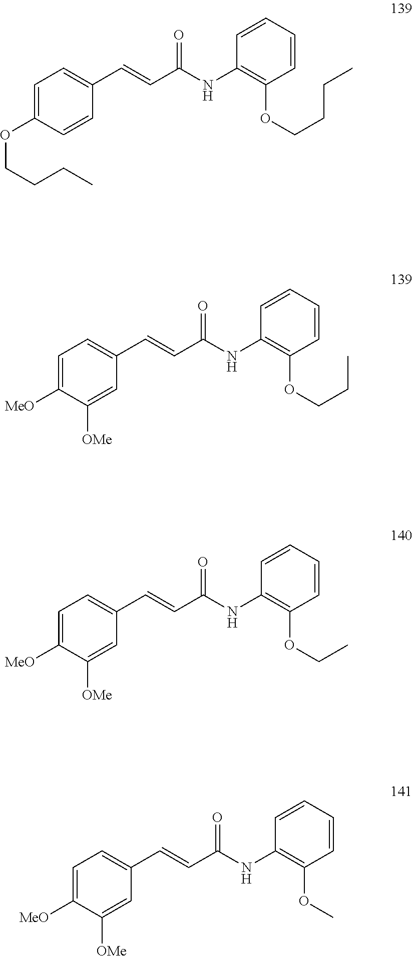 [SMILES: CCCCOc1ccc(/C=C/C(=O)Nc2ccccc2OCCCC)cc1 ; CCCOc1ccccc1NC(=O)/C=C/c1ccc(OC)c(OC)c1 ; CCOc1ccccc1NC(=O)/C=C/c1ccc(OC)c(OC)c1 ; COc1ccccc1NC(=O)/C=C/c1ccc(OC)c(OC)c1]

12. A pharmaceutical composition comprising the compound of claims 1 to 11 and at least one carrier.

13. The compound of claims 2 and 7 to 10 or the pharmaceutical composition of claim 12 for use in the treatment of fibrosis and neoplasia, preferably a fibrosis or neoplasia located in the heart, the lung, the renal tract, the liver, in the skin, in the pleura and retroperitoneum, more preferably the fibrosis is selected from pleural fibrosis, retroperitoneal fibrosis, atrial fibrillation, myocardial interstitial fibrosis, idiopathic pulmonary fibrosis (IPF), interstitial lung diseases, chronic kidney disease, non-alcoholic fat liver disease, skin scars, keloids, tumour-associated desmoplastic reaction.

14. The compound of claims 1 to 11 or the pharmaceutical composition of claim 12 for use in the treatment of inflammatory diseases, such as arthrolithiasis. familiar mediterranean fever and pericarditis.

15. A screening assay, comprising the steps
a) culturing adherent cells which deposit at least one protein in the presence of at least one test compound;
b) staining of at least one protein deposited by the adherent cells;
c) fixation of adherent cells and the at least one protein;
d) microscopic detection of a signal of the at least one stained deposited protein;
e) data analysis of signals detected in step d) comprising quantification of the amount of the at least one protein deposited in the presence of the at least one test compound.
wherein step b) is carried out before step c)
wherein optionally
i) the adherent cells are primary cells and/or
ii) the adherent cells are primary patient derived human cells, preferably human lung fibroblasts;
or primary animal derived cells; or any adherent immortalized cell lines.
iii) step a) is carried out for at least 24 h, preferably 60 to 90 hours, more preferably 65 to 80 hours, most preferably for 70 to 75 hours and/or
iv) wherein at least one protein is an extracellular matrix protein, preferably the at least one extracellular matrix protein is selected from the group consisting of collagen type 5, collagen type 1, and fibulin 1 and/or
v) step b) comprises the binding of at least one antibody to at least one protein or at least one probe binding to at least one protein and wherein optionally
the antibody or probe comprises at least one detectable label that is directly conjugated to the antibody and wherein optionally the detectable label has fluorescence property, preferably the detectable lable is a fluorophore selected from AlexaFluor 488, AlexaFluor 555, and AlexaFluor 637; AlexaFluor 647, AlexaFluor 568, AlexaFluor 568, and/or Qdots and/or
vi) step b) comprises the binding of at least one first antibody (FA) to at least one protein and the subsequent binding of at least one first antibody (FA) with at least one secondary antibody (SA) wherein,
the at least one second antibody (SA) comprises at least one detectable label that is conjugated to the antibody and wherein optionally the detectable label has fluorescence property, preferably the detectable label is a fluorophore selected from AlexaFluor 488, AlexaFluor 555, and AlexaFluor 637; AlexaFluor 647, AlexaFluor 568, AlexaFluor 568, and/or Qdots and/or
vii) in step b) at least one further co-staining is present and selected from the group consisting of cell-nuclei staining, live-dead staining, myofibroblast markers (e.g. αSMA-staining), apoptosis markers (e.g. Caspase3/7 staining) and/or
viii) the at least one test compound in step a) is a small molecule; and/or oligonucleotides, peptides, proteins, protacs, anticalins, antibodies, CRISPRs and/or
ix) in step d) 2D or 3D imaging is carried out by a conventional or confocal imaging apparatus and/or
x) the data analysis in step e) comprises using a machine learning model, such as neural networks.
